SEQUENCE LISTING (165 sequences)

SEQ ID NO: 1 - protein, 866 aa, Escherichia coli
MSTKPLFLLRKAKKSSGEPDVVLWASNDFESTCATLDYLIVKSGKKLSSYFKAVATNFPVVNDLPAEGEIDFTWSERYQLSKDSMTWELKPGAAPDNAHYQGNTNVNGEDMTEIEENMLLPISGQELPIRWLAQHGSEKPVTHVSRDGLQALHIARAEELPAVTALAVSHKTSLLDPLEIRELHKLVRDTDKVFPNPGNSNLGLITAFFEAYLNADYTDRGLLTKEWMKGNRVSHITRTASGANAGGGNLTDRGEGFVHDLTSLARDVATGVLARSMDLDIYNLHPAHAKRIEEIIAENKPPFSVFRDKFITMPGGLDYSRAIVVASVKEAPIGIEVIPAHVTEYLNKVLTETDHANPDPEIVDIACGRSSAPMPQRVTEEGKQDDEEKPQPSGTTAVEQGEAETMEPDATEHHQDTQPLDAQSQVNSVDAKYQELRAELHEARKNIPSKNPVDDDKLLAASRGEFVDGISDPNDPKWVKGIQTRDCVYQNQPETEKTSPDMNQPEPVVQQEPEIACNACGQTGGDNCPDCGAVMGDATYQETFDEESQVEAKENDPEEMEGAEHPHNENAGSDPHRDCSDETGEVADPVIVEDIEPGIYYGISNENYHAGPGISKSQLDDIADTPALYLWRKNAPVDTTKTKTLDLGTAFHCRVLEPEEFSNRFIVAPEFNRRTNAGKEEEKAFLMECASTGKTVITAEEGRKIELMYQSVMALPLGQWLVESAGHAESSIYWEDPETGILCRCRPDKIIPEFHWIMDVKTTADIQRFKTAYYDYRYHVQDAFYSDGYEAQFGVQPTFVFLVASTTIECGRYPVEIFMMGEEAKLAGQQEYHRNLRTLADCLNTDEWPAIKTLSLPRWAKEYAND

SEQ ID NO: 2 - protein, 280 aa, artificial sequence
  Synthetic
ADPVIVEDIEPGIYYGISNENYHAGPGVSKSQLDDIADTPALYLWRKNAPVDTTKTKTLDLGTAFHCRVLEPEEFSNRFIVAPEFNRRTNSGKEEEKAFLRECASTGKTVITAEEGRKIELMYQSVMALPLGQWLVESAGHAESSIYWEDPETAILCRCRPDKIIPEFHWIMDVKTTADIQRFKTAYYDYRYHVQDAFYSDGYEAQFGVQPTFVFLVASTTIECGRYPVEIFMMGEEAKLAGQLEYHRNLRTLADCLNTDEWPAIKTLSLPRWAKEYAND

SEQ ID NO: 3 - protein, 265 aa, artificial sequence
  Synthetic
GISNENYHAGPGVSKSQLDDIADTPALYLWRKNAPVDTTKTKTLDLGTAFHCRVLEPEEFSNRFIVAPEFNRRTNSGKEEEKAFLRECASTGKTVITAEEGRKIELMYQSVMALPLGQWLVESAGHAESSIYWEDPETAILCRCRPDKIIPEFHWIMDVKTTADIQRFKTAYYDYRYHVQDAFYSDGYEAQFGVQPTFVFLVASTTIECGRYPVEIFMMGEEAKLAGQLEYHRNLRTLADCLNTDEWPAIKTLSLPRWAKEYAND

SEQ ID NO: 4 - protein, 272 aa, Pantoea brenneri
MQPGIYYDISNEDYHRGAGISKSQLDDIAISPAIYQWRKHAPVDEEKTAALDLGTALHCLLLEPDEFSKRFQIGPEVNRRTTAGKEKEKEFIERCEAEGITPITHDDNRKLKLMRDSALAHPIARWMLEAQGNAEASIYWNDRDAGVLSRCRPDKIITEFNWCVDVKSTADIMKFQKDFYSYRYHVQDAFYSDGYESHFHETPTFAFLAVSTSIDCGRYPVQVFIMDQQAKDAGRAEYKRNIHTFAECLSRNEWPGIATLSLPFWAKELRNE

SEQ ID NO: 5 - protein, 272 aa, Type-F symbiont of Plautia stali
MQPGIYYDISNEDYHGGPGISKSQLDDIAISPAIYQWRKHAPVDEEKTAALDLGTALHCLLLEPDEFSKRFEIGPEVNRRTTAGKEKEKEFMERCEAEGVTPITHDDNRKLRLMRDSAMAHPIARWMLEAQGNAEASIYWNDRDTGVLSRCRPDKIITDFNWCVDVKSTADIIKFQKDFYSYRYHVQDAFYSDGYESHFDETPTFAFLAVSTSIDCGRYPVQVFIMDQQAKDAGRAEYKRNIHTFAECLSRNEWPGIATLSLPYWAKELRNE

SEQ ID NO: 6 - protein, 272 aa, Providencia sp. MGF014
MKEGIYYNISNEDYHNGLGISKSQLDLINEMPAEYIWSKEAPVDEEKIKPLEIGTALHCLLLEPDEYHKRYKIGPDVNRRTNVGKEKEKEFFDMCEKEGITPITHDDNRKLMIMRDSALAHPIAKWCLEADGVSESSIYWTDKETDVLCRCRPDRIITAHNYIIDVKSSGDIEKFDYEYYNYRYHVQDAFYSDGYKEVTGITPTFLFLVVSTKIDCGKYPVRTYVMSEEAKSAGRTAYKHNLLTYAECLKTDEWAGIRTLSLPRWAKELRNE

SEQ ID NO: 7 - protein, 648 aa, Shigella sonnei
DRGLLTKEWRKGNRVSRITRTASGANAGGGNLTDRGEGFVHDLTSLARDIATGVLARSMDVDIYNLHPAHAKRIEEIIAENKPPFSVFRDKFITMPGGLDYSRAIVVASVKEAPIGIEVIPAHVTAYLNKVLTETDHANPDPEIVDIACGRSSAPMPQRVTEEGKQDDEEKLQPSGTTADEQGEAETMEPDATKHHQDTQPLDAQSQVNSVDAKYQELRAELHEARKNIPSKNPVDADKLLAASRGEFVDGISDPNDPKWVKGIQTRDSVYQNQPETEKTSPDMKQPEPVVQQEPEIAFNACGQTGGDNCPDCGAVMGDATYQETFDEENQVEAKENDPEEMEGAEHPHNENAGSDPHRDCSDETGEVADPVIVEDIEPGIYYGISNENYHAGPGVSKSQLDDIADTPALYLWRKNAPVDTTKTKTLDLGTAFHCRVLEPEEFSNRFIVAPEFNRRTNAGKEEEKAFLMECASTGKMVITAEEGRKIELMYQSVMALPLGQWLVESAGHAESSIYWEDPETGILCRCRPDKIIPEFHWIMDVKTTADIQRFKTAYYDYRYHVQDAFYSDGYEAQFGVQPTFVFLVASTTIECGRYPVEIFMMGEEAKLAGQLEYHRNLRTLADCLNTDEWPAIKTLSLPRWAKEYAND

SEQ ID NO: 8 - protein, 300 aa, Pseudobacteriovorax antillogorgiicola
MSKLSNLKVSNSDVDTLSRIRMKEGVYRDLPIESYHQSPGYSKTSLCQIDKAPIYLKTKVPQKSTKSLNIGTAFHEAMEGVFKDKYVVHPDPGVNKTTKSWKDFVKRYPKHMPLKRSEYDQVLAMYDAARSYRPFQKYHLSRGFYESSFYWHDAVTNSLIKCRPDYITPDGMSVIDFKTTVDPSPKGFQYQAYKYHYYVSAALTLEGIEAVTGIRPKEYLFLAVSNSAPYLTALYRASEKEIALGDHFIRRSLLTLKTCLESGKWPGLQEEILELGLPFSGLKELREEQEVEDEFMELVG

SEQ ID NO: 9 - protein, 269 aa, Escherichia coli
MTKQPPIAKADLQKTQGNRAPAAVKNSDVISFINQPSMKEQLAAALPRHMTAERMIRIATTEIRKVPALGNCDTMSFVSAIVQCSQLGLEPGSALGHAYLLPFGNKNEKSGKKNVQLIIGYRGMIDLARRSGQIASLSARVVREGDEFSFEFGLDEKLIHRPGENEDAPVTHVYAVARLKDGGTQFEVMTRKQIELVRSLSKAGNNGPWVTHWEEMAKKTAIRRLFKYLPVSIEIQRAVSMDEKEPLTIDPADSSVLTGEYSVIDNSEE

SEQ ID NO: 10 - protein, 273 aa, Pantoea brenneri
MSNQPPIASADLQKTQQSKQVANKTPEQTLVGFMNQPAMKSQLAAALPRHMTADRMIRIVTTEIRKTPQLAQCDQSSFIGAVVQCSQLGLEPGSALGHAYLLPFGNGRSKSGQSNVQLIIGYRGMIDLARRSGQIVSLSARVVRADDEFSFEYGLDENLVHRPGENEDAPITHVYAVARLKDGGTQFEVMTVKQVEKVKAQSKASSNGPWVTHWEEMAKKTVIRRLFKYLPVSIEMQKAVVLDEKAESDVDQDNASVLSAEYSVLESGDEATN

SEQ ID NO: 11 - protein, 271 aa, Type-F symbiont of Plautia stali
MSNQPPIASADLQKTQQSKQVANKTPEQTLVGFMNQPAMKSQLAAALPRHMTADRMIRIVTTEIRKTPALATCDQSSFIGAVVQCSQLGLEPGSALGHAYLLPFGNGRSKSGQSNVQLIIGYRGMIDLARRSGQIVSLSARVVRADDEFSFEYGLDENLIHRPGDNEDAPITHVYAVARLKDGGTQFEVMTAKQVEKVKAQSKASSNGPWVTHWEEMAKKTVIRRLFKYLPVSIEMQKAVVLDEKAESDVDQDNASVLSAEYSVLEGDGGE

SEQ ID NO: 12 - protein, 268 aa, Providencia sp. MGF014
MSNPPLAQSDLQKTQGTEVKVKTKDQQLIQFINQPSMKAQLAAALPRHMTPDRMIRIVTTEIRKTPALATCDMQSFVGAVVQCSQLGLEPGNALGHAYLLPFGNGKAKSGQSNVQLIIGYRGMIDLARRSNQIISISARTVRQGDNFHFEYGLNEDLTHTPSENEDSPITHVYAVARLKDGGVQFEVMTYNQVEKVRASSKAGQNGPWVSHWEEMAKKTVIRRLFKYLPVSIEMQKAVVLDEKAEANVDQENATIFEGEYEEVGTDGN

SEQ ID NO: 13 - protein, 269 aa, Shigella sonnei
MTKQPPIAKADLQKTQENRAPAAIKNNDVISFINQPSMKEQLAAALPRHMTAERMIRIATTEIRKVPALGNCDTMSFVSAIVQCSQLGLEPGSALGHAYLLPFGNKNEKSGKKNVQLIIGYRGMIDLARRSGQIASLSARVVREGDEFNFEFGLDEKLIHRPGENEDAPVTHVYAVARLKDGGTQFEVMTRRQIELVRSQSKAGNNGPWVTHWEEMAKKTAIRRLFKYLPVSIEIQRAVSMDEKEPLTIDPADSSVLTGEYSVIDNSEE

SEQ ID NO: 14 - protein, 242 aa, Pseudobacteriovorax antillogorgiicola
MGHLVSKTEQDYIKQHYAKGATDQEFEHFIGVCRARGLNPAANQIYFVKYRSKDGPAKPAFILSIDSLRLIAHRTGDYAGCSEPIFTDGGKACTVTVRRNLKSGETGNFSGMAFYDEQVQQKNGRPTSFWQSKPRTMLEKCAEAKALRKAFPQDLGQFYIREEMPPQYDEPIQVHKPKALEEPRFSKSDLSRRKGLNRKLSALGVDPSRFDEVATFLDGTPDRELGQKLKLWLKEAGYGVNQ

SEQ ID NO: 15 - protein, 16 aa, artificial sequence
  Synthetic
SGSETPGTSESATPES

SEQ ID NO: 16 - protein, 7 aa, artificial sequence
  Synthetic
PKKKRKV

SEQ ID NO: 17 - protein, 39 aa, artificial sequence
  Synthetic
NSKKRSLEDNETEIKVSRDTWNTKNMRSLEPPRSKKRIH

SEQ ID NO: 18 - protein, 9 aa, artificial sequence
  Synthetic
PAAKRVKLD

SEQ ID NO: 19 - protein, 18 aa, artificial sequence
  Synthetic
KRTADGSEFESPKKKRKV

SEQ ID NO: 20 - protein, 29 aa, artificial sequence
  Synthetic
PEKKRRRPSGSVPVLARPSPPKAGKSSCI

SEQ ID NO: 21 - DNA, 20 bp, artificial sequence
  Synthetic
gtcacctccaatgactaggg

SEQ ID NO: 22 - DNA, 20 bp, artificial sequence
  Synthetic
ggtgagtgagtgtgtgcgtg

SEQ ID NO: 23 - DNA, 20 bp, artificial sequence
  Synthetic
aaggccataggctggactgc

SEQ ID NO: 24 - DNA, 20 bp, artificial sequence
  Synthetic
gtagactaatctctggctga

SEQ ID NO: 25 - DNA, 20 bp, artificial sequence
  Synthetic
tctcccatgcattcaaactg

SEQ ID NO: 26 - DNA, 20 bp, artificial sequence
  Synthetic
accccacagtggggccacta

SEQ ID NO: 27 - DNA, 20 bp, artificial sequence
  Synthetic
gtcacctccaatgactaggg

SEQ ID NO: 28 - DNA, 20 bp, artificial sequence
  Synthetic
gtcacctccaatgactaggg

SEQ ID NO: 29 - DNA, 20 bp, artificial sequence
  Synthetic
aaggccataggctggactgc

SEQ ID NO: 30 - DNA, 20 bp, artificial sequence
  Synthetic
ggcactgacgatgcagtaca

SEQ ID NO: 31 - DNA, 20 bp, artificial sequence
  Synthetic
gtagactaatctctggctga

SEQ ID NO: 32 - DNA, 20 bp, artificial sequence
  Synthetic
tcgtcatctccttcaagggg

SEQ ID NO: 33 - DNA, 20 bp, artificial sequence
  Synthetic
atgcatgggagagcccagag

SEQ ID NO: 34 - DNA, 20 bp, artificial sequence
  Synthetic
gcctgcccttctaggaatgg

SEQ ID NO: 35 - DNA, 28 bp, artificial sequence
  Synthetic
cattctgcctctctgtatggaaaagagc

SEQ ID NO: 36 - DNA, 26 bp, artificial sequence
  Synthetic
cccattgaactacctgggcctgattc

SEQ ID NO: 37 - DNA, 22 bp, artificial sequence
  Synthetic
aggtttgaatcatcacgcaggc

SEQ ID NO: 38 - DNA, 22 bp, artificial sequence
  Synthetic
attcaagtggggaatggcaagc

SEQ ID NO: 39 - DNA, 20 bp, artificial sequence
  Synthetic
tgccgtaaatgctgctctct

SEQ ID NO: 40 - DNA, 22 bp, artificial sequence
  Synthetic
agacttgccaaggttctttgtg

SEQ ID NO: 41 - DNA, 25 bp, artificial sequence
  Synthetic
agtgacctgtgtaattatgcagaag

SEQ ID NO: 42 - DNA, 24 bp, artificial sequence
  Synthetic
tgaaagtgccacaaaacaaagaga

SEQ ID NO: 43 - DNA, 19 bp, artificial sequence
  Synthetic
aagacaagtggcaacgcag

SEQ ID NO: 44 - DNA, 31 bp, artificial sequence
  Synthetic
cgtttatgatactatgcagactatgaagaac

SEQ ID NO: 45 - DNA, 23 bp, artificial sequence
  Synthetic
atgaagatgaccctactgctgat

SEQ ID NO: 46 - DNA, 26 bp, artificial sequence
  Synthetic
tactgtcttgaaagcagatagaaacc

SEQ ID NO: 47 - DNA, 20 bp, artificial sequence
  Synthetic
gcagcaaagaaacacctgga

SEQ ID NO: 48 - DNA, 24 bp, artificial sequence
  Synthetic
gttgtcatgccatacagacttttt

SEQ ID NO: 49 - DNA, 24 bp, artificial sequence
  Synthetic
agcattactagctctgctttagtg

SEQ ID NO: 50 - DNA, 20 bp, artificial sequence
  Synthetic
tccacaagactgggtctgag

SEQ ID NO: 51 - DNA, 21 bp, artificial sequence
  Synthetic
gcgactatgcacaacgagagg

SEQ ID NO: 52 - DNA, 26 bp, artificial sequence
  Synthetic
aagtgtgtctatctactgtgtcccag

SEQ ID NO: 53 - DNA, 20 bp, artificial sequence
  Synthetic
gatgctctttccggagcact

SEQ ID NO: 54 - DNA, 21 bp, artificial sequence
  Synthetic
gccaaggactcaaacccagaa

SEQ ID NO: 55 - DNA, 21 bp, artificial sequence
  Synthetic
tggtggatttcggactaccct

SEQ ID NO: 56 - DNA, 20 bp, artificial sequence
  Synthetic
ttcggactggaaccgtcagc

SEQ ID NO: 57 - DNA, 20 bp, artificial sequence
  Synthetic
agacgttccttagtgctggc

SEQ ID NO: 58 - DNA, 20 bp, artificial sequence
  Synthetic
aaaagtttcagtgcgacgcc

SEQ ID NO: 59 - DNA, 20 bp, artificial sequence
  Synthetic
aggaggtcccatcagatgct

SEQ ID NO: 60 - DNA, 20 bp, artificial sequence
  Synthetic
ggctggacagcaaacatgga

SEQ ID NO: 61 - DNA, 20 bp, artificial sequence
  Synthetic
ggctggacagcaaacatgga

SEQ ID NO: 62 - DNA, 21 bp, artificial sequence
  Synthetic
ttgctgccgtacatgaagctg

SEQ ID NO: 63 - DNA, 43 bp, artificial sequence
  Synthetic
ccatctcatccctgcgtgtctccagaagaagggctcccatcac

SEQ ID NO: 64 - DNA, 43 bp, artificial sequence
  Synthetic
cctctctatgggcagtcggtgatgagcagcaagcagcactctg

SEQ ID NO: 65 - DNA, 43 bp, artificial sequence
  Synthetic
ccatctcatccctgcgtgtctcccagcgtcttcgagagtgagg

SEQ ID NO: 66 - DNA, 44 bp, artificial sequence
  Synthetic
cctctctatgggcagtcggtgatgttggaatcctggagtgaccc

SEQ ID NO: 67 - DNA, 45 bp, artificial sequence
  Synthetic
ccatctcatccctgcgtgtctccacaaaagctccacatgctagga

SEQ ID NO: 68 - DNA, 44 bp, artificial sequence
  Synthetic
cctctctatgggcagtcggtgatggctgactttgggctccttct

SEQ ID NO: 69 - DNA, 43 bp, artificial sequence
  Synthetic
ccatctcatccctgcgtgtctccacacactccccaggatctca

SEQ ID NO: 70 - DNA, 44 bp, artificial sequence
  Synthetic
cctctctatgggcagtcggtgatgaatgtcagctgaagcaggct

SEQ ID NO: 71 - DNA, 43 bp, artificial sequence
  Synthetic
ccatctcatccctgcgtgtctccggctaccctgacaactgctt

SEQ ID NO: 72 - DNA, 44 bp, artificial sequence
  Synthetic
cctctctatgggcagtcggtgatgaggacagacatgacaaggca

SEQ ID NO: 73 - DNA, 43 bp, artificial sequence
  Synthetic
ccatctcatccctgcgtgtctccgcaggcaagctgtcaagggt

SEQ ID NO: 74 - DNA, 44 bp, artificial sequence
  Synthetic
cctctctatgggcagtcggtgatgccctcacacccacaccctca

SEQ ID NO: 75 - DNA, 43 bp, artificial sequence
  Synthetic
ccatctcatccctgcgtgtctccggaggggtgtcatcgttctg

SEQ ID NO: 76 - DNA, 44 bp, artificial sequence
  Synthetic
cctctctatgggcagtcggtgatgcaaattgcgccatagctggg

SEQ ID NO: 77 - DNA, 43 bp, artificial sequence
  Synthetic
ccatctcatccctgcgtgtctcctgagcgctcttcgtctttcc

SEQ ID NO: 78 - DNA, 45 bp, artificial sequence
  Synthetic
cctctctatgggcagtcggtgatggccaggaacacaggaatgcta

SEQ ID NO: 79 - DNA, 1400 bp, artificial sequence
  Synthetic
cattctgcctctctgtatggaaaagagcatggggctggcccgtggggtggtgtccactttaggccctgtgggagatcatgggaacccacgcagtgggtcataggctctctcatttactactcacatccactctgtgaagaagcgattatgatctctcctctagaaactcgtagagtcccatgtctgccggcttccagagcctgcactcctccaccttggcttggctttgctggggctagaggagctaggatgcacagcagctctgtgaccctttgtttgagaggaacaggaaaaccacccttctctctggcccactgtgtcctcttcctgccctgccatccccttctgtgaatgttagacccatgggagcagctggtcagaggggaccccggcctggggcccctaaccctatgtagcctcagtcttcccatcaggctctcagctcagcctgagtgttgaggccccagtggctgctctgggggcctcctgagtttctcatctgtgcccctccctccctggcccaggtgaaggtgtggttccagaaccggaggacaaagtacaaacggcagaagctggaggaggaagggcctgagtccgagcagaagaagaagggctcccatcacatcaaccggtggcgcattgccacgaagcaggccaatggggaggacatcgatgtcacctccaatgactcggatgtacacggtctgcaaccacaaacccacgagggcagagtgctgcttgctgctggccaggcccctgcgtgggcccaagctggactctggccactccctggccaggctttggggaggcctggagtcatggccccacagggcttgaagcccggggccgccattgacagagggacaagcaatgggctggctgaggcctgggaccacttggccttctcctcggagagcctgcctgcctgggcgggcccgcccgccaccgcagcctcccagctgctctccgtgtctccaatctcccttttgttttgatgcatttctgttttaatttattttccaggcaccactgtagtttagtgatccccagtgtcccccttccctatgggaataataaaagtctctctcttaatgacacgggcatccagctccagccccagagcctggggtggtagattccggctctgagggccagtgggggctggtagagcaaacgcgttcagggcctgggagcctggggtggggtactggtggagggggtcaagggtaattcattaactcctctcttttgttgggggaccctggtctctacctccagctccacagcaggagaaacaggctagacatagggaagggccatcctgtatcttgagggaggacaggcccaggtctttcttaacgtattgagaggtgggaatcaggcccaggtagttcaatggg

SEQ ID NO: 80 - DNA, 1390 bp, artificial sequence
  Synthetic
aggtttgaatcatcacgcaggccctggcctccacccgcccccaccagccccctggcctcagttccctggcaacatctggggttgggggggcagcaggaacaagggcctctgtctgcccagctgcctccccctttgggttttgccagactccacagtgcatacgtgggctccaacaggtcctcttccctcccagtcactgactaaccccggaaccacacagcttcccgttctcagctccacaaacttggtgccaaattcttctcccctgggaagcatccctggacacttcccaaaggaccccagtcactccagcctgttggctgccgctcactttgatgtctgcaggccagatgagggctccagatggcacattgtcagagggacacactgtggcccctgtgcccagccctgggctctctgtacatgaagcaactccagtcccaaatatgtagctgtttgggaggtcagaaatagggggtccaggagcaaactccccccaccccctttccaaagcccattccctctttagccagagccggggtgtgcagacggcagtcactagggggcgctcggccaccacagggaagctgggtgaatggagcgagcagcgtcttcgagagtgaggacgtgtgtgtctgtgtgggtgagtgagtgtgcgcactctagaggtgtcgtgttgagggcgttggagcggggagaaggccaggggtcactccaggattccaatagatctgtgtgtccctctccccacccgtccctgtccggctctccgccttcccctgcccccttcaatattcctagcaaagagggaacggctctcaggccctgtccgcacgtaacctcactttcctgctccctcctcgccaatgccccgcgggcgcgtgtctctggacagagtttccgggggcggatgggtaattttcaggctgtgaaccttggtgggggtcgagcttccccttcattgcggcgggctgcgggccaggcttcactgagcgtccgcagagcccgggcccgagccgcgtgtggaagggctgaggctcgcctgtccccgccccccggggcgggccgggggcggggtcccggcggggcggagccatgcgccccccccttttttttttaaaagtcggctggtagcggggaggatcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaat

SEQ ID NO: 81 - DNA, 1551 bp, artificial sequence
  Synthetic
agtgacctgtgtaattatgcagaagaatggagctggattacacacagcaagttcctgcttctgggacagctctactgacggtatgattttcattcatgtttgtgaagttttgttgtgtgaaatatatgactggaagtttcctatctttgaatgcaatgcatgtttatcaccttttaaaacatttaataatagacttgccaaggttctttgtgtagcatagagatgggtacttgaatgttggccttattgtgagtaaaacgtcgtcccccagctttccctgccgtaaatgctgctctcttccctcccgcagggagctgcactgtgcgatgggagaataagaccatgtactgcatcgtcagtgccttcggactgtctattggaagcggagctactaacttcagcctgctgaagcaggctggagacgtggaggagaaccctggacctgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaaccagctgtcctgcctatggcctttctccttttgtctctagttcatcctctaaccaccagccatgaattcagtgaactcttttctcattctctttgttttgtggcactttcacaatgtagaggaaaaaaccaaatgaccgcactgtgatgtgaatggcaccgaagtcagatgagtatccctgtaggtcacctgcagcctgcgttgccacttgtcttaactctgaatatttcatttcaaaggtgctaaaatctgaaatctgctagtgtgaaacttgctctactctctgaaatgattcaaatacactaattttccatactttatacttttgttagaataaattattcaaatctaaagtctgttgtgttcttcatagtctgcatagtatcataaacg

SEQ ID NO: 82 - DNA, 1973 bp, artificial sequence
  Synthetic
gcagcaaagaaacacctggagataaaccctgaccattccattattgagaccttaaggcaaaaggcagaggctgataagaacgacaagtctgtgaaggatctggtcatcttgctttatgaaactgcgctcctgtcttctggcttcagtctggaagatccccagacacatgctaacaggatctacaggatgatcaaacttggtctgggtaagccttatactatgtaatgttaaaaagaaaataaacacacgtgacattgaagaaaatggtgaactttcagttatccaaacttggagcaccttgtcctgcttgctgcttggaggtattaaagtatgttttttttagggataagtaaggtcttacaagagcaaagaaatgaaattgagactcatatgtcctgtaatactgtcttgaaagcagatagaaaccaagagtattaccctaatagctggctttaagaaatctttgtaatatgaggattttattttggaaacaggtattgatgaagatgaccctactgctgatgataccagtgctgctgtaactgaagaaatgccaccccttgaaggagatgacgacacatcacgcatggaagaagtagacggaagcggagctactaacttcagcctgctgaagcaggctggagacgtggaggagaaccctggacctgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaaatctgtggctgagggatgacttacctgttcagtactctacaattcctctgataatatattttcaaggatgtttttctttatttttgttaatattaaaaagtctgtatggcatgacaactactttaaggggaagataagatttctgtctactaagtgatgctgtgataccttaggcactaaagcagagctagtaatgctttttgagtttcatgttggtttattttcacagattggggtaacgtgcactgtaagacgtatgtaacatgatgttaactttgtggtctaaagtgtttagctgtcaagccggatgcctaagtagaccaaatcttgttattgaagtgttctgagctgtatcttgatgtttagaaaagtattcgttacatcttgtaggatctactttttgaacttttcattccctgtagttgacaattctgcatgtactagtcctctagaaataggttaaactgaagcaacttgatggaaggatctctccacagggcttgttttccaaagaaaagtattgtttggaggagcaaagttaaaagcctacctaagcatatcgtaaagctgttcaaaaataactcagacccagtcttgtgga

SEQ ID NO: 83 - DNA, 2193 bp, artificial sequence
  Synthetic
gatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggcaagcttctgacctcttctcttcctcccacagggcctcgagagatctggcagcggaggaagcggagctactaacttcagcctgctgaagcaggctggagacgtggaggagaaccctggacctgtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaaactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggc

SEQ ID NO: 84 - DNA, 1245 bp, artificial sequence
  Synthetic
gcgactatgcacaacgagaggattttgaggctgctgggtctcctttctcagggggaccagtgtcctttcctctggccccagggccccattttggtaccccaggctatgggagccctcacttcactgcactgtactcctcggtccctttccctgagggggaagcctttccccctgtctccgtcaccactctgggctctcccatgcattcaaatggaagcggagctactaacttcagcctgctgaagcaggctggagacgtggaggagaaccctggacctgccaccatggtgagcgagctgattaaggagaacatgcacatgaagctgtacatggagggcaccgtgaacaaccaccacttcaagtgcacatccgagggcgaaggcaagccctacgagggcacccagaccatgagaatcaaggcggtcgagggcggccctctccccttcgccttcgacatcctggctaccagcttcatgtacggcagcaaaaccttcatcaaccacacccagggcatccccgacttctttaagcagtccttccccgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagacactgtaccccgctgacggcggcctggaaggcagagccgacatggccctgaagctcgtgggcgggggccacctgatctgcaaccttaagaccacatacagatccaagaaacccgctaagaacctcaagatgcccggcgtctactatgtggacaggagactggaaagaatcaaggaggccgacaaagagacatacgtcgagcagcacgaggtggctgtggccagatactgcgacctccctagcaaactggggcacaaacttaattcctaatgactaggaatgggggacagggggaggggaggagctagggaaagaaaacctggagtttgtgccagggtttttgggattaagttcttcattcactaaggaaggaattgggaacacaaagggtgggggcaggggagtttggggcaactggttggagggaaggtgaagttcaatgatgctcttgattttaatcccacatcatgtatcacttttttcttaaataaagaagcctgggacacagtagatagacacactt

SEQ ID NO: 85 - DNA, 810 bp, Pantoea stewartii
agcaaccagccccctatcgcctccgccgatctgcagaaggccaacaccggcaagcaggtggccaataagacccctgagcagacactggtgggcttcatgaatcagccagcaatgaagagccagctggccgccgccctgccaaggcacatgacagccgatcggatgatcagaatcgtgaccacagagatccgcaagacccccgccctggccacatgcgaccagagctccttcatcggcgccgtggtgcagtgttctcagctgggcctggagcctggcagcgccctgggccacgcctacctgctgccatttggcaacggccggagcaagtccggacagtccaatgtgcagctgatcatcggctatagaggcatgatcgatctggcccggagatctggccagatcgtgtctctgagcgccagggtggtgcgcgcagacgatgagttctcctttgagtacggcctggatgagaacctgatccaccggccaggcgagaatgaggacgcacccatcacccacgtgtatgcagtggcaagactgaaggacggaggcacccagttcgaagtgatgacagtgaagcagatcgagaaggtgaaggcccagtccaaggcctctagcaacggaccctgggtgacccactgggaggagatggccaagaaaaccgtgatcaggcgcctgtttaagtacctgcccgtgagcatcgagatgcagaaggccgtgatcctggatgagaaggccgagtctgacgtggatcaggacaatgcctccgtgctgtctgccgagtatagcgtgctggacggctcctctgaggag

SEQ ID NO: 86 - DNA, 813 bp, Pantoea stewartii
cagcccggcgtgtactatgacatctccaacgaggagtatcacgccggccctggcatcagcaagtcccagctggacgacatcgccgtgtccccagccatcttccagtggagaaagtctgcccccgtggacgatgagaaaaccgccgccctggacctgggcacagccctgcactgcctgctgctggagcctgatgagttctccaagaggtttatgatcggcccagaggtgaaccggagaaccaatgccggcaagcagaaggagcaggacttcctggatatgtgcgagcagcagggcatcacccctatcacacacgacgataaccggaagctgagactgatgagggactctgcctttgcccacccagtggccagatggatgctggagacagagggcaaggccgaggcctctatctactggaatgacagggatacacagatcctgagcaggtgccgccccgacaagctgatcaccgagttctcttggtgcgtggacgtgaagagcacagccgacatcggcaagttccagaaggacttctacagctatcgctaccacgtgcaggacgccttctattccgatggctacgaggcccagttttgcgaggtgccaaccttcgcctttctggtggtgagctcctctatcgattgtggccggtatcccgtgcaggtgtttatcatggaccagcaggcaaaggatgcaggaagggccgagtataagcggaacctgaccacatacgccgagtgccaggcaaggaatgagtggcctggcatcgccacactgagcctgccttactgggccaaggagatccggaatgtg

SEQ ID NO: 87 - DNA, 816 bp, Pantoea brenneri
agcaaccagccccctatcgcctccgccgatctgcagaaaacccagcagtccaagcaggtggccaacaagacccctgagcagacactggtgggcttcatgaatcagccagcaatgaagagccagctggccgccgccctgccaaggcacatgaccgccgatcggatgatcagaatcgtgaccacagagatccgcaagacaccacagctggcccagtgcgaccagagctccttcatcggcgccgtggtgcagtgttctcagctgggcctggagcctggcagcgccctgggccacgcctacctgctgccatttggcaacggccggtccaagtctggccagagcaatgtgcagctgatcatcggctatagaggcatgatcgatctggcccggagatccggacagatcgtgagcctgtccgccagggtggtgcgcgcagacgatgagttctcttttgagtacggcctggatgagaacctggtgcaccggccaggcgagaatgaggacgcacccatcacccacgtgtatgcagtggcaagactgaaggacggaggcacccagttcgaagtgatgacagtgaagcaggtggagaaggtgaaggcccagtccaaggcctctagcaatggcccctgggtgacccactgggaggagatggccaagaaaaccgtgatcaggcgcctgtttaagtacctgcccgtgagcatcgagatgcagaaggccgtggtgctggatgagaaggccgagtctgacgtggatcaggacaacgcctctgtgctgagcgccgagtattccgtgctggagtctggcgacgaggccacaaat

SEQ ID NO: 88 - DNA, 813 bp, Pantoea brenneri
cagcctggcatctactatgacatcagcaacgaggattatcacaggggagcaggcatcagcaagtcccagctggacgacatcgccatctccccagccatctaccagtggagaaagcacgcccccgtggacgaggagaaaaccgccgccctggatctgggcacagccctgcactgcctgctgctggagcctgacgagttctctaagaggtttcagatcggcccagaggtgaaccggagaaccacagccggcaaggagaaggagaaggagttcatcgagcggtgcgaggcagagggaatcaccccaatcacacacgacgataataggaagctgaagctgatgagggattccgccctggcccacccaatcgcaaggtggatgctggaggcacagggaaacgcagaggcctctatctattggaatgacagagatgccggcgtgctgagcaggtgccgccccgacaagatcatcaccgagttcaactggtgcgtggacgtgaagtccacagccgacatcatgaagttccagaaggacttctactcttacagataccacgtgcaggacgccttctattccgatggctacgagtctcactttcacgagacacccacattcgcctttctggccgtgtctaccagcatcgactgcggcaggtatcctgtgcaggtgtttatcatggaccagcaggcaaaggatgcaggaagggccgagtacaagagaaacatccacaccttcgccgagtgtctgagcaggaatgagtggcctggcatcgccacactgtccctgcctttttgggccaaggagctgcgcaatgag

SEQ ID NO: 89 - DNA, 807 bp, Pantoea dispersa
tccaaccagccacctctggccaccgcagatctgcagaaaacccagcagtctaaccaggtggccaagacccctgagcagacactggtgggcttcatgaatcagccagcaatgaagagccagctggccgccgccctgccaaggcacatgaccgccgatcggatgatcagaatcgtgaccacagagatccgcaagacacccgccctggcccagtgcgaccagagctccttcatcggagcagtggtgcagtgtagccagctgggcctggagcctggctccgccctgggccacgcctacctgctgccatttggcaacggccggtccaagtctggccagagcaatgtgcagctgatcatcggctatagaggcatgatcgatctggcccggagatccggacagatcgtgagcctgtccgccagggtggtgcgcgcagacgatgagttctcttttgagtacggcctggatgagaacctgatccaccggccaggcgacaatgagtccgcccccatcacccacgtgtatgcagtggcaagactgaaggacggaggcacccagttcgaagtgatgacagccaagcaggtggagaaggtgaaggcccagtccaaggcctctagcaacggaccctgggtgacccactgggaggagatggccaagaaaaccgtgatcaggcgcctgtttaagtacctgcccgtgagcatcgagatgcagaaggccgtggtgctggacgagaaggccgagagcgacgtggatcaggacaatgcctctgtgctgagcgccgagtattccgtgctggagtctggcacaggcgag

SEQ ID NO: 90 - DNA, 813 bp, Pantoea dispersa
gagccaggcatctactatgacatcagcaacgaggcctaccactccggccccggcatcagcaagtcccagctggacgacatcgccaggagccctgccatcttccagtggcgcaaggacgccccagtggataccgagaaaaccaaggccctggacctgggcaccgatttccactgcgccgtgctggagccagagaggtttgcagacatgtatcgcgtgggccctgaagtgaatcggagaaccacagccggcaaggccgaggagaaggagttctttgagaagtgtgagaaggatggagccgtgcccatcacccacgacgatgcacggaaggtggagctgatgagaggctccgtgatggcccaccctatcgccaagcagatgatcgcagcacagggacacgcagaggcctctatctactggcacgacgagagcacaggcaacctgtgccggtgtagacccgacaagtttatccctgattggaattggatcgtggacgtgaaaaccacagccgatatgaagaagttcaggcgcgagttttacgatctgcggtatcacgtgcaggacgccttctacaccgatggctatgccgcccagtttggcgagcggcctaccttcgtgtttgtggtgacatccaccacaatcgactgcggcagataccccaccgaggtgttctttctggatgaggagacaaaggccgccggcaggtctgagtaccagagcaacctggtgacctattccgagtgtctgtctcgcaatgagtggccaggcatcgccacactgtctctgccccactgggccaaggagctgaggaacgtg

SEQ ID NO: 91 - DNA, 810 bp, Type-F symbiont of Plautia stali
tccaaccagccccctatcgcctctgccgatctgcagaaaacccagcagtctaagcaggtggccaacaagacccctgagcagacactggtgggcttcatgaatcagccagcaatgaagtcccagctggccgccgccctgccaaggcacatgacagccgatcggatgatcagaatcgtgaccacagagatccgcaagacccccgccctggccacatgcgaccagagctccttcatcggagcagtggtgcagtgtagccagctgggcctggagcctggctccgccctgggccacgcctacctgctgccatttggcaacggccggtccaagtctggccagtctaatgtgcagctgatcatcggctatagaggcatgatcgacctggcccggagaagcggacagatcgtgagcctgtccgccagggtggtgcgcgcagacgatgagttctcctttgagtacggcctggatgagaacctgatccaccggccaggcgataatgaggacgcccccatcacccacgtgtatgcagtggcaagactgaaggacggaggcacccagttcgaagtgatgacagccaagcaggtggagaaggtgaaggcccagagcaaggcctctagcaacggaccctgggtgacccactgggaggagatggccaagaaaaccgtgatcaggcgcctgtttaagtacctgcccgtgagcatcgagatgcagaaggccgtggtgctggatgagaaggccgagagcgacgtggatcaggacaatgcctctgtgctgagcgccgagtattccgtgctggagggcgacggcggcgag

SEQ ID NO: 92 - DNA, 813 bp, Type-F symbiont of Plautia stali
cagcctggcatctactatgacatcagcaacgaggattatcacggcggccctggcatcagcaagtcccagctggacgacatcgccatctccccagccatctaccagtggaggaagcacgcccccgtggacgaggagaaaaccgccgccctggatctgggcacagccctgcactgcctgctgctggagcctgacgagttctctaagagatttgagatcggcccagaggtgaaccggagaaccacagccggcaaggagaaggagaaggagttcatggagaggtgtgaggcagagggagtgacccctatcacacacgacgataatcggaagctgagactgatgagggatagcgcaatggcccacccaatcgccagatggatgctggaggcacagggaaacgcagaggcctctatctattggaatgacagggataccggcgtgctgagcaggtgccgccccgacaagatcatcaccgacttcaactggtgcgtggacgtgaagtccacagccgacatcatcaagttccagaaggacttttactcttatcgctaccacgtgcaggacgccttctattccgatggctacgagtctcactttgacgagacaccaacattcgcctttctggccgtgtctacaagcatcgattgcggccggtatcccgtgcaggtgttcatcatggaccagcaggcaaaggatgcaggaagggccgagtacaagcggaacatccacacctttgccgagtgtctgagccgcaatgagtggcctggcatcgccacactgtccctgccttactgggccaaggagctgcggaatgag

SEQ ID NO: 93 - DNA, 801 bp, Providencia stuartii
agcaacccacctctggcccaggcagacctgcagaaaacccagggcacagaggtgaaggagaaaaccaaggatcagatgctggtggagctgatcaataagccttccatgaaggcacagctggccgccgccctgccaaggcacatgacacccgaccggatgatcagaatcgtgaccacagagatcagaaagacccccgccctggccacatgcgatatgcagagcttcgtgggagcagtggtgcagtgttcccagctgggcctggagcctggcaacgccctgggacacgcctacctgctgccttttggcaacggcaagtctaagagcggccagtctaatgtgcagctgatcatcggctatcggggcatgatcgacctggcccggagaagcggccagatcgtgtccatctctgccaggaccgtgcgccagggcgataacttccactttgagtacggcctgaacgagaatctgacccacgtgcctggcgagaatgaggactctccaatcacacacgtgtacgcagtggcaaggctgaaggatggaggcgtgcagttcgaagtgatgacctataaccagatcgagaaggtgcgcgccagctccaaggcaggacagaatggaccctgggtgagccactgggaggagatggccaagaaaaccgtgatcaggcgcctgttcaagtacctgcccgtgtctatcgagatgcagaaggccgtgatcctggacgagaaggccgaggccaacatcgatcaggagaatgccaccatctttgagggcgagtatgaggaagtgggcacagacggcaag

SEQ ID NO: 94 - DNA, 810 bp, Providencia stuartii
gagggcatctactataacatcagcaatgaggactaccacaacggcctgggcatctccaagtctcagctggatctgatcaatgagatgcctgccgagtatatctggtccaaggaggcccccgtggacgaggagaagatcaagcctctggagatcggcaccgccctgcactgcctgctgctggagccagacgagtaccacaagagatataagatcggccccgatgtgaaccggagaacaaatgccggcaaggagaaggagaaggagttctttgatatgtgcgagaaggagggcatcacccccatcacacacgacgataaccggaagctgatgatcatgagagactctgccctggcccaccctatcgccaagtggtgtctggaggccgatggcgtgagcgagagctccatctactggaccgacaaggagacagatgtgctgtgcaggtgtcgcccagaccgcatcatcaccgcccacaactacatcgtggatgtgaagtctagcggcgacatcgagaagttcgattacgagtactacaactacagataccacgtgcaggacgccttttactccgatggctataaggaggtgaccggcatcacccctacattcctgtttctggtggtgtctaccaagatcgactgcggcaagtaccccgtgcggacctacgtgatgagcgaggaggcaaagtccgccggaaggaccgcctacaagcacaacctgctgacctatgccgagtgtctgaaaaccgatgagtgggccggcatcaggacactgtctctgcccagatgggcaaaggagctgcggaatgag

SEQ ID NO: 95 - DNA, 801 bp, Providencia sp. MGF014
tctaacccccctctggcccagagcgacctgcagaaaacccagggcacagaggtgaaggtgaaaaccaaggatcagcagctgatccagttcatcaatcagccttctatgaaggcacagctggccgccgccctgccaaggcacatgacacccgaccggatgatcagaatcgtgaccacagagatcagaaagacccccgccctggccacatgcgatatgcagtccttcgtgggcgccgtggtgcagtgttctcagctgggcctggagcctggcaacgccctgggacacgcctacctgctgccttttggcaacggcaaggccaagtccggccagtctaatgtgcagctgatcatcggctatcggggcatgatcgacctggcccggagatccaaccagatcatctctatcagcgccaggaccgtgcgccagggcgataacttccactttgagtacggcctgaatgaggacctgacccacacacctagcgagaatgaggattccccaatcacccacgtgtacgcagtggcaaggctgaaggacggaggcgtgcagtttgaagtgatgacatataaccaggtggagaaggtgcgcgccagctccaaggcaggacagaatggaccctgggtgagccactgggaggagatggccaagaaaaccgtgatcaggcgcctgttcaagtacctgcccgtgtccatcgagatgcagaaggcagtggtgctggacgagaaggcagaggccaacgtggatcaggagaatgccaccatctttgagggcgagtatgaggaagtgggcacagatggcaat

SEQ ID NO: 96 - DNA, 813 bp, Providencia sp. MGF014
aaggagggcatctactataacatcagcaatgaggactaccacaacggcctgggcatctccaagtctcagctggatctgatcaatgagatgcctgccgagtatatctggtccaaggaggcccccgtggacgaggagaagatcaagcctctggagatcggcaccgccctgcactgcctgctgctggagccagacgagtaccacaagagatataagatcggccccgatgtgaaccggagaacaaatgtgggcaaggagaaggagaaggagttctttgatatgtgcgagaaggagggcatcacccccatcacacacgacgataaccggaagctgatgatcatgagagactctgccctggcccaccctatcgccaagtggtgtctggaggccgatggcgtgagcgagagctccatctactggaccgacaaggagacagatgtgctgtgcaggtgtcgcccagaccgcatcatcaccgcccacaactacatcatcgatgtgaagtctagcggcgacatcgagaagttcgattacgagtactacaactacagataccacgtgcaggacgccttttactccgatggctataaggaggtgaccggcatcacccctacattcctgtttctggtggtgtctaccaagatcgactgcggcaagtaccccgtgcggacctacgtgatgagcgaggaggcaaagtccgccggaaggaccgcctacaagcacaacctgctgacctatgccgagtgtctgaaaaccgatgagtgggccggcatcaggacactgtctctgcccagatgggcaaaggagctgcggaatgag

SEQ ID NO: 97 - DNA, 711 bp, Shewanella putrefaciens
cagaccgcacaggtgaagctgagcgtgccccaccagcaggtgtaccaggacaacttcaattatctgagctcccaggtggtgggccacctggtggatctgaacgaggagatcggctacctgaaccagatcgtgtttaattctctgagcaccgcctctcccctggacgtggcagcaccttggagcgtgtacggcctgctgctgaacgtgtgccggctgggcctgtccctgaatccagagaagaagctggcctatgtgatgccctcctggtctgagacaggcgagatcatcatgaagctgtaccccggctataggggcgagatcgccatcgcctctaacttcaatgtgatcaagaacgccaatgccgtgctggtgtatgagaacgatcacttccgcatccaggcagcaaccggcgagatcgagcactttgtgacaagcctgtccatcgaccctagggtgcgcggagcatgcagcggaggctactgtcggtccgtgctgatggataatacaatccagatctcttatctgagcatcgaggagatgaacgccatcgcccagaatcagatcgaggccaacatgggcaataccccttggaactccatctggcggacagagatgaatagagtggccctgtaccggagagcagcaaaggactggaggcagctgatcaaggccaccccagagatccagtccgccctgtctgatacagagtat

SEQ ID NO: 98 - DNA, 1173 bp, Shewanella putrefaciens
ggcaccgccctggcccagacaatcagcctggactggcaggataccatccagccagcatacacagcctccggcaagcctaacttcctgaatgcccagggcgagatcgtggagggcatctacaccgatctgcctaattccgtgtatcacgccctggacgcacacagctccaccggcatcaagacattcgccaagggccgccaccactactttcggcagtatctgtctgacgtgtgccggcagagaacaaagcagcaggagtacaccttcgacgccggcacctacggccacatgctggtgctggagccagagaacttccacggcaacttcatgaggaaccccgtgcctgacgattttccagacatcgagctgatcgagagcatcccacagctgaaggccgccctggccaagagcaacctgcccgtgtccggagcaaaggccgccctgatcgagagactgtacgccttcgacccatccctgcccctgtttgagaagatgagggagaaggccatcaccgactatctggatctgcgctacgccaagtatctgcggaccgacgtggagctggatgagatggccacattctacggcatcgatacctctcagacacgggagaagaagatcgaggagatcctggccatctctcctagccagccaatctgggagaagctgatcagccagcacgtgatcgaccacatcgtgtgggacgatgccatgagggtggagagatccaccagggcccaccctaaggcagactggctgatctctgatggctatgccgagctgacaatcatcgcaaggtgcccaaccaccggcctgctgctgaaggtgcggtttgactggctgaggaatgatgccatcggcgtggacttcaagaccacactgtctaccaaccccacaaagtttggctaccagatcaaggacctgcggtatgatctgcagcaggtgttctactgttatgtggccaatctggccggcatccctgtgaagcacttctgctttgtggccaccgagtacaaggacgccgataactgtgagacatttgagctgtctcacaagaaagtgatcgagagcaccgaggagatgttcgacctgctggatgagtttaaggaggccctgacctccggcaattggtatggccacgacaggtcccgctctacatgggtcatcgaggtg

SEQ ID NO: 99 - DNA, 888 bp, Bacillus sp. MUM 116
agcaagcagctgaccacagtgaatacccaggccgtggtgggcacattctcccaggccgagctggataccctgaagcagacaatcgccaagggcaccacaaacgagcagttcgccctgtttgtgcagacctgcgccaactctaggctgaatccatttctgaaccacatccactgtatcgtgtataacggcaaggagggcgccaccatgagcctgcagatcgcagtggagggcatcctgtacctggcacgcaagacagacggctataagggcatcgagtgccagctgatccacgagaatgacgagttcaagtttgatgccaagtccaaggaggtggatcaccagatcggattccccaggggcaacgtgatcggaggatatgcaatcgcaaagagggagggctttgacgatgtggtggtgctgatggagtctaacgaggtggaccacatgctgaagggccggaatggccacatgtggagagactggttcaacgatatgtttaagaagcacatcatgaagcgggccgccaagctgcagtacggcatcgagatcgcagaggacgagacagtgagcagcggacctagcgtggataatatcccagagtataagccacagccccggaaggacatcacacccaaccaggacgtgatcgatgccccccctcagcagcctaagcaggacgatgaggccgccaagctgaaggccgccagatctgaggtgagcaagaagttcaagaagctgggcatcgtgaaggaggatcagaccgagtacgtggagaagcacgtgcctggcttcaagggcacactgtccgactttatcggcctgtctcagctgctggatctgaatatcgaggcccaggaggcccagtccgccgacggcgatctgctggac

SEQ ID NO: 100 - DNA, 1323 bp, Bacillus sp. MUM 116
acctacgccgccgacgagacactggtgcagctgctgctgtccgtggatggcaagcagctgctgctgggaaggggcctgaagaagggcaaggcccagtactatatcaatgaggtgccatctaaggccaaggagttcgaggagatccgggaccagctgtttgacaaggatctgttcatgtccctgtttaacccctcttacttctttaccctgcactgggagaagcagagggccatgatgctgaagtatgtgacagcccccgtgtctaaggaggtgctgaagaatctgcctgaggcccagtccgaggtgctggagagatacctgaagaagcactctctggtggatctggagaagatccacaaggacaacaagaataagcaggataaggcctatatctctgcccagagcaggaccaacacactgaaggagcagctgatgcagctgaccgaggagaagctggacatcgattccatcaaggccgagctggcccacatcgacatgcaggtcatcgagctggagaagcagatggatacagccttcgagaagaaccaggcctttaatctgcaggcccagatcaggaatctgcaggacaagatcgagatgagcaaggagcggtggccctccctgaagaacgaagtgatcgaggatacctgccggacatgcaagcggcccctggacgaggatagcgtggaggccgtgaaggccgacaaggataatcggatcgccgagtacaaggccaagcacaactccctggtgtctcagagaaatgagctgaaggagcagctgaacaccatcgagtatatcgacgtgacagagctgagagagcagatcaaggagctggatgagtccggacagcctctgagggagcaggtgcgcatctacagccagtatcagaatctggacacccaggtgaagtccgccgaggcagacgagaacggcatcctgcaggatctgaaggcctctatcttcatcctggatagcatcaaggcctttaggggcaaggaggccgagatgcaggccgagaaggtgcaggccctgttcaccacactgagcgtgcgcctgtttaagcagaataagggcgacggcgagatcaagccagatttcgagatcgagatgaacgacaagccctatcggaccctgagcctgtccgagggcatccgggcaggcctggagctgcgggacgtgctgagccagcagtccgagctggtgacccctacattcgtggataatgccgagtctatcaccagcttcaagcagccaaacggccagctgatcatcagccgggtggtggcaggacaggagctgaagatcgaggccgtgagcgag

SEQ ID NO: 101 - DNA, 804 bp, Shigella sonnei
accaagcagccccctatcgccaaggccgacctgcagaaaacccaggagaacagggcaccagcagccatcaagaacaatgatgtgatctcctttatcaatcagccctctatgaaggagcagctggccgccgccctgcctaggcacatgaccgccgagaggatgatccgcatcgccaccacagagatccgcaaggtgcctgccctgggcaactgcgacacaatgagcttcgtgagcgccatcgtgcagtgtagccagctgggcctggagccaggctccgccctgggccacgcctacctgctgcccttcggcaacaagaatgagaagtccggcaagaagaatgtgcagctgatcatcggctataggggcatgatcgatctggcccggagatctggccagatcgcctctctgagcgccagagtggtgcgggagggcgacgagttcaactttgagttcggcctggatgagaagctgatccaccggcctggcgagaatgaggacgccccagtgacccacgtgtacgcagtggccagactgaaggatggcggcacccagtttgaagtgatgacaaggcgccagatcgagctggtgaggtcccagtctaaggccggcaacaatggcccttgggtgacccactgggaggagatggccaagaaaaccgccatccggagactgttcaagtacctgccagtgtctatcgagatccagcgcgccgtgagcatggacgagaaggagccactgaccatcgaccccgccgatagctccgtgctgacaggcgagtattctgtgatcgataacagcgaggag

SEQ ID NO: 102 - DNA, 1944 bp, Shigella sonnei
gatcgcggcctgctgacaaaggagtggaggaagggaaaccgggtgagccggatcaccaggacagccagcggagcaaacgcaggaggaggaaatctgaccgacagaggcgagggcttcgtgcacgatctgacaagcctggcccgcgacatcgcaaccggcgtgctggcccggagcatggacgtggacatctacaacctgcaccctgcccacgccaagaggatcgaggagatcatcgccgagaataagccccctttcagcgtgtttagagacaagtttatcacaatgccaggcggcctggactactccagggccatcgtggtggcctctgtgaaggaggccccaatcggcatcgaagtgatccccgcccacgtgaccgcctatctgaacaaggtgctgaccgagacagaccacgccaatccagatcccgagatcgtggacatcgcatgcggcagaagctccgcccctatgccacagagggtgaccgaggagggcaagcaggacgatgaggagaagctgcagccttctggcaccacagcagatgagcagggagaggcagagacaatggagccagacgccacaaagcaccaccaggatacccagcctctggacgcccagagccaggtgaacagcgtggatgccaagtatcaggagctgagagccgagctgcacgaggccaggaagaacatcccttccaagaatccagtggacgcagataagctgctggccgcctctcgcggcgagttcgtggacggcatcagcgacccaaacgatcccaagtgggtgaagggcatccagacacgggattccgtgtaccagaatcagcctgagacagagaaaaccagccccgacatgaagcagccagagcctgtggtgcagcaggagcctgagatcgccttcaacgcctgcggacagaccggcggcgacaattgcccagattgtggcgccgtgatgggcgatgccacctatcaggagacatttgacgaggagaaccaggtggaggccaaggagaatgatcctgaggagatggagggcgccgagcacccacacaacgagaatgccggcagcgacccccacagagactgttccgatgagacaggcgaggtggccgatcccgtgatcgtggaggacatcgagcctggcatctactatggcatcagcaacgagaattaccacgcaggccccggcgtgtccaagtctcagctggacgacatcgccgacacacctgccctgtatctgtggaggaagaacgccccagtggataccacaaagaccaagacactggacctgggcaccgcattccactgccgcgtgctggagccagaggagttcagcaatcggtttatcgtggcccccgagttcaaccggagaacaaatgccggcaaggaggaggagaaggcctttctgatggagtgtgcctccacaggcaagatggtcatcaccgccgaggagggcagaaagatcgagctgatgtaccagtctgtgatggcactgccactgggacagtggctggtggagagcgccggacacgcagagtctagcatctattgggaggaccccgagacaggcatcctgtgcaggtgtcgccccgacaagatcatccctgagttccactggatcatggacgtgaaaaccacagccgacatccagcggttcaagacagcctactatgattacaggtatcacgtgcaggatgccttctactccgacggctatgaggcccagtttggcgtgcagcccaccttcgtgtttctggtggcctctaccacaatcgagtgcggcagataccccgtggagatctttatgatgggagaggaggcaaagctggccggacagctggagtatcaccgcaacctgcggacactggccgattgtctgaataccgacgagtggccagccatcaagaccctgtccctgcccagatgggcaaaggagtacgccaacgac

SEQ ID NO: 103 - DNA, 828 bp, Salmonella enterica
accaagcagccccctatcgccaaggccgacctgcagaaaacccagggaaacagggcacctgcagcagtgaatgacaaggatgtgctgtgcgtgatcaacagccctgccatgaaggcacagctggccgccgccctgccaaggcacatgaccgccgagaggatgatccgcatcgccaccacagagatcaggaaggtgccagagctgcgcaactgcgacagcaccagcttcatcggcgccatcgtgcagtgttctcagctgggcctggagcccggcagcgccctgggccacgcctacctgctgccttttggcaatggcaaggccaagaacggcaagaagaatgtgcagctgatcatcggctatcggggcatgatcgatctggcccggagatctggccagatcatctccctgagcgccagagtggtgcgggagtgtgacgagttctcctacgagctgggcctggatgagaagctggtgcaccggccaggcgagaacgaggacgcacccatcacccacgtgtatgccgtggccaagctgaaggatggcggcgtgcagtttgaagtgatgaccaagaagcaggtggagaaggtgagagatacacactccaaggccgccaagaatgccgcctctaagggcgccagctccatctgggacgagcacttcgaggatatggccaagaaaaccgtgatccggaagctgtttaagtacctgcccgtgagcatcgagatccagagagccgtgagcatggacggcaaggaggtggagacaatcaacccagacgacatcagcgtgatcgccggcgagtattccgtgatcgataatcccgaggag

SEQ ID NO: 104 - DNA, 1944 bp, Salmonella enterica
gatcgcggcctgctgacaaaggagtggaggaagggaaaccgggtgagccggatcaccaggacagccagcggagcaaacgcaggaggaggaaatctgaccgacagaggcgagggcttcgtgcacgatctgacaagcctggcccgcgacgtggcaaccggcgtgctggcccggagcatggacgtggacatctacaacctgcaccctgcccacgccaagagggtggaggagatcatcgccgagaataagccccctttcagcgtgtttagagacaagtttatcacaatgcctggcggcctggactactccagggccatcgtggtggcctctgtgaaggaggcccctatcggcatcgaagtgatcccagcccacgtgaccgagtatctgaacaaggtgctgaccgagacagaccacgccaatccagatcccgagatcgtggacatcgcatgcggcagaagctccgcccctatgccacagagggtgaccgaggagggcaagcaggacgatgaggagaagccccagccttctggagctatggccgacgagcaggcaaccgcagagacagtggagccaaacgccacagagcaccaccagaatacccagcccctggatgcccagagccaggtgaactccgtggacgccaagtatcaggagctgagagccgagctgcaggaggccaggaagaacatcccctccaagaatcctgtggacgcagataagctgctggccgcctctcgcggcgagttcgtggatggcatcagcgaccctaacgatccaaagtgggtgaagggcatccagacacgggattccgtgtaccagaatcagcccgagacagagaagatctctcctgacgccaagcagccagagcccgtggtgcagcaggagcccgagacagtgtgcaacgcctgtggacagaccggcggcgacaattgccctgattgtggcgccgtgatgggcgacgccacatatcaggagacattcggcgaggagaatcaggtggaggccaaggagaaggaccccgaggagatggagggagcagagcaccctcacaacgagaatgccggcagcgacccacacagagactgttccgatgagacaggcgaggtggccgatccagtgatcgtggaggacatcgagcctggcatctactatggcatcagcaacgagaattaccacgcaggccccggcgtgtccaagtctcagctggacgacatcgccgacacacccgccctgtatctgtggaggaagaacgcccctgtggataccacaaagaccaagacactggacctgggcaccgcattccactgccgcgtgctggagcctgaggagttcagcaatcggtttatcgtggccccagagttcaaccggagaacaaatgccggcaaggaggaggagaaggcctttctgatggagtgtgcctccaccggcaagacagtgatcaccgccgaggagggcagaaagatcgagctgatgtaccagtctgtgatggcactgcctctgggacagtggctggtggagagcgccggacacgcagagtctagcatctattgggaggaccccgagacaggcatcctgtgcaggtgtcgcccagacaagatcatccccgagttccactggatcatggacgtgaaaaccacagccgacatccagcggttcaagacagcctactatgattacaggtatcacgtgcaggatgccttctactccgacggctatgaggcccagtttggcgtgcagccaaccttcgtgtttctggtggcctctaccacagtggagtgcggcagataccccgtggagatctttatgatgggagaggaggcaaagctggccggacagcaggagtatcaccgcaacctgcggacactggccgattgtctgaataccgacgagtggcctgccatcaagaccctgtccctgccacggtgggccaaggagtacgccaacgac

SEQ ID NO: 105 - DNA, 993 bp, Acetobacter
aacgccccccagaagcagaataccagagccgccgtgaagaagatcagccctcaggagttcgccgagcagtttgccgccatcatcccacaggtgaagtccgtgctgcccgcccacgtgaccttcgagaagtttgagcgggtggtgagactggccgtgcggaagaaccctgacctgctgacatgctccccagcctctctgttcatggcatgtatccaggcagcctccgacggcctgctgcctgatggaagggagggagcaatcgtgagccggtggagctccaagaagagctgcaacgaggcctcctggatgccaatggtggccggcctgatgaagctggcccggaacagcggcgacatcgccagcatctctagccaggtggtgttcgagggcgagcactttagagtggtgctgggcgacgaggagaggatcgagcacgagcgcgatctgggcaagaccggcggcaagatcgtggcagcctacgccgtggcaaggctgaaggacggcagcgatccaatccgcgagatcatgtcctggggccagatcgagaagatcagaaacacaaataagaagtgggagtggggaccctggaaggcctgggaggacgagatggccagaaagaccgtgatccggagactggccaagagactgcccatgtctacagataaggagggagagaggctgcgcagcgccatcgagaggatcgactccctggtggacatctctgccaacgtggacgcacctcagatcgcagcagacgatgagtttgccgccgccgcccacggcgtggagccacagcagatcgcagcacctgacctgatcggccgcctggcccagatgcagtccctggagcaggtgcaggacatcgagccccaggtgtctcacgccatccaggaggccgacaagaggggcgacagcgatacagccaatgccctggatgccgccctgcagagcgccctgtcccgcacctctacagccaaggaggaggtgcctgcc

SEQ ID NO: 106 - DNA, 897 bp, Acetobacter
gtgatctctaagagcggcatctacgacctgaccaacgagcagtatcacgccgatccttgcccagagatgtccctgagctcctctggagccagggacctgctgagctcctgtcctgccaagttcatcgccgccaagcagctgccacagcagaataagaggtgctttgacatcggctctgccggacacctgatggtgctggagccacacctgttcgaccagaaggtgtgcgagatcaagcaccctgattggcgcacaaaggcagcaaaggaggagcgggacgccgcctacgccgagggaagaatccccctgctgagccgcgaggtggaggacatcagggcaatgcactccgtggtgtggagagattctctgggagccagggccttcagcggaggcaaggcagagcagtccctggtgtggcgcgacgaggagtttggcatctggtgccggctgcggcccgattacgtgcctaacaatgccgtgcggatcttcgactataagaccgccacaaacggctcccccgatgcctttatgaaggagatctacaatcggggctatcaccagcaggccgcctggtatctggacggatatgaggcagtgaccggccacaggccacgcgagttctggtttgtggtgcaggagaaaaccgcccccttcctgctgtctttctttcagatggatgagatgagcctggagatcggccggaccctgaacagacaggccaagggcatctttgcctggtgcctgcgcaacaattgttggccaggctatcagcccgaggtggatggcaaggtgagattctttaccacatctccccctgcctggctggtgagggagtacgagtttaagaatgagcacggcgcctatgagccacccgagatcaagcggaaggaggtggcc

SEQ ID NO: 107 - DNA, 825 bp, Salmonella enterica subsp. enterica serovar Javiana str. 10721+C107
ccaaagcagccccctatcgccaaggcagacctgcagaaaacccagggagcacggaccccaacagcagtgaagaacaataacgatgtgatctcctttatcaatcagccttctatgaaggagcagctggccgccgccctgccaaggcacatgaccgccgagcggatgatcagaatcgccaccacagagatcaggaaggtgcccgccctgggcgactgcgatacaatgtcttttgtgagcgccatcgtgcagtgtagccagctgggcctggagcctggcggcgccctgggccacgcctacctgctgcctttcggcaatcggaacgagaagtccggcaagaagaatgtgcagctgatcatcggctatagaggcatgatcgacctggcccggagatccggacagatcgccagcctgtccgccagggtggtgcgcgagggcgacgatttctcttttgagttcggcctggaggagaagctggtgcacaggccaggcgagaacgaggacgcccccgtgacccacgtgtacgcagtggcacgcctgaaggatggaggcacccagtttgaagtgatgacacggaagcagatcgagctggtgagagcccagtctaaggccggcaataacggcccttgggtgacccactgggaggagatggccaagaaaaccgccatcaggcgcctgttcaagtacctgcccgtgagcatcgagatccagagggccgtgagcatggatgagaaggagacactgacaatcgacccagccgatgccagcgtgatcaccggcgagtattccgtggtggagaatgccggcgtggaggagaacgtgacagcc

SEQ ID NO: 108 - DNA, 801 bp, Salmonella enterica subsp. enterica serovar Javiana str. 10721
tactatgacatcccaaacgaggcctaccacgcaggccccggcgtgtctaagagccagctggacgacatcgccgatacccccgccatctatctgtggcggaagaatgcccctgtggacaccgagaaaaccaagtccctggataccggcacagccttccactgcagggtgctggagccagaggagttcagcaagcggttcatcatcgcccccgagttcaaccggagaacctccgccggcaaggaggaggagaaaaccttcctggaggagtgtacccggacaggcagaaccgtgctgacagccgaggagggcaggaagatcgagctgatgtaccagtccgtgatggcactgccactgggacagtggctggtggagtctgccggctacgccgagagctccgtgtattgggaggaccctgagacaggcatcctgtgccggtgtagacccgataagatcatccctgagttccactggatcatggacgtgaaaaccacagccgacatccagaggtttcgcaccgcctactatgactacagataccacgtgcaggacgccttctactctgatggctatagagcccagtttggcgagatccctacattcgtgtttctggtggccagcaccacagcagagtgcggcagataccccgtggagatctttatgatgggagaggacgcaaagctggccggacagcgcgagtataggcgcaatctgcagaccctggccgagtgtctgaacaatgatgagtggcctgccatcaagacactgtctctgccacggtgggccaaggagaacgccaatgcc

SEQ ID NO: 109 - DNA, 723 bp, Pseudobacteriovorax antillogorgiicola
ggccacctggtgagcaagaccgagcaggattacatcaagcagcactatgccaagggcgccacagaccaggagttcgagcactttatcggcgtgtgcagggccagaggcctgaacccagccgccaatcagatctacttcgtgaagtatcggtccaaggatggaccagcaaagccagcctttatcctgtctatcgacagcctgaggctgatcgcacaccgcaccggcgattacgcaggatgctctgagcccatcttcacagacggcggcaaggcctgtaccgtgacagtgcggagaaacctgaagagcggcgagacaggcaatttctccggcatggccttttatgacgagcaggtgcagcagaagaacggccggcctacctccttttggcagtctaagccaagaacaatgctggagaagtgtgcagaggcaaaggccctgaggaaggccttccctcaggatctgggccagttttacatcagagaggagatgccccctcagtatgacgagcctatccaggtgcacaagccaaaggccctggaggagcccaggttcagcaagtccgatctgtccaggcgcaagggcctgaacaggaagctgtctgccctgggagtggaccccagccgcttcgatgaggtggccacctttctggacggcacacctgatcgcgagctgggccagaagctgaagctgtggctgaaggaggccggctacggcgtgaatcag

SEQ ID NO: 110 - DNA, 897 bp, Pseudobacteriovorax antillogorgiicola
agcaagctgtccaacctgaaggtgtctaatagcgacgtggatacactgagccggatcagaatgaaggagggcgtgtatcgggacctgccaatcgagagctaccaccagtcccccggctattctaagaccagcctgtgccagatcgataaggcccctatctacctgaaaaccaaggtgccacagaagtccacaaagtctctgaacatcggcaccgccttccacgaggctatggagggcgtgtttaaggacaagtatgtggtgcaccccgatcctggcgtgaataagaccacaaagtcttggaaggacttcgtgaagaggtatcctaagcacatgccactgaagcgcagcgagtacgaccaggtgctggccatgtacgatgccgcccggtcttatagaccttttcagaagtaccacctgagccggggcttctacgagagctccttttattggcacgatgccgtgacaaacagcctgatcaagtgcagacccgactatatcacccctgatggcatgagcgtgatcgacttcaagaccacagtggaccccagccccaagggctttcagtaccaggcctacaagtatcactactacgtgagcgccgccctgaccctggagggaatcgaggcagtgaccggcatcaggccaaaggagtacctgttcctggccgtgtccaattctgccccatacctgaccgccctgtatcgcgcctctgagaaggagatcgccctgggcgaccactttatccggcggagcctgctgaccctgaaaacctgtctggagtctggcaagtggcccggcctgcaggaggagatcctggagctgggcctgcctttctccggcctgaaggagctgagagaggagcaggaggtggaggatgagtttatggagctggtgggc

SEQ ID NO: 111 - DNA, 669 bp, Photobacterium sp. JCM 19050
aacaccgacatgatcgccatgcccccttctccagccatcagcatgctggacacaagcaagctggatgtgatggtgcgggcagcagagctgatgtcccaggccgtggtcatggtgcccgaccacttcaagggcaagccagccgattgcctggcagtggtcatgcaggcagaccagtggggcatgaacccctttaccgtggcccagaaaacccacctggtgagcggcaccctgggatacgagtcccagctggtgaatgccgtgatcagctcctctaaggccatcaagggccggttccactatgagtggtctgatggctgggagagactggccggcaaggtgcagtacgtgaaggagtctcggcagagaaagggccagcagggcagctatcaggtgaccgtggccaagccaacatggaagccagaggacgagcagggcctgtgggtgcggtgtggagccgtgctggccggagagaaggacatcacatggggccctaagctgtacctggccagcgtgctggtgcggaacagcgagctgtggaccacaaagccctaccagcaggccgcctataccgccctgaaggattggtcccgcctgtatacacctgccgtgatgcagggctctatgaccggcaagagctggtccctgacaggcaggctgatcagcccccgc

SEQ ID NO: 112 - DNA, 663 bp, Photobacterium sp. JCM 19050
gccgagcgggtgagaacctatcagcgggacgccgtgttcgcacacgagctgaaggccgagtttgatgaggccgtggagaacggcaagaccggcgtgacactggaggaccaggccagggccaagaggatggtgcacgaggccaccacaaaccccgcctctcggaattggttcagatacgacggagagctggccgcatgcgagaggagctatttttggcgcgatgaggaggcaggcctggtgctgaaggccaggcctgacaaggagatcggcaacaatctgatcgatgtgaagtccatcgaggtgccaaccgacgtgtgcgcctgtgatctgaacgcctatatcaatcggcagatcgagaagagaggctaccacatctccgccgcccactatctgtctggcacaggcaaggaccgcttcttttggatcttcatcaataaggtgaagggctacgagtgggtggcaatcgtggaggcctctcccctgcacatcgagctgggcacctatgaggtgctggagggcctgcggagcatcgccagctccacaaaggaggcagattacccagcacctctgtcccaccctgtgaacgagagaggcatcccacagcccctgatgtctaatctgagcacatacgccatgaagaggctggagcagtttcgcgagctg

SEQ ID NO: 113 - DNA, 693 bp, Providencia alcalifaciens DSM 30120
aaggcacagctggccgccgccctgcctaagcacatcaccagcgaccggatgatcagaatcgtgtccaccgagatcagaaagaccccatctctggccaactgcgacatccagagcttcatcggcgccgtggtgcagtgttctcagctgggcctggagccaggcaacgccctgggacacgcctacctgctgccctttggcaatggcaagtccgacaacggcaagtctaatgtgcagctgatcatcggctatcggggcatgatcgatctggcccggagaagcggccagatcatctctatcagcgccaggaccgtgcgccagggcgacaacttccactttgagtacggcctgaacgagaatctgacccacatccccgagggcaatgaggactcccctatcacacacgtgtacgcagtggcacggctgaaggatgagggcgtgcagttcgaagtgatgacatataaccagatcgagaaggtgagagatagctccaaggccggcaagaatggcccctgggtgacccactgggaggagatggccaagaaaaccgtgatcaggcgcctgtttaagtacctgcccgtgagcatcgagatgcagaaggccgtgatcctggacgagaaggccgaggccaatatcgagcaggatcactccgccatcttcgaggccgagtttgaggaggtggactctaacggcaat

SEQ ID NO: 114 - DNA, 828 bp, Providencia alcalifaciens DSM 30120
aacgagggcatctactatgacatctctaatgaggactatcaccacggcctgggcatctctaagagccagctggatctgatcgacgagagccccgccgatttcatctggcaccgggatgcccctgtggacaacgagaaaaccaaggccctggattttggcacagccctgcactgcctgctgctggagccagacgagttccagaagaggtttcgcatcgcccccgaggtgaaccggagaacaaatgccggcaaggagcaggagaaggagttcctggagatgtgcgagaaggagaatatcacccccatcacaaacgaggataataggaagctgtctctgatgaaggacagcgcaatggcccaccctatcgcccgctggtgtctggaggccaagggcatcgccgagagctccatctattggaaggacaaggatacagacatcctgtgccggtgtagaccagacaagctgatcgaggagcaccactggctggtggatgtgaagtccaccgccgacatccagaagttcgagcggtctatgtacgagtatagataccacgtgcaggattccttttattctgacggctacaagagcctgacaggcgagatgcccgtgttcgtgttcctggccgtgtccaccgtgatcaactgcggcagataccccgtgcgggtgttcgtgctggacgagcaggcaaagtccgtgggacggatcacctataagcagaatctgtttacatacgccgagtgtctgaaaaccgacgagtgggccggcatcagaaccctgagcctgccctcctgggcaaaggagctgaagcacgagcacaccacagcctct

SEQ ID NO: 115 - protein, 271 aa, Pantoea stewartii
MSNQPPIASADLQKANTGKQVANKTPEQTLVGFMNQPAMKSQLAAALPRHMTADRMIRIVTTEIRKTPALATCDQSSFIGAVVQCSQLGLEPGSALGHAYLLPFGNGRSKSGQSNVQLIIGYRGMIDLARRSGQIVSLSARVVRADDEFSFEYGLDENLIHRPGENEDAPITHVYAVARLKDGGTQFEVMTVKQIEKVKAQSKASSNGPWVTHWEEMAKKTVIRRLFKYLPVSIEMQKAVILDEKAESDVDQDNASVLSAEYSVLDGSSEE

SEQ ID NO: 116 - protein, 272 aa, Pantoea stewartii
MQPGVYYDISNEEYHAGPGISKSQLDDIAVSPAIFQWRKSAPVDDEKTAALDLGTALHCLLLEPDEFSKRFMIGPEVNRRTNAGKQKEQDFLDMCEQQGITPITHDDNRKLRLMRDSAFAHPVARWMLETEGKAEASIYWNDRDTQILSRCRPDKLITEFSWCVDVKSTADIGKFQKDFYSYRYHVQDAFYSDGYEAQFCEVPTFAFLVVSSSIDCGRYPVQVFIMDQQAKDAGRAEYKRNLTTYAECQARNEWPGIATLSLPYWAKEIRNV

SEQ ID NO: 117 - protein, 273 aa, Pantoea brenneri; identical to SEQ ID NO: 10

SEQ ID NO: 118 - protein, 272 aa, Pantoea brenneri; identical to SEQ ID NO: 4

SEQ ID NO: 119 - protein, 270 aa, Pantoea dispersa
MSNQPPLATADLQKTQQSNQVAKTPEQTLVGFMNQPAMKSQLAAALPRHMTADRMIRIVTTEIRKTPALAQCDQSSFIGAVVQCSQLGLEPGSALGHAYLLPFGNGRSKSGQSNVQLIIGYRGMIDLARRSGQIVSLSARVVRADDEFSFEYGLDENLIHRPGDNESAPITHVYAVARLKDGGTQFEVMTAKQVEKVKAQSKASSNGPWVTHWEEMAKKTVIRRLFKYLPVSIEMQKAVVLDEKAESDVDQDNASVLSAEYSVLESGTGE

SEQ ID NO: 120 - protein, 272 aa, Pantoea dispersa
MEPGIYYDISNEAYHSGPGISKSQLDDIARSPAIFQWRKDAPVDTEKTKALDLGTDFHCAVLEPERFADMYRVGPEVNRRTTAGKAEEKEFFEKCEKDGAVPITHDDARKVELMRGSVMAHPIAKQMIAAQGHAEASIYWHDESTGNLCRCRPDKFIPDWNWIVDVKTTADMKKFRREFYDLRYHVQDAFYTDGYAAQFGERPTFVFVVTSTTIDCGRYPTEVFFLDEETKAAGRSEYQSNLVTYSECLSRNEWPGIATLSLPHWAKELRNV

SEQ ID NO: 121 - protein, 271 aa, Type-F symbiont of Plautia stali; identical to SEQ ID NO: 11

SEQ ID NO: 122 - protein, 272 aa, Type-F symbiont of Plautia stali; identical to SEQ ID NO: 5

SEQ ID NO: 123 - protein, 268 aa, Providencia stuartii
MSNPPLAQADLQKTQGTEVKEKTKDQMLVELINKPSMKAQLAAALPRHMTPDRMIRIVTTEIRKTPALATCDMQSFVGAVVQCSQLGLEPGNALGHAYLLPFGNGKSKSGQSNVQLIIGYRGMIDLARRSGQIVSISARTVRQGDNFHFEYGLNENLTHVPGENEDSPITHVYAVARLKDGGVQFEVMTYNQIEKVRASSKAGQNGPWVSHWEEMAKKTVIRRLFKYLPVSIEMQKAVILDEKAEANIDQENATIFEGEYEEVGTDGK

SEQ ID NO: 124 - protein, 270 aa, Providencia stuartii
EGIYYNISNEDYHNGLGISKSQLDLINEMPAEYIWSKEAPVDEEKIKPLEIGTALHCLLLEPDEYHKRYKIGPDVNRRTNAGKEKEKEFFDMCEKEGITPITHDDNRKLMIMRDSALAHPIAKWCLEADGVSESSIYWTDKETDVLCRCRPDRIITAHNYIVDVKSSGDIEKFDYEYYNYRYHVQDAFYSDGYKEVTGITPTFLFLVVSTKIDCGKYPVRTYVMSEEAKSAGRTAYKHNLLTYAECLKTDEWAGIRTLSLPRWAKELRNE

SEQ ID NO: 125 - protein, 268 aa, Providencia sp. MGF014; identical to SEQ ID NO: 12

SEQ ID NO: 126 - protein, 272 aa, Providencia sp. MGF014; identical to SEQ ID NO: 6

SEQ ID NO: 127 - protein, 238 aa, Shewanella putrefaciens
MQTAQVKLSVPHQQVYQDNFNYLSSQVVGHLVDLNEEIGYLNQIVFNSLSTASPLDVAAPWSVYGLLLNVCRLGLSLNPEKKLAYVMPSWSETGEIIMKLYPGYRGEIAIASNFNVIKNANAVLVYENDHFRIQAATGEIEHFVTSLSIDPRVRGACSGGYCRSVLMDNTIQISYLSIEEMNAIAQNQIEANMGNTPWNSIWRTEMNRVALYRRAAKDWRQLIKATPEIQSALSDTEY

SEQ ID NO: 128 - protein, 392 aa, Shewanella putrefaciens
MGTALAQTISLDWQDTIQPAYTASGKPNFLNAQGEIVEGIYTDLPNSVYHALDAHSSTGIKTFAKGRHHYFRQYLSDVCRQRTKQQEYTFDAGTYGHMLVLEPENFHGNFMRNPVPDDFPDIELIESIPQLKAALAKSNLPVSGAKAALIERLYAFDPSLPLFEKMREKAITDYLDLRYAKYLRTDVELDEMATFYGIDTSQTREKKIEEILAISPSQPIWEKLISQHVIDHIVWDDAMRVERSTRAHPKADWLISDGYAELTIIARCPTTGLLLKVRFDWLRNDAIGVDFKTTLSTNPTKFGYQIKDLRYDLQQVFYCYVANLAGIPVKHFCFVATEYKDADNCETFELSHKKVIESTEEMFDLLDEFKEALTSGNWYGHDRSRSTWVIEV

SEQ ID NO: 129 - protein, 297 aa, Bacillus sp. MUM 116
MSKQLTTVNTQAVVGTFSQAELDTLKQTIAKGTTNEQFALFVQTCANSRLNPFLNHIHCIVYNGKEGATMSLQIAVEGILYLARKTDGYKGIECQLIHENDEFKFDAKSKEVDHQIGFPRGNVIGGYAIAKREGFDDVVVLMESNEVDHMLKGRNGHMWRDWFNDMFKKHIMKRAAKLQYGIEIAEDETVSSGPSVDNIPEYKPQPRKDITPNQDVIDAPPQQPKQDDEAAKLKAARSEVSKKFKKLGIVKEDQTEYVEKHVPGFKGTLSDFIGLSQLLDLNIEAQEAQSADGDLLD

SEQ ID NO: 130 - protein, 442 aa, Bacillus sp. MUM 116
MTYAADETLVQLLLSVDGKQLLLGRGLKKGKAQYYINEVPSKAKEFEEIRDQLFDKDLFMSLFNPSYFFTLHWEKQRAMMLKYVTAPVSKEVLKNLPEAQSEVLERYLKKHSLVDLEKIHKDNKNKQDKAYISAQSRTNTLKEQLMQLTEEKLDIDSIKAELAHIDMQVIELEKQMDTAFEKNQAFNLQAQIRNLQDKIEMSKERWPSLKNEVIEDTCRTCKRPLDEDSVEAVKADKDNRIAEYKAKHNSLVSQRNELKEQLNTIEYIDVTELREQIKELDESGQPLREQVRIYSQYQNLDTQVKSAEADENGILQDLKASIFILDSIKAFRGKEAEMQAEKVQALFTTLSVRLFKQNKGDGEIKPDFEIEMNDKPYRTLSLSEGIRAGLELRDVLSQQSELVTPTFVDNAESITSFKQPNGQLIISRVVAGQELKIEAVSE

SEQ ID NO: 131 - protein, 269 aa, Shigella sonnei; identical to SEQ ID NO: 13

SEQ ID NO: 132 - protein, 648 aa, Shigella sonnei; identical to SEQ ID NO: 7

SEQ ID NO: 133 - protein, 277 aa, Salmonella enterica
MTKQPPIAKADLQKTQGNRAPAAVNDKDVLCVINSPAMKAQLAAALPRHMTAERMIRIATTEIRKVPELRNCDSTSFIGAIVQCSQLGLEPGSALGHAYLLPFGNGKAKNGKKNVQLIIGYRGMIDLARRSGQIISLSARVVRECDEFSYELGLDEKLVHRPGENEDAPITHVYAVAKLKDGGVQFEVMTKKQVEKVRDTHSKAAKNAASKGASSIWDEHFEDMAKKTVIRKLFKYLPVSIEIQRAVSMDGKEVETINPDDISVIAGEYSVIDNPEE

SEQ ID NO: 134 - protein, 648 aa, Salmonella enterica
DRGLLTKEWRKGNRVSRITRTASGANAGGGNLTDRGEGFVHDLTSLARDVATGVLARSMDVDIYNLHPAHAKRVEEIIAENKPPFSVFRDKFITMPGGLDYSRAIVVASVKEAPIGIEVIPAHVTEYLNKVLTETDHANPDPEIVDIACGRSSAPMPQRVTEEGKQDDEEKPQPSGAMADEQATAETVEPNATEHHQNTQPLDAQSQVNSVDAKYQELRAELQEARKNIPSKNPVDADKLLAASRGEFVDGISDPNDPKWVKGIQTRDSVYQNQPETEKISPDAKQPEPVVQQEPETVCNACGQTGGDNCPDCGAVMGDATYQETFGEENQVEAKEKDPEEMEGAEHPHNENAGSDPHRDCSDETGEVADPVIVEDIEPGIYYGISNENYHAGPGVSKSQLDDIADTPALYLWRKNAPVDTTKTKTLDLGTAFHCRVLEPEEFSNRFIVAPEFNRRTNAGKEEEKAFLMECASTGKTVITAEEGRKIELMYQSVMALPLGQWLVESAGHAESSIYWEDPETGILCRCRPDKIIPEFHWIMDVKTTADIQRFKTAYYDYRYHVQDAFYSDGYEAQFGVQPTFVFLVASTTVECGRYPVEIFMMGEEAKLAGQQEYHRNLRTLADCLNTDEWPAIKTLSLPRWAKEYAND

SEQ ID NO: 135 - protein, 332 aa, Acetobacter
MNAPQKQNTRAAVKKISPQEFAEQFAAIIPQVKSVLPAHVTFEKFERVVRLAVRKNPDLLTCSPASLFMACIQAASDGLLPDGREGAIVSRWSSKKSCNEASWMPMVAGLMKLARNSGDIASISSQVVFEGEHFRVVLGDEERIEHERDLGKTGGKIVAAYAVARLKDGSDPIREIMSWGQIEKIRNTNKKWEWGPWKAWEDEMARKTVIRRLAKRLPMSTDKEGERLRSAIERIDSLVDISANVDAPQIAADDEFAAAAHGVEPQQIAAPDLIGRLAQMQSLEQVQDIEPQVSHAIQEADKRGDSDTANALDAALQSALSRTSTAKEEVPA

SEQ ID NO: 136 - protein, 300 aa, Acetobacter
MVISKSGIYDLTNEQYHADPCPEMSLSSSGARDLLSSCPAKFIAAKQLPQQNKRCFDIGSAGHLMVLEPHLFDQKVCEIKHPDWRTKAAKEERDAAYAEGRIPLLSREVEDIRAMHSVVWRDSLGARAFSGGKAEQSLVWRDEEFGIWCRLRPDYVPNNAVRIFDYKTATNGSPDAFMKEIYNRGYHQQAAWYLDGYEAVTGHRPREFWFVVQEKTAPFLLSFFQMDEMSLEIGRTLNRQAKGIFAWCLRNNCWPGYQPEVDGKVRFFTTSPPAWLVREYEFKNEHGAYEPPEIKRKEVA

SEQ ID NO: 137 - protein, 276 aa, Salmonella enterica subsp. enterica serovar Javiana str. 10721
MPKQPPIAKADLQKTQGARTPTAVKNNNDVISFINQPSMKEQLAAALPRHMTAERMIRIATTEIRKVPALGDCDTMSFVSAIVQCSQLGLEPGGALGHAYLLPFGNRNEKSGKKNVQLIIGYRGMIDLARRSGQIASLSARVVREGDDFSFEFGLEEKLVHRPGENEDAPVTHVYAVARLKDGGTQFEVMTRKQIELVRAQSKAGNNGPWVTHWEEMAKKTAIRRLFKYLPVSIEIQRAVSMDEKETLTIDPADASVITGEYSVVENAGVEENVTA

SEQ ID NO: 138 - protein, 268 aa, Salmonella enterica subsp. enterica serovar Javiana str. 10721
MYYDIPNEAYHAGPGVSKSQLDDIADTPAIYLWRKNAPVDTEKTKSLDTGTAFHCRVLEPEEFSKRFIIAPEFNRRTSAGKEEEKTFLEECTRTGRTVLTAEEGRKIELMYQSVMALPLGQWLVESAGYAESSVYWEDPETGILCRCRPDKIIPEFHWIMDVKTTADIQRFRTAYYDYRYHVQDAFYSDGYRAQFGEIPTFVFLVASTTAECGRYPVEIFMMGEDAKLAGQREYRRNLQTLAECLNNDEWPAIKTLSLPRWAKENANA

SEQ ID NO: 139 - protein, 242 aa, Pseudobacteriovorax antillogorgiicola; identical to SEQ ID NO: 14

SEQ ID NO: 140 - protein, 300 aa, Pseudobacteriovorax antillogorgiicola; identical to SEQ ID NO: 8

SEQ ID NO: 141 - protein, 224 aa, Photobacterium sp. JCM 19050
MNTDMIAMPPSPAISMLDTSKLDVMVRAAELMSQAVVMVPDHFKGKPADCLAVVMQADQWGMNPFTVAQKTHLVSGTLGYESQLVNAVISSSKAIKGRFHYEWSDGWERLAGKVQYVKESRQRKGQQGSYQVTVAKPTWKPEDEQGLWVRCGAVLAGEKDITWGPKLYLASVLVRNSELWTTKPYQQAAYTALKDWSRLYTPAVMQGSMTGKSWSLTGRLISPR

SEQ ID NO: 142 - protein, 222 aa, Photobacterium sp. JCM 19050
MAERVRTYQRDAVFAHELKAEFDEAVENGKTGVTLEDQARAKRMVHEATTNPASRNWFRYDGELAACERSYFWRDEEAGLVLKARPDKEIGNNLIDVKSIEVPTDVCACDLNAYINRQIEKRGYHISAAHYLSGTGKDRFFWIFINKVKGYEWVAIVEASPLHIELGTYEVLEGLRSIASSTKEADYPAPLSHPVNERGIPQPLMSNLSTYAMKRLEQFREL

SEQ ID NO: 143 - protein, 232 aa, Providencia alcalifaciens DSM 30120
MKAQLAAALPKHITSDRMIRIVSTEIRKTPSLANCDIQSFIGAVVQCSQLGLEPGNALGHAYLLPFGNGKSDNGKSNVQLIIGYRGMIDLARRSGQIISISARTVRQGDNFHFEYGLNENLTHIPEGNEDSPITHVYAVARLKDEGVQFEVMTYNQIEKVRDSSKAGKNGPWVTHWEEMAKKTVIRRLFKYLPVSIEMQKAVILDEKAEANIEQDHSAIFEAEFEEVDSNGN

SEQ ID NO: 144 - protein, 277 aa, Providencia alcalifaciens DSM 30120
MNEGIYYDISNEDYHHGLGISKSQLDLIDESPADFIWHRDAPVDNEKTKALDFGTALHCLLLEPDEFQKRFRIAPEVNRRTNAGKEQEKEFLEMCEKENITPITNEDNRKLSLMKDSAMAHPIARWCLEAKGIAESSIYWKDKDTDILCRCRPDKLIEEHHWLVDVKSTADIQKFERSMYEYRYHVQDSFYSDGYKSLTGEMPVFVFLAVSTVINCGRYPVRVFVLDEQAKSVGRITYKQNLFTYAECLKTDEWAGIRTLSLPSWAKELKHEHTTAS

SEQ ID NO: 145 - RNA, 26 nt, artificial sequence
  Synthetic
aacaugaggaucacccaugucugcag

SEQ ID NO: 146 - RNA, 26 nt, artificial sequence
  Synthetic
agcaugaggaucacccaugucugcag

SEQ ID NO: 147 - RNA, 26 nt, artificial sequence
  Synthetic
agcgugaggaucacccaugccugcag

SEQ ID NO: 148 - protein, 37 aa, artificial sequence
  Synthetic
SASGGSSGGSSGSETPGTSESATPESSGGSSGGSGGS

SEQ ID NO: 149 - protein, 22 aa, artificial sequence
  Synthetic
GNARTRRRERRAEKQAQWKAAN

SEQ ID NO: 150 - RNA, 15 nt, artificial sequence
  Synthetic
gcccugaaaaagggc

SEQ ID NO: 151 - RNA, 15 nt, artificial sequence
  Synthetic
gcccugaagaagggc

SEQ ID NO: 152 - RNA, 15 nt, artificial sequence
  Synthetic
gcgcugaaaaagcgc

SEQ ID NO: 153 - RNA, 15 nt, artificial sequence
  Synthetic
gcccugacaaagggc

SEQ ID NO: 154 - RNA, 15 nt, artificial sequence
  Synthetic
gcgcugacaaagcgc

SEQ ID NO: 155 - protein, 21 aa, artificial sequence
  Synthetic
GNAKTRRHERRRKLAIERDTI

SEQ ID NO: 156 - RNA, 15 nt, artificial sequence
  Synthetic
gcgcugacaaagcgc

SEQ ID NO: 157 - RNA, 15 nt, artificial sequence
  Synthetic
ccgccgacaacgcgg

SEQ ID NO: 158 - RNA, 17 nt, artificial sequence
  Synthetic
ugcgcugacaaagcgcg

SEQ ID NO: 159 - RNA, 17 nt, artificial sequence
  Synthetic
accgccgacaacgcggu

SEQ ID NO: 160 - DNA, 200 bp, artificial sequence
  Synthetic
caccttcagattttcctgtaacgatcgggaactggcatcttcagggagtagctgacctcttctcttcctcccacaggatcctggagccacccgcagttcgaaaagctcagtgaagagaagaacaaaaagcagcatattacagttagttgtcttcatcaatctttaaatatgttgtgtggtttttctctccctgtttccac

SEQ ID NO: 161 - DNA, 200 bp, artificial sequence
  Synthetic
gtggaaacagggagagaaaaaccacacaacatatttaaagattgatgaagacaactaactgtaatatgctgctttttgttcttctcttcactgagcttttcgaactgcgggtggctccaggatcctgtgggaggaagagaagaggtcagctactccctgaagatgccagttcccgatcgttacaggaaaatctgaaggtg

SEQ ID NO: 162 - DNA, 121 bp, artificial sequence
  Synthetic
actttgagtgtagcagagaggaaccattgccaccttcagattttcctgtaacgatcgggaactggcatcttcagggagtagctgacctcttctcttcctcccacaggatcctggagccacc

SEQ ID NO: 163 - DNA, 54 bp, artificial sequence
  Synthetic
tccaccccacagtggggcaagcttctgacctcttctcttcctcccacagggcct

SEQ ID NO: 164 - DNA, 23 bp, artificial sequence
  Synthetic
  misc_feature (21)..(21): n is a, c, g, or t
ttgacctgcagtccagcctangg

SEQ ID NO: 165 - DNA, 23 bp, artificial sequence
  Synthetic
  misc_feature (21)..(21): n is a, c, g, or t
ccaccgcaaatgcttctaggngg

(19) United States
(12) Patent Application Publication
(10) Pub. No.: US 2023/0091242 A1
(43) Pub. Date: Mar. 23, 2023

(54) RNA-GUIDED GENOME RECOMBINEERING AT KILOBASE SCALE

(71) Applicant: The Board of Trustees of the Leland Stanford Junior University, Stanford, CA (US)

(72) Inventor: Le Cong, Stanford, CA (US)

(21) Appl. No.: 17/905,457

(22) PCT Filed: Mar. 2, 2021
(86) PCT No.: PCT/US2021/020513

§ 371 Date: Sep. 1, 2022

Related U.S. Application Data
(60) Provisional application No. 62/984,618, filed on Mar. 3, 2020.
(60) Provisional application No. 63/146,447, filed on Feb. 5, 2021.

(51) Int. Cl. C12N 9/22; C12N 15/11; C12N 15/62; C12N 15/115; C12N 15/90; C07K 14/245

(52) U.S. Cl. CPC C12N 9/22; C12N 15/11; C12N 15/625; C12N 15/115; C12N 15/907; C07K 14/245; C12N 2310/20; C12N 2800/80; C12N 2310/16; C07K 2319/09

(57) ABSTRACT

The present disclosure provides recombineering-editing systems using CRISPR and recombination enzymes as well as methods, vectors, nucleic acid compositions, and kits thereof. The methods and systems provide means for altering target DNA, including genomic DNA in a host cell.

### CROSS-REFERENCE TO RELATED APPLICATIONS

This application claims the benefit of U.S. Provisional Application No. 62/984,618, filed Mar. 3, 2020, and U.S. Provisional Application No. 63/146,447, filed Feb. 5, 2021, the contents of each are incorporated herein by reference.

### FIELD

The present invention relates to RNA-guided recombineering-editing systems using phage recombination enzymes as well as methods, vectors, nucleic acid compositions, and kits thereof.

### BACKGROUND

The Clustered Regularly Interspaced Short Palindromic Repeats (CRISPR) system, originally found in bacteria and archaea as part of the immune system to defend against invading viruses, forms the basis for genome editing technologies that can be programmed to target specific stretches of a genome or other DNA for editing at precise locations. While various CRISPR-based tools are available, the majority are geared towards editing short sequences. Long-sequence editing is highly sought after in the engineering of model systems, therapeutic cell production and gene therapy. Prior studies have developed technologies to improve Cas9-mediated homology-5 directed repair (HDR), and tools leveraging nucleic acid modification enzymes with Cas9, e.g., prime-editing, demonstrated editing up to 80 base-pairs (bp) in length. Despite these progresses, there are continued demands for large-scale mammalian genome engineering with high efficiency and fidelity.

### SUMMARY

Provided herein are systems and methods that facilitate nucleic acid editing in a manner that allows large-scale nucleic acid editing with high accuracy and low off-target errors. These systems and methods employ a combination of microbial recombination components with CRISPR recombination components.

For example, disclosed herein are systems comprising a protein, a nucleic acid molecule comprising a guide RNA sequence that is complementary to a target DNA sequence, and a microbial recombination protein. The microbial recombination protein may be, for example, RecE, RecT, lambda exonuclease (Exo), Bet protein (betA, redB), exonuclease gp6, single-stranded DNA-binding protein gp2.5, or a derivative or variant thereof. In some embodiments, the system further comprises donor DNA. In some embodiments, the target DNA sequence is a genomic DNA sequence in a host cell.

In some embodiments, the system further comprises a recruitment system comprising at least one aptamer sequence and an aptamer binding protein functionally linked to the microbial recombination protein as part of a fusion protein. In some embodiments, the aptamer sequence is an RNA aptamer sequence or a peptide aptamer sequence. In some embodiments, the RNA aptamer sequence is part of the nucleic acid molecule. In some embodiments, the nucleic acid molecule comprises two RNA aptamer sequences. In some embodiments, the microbial recombination protein is functionally linked to the aptamer binding protein as a fusion protein. In some embodiments, the binding protein comprises a MS2 coat protein, a lambda N22 peptide, or a functional derivative, fragment, or variant thereof. In some embodiments, the fusion protein further comprises a linker and/or a nuclear localization sequence.

Disclosed herein are compositions comprising a nucleic acid sequence encoding a fusion protein comprising a microbial recombination protein functionally linked to an aptamer binding protein. The microbial recombination protein may be RecE, RecT, lambda exonuclease (Exo), Bet protein (betA, redB), exonuclease gp6, single-stranded DNA-binding protein gp2.5, or a derivative or variant thereof. The compositions may further comprise one or both of a polynucleotide comprising a nucleic acid sequence encoding a Cas protein and a nucleic acid molecule comprising a guide RNA sequence that is complementary to a target DNA sequence. In some embodiments, the nucleic acid molecule further comprises at least one RNA aptamer sequence. In some embodiments, the polynucleotide comprising a nucleic acid sequence encoding a Cas protein further comprises a sequence encoding at least one peptide aptamer sequence.

Also disclosed herein are vectors comprising a nucleic acid sequence encoding a fusion protein comprising a microbial recombination protein functionally linked to an aptamer binding protein. The microbial recombination protein may be RecE, RecT, lambda exonuclease (Exo), Bet protein (betA, redB), exonuclease gp6, single-stranded DNA-binding protein gp2.5, or a derivative or variant thereof. The vectors may further comprise one or both of a polynucleotide comprising a nucleic acid sequence encoding a Cas protein and a nucleic acid molecule comprising a guide RNA sequence that is complementary to a target DNA sequence. In some embodiments, the nucleic acid molecule further comprises at least one RNA aptamer sequence. In some embodiments, the polynucleotide comprising a nucleic acid sequence encoding a Cas protein further comprises a sequence encoding at least one peptide aptamer sequence.

In some embodiments, the RecE and RecT recombination protein is derived from E. coli. In some embodiments, the RecE, or derivative or variant thereof, comprises an amino acid sequence with at least 70% similarity to amino acid sequences selected from the group consisting of SEQ ID NOs: 1-8. In some embodiments, the RecT, or derivative or variant thereof, comprises an amino acid sequence with at least 70% similarity to amino acid sequences selected from the group consisting of SEQ ID NO: 9.

In some embodiments, the Cas protein is Cas9 or Cas12a. In some embodiments, the Cas protein is a catalytically dead. In some embodiments, the Cas9 protein is wild-type Streptococcus pyogenes Cas9 or a wild-type Staphylococcus aureus Cas9. In some embodiments, the Cas9 protein is a Cas9 nickase (e.g., wild-type Streptococcus pyogenes Cas9 with an amino acid substation at position 10 of D10A).

Also disclosed is a eukaryotic cell comprising the systems or vectors disclosed herein.

Further disclosed herein are methods of altering a target genomic DNA sequence in a host cell. The methods comprise contacting the systems, compositions, or vectors described herein with a target DNA sequence (e.g., introducing the systems, compositions, or vectors described herein into a host cell comprising a target genomic DNA sequence). Kits containing one or more reagents or other components useful, necessary, or sufficient for practicing any of the methods are also disclosed herein.

Other aspects and embodiments of the disclosure will be apparent in light of the following detailed description and accompanying figures.

### BRIEF DESCRIPTION OF THE DRAWINGS

FIG. 1A and FIG. 1B are the reconstructed RecE (FIG. 1A) and RecT (FIG. 1B) phylogenetic trees with eukaryotic recombination enzymes from yeast and human.

FIG. 2A is a phylogenetic tree and length distribution of RecE/RecT homologs. FIG. 2B is the metagenomics distribution of RecE/T. FIG. 2C is a schematic showing central models disclosed herein. FIG. 2D are graphs of the genome knock-in efficiency of RecE/T homologs.

FIGS. 3A and 3B are graphs of the high-throughput sequencing (HTS) reads of homology directed repair (HDR) at the EMX1 (FIG. 3A) locus and the VEGFA (FIG. 3B) locus. FIGS. 3C-3D are graphs of the mKate knock-in efficiency at HSP90AA1 (FIG. 3C), DYNLT1 (FIG. 3D), and AAVS1 (FIG. 3E) loci in HEK293T cells. FIG. 3F is images of mKate knock-in efficiency in HEK293T cells with RecT. FIG. 3G is a schematic of an exemplary AAVS1 knock-in strategy and chromatogram trace from RecT knock-in group. FIG. 3H is schematics and graphs of the recruitment control experiment and corresponding knock-in efficiency. All results are normalized to NR. (NC, no cutting; NR, no recombinator).

FIGS. 4A-4C are graphs of the relative mKate knock-in efficiencies to the NE group at HSP90AA1 (FIG. 4A), DYNLT1 (FIG. 4B), and AAVS1 (FIG. 4C) loci in HEK293T cells. (NC, no cutting control group. NR, no recombinator control group.) FIG. 4D is an image of an exemplary agarose gel of junction PCR that validates mKate knock-in at AAVS1 locus. FIGS. 4E and 4F are graphs of the absolute and (FIG. 4E) and relative (FIG. 4F) LOV knock-in efficiencies at AAVS1 locus.

FIGS. 5A-5D are graphs of the genomic knock-in efficiencies at different loci across cell lines A549 (FIG. 5A), HepG2 (FIG. 5B), HeLa (FIG. 5C), and hESCs (H9) (FIG. 5D). FIG. 5E is images of mKate knock-ins in hESCs. FIGS. 5F and 5G are genomic-wide off-target site (OTS) counts (FIG. 5F) and OTS chromosomal distribution (FIG. 5G) of REDITv1 tools.

FIGS. 6A-6D are graphs of the relative mKate knock-in efficiency at the AAVS1 locus and the DYNT1 locus in A549 cell line (FIG. 6A), the DYNLT1 locus and the HSP90AA1 locus in HepG2 cell line (FIG. 6B), the DYNLT1 locus and the HSP90AA1 locus in Hela cell line (FIG. 6C), and the HSP90AA1 locus and the OCT4 locus in hES-H9 cell line (FIG. 6D). (NC, no cutting control group. NR, no recombinator control group. All data normalized to NR group.) FIG. 6E is representative FACS results of HSP90AA1 mKate knock-in in hES-H9 cells.

FIGS. 7A-7D are graphs of the absolute mKate knock-in efficiencies of different homology arm lengths at the DYNLT1 (FIG. 7A) and HSP90AA1 (FIG. 7B) loci and the no recombinator controls for DYNLT1 (FIG. 7C) and HSP90AA1 (FIG. 7D).

FIGS. 8A-8E are graphs of the indel rates of the top 3 predicted off-target loci associated with sgEMX1 (FIGS. 8A-8C) or sgVEGFA (FIGS. 8D-8E) in the REDITv1 system.

FIG. 9A is a schematic of select embodiments of REDITv2N and corresponding knock-in efficiencies in HEK293 T cells. FIGS. 9B and 9C are graphs of genomic-wide off-target site (OTS) counts (FIG. 9B) and OTS chromosomal distribution (FIG. 9C) comparing REDITv2N against REDITv1. FIG. 9D is a schematic of select embodiments of REDITv2D and corresponding knock-in efficiencies. FIG. 9E is a graph of editing efficiency of REDITv1, REDITv2N, and REDITv2D under serum starvation conditions. FIG. 9F is the knock-in efficiencies of REDITv3 in hESCs. FIG. 9G is images of mKate knock in using REDITv3 in hESCs.

FIGS. 10A and 10B are schematics and graphs of the relative mKate knock-in efficiencies of select embodiments of REDITv2N (FIG. 10A) and REDITv2D (FIG. 10B) at the DYNLT1 locus and the HSP90AA1 locus.

FIGS. 11A-11D are images of agarose gels showing junction PCR of mKate knock-in at the DYNLT1 locus and the HSP90AA1 locus for a select REDITv2N system.

FIGS. 12A and 12B are graphs of the genomic distribution of detected off-target cleavages of select embodiments of REDITv2 (FIG. 12A) and REDITv2N (FIG. 12B). A pileup includes alignments that have two or more reads overlapping with each other. Flanking pairs include alignments that show up on opposite strands within 200 bp upstream of each other. Target matched includes alignments that match to a treated target in the upstream sequence (up to 6 mismatches, including 1 mismatch in the PAM, are allowed in the target sequence). FIG. 12C is a graph of the HTS HDR and indel reads at EMX1 locus for REDITv2N system.

FIG. 13A is an image of an agarose gel showing junction PCR of mKate knock-ins at the DYNLT1 locus for REDITv2D system.

FIGS. 14A-14C are graphs of the mKate knock-in efficiencies at the HSP90AA1 locus in REDITv2 (FIG. 14A), REDITv2N (FIG. 14B) and REVITv2D (FIG. 14C) when treated with different FBS concentrations. FIGS. 14D-14F are graphs of the mKate knock-in efficiencies at the HSP90AA1 locus in REDITv2 (FIG. 14D), REDITv2N (FIG. 14E) and REVITv2D (FIG. 14F) when treated with different serum FBS concentrations.

FIG. 15 is images of the nuclear localization of RecE_587 and RecT following EGFP fusion to the REDITv1 systems. Nuclei were stained with NucBlue Live Ready Probes Reagent.

FIGS. 16A and 16B are the relative mKate knock-in efficiencies at HSP90AA1 and DYNLT1 loci following fusion of different nuclear localization sequences to either the N- or C-terminus of RecT and RecE_587. FIGS. 16C and 16D are graphs of the absolute mKate knock-in efficiencies of the constructs from FIGS. 16A and 16B for the DYNLT1 locus (FIG. 16C) and the HSP90AA1 locus (FIG. 16D).

FIGS. 17A-17D are graphs of the relative (FIGS. 17A and 17B) and absolute (FIGS. 17C and 17D) mKate knock-in efficiencies for the DYNLT1 locus (FIGS. 17A and 17C) and the HSP90AA1 locus (FIGS. 17B and 17D) following fusion new NLS sequences as well as optimal linkers to REDITv2 and REDITv3 variants. The REDITv2 versions using REDITv2N (D10A or H840A) and REDITv2D (dCas9) are indicated in the horizonal axis, along with the number of guides used. The different colors represent the different control groups and REDIT versions.

FIG. 18 is a graph of the relative editing efficiency of REDITv3N system at HSP90AA1 locus in hES-H9 cells.

FIG. 19A is a diagram of an exemplary saCas9 expression vector. FIGS. 19B-19E are graphs of the relative mKate knock-in efficiencies at the AAVS1 locus (FIG. 19D) and HSP90AA1 locus (FIG. 19E) of different effectors in saCas9 system and the respective absolute efficiencies (FIGS. 19B and 19C, respectively). NC, no cutting control group. NR, no recombinator control group.

FIG. 20A is a schematic of RecT truncations. FIGS. 20B and 20C are graphs of the relative mKate knock-in efficiencies at the DYNLT1 locus for wild-type Streptococcus pyogenes Cas9 and Streptococcus pyogenes Cas9n(D10A) with single- and double-nicking.

FIG. 21A is a schematic of RecE_587 truncations. FIGS. 21B and 21C are graphs of the relative mKate knock-in efficiencies at the DYNLT1 locus for wild-type Streptococcus pyogenes Cas9 and Streptococcus pyogenes Cas9n(D10A) with single- and double-nicking.

FIGS. 22A and 22B are graphs of comparison of efficiency to perform recombineering-based editing with various exonucleases (FIG. 22A) and single-strand DNA annealing protein (SSAP) (FIG. 22B) from naturally occurring recombineering systems, including NR (no recombinator) as negative control. The gene-editing activity was measured using mKate knock-in assay at genomic loci (DYNLT1 and HSP90AA1). The data shown are percentage of successful mKate knock-in using human HEK293 cells, each experiments were performed in triplicate (n=3).

FIGS. 23A-23E show a compact recruitment system using boxB and N22. The REDIT recombinator proteins were fused to N22 peptide and within the sgRNA was boxB, the short cognizant sequence of N22 peptide (FIG. 23A). FIGS. 23B-23E are graphs of the gene-editing efficiency using mKate knock-in assay, with wildtype SpCas9, with side-by-side comparisons to the MS2-MCP recruitment system. FIGS. 23B and 23D are absolute mKate knock-in efficiency at DYNLT1, HSP90AA1 loci and FIGS. 23C and 23E are relative efficiencies. The data shown are percentage of successful mKate knock-in using HEK293 human cells, each experiments were performed in triplicate (n=3).

FIGS. 24A-24C show a SunTag recruitment system. The REDIT recombinator proteins were fused to scFV antibody and the GCN4 peptide in tandem fashion (10 copies of GCN4 peptide separated by linkers) was fused to the Cas9 protein (FIG. 24A). An mKate knock-in experiment (FIG. 24B) with the DYNLT1 locus was used to measure the gene-editing knock-in efficiency (FIG. 24C). All data are measurements of gene-editing efficiency using mKate knock-in assay, with wildtype SpCas9. Absolute mKate knock-in efficiency at DYNLT1 are shown in the bottom right corner of each flow cytometry plot, where the control is without recombinator (NR), which included scFV fused to GFP protein as negative control, all experiments done in HEK293 human cells.

FIGS. 25A and 25B exemplify REDIT with a Cas12A system. A Cpf1/Cas12a based REDIT system via the SunTag recruitment design was created (FIG. 25A) for two different Cpf1/Cas12a proteins. Using the mKate knock-in assay, the efficiencies at two endogenous loci (DYNLT1 and AAS1) were measured. (FIG. 25B). Shown are absolute mKate knock-in efficiency as measured by mKate+ cell percentage using HEK293 human cells, each experiment was performed in triplicate (n=3), where the negative control is without recombinator (NR).

FIGS. 26A and 26B are the measurements of precision recombineering activity via mKate knock-in gene-editing assay using RecE and RecT homologs at the DYNLT1 locus (A) and the HSP90AA1 locus (B). Shown are absolute mKate knock-in efficiency as measured by mKate+ cell percentage using HEK293 human cells, each experiments were performed in triplicate (n=3), where the negative control is without recombinator (NR) and no cutting (NC). The original RecE and RecT from E. coli were also included as positive controls.

FIGS. 27A and 27B is a schematic showing the SunTag-based recruitment of SSAP RecT to Cas9-gRNA complex for gene-editing (FIG. 27A) and a graph quantifying the editing efficiencies of SunTag compared to MS2-based strategies (FIG. 27B).

FIGS. 28A-28C show comparisons of REDIT with alternative HDR-enhancing gene-editing approaches. FIG. 28A is schematics showing alternative HDR-enhancing approaches via fusing functional domains, CtIP or Geminin (Gem), to Cas9 protein (left) and when combined with REDIT (right). FIG. 28B is an alternative small-molecule HDR-enhancing approach through cell cycle control. Nocodazole was used to synchronize cells at the G2/M boundary (left) according to the timeline shown (right). FIG. 28C is comparisons of gene-editing efficiencies using REDIT and alternative HDR-enhancing tools, Cas9-HE (CtIP fusion), Cas9-Gem (Geminin fusion), and Nocodazole (noc), along with combination of REDIT with these methods (Cas9-HE/Cas9-Gem/noc+REDIT). Donor DNAs have 200+400 bp (DYNLT1) or 200+200 bp (HSP90AA1) of HAs. All assays performed with no donor, NTC and Cas9 (no enhancement) controls. #P<0.05, compared to REDIT; ##P<0.01, compared to REDIT.

FIGS. 29A-29D show template design guideline, junction precision, and capacity of REDIT gene-editing methods. FIG. 29A is graphs of a homology arm (HA) length test comparing different template designs of HDR donors (longer HAs) or NHEJ/MMEJ donors (zero/shorter HAs) using REDIT and Cas9 references. Top and bottom are two genomic loci tested using mKate knock-in assay. FIG. 29B is a design of an exemplary junction profiling assay through isolation of knock-in clones, followed by genomic PCR using primers (fwd, rev) binding outside donor to avoid template amplification. Paired Sanger sequencing of the PCR products reveal homologous and non-homologous edits at the 5′- and 3′-junctions. FIG. 29C is a graph of the percentage of colonies with indicated junction profiles from the Sanger sequencing of knock-in clones as in FIG. 29B. Editing methods and donor DNA are listed at the bottom (HA lengths indicated in bracket). FIG. 29D is a graph of knock-in efficiencies using a 2-kb cassette to insert dual-GFP/mKate tags to validate REDIT methods with Cas9. HA lengths of donor DNAs indicated at the bottom.

FIGS. 30A-30C show GISseq results indicating that REDIT is an efficient method with the ability to insert kilobase-length sequences with less unwanted editing events. FIG. 30A is a schematic showing the design, procedures, and analysis steps for GIS-seq to measure genome-wide insertion sites of the knock-in cassettes. High-molecular-weight (HMW) genomic DNA purification was needed to remove potential contamination from donor DNAs. Donor DNAs had 200 bp HAs each side. FIG. 30B is representative GIS-seq results showing plus/minus reads at on-target locus DYNLT1 The expected 2A-mKate knock-in site before the stop codon of the last exon are the center of the trimmed reads (reads clipped to remove 2A-mKate cassette). The template mutations help to avoid gRNA targeting and distinguish genomic and edited reads are labeled. FIG. 30C is a summary of top GIS-seq insertion sites comparing Cas9dn and REDITdn groups, showing the expected on-target insertion site (highlighted) and reduced number of identified off-target insertion sites when using REDITdn. (Left) DYNLT1 and (Right) ACTB loci with MLE calculated from the distribution of filtered and trimmed GIS-seq reads.

FIGS. 31A-31F show the dependence of REDIT gene-editing on endogenous DNA repair and applying REDIT methods for human stem cell engineering. FIG. 31A is a model showing the editing process and major repair pathways involved when using REDIT or Cas9 for gene-editing, the HDR pathway are highlighted for chemical perturbation (inhibition of RAD51). Donor DNAs with 200+200 bp HAs are used for all inhibitor experiments. FIGS. 31B and 31C are graphs showing the relative knock-inefficiency of REDIT tools compared with Cas9 reference treated with RAD51 inhibitor B02 and RI-1, or vehicle-treated, for the wtCas9-based REDIT and Cas9 (FIG. 31B) and for Cas9 nickase-based REDITdn and Cas9dn (FIG. 31C). All conditions were measured with 1-kb knock-in assay at two genomic loci (DYNLT1 and HSP90AA1). FIG. 31D are graphs of knock-in efficiencies in hESCs (H9) using REDIT and REDITdn tested across three genomic loci, compared with corresponding Cas9 and Cas9dn references. FIGS. 31E and 31F are flow cytometry plots of mKate knock-in results in hESCs using REDIT, REDITdn with Cas9, Cas9dn, and NTC controls. Donor DNAs in the hESC experiments have 200+200 bp HAs across all loci tested.

FIGS. 32A-32B show chemical perturbations to dCas9 REDIT. Gene editing efficiencies were determined when treated with mammalian DNA repair pathway inhibitors (Mirin, RI-1, and B02) with (FIG. 32A) and without (FIG. 32B) cell cycle inhibitor (Thy, doubly Thymidine) blocking. Statistical analyses are from t-test results with 1% FDR via a two-stage step-up method.

FIGS. 33A and 33B are schematics of the DNA components (gene-editing vectors and template DNA) and tail vein injection of mice, respectively.

FIGS. 34A-34C are results from the tail vein injection of mice with gene-editing vectors. FIG. 34A is a schematic and gel electrophoresis of PCR analysis of liver hepatocytes from the injected mice.

FIG. 34B is the Sanger sequencing results of the PCR amplicon (SEQ ID NO: 162). FIG. 34C is a schematic of next-generation sequencing and a graph of the quantification of knock-in junction errors.

FIGS. 35A and 35B are schematics of the DNA components (gene-editing and control vector) and adeno-associated virus (AAV) treatment, respectively. FIG. 35C is fluorescent images of lungs from AAV treated mice and graphs of corresponding quantitation of tumor number.

### DETAILED DESCRIPTION OF THE INVENTION

The present disclosure is directed to a system and the components for DNA editing. In particular, the disclosed system based on CRISPR targeting and homology directed repair by phage recombination enzymes. The system results in superior recombination efficiency and accuracy at a kilobase scale.

### 1. Definitions

To facilitate an understanding of the present technology, a number of terms and phrases are defined below. Additional definitions are set forth throughout the detailed description.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclature used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%. 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

A cell has been “genetically modified,” “transformed,” or “transfected” by exogenous DNA, e.g., a recombinant expression vector, when such DNA has been introduced inside the cell. The presence of the exogenous DNA results in permanent or transient genetic change. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones that comprise a population of daughter cells containing the transforming DNA. A “clone” is a population of cells derived from a single cell or common ancestor by mitosis. A “cell line” is a clone of a primary cell that is capable of stable growth in vitro for many generations.

As used herein, a “nucleic acid” or a “nucleic acid sequence” refers to a polymer or oligomer of pyrimidine and/or purine bases, preferably cytosine, thymine, and uracil, and adenine and guanine, respectively. The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506, incorporated herein by reference), locked nucleic acid (LNA; see Wahlestedt et al., Proc. Natl. Acad. Sci. U.S.A., 97: 5633-5638 (2000), incorporated herein by reference), cyclohexenyl nucleic acids (see Wang, J. Am. Chem. Soc., 122: 8595-8602 (2000), incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “wild-type” refers to a gene or a gene product that has the characteristics of that gene or gene product when isolated from a naturally occurring source. A wild-type gene is that which is most frequently observed in a population and is thus arbitrarily designated the “normal” or “wild-type” form of the gene. In contrast, the term “modified,” “mutant,” or “polymorphic” refers to a gene or gene product that displays modifications in sequence and or functional properties (e.g., altered characteristics) when compared to the wild-type gene or gene product. It is noted that naturally occurring mutants can be isolated; these are identified by the fact that they have altered characteristics when compared to the wild-type gene or gene product.

### 2. RNA-Guided CRISPR Recombineering System

In bacteria and archaea, CRISPR/Cas systems provide immunity by incorporating fragments of invading phage, virus, and plasmid DNA into CRISPR loci and using corresponding CRISPR RNAs (“crRNAs”) to guide the degradation of homologous sequences. Each CRISPR locus encodes acquired “spacers” that are separated by repeat sequences. Transcription of a CRISPR locus produces a “pre-crRNA,” which is processed to yield crRNAs containing spacer-repeat fragments that guide effector nuclease complexes to cleave dsDNA sequences complementary to the spacer. Three different types of CRISPR systems are known, type I, type II, or type III, and classified based on the Cas protein type and the use of a proto-spacer-adjacent motif (PAM) for selection of proto-spacers in invading DNA. The endogenous type II systems comprise the Cas9 protein and two noncoding crRNAs: trans-activating crRNA (tracrRNA) and a precursor crRNA (pre-crRNA) array containing nuclease guide sequences (also referred to as “spacers”) interspaced by identical direct repeats (DRs). tracrRNA is important for processing the pre-crRNA and formation of the Cas9 complex. First, tracrRNAs hybridize to repeat regions of the pre-crRNA. Second, endogenous RNaseIII cleaves the hybridized crRNA-tracrRNAs, and a second event removes the 5′ end of each spacer, yielding mature crRNAs that remain associated with both the tracrRNA and Cas9. Third, each mature complex locates a target double stranded DNA (dsDNA) sequence and cleaves both strands using the nuclease activity of Cas9.

CRISPR/Cas gene editing systems have been developed to enable targeted modifications to a specific gene of interest in eukaryotic cells. CRISPR/Cas gene editing systems are commonly based on the RNA-guided Cas9 nuclease from the type II prokaryotic clustered regularly interspaced short palindromic repeats (CRISPR) adaptive immune system. Engineering CRISPR/Cas systems for use in eukaryotic cells typically involves reconstitution of the crRNA-tracrRNA-Cas9 complex. In human cells, for example, the Cas9 amino acid sequence may be codon-optimized and modified to include an appropriate nuclear localization signal, and the crRNA and tracrRNA sequences may be expressed individually or as a single chimeric molecule via an RNA polymerase II promoter. Typically, the crRNA and tracrRNA sequences are expressed as a chimera and are referred to collectively as “guide RNA” (gRNA) or single guide RNA (sgRNA). Thus, the terms “guide RNA,” “single guide RNA,” and “synthetic guide RNA,” are used interchangeably herein and refer to a nucleic acid sequence comprising a tracrRNA and a pre-crRNA array containing a guide sequence. The terms “guide sequence,” “guide,” and “spacer,” are used interchangeably herein and refer to the about 20 nucleotide sequence within a guide RNA that specifies the target site. In CRISPR/Cas9 systems, the guide RNA contains an approximate 20-nucleotide guide sequence followed by a protospacer adjacent motif (PAM) that directs Cas9 via Watson-Crick base pairing to a target sequence.

In some embodiments, the disclosure provides a system for RNA-guided recombineering utilizing tools from CRISPR gene editing systems. The system comprises: a Cas protein, a nucleic acid molecule comprising a guide RNA sequence that is complementary to a target DNA sequence and a microbial recombination protein.

Cas protein families are described in further detail in, e.g., Haft et al., PLoS Comput. Biol., 1(6): e60 (2005), incorporated herein by reference. The Cas protein may be any Cas endonucleases. In some embodiments, the Cas protein is Cas9 or Cas12a, otherwise referred to as Cpf1. In one embodiment, the Cas9 protein is a wild-type Cas9 protein. The Cas9 protein can be obtained from any suitable microorganism, and a number of bacteria express Cas9 protein orthologs or variants. In some embodiments, the Cas9 is from Streptococcus pyogenes or Staphylococcus aureus. Cas9 proteins of other species are known in the art (see, e.g., U.S. Patent Application Publication 2017/0051312, incorporated herein by reference) and may be used in connection with the present disclosure. The amino acid sequences of Cas proteins from a variety of species are publicly available through the GenBank and UniProt databases.

In some embodiments, the Cas9 protein is a Cas9 nickase (Cas9n). Wild-type Cas9 has two catalytic nuclease domains facilitating double-stranded DNA breaks. A Cas9 nickase protein is typically engineered through inactivating point mutation(s) in one of the catalytic nuclease domains causing Cas9 to nick or enzymatically break only one of the two DNA strands using the remaining active nuclease domain. Cas9 nickases are known in the art (see, e.g., U.S. Patent Application Publication 2017/0051312, incorporated herein by reference) and include, for example, Streptococcus pyogenes with point mutations at D10 or H840. In select embodiments, the Cas9 nickase is Streptococcus pyogenes Cas9n (D10A).

In some embodiments, the Cas protein is a catalytically dead Cas. For example, catalytically dead Cas9 is essentially a DNA-binding protein due to, typically, two or more mutations within its catalytic nuclease domains which renders the protein with very little or no catalytic nuclease activity. Streptococcus pyogenes Cas9 may be rendered catalytically dead by mutations of D10 and at least one of E762, H840, N854, N863, or D986, typically H840 and/or N863 (see, e.g., U.S. Patent Application Publication 2017/0051312, incorporated herein by reference). Mutations in corresponding orthologs are known, such as N580 in Staphylococcus aureus Cas9. Oftentimes, such mutations cause catalytically dead Cas proteins to possess no more than 3% of the normal nuclease activity.

In some embodiments, the system comprises a nucleic acid molecule comprising a guide RNA sequence complementary to a target DNA sequence. The guide RNA sequence, as described above, specifies the target site with an approximate 20-nucleotide guide sequence followed by a protospacer adjacent motif (PAM) that directs Cas9 via Watson-Crick base pairing to a target sequence.

The terms “target DNA sequence,” “target nucleic acid,” “target sequence,” and “target site” are used interchangeably herein to refer to a polynucleotide (nucleic acid, gene, chromosome, genome, etc.) to which a guide sequence (e.g., a guide RNA) is designed to have complementarity, wherein hybridization between the target sequence and a guide sequence promotes the formation of a Cas9/CRISPR complex, provided sufficient conditions for binding exist. In some embodiments, the target sequence is a genomic DNA sequence. The term “genomic,” as used herein, refers to a nucleic acid sequence (e.g., a gene or locus) that is located on a chromosome in a cell. The target sequence and guide sequence need not exhibit complete complementarity, provided that there is sufficient complementarity to cause hybridization and promote formation of a CRISPR complex. A target sequence may comprise any polynucleotide, such as DNA or RNA. Suitable DNA/RNA binding conditions include physiological conditions normally present in a cell. Other suitable DNA/RNA binding conditions (e.g., conditions in a cell-free system) are known in the art; see, e.g., Sambrook, referenced herein and incorporated by reference. The strand of the target DNA that is complementary to and hybridizes with the DNA-targeting RNA is referred to as the “complementary strand” and the strand of the target DNA that is complementary to the “complementary strand” (and is therefore not complementary to the DNA-targeting RNA) is referred to as the “noncomplementary strand” or “non-complementary strand.”

The target genomic DNA sequence may encode a gene product. The term “gene product,” as used herein, refers to any biochemical product resulting from expression of a gene. Gene products may be RNA or protein. RNA gene products include non-coding RNA, such as tRNA, rRNA, micro RNA (miRNA), and small interfering RNA (siRNA), and coding RNA, such as messenger RNA (mRNA). In some embodiments, the target genomic DNA sequence encodes a protein or polypeptide.

In some embodiments, for instance, when the system includes a Cas9 nickase or a catalytically dead Cas 9, two nucleic acid molecules comprising a guide RNA sequence may be utilized. The two nucleic acid molecules may have the same or different guide RNA sequences, thus complementary to the same or different target DNA sequence. In some embodiments, the guide RNA sequences of the two nucleic acid molecules are complementary to a target DNA sequences at opposite ends (e.g., 3′ or 5′) and/or on opposite strands of the insert location.

In some embodiments, the system further comprises a recruitment system comprising at least one aptamer sequence and an aptamer binding protein functionally linked to the microbial recombination protein as part of a fusion protein.

In some embodiments, the aptamer sequence is an RNA aptamer sequence. In some embodiments, the nucleic acid molecule comprising the guide RNA also comprises one or more RNA aptamers, or distinct RNA secondary structures or sequences that can recruit and bind another molecular species, an adaptor molecule, such as a nucleic acid or protein. The RNA aptamers can be naturally occurring or synthetic oligonucleotides that have been engineered through repeated rounds of in vitro selection or SELEX (systematic evolution of ligands by exponential enrichment) to bind to a specific target molecular species. In some embodiments, the nucleic acid comprises two or more aptamer sequences. The aptamer sequences may be the same or different and may target the same or different adaptor proteins. In select embodiments, the nucleic acid comprises two aptamer sequences.

Any RNA aptamer/aptamer binding protein pair known may be selected and used in connection with the present disclosure (see, e.g., Jayasena, S. D., Clinical Chemistry, 1999. 45(9): p. 1628-1650; Gelinas, et al., Current Opinion in Structural Biology, 2016. 36: p. 122-132; and Hasegawa, H., Molecules, 2016; 21(4): p. 421, incorporated herein by reference).

A number of RNA aptamer binding, or adaptor, proteins exist, including a diverse array of bacteriophage coat proteins. Examples of such coat proteins include but are not limited to: MS2, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. In some embodiments, the RNA aptamer binds MS2 bacteriophage coat protein or a functional derivative, fragment or variant thereof. MS2 binding RNA aptamers commonly have a simple stem-loop structure, classically defined by a 19 nucleotide RNA molecule with a single bulged adenine on the 5′ leg of the stem (Witherall G. W., et al., (1991) Prog. Nucleic Acid Res. Mol. Biol., 40, 185-220, incorporated herein by reference). However, a number of vastly different primary sequences were found to be able to bind the MS2 coat protein (Parrott A M, et al., Nucleic Acids Res. 2000; 28(2):489-497, Buenrostro J D, et al. Natura Biotechnology 2014; 32, 562-568, and incorporated herein by reference). Any of the RNA aptamer sequence known to bind the MS2 bacteriophage coat protein may be utilized in connection with the present disclosure. In select embodiments, the MS2 RNA aptamer sequence comprises: AACAUGAGGAUCACCCAUGUCUGCAG (SEQ ID NO:145), AGCAUGAGGAUCACCCAUGUCUGCAG (SEQ ID NO:146), or AGCGUGAGGAUCACCCAUGCCUGCAG (SEQ ID NO:147).

N-proteins (Nut-utilization site proteins) of bacteriophages contain arginine-rich conserved RNA recognition motifs of ˜20 amino acids, referred to as N peptides. The RNA aptamer may bind a phage N peptide or a functional derivative, fragment or variant thereof. In some embodiments, the phage N peptide is the lambda or P22 phage N peptide or a functional derivative, fragment or variant thereof.

In select embodiments, the N peptide is lambda phage N22 peptide, or a functional derivative, fragment or variant thereof. In some embodiments, the N22 peptide comprises an amino acid sequence with at least 70% similarity to the amino acid sequence GNARTRRRERRAEKQAQWKAAN (SEQ ID NO: 149). N22 peptide, the 22 amino acid RNA-binding domain of the λ bacteriophage antiterminator protein N (λN-(1-22) or λN peptide), is capable of specifically binding to specific stem-loop structures, including but not limited to the BoxB stem-loop. See, for example Cilley and Williamson, RNA 1997; 3(1):57-67, incorporated herein by reference. A number of different BoxB stem-loop primary sequences are known to bind the N22 peptide and any of those may be utilized in connection with the present disclosure. In some embodiments, the N22 peptide RNA aptamer sequence comprises a nucleotide sequence with at least 70% similarity to an RNA sequence selected from the group consisting of GCCCUGAAAAAGGGC (SEQ ID NO: 150), GCCCUGAAGAAGGGC (SEQ ID NO: 151), GCGCUGAAAAAGCGC (SEQ ID NO: 152), GCCCUGACAAAGGGC (SEQ ID NO: 153), and GCGCUGACAAAGCGC (SEQ ID NO: 154). In some embodiments, the N22 peptide RNA aptamer sequence is selected from the group consisting of SEQ ID NOs: 150-154.

In select embodiments, the N peptide is the P22 phage N peptide, or a functional derivative, fragment or variant thereof. A number of different BoxB stem-loop primary sequences are known to bind the P22 phage N peptide and variants thereof and any of those may be utilized in connection with the present disclosure. See, for example Cocozaki, Ghattas, and Smith, Journal of Bacteriology 2008; 190(23):7699-7708, incorporated herein by reference. In some embodiments, the P22 phage N peptide comprises an amino acid sequence with at least 70% similarity to the amino acid sequence GNAKTRRHERRRKLAIERDTI (SEQ ID NO: 155). In some embodiments, the P22 phage N peptide RNA aptamer sequence comprises a sequence with at least 70% similarity to an RNA sequence selected from the group consisting of GCGCUGACAAAGCGC (SEQ ID NO: 156) and CCGCCGACAACGCGG (SEQ ID NO: 157). In some embodiments, the P22 phage N peptide RNA aptamer sequence is selected from the group consisting of SEQ ID NOs: 156-157, UGCGCUGACAAAGCGCG (SEQ ID NO: 158) or ACCGCCGACAACGCGGU (SEQ ID NO: 159).

In some embodiments, the aptamer sequence is a peptide aptamer sequence. The peptide aptamers can be naturally occurring or synthetic peptides that are specifically recognized by an affinity agent. Such aptamers include, but are not limited to, a c-Myc affinity tag, an HA affinity tag, a His affinity tag, an S affinity tag, a methionine-His affinity tag, an RGD-His affinity tag, a 7× His tag, a FLAG octapeptide, a strep tag or strep tag II, a V5 tag, or a VSV-G epitope. Corresponding aptamer binding proteins are well-known in the art and include, for example, primary antibodies, biotin, affimers, single domain antibodies, and antibody mimetics.

An exemplary peptide aptamer includes a GCN4 peptide (Tanenbaum et al., Cell 2014; 159(3):635-646, incorporated herein by reference). Antibodies, or GCN4 binding protein can be used as the aptamer binding proteins.

In some embodiments, the peptide aptamer sequence is conjugated to the Cas protein. The peptide aptamer sequence may be fused to the Cas in any orientation (e.g., N-terminus to C-terminus, C-terminus to N-terminus, N-terminus to N-terminus). In select embodiments, the peptide aptamer is fused to the C-terminus of the Cas protein.

In some embodiments, between 1 and 24 peptide aptamer sequences may be conjugated to the Cas protein. The aptamer sequences may be the same or different and may target the same or different aptamer binding proteins. In select embodiments, 1 to 24 tandem repeats of the same peptide aptamer sequence are conjugated to the Cas protein. In preferred embodiments between 4 and 18 tandem repeats are conjugated to the Cas protein. The individual aptamers may be separated by a linker region. Suitable linker regions are known in the art. The linker may be flexible or configured to allow the binding of affinity agents to adjacent aptamers without or with decreased steric hindrance. The linker sequences may provide an unstructured or linear region of the polypeptide, for example, with the inclusion of one or more glycine and/or serine residues. The linker sequences can be at least about 2, 3, 4, 5, 6, 7, 8, 9, 10 or more amino acids in length.

In some embodiments, the fusion protein comprises a microbial recombination protein functionally linked to an aptamer binding protein. The microbial recombination protein may be RecE, RecT, lambda exonuclease (Exo), Bet protein (betA, redB), exonuclease gp6, single-stranded DNA-binding protein gp2.5, or a derivative or variant thereof.

In select embodiments, the microbial recombination protein is RecE or RecT, or a derivative or variant thereof. Derivatives or variants of RecE and RecT are functionally equivalent proteins or polypeptides which possess substantially similar function to wild type RecE and RecT. RecE and RecT derivatives or variants include biologically active amino acid sequences similar to the wild-type sequences but differing due to amino acid substitutions, additions, deletions, truncations, post-translational modifications, or other modifications. In some embodiments, the derivatives may improve translation, purification, biological half-life, activity, or eliminate or lessen any undesirable side effects or reactions. The derivatives or variants may be naturally occurring polypeptides, synthetic or chemically synthesized polypeptides or genetically engineered peptide polypeptides. RecE and RecT bioactivities are known to, and easily assayed by, those of ordinary skill in the art, and include, for example exonuclease and single-stranded nucleic acid binding, respectively.

The RecE or RecT may be from a number of microbial organisms, including Escherichia coli, Pantoea breeneri, Type-F symbiont of Plautia stali, Providencia sp. MGF014, Shigella sonnei, Pseudobacteriovorax antillogorgiicola, among others. In preferred embodiments, the RecE and RecT protein is derived from Escherichia coli.

In some embodiments, the fusion protein comprises RecE, or a derivative or variant thereof. The RecE, or derivative or variant thereof, may comprise an amino acid sequence selected from the group consisting of SEQ ID NOs: 1-8. The RecE, or derivative or variant thereof, may comprise an amino acid sequences with at least 70% (e.g., 75%, 80%, 85%, 90%, 91%, 92%, 93%, 94%, 95%, 96%, 97%, 98%, 99%, or 100%) similarity to amino acid sequences selected from the group consisting of SEQ ID NOs: 1-8. In select embodiments, the RecE, or derivative or variant thereof, comprises an amino acid sequences with at least 90% similarity to amino acid sequences selected from the group consisting of SEQ ID NOs: 1-8. In exemplary embodiments, the RecE, or derivative or variant thereof, comprises an amino acid sequences with at least 90% similarity to amino acid sequences selected from the group consisting of SEQ ID NOs: 1-3.

In some embodiments, the fusion protein comprises RecT, or a derivative or variant thereof. The RecT, or derivative or variant thereof, may comprise an amino acid sequence selected from the group consisting of SEQ ID NOs: 9-14. The RecT, or derivative or variant thereof, may comprise an amino acid sequences with at least 70% (e.g., 75%., 80%, 85%, 90%, 91%, 92%, 93%, 94%, 95%, 96%, 97%, 98%, 99%, or 100%) similarity to amino acid sequences selected from the group consisting of SEQ ID NOs: 9-14. In select embodiments, the RecT, or derivative or variant thereof, comprises an amino acid sequences with at least 90% similarity to amino acid sequences selected from the group consisting of SEQ ID NOs: 9-14. In exemplary embodiments, the RecT, or derivative or variant thereof, comprises an amino acid sequences with at least 90% similarity to amino acid sequences selected from the group consisting of SEQ ID NO: 9.

Truncations may be from either the C-terminal or N-terminal ends, or both. For example, as demonstrated in Example 6 below, a diverse set of truncations from either end or both provided a functional product. In some embodiments, one or more (2, 3, 4, 5, 10, 20, 30, 40, 50, 60, 100, 120 or more) amino acids may be truncated from the C-terminal, N-terminal ends as compared to the wild-type sequence.

In the fusion protein, the microbial recombination protein may be linked to either terminus of the aptamer binding protein in any orientation (e.g., N-terminus to C-terminus, C-terminus to N-terminus, N-terminus to N-terminus). In select embodiments, the microbial recombination protein N-terminus is linked to the aptamer binding protein C-terminus. Thus, the overall fusion protein from N- to C-terminus comprises the aptamer binding protein (N- to C-terminus) linked to the microbial recombination protein (N- to C-terminus).

In some embodiments, the fusion protein further comprises a linker between the microbial recombination protein and the aptamer binding protein. The linkers may comprise any amino acid sequence of any length. The linkers may be flexible such that they do not constrain either of the two components they link together in any particular orientation. The linkers may essentially act as a spacer. In select embodiments, the linker links the C-terminus of the microbial recombination protein to the N-terminus of the aptamer binding protein. In select embodiments, the linker comprises the amino acid sequence of the 16-residue XTEN linker, SGSETPGTSESATPES (SEQ ID NO: 15) or the 37-residue EXTEN linker, SASGGSSGGSSGSETPGTSESATPESSGGSSGGSGGS (SEQ ID NO: 148).

In some embodiments, the fusion protein further comprises a nuclear localization sequence (NLS). The nuclear localization sequence may be at any location within the fusion protein (e.g., C-terminal of the aptamer binding protein, N-terminal of the aptamer binding protein, C-terminal of the microbial recombination protein). In select embodiments, the nuclear localization sequence is linked to the C-terminus of the microbial recombination protein. A number of nuclear localization sequences are known in the art (see, e.g., Lange, A., et al., J Biol Chem. 2007; 282(8): 5101-5105, incorporated herein by reference) and may be used in connection with the present disclosure. The nuclear localization sequence may be the SV40 NLS, PKKKRKV (SEQ ID NO:16); the Ty1 NLS, NSKKRSLEDNETEIKVSRDTWNTKNMRSLEPPRSKKRIH (SEQ ID NO: 17); the c-Myc NLS, PAAKRVKLD (SEQ ID NO:18); the biSV40 NLS, KRTADGSEFESPKKKRKV (SEQ ID NO: 19); and the Mut NLS, PEKKRRRPSGSVPVLARPSPPKAGKSSCI (SEQ ID NO: 20). In select embodiments, the nuclear localization sequence is the SV40 NLS, PKKKRKV (SEQ ID NO: 16).

The Cas protein and the fusion protein are desirably included in a single composition alone, in combination with each other, and/or the polynucleotide(s) (e.g., a vector) comprising the guide RNA sequence and the aptamer sequence. The Cas protein and/or the fusion protein may or may not be physically or chemically bound to the polynucleotide. The Cas protein and/or the microbial recombination protein can be associated with a polynucleotide using any suitable method for protein-protein linking or protein-virus linking known in the art.

The disclosure further provides compositions and vectors comprising a polynucleotide comprising a nucleic acid sequence encoding a fusion protein comprising a microbial recombination protein functionally linked to an RNA aptamer binding protein.

The compositions or vectors may further comprise at least one or both of a polynucleotide comprising a nucleic acid sequence encoding a Cas protein and a nucleic acid molecule comprising a guide RNA sequence that is complementary to a target DNA sequence. In some embodiments, the nucleic acid molecule comprising a guide RNA sequence further comprises at least one RNA aptamer sequence. In some embodiments, the polynucleotide comprising a nucleic acid sequence encoding a Cas protein further comprises a sequence encoding at least one peptide aptamer sequence.

Descriptions of the nucleic acid molecule comprising a guide RNA sequence, the aptamer sequences, the Cas proteins, the microbial recombination proteins, and the aptamer binding proteins set forth above in connection with the inventive system also are applicable to the polynucleotides of the recited compositions and vectors.

The nucleic acid sequence encoding the Cas protein and/or the nucleic acid sequence encoding a fusion protein comprising a microbial recombination protein functionally linked to an aptamer binding protein can be provided to a cell on the same vector (e.g., in cis) as the nucleic acid molecule comprising the guide RNA sequence and/or the RNA aptamer sequence. In such embodiments, a unidirectional promoter can be used to control expression of each nucleic acid sequence. In another embodiment, a combination of bidirectional and unidirectional promoters can be used to control expression of multiple nucleic acid sequences.

In other embodiments, a nucleic acid sequence encoding the Cas protein, the nucleic acid sequence encoding a fusion protein comprising a microbial recombination protein functionally linked to an aptamer binding protein, and the nucleic acid molecule comprising the guide RNA sequence and/or the RNA aptamer sequence can be provided to a cell on separate vectors (e.g., in trans). Each of the nucleic acid sequences in each of the separate vectors can comprise the same or different expression control sequences. The separate vectors can be provided to cells simultaneously or sequentially.

The vector(s) comprising the nucleic acid sequences encoding the Cas protein and encoding a fusion protein comprising a microbial recombination protein functionally linked to an aptamer binding protein can be introduced into a host cell that is capable of expressing the polypeptide encoded thereby, including any suitable prokaryotic or eukaryotic cell. As such, the disclosure provides an isolated cell comprising the vector or nucleic acid sequences disclosed herein. Preferred host cells are those that can be easily and reliably grown, have reasonably fast growth rates, have well characterized expression systems, and can be transformed or transfected easily and efficiently. Examples of suitable prokaryotic cells include, but are not limited to, cells from the genera Bacillus (such as Bacillus subtilis and Bacillus brevis), Escherichia (such as E. coli), Pseudomonas, Streptomyces, Salmonella, and Envinia. Suitable eukaryotic cells are known in the art and include, for example, yeast cells, insect cells, and mammalian cells. Examples of suitable yeast cells include those from the genera Kluyveromyces, Pichia, Rhino-sporidium, Saccharomyces, and Schizosaccharomyces. Exemplary insect cells include Sf-9 and HIS (Invitrogen, Carlsbad, Calif.) and are described in, for example, Kitts et al., Biotechniques, 14: 810-817 (1993); Lucklow, Curr. Opin. Biotechnol., 4: 564-572 (1993); and Lucklow et al., J. Virol., 67: 4566-4579 (1993), incorporated herein by reference. Desirably, the host cell is a mammalian cell, and in some embodiments, the host cell is a human cell. A number of suitable mammalian and human host cells are known in the art, and many are available from the American Type Culture Collection (ATCC, Manassas, Va.). Examples of suitable mammalian cells include, but are not limited to, Chinese hamster ovary cells (CHO) (ATCC No. CCL61), CHO DHFR-cells (Urlaub et al., Proc. Natl. Acad. Sci. USA, 97: 4216-4220 (1980)), human embryonic kidney (HEK) 293 or 293T cells (ATCC No. CRL1573), and 3T3 cells (ATCC No. CCL92). Other suitable mammalian cell lines are the monkey COS-1 (ATCC No. CRL1650) and COS-7 cell lines (ATCC No. CRL1651), as well as the CV-1 cell line (ATCC No. CCL70). Further exemplary mammalian host cells include primate, rodent, and human cell lines, including transformed cell lines. Normal diploid cells, cell strains derived from in vitro culture of primary tissue, as well as primary explants, are also suitable. Other suitable mammalian cell lines include, but are not limited to, mouse neuroblastoma N2A cells, HeLa, HEK, A549, HepG2, mouse L-929 cells, and BHK or HaK hamster cell lines. Methods for selecting suitable mammalian host cells and methods for transformation, culture, amplification, screening, and purification of cells are known in the art.

### 3. Methods of Altering Target DNA

The disclosure also provides a method of altering a target DNA. In some embodiments, the method alters genomic DNA sequence in a cell, although any desired nucleic acid may be modified. When applied to DNA contained in cells, the method comprises introducing the systems, compositions, or vectors described herein into a cell comprising a target genomic DNA sequence. Descriptions of the nucleic acid molecule comprising a guide RNA sequence, the Cas proteins, the microbial recombination proteins, the recruitment systems, and polynucleotides encoding thereof, the cell, the target genomic DNA sequence, and components thereof, set forth above in connection with the inventive system are also applicable to the method of altering a target genomic DNA sequence in a cell. The systems, composition or vectors may be introduced in any manner known in the art including, but not limited to, chemical transfection, electroporation, microinjection, biolistic delivery via gene guns, or magnetic-assisted transfection, depending on the cell type.

Upon introducing the systems described herein into a cell comprising a target genomic DNA sequence, the guide RNA sequence binds to the target genomic DNA sequence in the cell genome, the Cas protein associates with the guide RNA and may induce a double strand break or single strand nick in the target genomic DNA sequence and the aptamer recruits the microbial recombination proteins to the target genomic DNA sequence through the aptamer binding protein of the fusion protein, thereby altering the target genomic DNA sequence in the cell. When introducing the compositions, or vectors described herein into the cell, the nucleic acid molecule comprising a guide RNA sequence, the Cas9 protein, and the fusion protein are first expressed in the cell.

In some embodiments, the cell is in an organism or host, such that introducing the disclosed systems, compositions, vectors into the cell comprises administration to a subject. The method may comprise providing or administering to the subject, in vivo, or by transplantation of ex vivo treated cells, systems, compositions, vectors of the present system.

A “subject” may be human or non-human and may include, for example, animal strains or species used as “model systems” for research purposes, such a mouse model as described herein. Likewise, subject may include either adults or juveniles (e.g., children). Moreover, subject may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

As used herein, the terms “providing”, “administering,” “introducing,” are used interchangeably herein and refer to the placement of the systems of the disclosure into a subject by a method or route which results in at least partial localization of the system to a desired site. The systems can be administered by any appropriate route which results in delivery to a desired location in the subject.

The phrase “altering a DNA sequence,” as used herein, refers to modifying at least one physical feature of a DNA sequence of interest. DNA alterations include, for example, single or double strand DNA breaks, deletion, or insertion of one or more nucleotides, and other modifications that affect the structural integrity or nucleotide sequence of the DNA sequence. The modifications of a target sequence in genomic DNA may lead to, for example, gene correction, gene replacement, gene tagging, transgene insertion, nucleotide deletion, gene disruption, gene mutation, gene knock-down, and the like.

In some embodiments, the systems and methods described herein may be used to correct one or more defects or mutations in a gene (referred to as “gene correction”). In such cases, the target genomic DNA sequence encodes a defective version of a gene, and the system further comprises a donor nucleic acid molecule which encodes a wild-type or corrected version of the gene. Thus, in other words, the target genomic DNA sequence is a “disease-associated” gene. The term “disease-associated gene,” refers to any gene or polynucleotide whose gene products are expressed at an abnormal level or in an abnormal form in cells obtained from a disease-affected individual as compared with tissues or cells obtained from an individual not affected by the disease. A disease-associated gene may be expressed at an abnormally high level or at an abnormally low level, where the altered expression correlates with the occurrence and/or progression of the disease. A disease-associated gene also refers to a gene, the mutation or genetic variation of which is directly responsible or is in linkage disequilibrium with a gene(s) that is responsible for the etiology of a disease. Examples of genes responsible for such “single gene” or “monogenic” diseases include, but are not limited to, adenosine deaminase, α-1 antitrypsin, cystic fibrosis transmembrane conductance regulator (CFTR), β-hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). Other single gene or monogenic diseases are known in the art and described in, e.g., Chial, H. Rare Genetic Disorders: Learning About Genetic Disease Through Gene Mapping, SNPs, and Microarray Data, Nature Education 1(1):192 (2008), incorporated herein by reference; Online Mendelian Inheritance in Man (OMIM); and the Human Gene Mutation Database (HGMD).

In another embodiment, the target genomic DNA sequence can comprise a gene, the mutation of which contributes to a particular disease in combination with mutations in other genes. Diseases caused by the contribution of multiple genes which lack simple (e.g., Mendelian) inheritance patterns are referred to in the art as a “multifactorial” or “polygenic” disease. Examples of multifactorial or polygenic diseases include, but are not limited to, asthma, diabetes, epilepsy, hypertension, bipolar disorder, and schizophrenia. Certain developmental abnormalities also can be inherited in a multifactorial or polygenic pattern and include, for example, cleft lip/palate, congenital heart defects, and neural tube defects.

In another embodiment, the method of altering a target genomic DNA sequence can be used to delete nucleic acids from a target sequence in a cell by cleaving the target sequence and allowing the cell to repair the cleaved sequence in the absence of an exogenously provided donor nucleic acid molecule. Deletion of a nucleic acid sequence in this manner can be used in a variety of applications, such as, for example, to remove disease-causing trinucleotide repeat sequences in neurons, to create gene knock-outs or knock-downs, and to generate mutations for disease models in research.

The term “donor nucleic acid molecule” refers to a nucleotide sequence that is inserted into the target DNA (e.g., genomic DNA). As described above the donor DNA may include, for example, a gene or part of a gene, a sequence encoding a tag or localization sequence, or a regulating element. The donor nucleic acid molecule may be of any length. In some embodiments, the donor nucleic acid molecule is between 10 and 10,000 nucleotides in length. For example, between about 100 and 5,000 nucleotides in length, between about 200 and 2,000 nucleotides in length, between about 500 and 1,000 nucleotides in length, between about 500 and 5,000 nucleotides in length, between about 1,000 and 5,000 nucleotides in length, or between about 1,000 and 10,000 nucleotides in length,

The disclosed systems and methods overcome challenges encountered during conventional gene editing, including low efficiency and off-target events, particularly with kilobase-scale nucleic acids. In some embodiments, the disclosed systems and methods improve the efficiency of gene editing. For example, the disclosed systems and methods can have a 2- to 10-fold increase in efficiency over conventional CRISPR-Cas9 systems and methods, as shown in Examples 2, 3, and 5. In some embodiments, the improvement in efficiency is accompanied by a reduction in off-target events. The off-target events may be reduced by greater than 50% compared to conventional CRISPR-Cas9 systems and methods, for example, a reduction of off-target events by about 90% is shown in Example 3. Another aspect of increasing the overall accuracy of a gene editing system is reducing the on-target insertion-deletions (indels), a byproduct of HDR editing. In some embodiments, the disclosed systems and methods reduce the on-target indels by greater than 90% compared to conventional CRISPR-Cas9 systems and methods, as shown in Example 3.

The disclosure further provides kits containing one or more reagents or other components useful, necessary, or sufficient for practicing any of the methods described herein. For example, kits may include CRISPR reagents (Cas protein, guide RNA, vectors, compositions, etc.), recombineering reagents (recombination protein-aptamer binding protein fusion protein, the aptamer sequence, vectors, compositions, etc.) transfection or administration reagents, negative and positive control samples (e.g., cells, template DNA), cells, containers housing one or more components (e.g., microcentrifuge tubes, boxes), detectable labels, detection and analysis instruments, software, instructions, and the like.

Any element of any suitable CRISPR/Cas gene editing system known in the art can be employed in the systems and methods described herein, as appropriate. CRISPR/Cas gene editing technology is described in detail in, for example, U.S. Pat. Nos. 8,546,553, 8,697,359; 8,771,945; 8,795,965; 8,865,406; 8,871,445; 8,889,356; 8,889,418; 8,895,308; 8,9066,616; 8,932,814; 8,945,839; 8,993,233; 8,999,641; 9,115,348; 9,149,049; 9,493,844; 9,567,603; 9,637,739; 9,663,782; 9,404,098; 9,885,026; 9,951,342; 10,087,431; 10,227,610; 10,266,850; 10,601,748; 10,604,771; and 10,760,064; and U.S. Patent Application Publication Nos. US2010/0076057; US2014/0113376; US2015/0050699; US2015/0031134; US2014/0357530; US2014/0349400; US2014/0315985; US2014/0310830; US2014/0310828; US2014/0309487; US2014/0294773; US2014/0287938; US2014/0273230; US2014/0242699; US2014/0242664; US2014/0212869; US2014/0201857; US2014/0199767; US2014/0189896; US2014/0186919; US2014/0186843; and US2014/0179770, each incorporated herein by reference.

The following examples further illustrate the invention but should not be construed as in any way limiting its scope.

### EXAMPLES

Materials and Methods

RecE/T Homolog Screening RefSeq non-redundant protein database was downloaded from NCBI on Oct. 29, 2019. The database was searched with E. coli Rac prophage RecT (NP_415865.1) and RecE (NP_415866.1) as queries using position-specific iterated (PSI)-BLAST′ to retrieve protein homologs. Hits were clustered with CD-HIT2 and representative sequences were selected from each cluster for multiple alignment with MUSCLE³. Then, FastTree4 was used for maximum likelihood tree reconstruction with default parameters. A diverse set of RecET homologs were selected, synthesized by GenScript, and cloned into pMPH MCP vectors for testing.

Plasmids construction pX330, pMPH and pU6-(BbsI)_CBh-Cas9-T2A-BFP plasmids were obtained from Addgene. Tested effector DNA fragments were ordered from IDT, Genewiz, and GenScript. The fragments were Gibson assembled into the backbones using NEBuilder HiFi DNA Assembly Master Mix (New England BioLabs). All sgRNAs (Table 1) were inserted into backbones using Golden Gate cloning. All constructs were sequence-verified with Sanger sequencing of prepped plasmids.

TABLE 1
Sequence for sgRNAs

| Primer Name | Genomic Target | Sequence |
|---|---|---|
| sp-EMX1 | EMX1 | GTCACCTCCAATGACTAGGG |
| | | (SEQ ID |
| | | NO: 21) |
| sp-VEGFA | VEGFA | GGTGAGTGAGTGTGTGCGTG |
| | | (SEQ ID |
| | | NO: 22) |
| sp-DYNLT1 | DYNLT1 | AAGGCCATAGGCTGGACTGC |
| | | (SEQ ID |
| | | NO: 23) |
| sp-HSP90AA1 | HSP90AA1 | GTAGACTAATCTCTGGCTGA |
| | | (SEQ ID |
| | | NO: 24) |
| sp-OCT4 | OCT4 | TCTCCCATGCATTCAAACTG |
| | | (SEQ ID NO: 25) |
| sp-AAVS1 | AAVS1 | ACCCCACAGTGGGGCCACTA |
| | | (SEQ ID |
| | | NO: 26) |
| nsp-EMX1- | EMX1 | GTCACCTCCAATGACTAGGG |
| guide1 | | (SEQ ID |
| | | NO: 27) |
| nsp-EMX1- | EMX1 | GTCACCTCCAATGACTAGGG |
| guide2 | | (SEQ ID |
| | | NO: 28) |
| nsp-DYNLT1- | DYNLT1 | AAGGCCATAGGCTGGACTGC |
| guide1 | | (SEQ ID |
| | | NO: 29) |
| nsp-DYNLT1- | DYNLT1 | GGCACTGACGATGCAGTACA |
| guide2 | | (SEQ ID |
| | | NO: 30) |
| nsp-HSP90AA1- | HSP90AA1 | GTAGACTAATCTCTGGCTGA |
| guide1 | | (SEQ ID |
| | | NO: 31) |
| nsp-HSP90AA1- | HSP90AA1 | TCGTCATCTCCTTCAAGGGG |
| guide2 | | (SEQ ID NO: 32) |
| nsp-OCT4- | OCT4 | ATGCATGGGAGAGCCCAGAG |
| guide1 | | (SEQ ID |
| | | NO: 33) |
| nsp-OCT4- | OCT4 | GCCTGCCCTTCTAGGAATGG |
| guide2 | | (SEQ ID |
| | | NO: 34) |

Cell culture Human Embryonic Kidney (HEK) 293T, HeLa and HepG2 were maintained in Dulbecco's Modified Eagle's Medium (DMEM, Life Technologies), with 10% fetal bovine serum (FBS, HyClone), 100 U/mL penicillin, and 100 μg/mL streptomycin (Life Technologies) at 37° C. with 5% CO₂.

hES-H9 cells were maintained in mTeSR1 medium (StemCell Technologies) at 37° C. with 5% CO₂. Culture plates were pre-coated with Matrigel (Corning) 12 hours prior to use, and cells were supplemented with 10 μM Y27632 (Sigma) for the first 24 hours after passaging. Culture media was changed every 24 hours.

Transfection HEK293T cells were seeded into 96-well plates (Corning) 12-24 hours prior to transfection at a density of 30,000 cells/well, and 250 ng of total DNA was transfected per well. HeLa and HepG2 cells were seeded into 48-well plates (Corning) one day prior to transfection at a density of 50,000 and 30,000 cells/well respectively, and 400 ng of total DNA was transfected per well. Transfections were performed with Lipofectamine 3000 (Life Technologies) following the manufacturer's instructions.

Electroporation For hES-H9 related transfection experiments, P3 Primary Cell 4D-Nucleofector™ X Kit S (Lonza) was used following the manufacturer's protocol. For each reaction, 300,000 cells were nucleofected with 4 μg total DNA using the DC100 Nucleofector Program.

Fluorescence-activated cell sorting (FACS) mKate knock-in efficiency was analyzed on a CytoFLEX flow cytometer (Beckman Coulter; Stanford Stem Cell FACS Core). 72 hours after transfection, cells were washed once with PBS and dissociated with TrypLE Express Enzyme (Thermo Fisher Scientific). Cell suspension was then transferred to a 96-well U-bottom plate (Thermo Fisher Scientific) and centrifuged at 300×G for 5 minutes. After removing the supernatant, pelleted cells were resuspended with 50 μl 4% FBS in PBS, and cells were sorted within 30 minutes of preparation.

RFLP HEK293T cells were transfected with plasmid DNA and PCR templates and harvested after 72 hours for genomic DNA using the QuickExtract DNA Extraction Solution (Biosearch Technologies) following the manufacturer's protocol. The target genomic region was amplified using specific primers outside of the homology arms of the PCR template. PCR products were purified with Monarch PCR & DNA Cleanup Kit (New England BioLabs). 300 ng of purified product was digested with BsrGI (EMX1, New England BioLabs) or XbaI (VEGFA, NEB), and the digested products were analyzed on a 5% Mini-PROTEAN TBE gel (Bio-Rad).

Next-Generation Sequencing Library Preparation 72 hours after transfection, genomic DNA was extracted using QuickExtract DNA Extraction Solution (Biosearch Technologies). 200 ng total DNA was used for NGS library preparation. Genes of interest were amplified using specific primers (Table 2) for the first round PCR reaction. Illumina adapters and index barcodes were added to the fragments with a second round PCR using the primers listed in Table 2. Round 2 PCR products were purified by gel electrophoresis on a 2% agarose gel using the Monarch DNA Gel Extraction Kit (NEB). The purified product was quantified with Qubit dsDNA HS Assay Kit (Thermo Fisher) and sequenced on an Illumina MiSeq according to the manufacturer's instructions.

TABLE 2
Sequence for primers used for PCR template, RFLP and NGS

| | | Genomic | |
|---|---|---|---|
| Primer Name | Usage | Target | Sequence |
| EMX1-PCR-F | PCR | EMX1 | CATTCTGCCTCTCTGTATGGAAAAGAGC |
| | template | | (SEQ ID NO: 35) |
| EMX1-PCR-R | PCR | EMX1 | CCCATTGAACTACCTGGGCCTGATTC (SEQ |
| | template | | ID NO: 36) |
| VEGFA-PCR- | PCR | VEGFA | AGGTTTGAATCATCACGCAGGC (SEQ ID |
| F | template | | NO: 37) |
| VEGFA-PCR- | PCR | VEGFA | ATTCAAGTGGGGAATGGCAAGC (SEQ ID |
| R | template | | NO: 38) |
| DYNLT1- | PCR | DYNLT1 | TGCCGTAAATGCTGCTCTCT |
| PCR-100bp-F | template | | (SEQ ID NO: 39) |
| DYNLT1- | PCR | DYNLT1 | AGACTTGCCAAGGTTCTTTGTG (SEQ ID |
| PCR-200bp-F | template | | NO: 40) |
| DYNLT1- | PCR | DYNLT1 | AGTGACCTGTGTAATTATGCAGAAG (SEQ |
| PCR-400bp-F | template | | ID NO: 41) |
| DYNLT1- | PCR | DYNLT1 | TGAAAGTGCCACAAAACAAAGAGA (SEQ |
| PCR-100bp-R | template | | ID NO: 42) |
| DYNLT1- | PCR | DYNLT1 | AAGACAAGTGGCAACGCAG (SEQ ID |
| PCR-200bp-R | template | | NO: 43) |
| DYNLT1- | PCR | DYNLT1 | CGTTTATGATACTATGCAGACTATGAAGAA |
| PCR-400bp-R | template | | C (SEQ ID NO: 44) |
| HSP90AA1- | PCR | HSP90AA1 | ATGAAGATGACCCTACTGCTGAT (SEQ ID |
| PCR-100bp-F | template | | NO: 45) |
| HSP90AA1- | PCR | HSP90AA1 | TACTGTCTTGAAAGCAGATAGAAACC (SEQ |
| PCR-bp-F | template | | ID NO: 46) |
| HSP90AA1- | PCR | HSP90AA1 | GCAGCAAAGAAACACCTGGA (SEQ ID |
| PCR-600bp-R | template | | NO: 47) |
| HSP90AA1- | PCR | HSP90AA1 | GTTGTCATGCCATACAGACTTTTT (SEQ ID |
| PCR-100bp-R | template | | NO: 48) |
| HSP90AA1- | PCR | HSP90AA1 | AGCATTACTAGCTCTGCTTTAGTG (SEQ ID |
| PCR-200bp-R | template | | NO: 49) |
| HSP90AA1- | PCR | HSP90AA1 | TCCACAAGACTGGGTCTGAG (SEQ ID |
| PCR-600bp-R | template | | NO: 50) |
| OCT4-PCR-F | PCR | OCT4 | GCGACTATGCACAACGAGAGG (SEQ ID |
| | template | | NO: 51) |
| OCT4-PCR-R | PCR | OCT4 | AAGTGTGTCTATCTACTGTGTCCCAG (SEQ |
| | template | | ID NO: 52) |
| AAVS1-PCR-F | PCR | AAVS1 | GATGCTCTTTCCGGAGCACT (SEQ ID |
| | template | | NO: 53) |
| AAVS1-PCR-R | PCR | AAVS1 | GCCAAGGACTCAAACCCAGAA (SEQ ID |
| | template | | NO: 54) |
| EMX1-RFLP-F | RFLP | EMX1 | TGGTGGATTTCGGACTACCCT (SEQ ID |
| | | | NO: 55) |
| EMX1-RFLP-R | RFLP | EMX1 | TTCGGACTGGAACCGTCAGC (SEQ ID |
| | | | NO: 56) |
| VEGFA-RFLP- | RFLP | VEGFA | AGACGTTCCTTAGTGCTGGC (SEQ ID |
| F | | | NO: 57) |
| VEGFA-RFLP- | RFLP | VEGFA | AAAAGTTTCAGTGCGACGCC (SEQ ID |
| R | | | NO: 58) |
| DYNLT1 KI | Junction | DYNLT1 | AGGAGGTCCCATCAGATGCT (SEQ ID |
| PCR-F | PCR | | NO: 59) |
| HSP90AA1 | Junction | HSP90AA1 | GGCTGGACAGCAAACATGGA (SEQ ID |
| KI PCR-F | PCR | | NO: 60) |
| AAVS1 KI | Junction | AAVS1 | GATGCTCTTTCCGGAGCACT (SEQ ID |
| PCR-F | PCR | | NO: 61) |
| Junction PCR | Junction | mKate | TTGCTGCCGTACATGAAGCTG (SEQ ID |
| universal-R | PCR | | NO: 62) |
| EMX1-NGS-F | NGS | EMX1 | CCATCTCATCCCTGCGTGTCTCCAGAAGA |
| | | | AGGGCTCCCATCAC (SEQ ID NO: 63) |
| EMX1-NGS-R | NGS | EMX1 | CCTCTCTATGGGCAGTCGGTGATgAGCAG |
| | | | CAAGCAGCACTCTG (SEQ ID NO: 64) |
| VEGFA-NGS- | NGS | VEGFA | CCATCTCATCCCTGCGTGTCTCCCAGCGT |
| F | | | CTTCGAGAGTGAGG (SEQ ID NO: 65) |
| VEGFA-NGS- | NGS | VEGFA | CCTCTCTATGGGCAGTCGGTGATgTTGGA |
| R | | | ATCCTGGAGTGACCC (SEQ ID NO: 66) |
| EMX-OT1-F | Off | EMX1 OT- | CCATCTCATCCCTGCGTGTCTCCACAAAA |
| | Target | 1 | GCTCCACATGCTAGGA (SEQ ID NO: 67) |
| EMX-OT1-R | Off | EMX1 OT- | CCTCTCTATGGGCAGTCGGTGATgGCTGA |
| | Target | 1 | CTTTGGGCTCCTTCT (SEQ ID NO: 68) |
| EMX-OT2-F | Off | EMX1 OT- | CCATCTCATCCCTGCGTGTCTCCACACAC |
| | Target | 2 | TCCCCAGGATCTCA (SEQ ID NO: 69) |
| EMX-OT2-R | Off | EMX1 OT- | CCTCTCTATGGGCAGTCGGTGATgAATGT |
| | Target | 2 | CAGCTGAAGCAGGCT (SEQ ID NO: 70) |
| EMX-0T3-F | Off | EMX1 OT- | CCATCTCATCCCTGCGTGTCTCCGGCTAC |
| | Target | 3 | CCTGACAACTGCTT (SEQ ID NO: 71) |
| EMX-OT3-R | Off | EMX1 OT- | CCTCTCTATGGGCAGTCGGTGATgAGGAC |
| | Target | 3 | AGACATGACAAGGCA (SEQ ID NO: 72) |
| VEGFA-OTI- | Off | VEGFA 0T- | CCATCTCATCCCTGCGTGTCTCCGCAGGC |
| F | Target | 1 | AAGCTGTCAAGGGT (SEQ ID NO: 73) |
| VEGFA-OTI- | Off | VEGFA 0T- | CCTCTCTATGGGCAGTCGGTGATgCCCTC |
| R | Target | 1 | ACACCCACACCCTCA (SEQ ID NO: 74) |
| VEGFA-OT2- | Off | VEGFA 0T- | CCATCTCATCCCTGCGTGTCTCCGGAGG |
| F | Target | 2 | GGTGTCATCGTTCTG (SEQ ID NO: 75) |
| VEGFA-OT2- | Off | VEGFA 0T- | CCTCTCTATGGGCAGTCGGTGATgCAAAT |
| R | Target | 2 | TGCGCCATAGCTGGG (SEQ ID NO: 76) |
| VEGFA-OT3- | Off | VEGFA 0T- | CCATCTCATCCCTGCGTGTCTCCTGAGCG |
| F | Target | 3 | CTCTTCGTCTTTCC (SEQ ID NO: 77) |
| VEGFA-OT3- | Off | VEGFA 0T- | CCTCTCTATGGGCAGTCGGTGATgGCCAG |
| R | Target | 3 | GAACACAGGAATGCTA (SEQ ID NO: 78) |

High-throughput Sequencing Data Analysis Processed (demultiplexed, trimmed, and merged) sequencing reads were analyzed to determine editing outcomes using CRISPPResso2⁵ by aligning sequenced amplicons to reference and expected HDR amplicons. The quantification window was increased to 10 bp surrounding the expected cut site to better capture diverse editing outcomes, but substitutions were ignored to avoid inclusion of sequencing errors. Only reads containing no mismatches to the expected amplicon were considered for HDR quantification; reads containing indels that partially matched the expected amplicons were included in the overall reported indel frequency.

Statistical Analysis Unless otherwise stated, all statistical analysis and comparison were performed using t-test, with 1% false-discovery-rate (FDR) using two-stage step-up method of Benjamini, Krieger and Yekutieli (Benjamini, Y., et. al, Biometrika 93, 491-507 (2006), incorporated herein by reference). All experiments were performed in triplicates unless otherwise noted to ensure sufficient statistical power in the analysis.

Determination of editing at predicted Cas9 off-target sites To evaluate RecT/RecE off-target editing activity at known Cas9 off-target sites, same genomic DNA extracts for knock-in analysis were used as template for PCR amplification of top predicted off-targets sites (high scored as predicted CRISPOR, a web-based analysis tool) for the EMX1, VEGFA guides, primer sequences are listed in Table 2.

iGUIDE Off-target Analysis Genome-wide, unbiased off-target analysis was performed following the iGUIDE pipeline (Nobles, C. L., et al. Genome Biol 20, 14 (2019), incorporated herein by reference) based on Guide-seq invented previously (Tsai, S., et al. Nat Biotechnol 33, 187-197 (2015), incorporated herein by reference). HEK293T cells were transfected in 20 uL Lonza SF Cell Line Nucleofector Solution on a Lonza Nucleofector 4-D with program DS-150 according to the manufacturer's instructions. 300 ng of gRNA-Cas9 plasmids (or 150 ng of each gRNACas9n plasmid for the double nickase), 150 ng of the effector plasmids, and 5 pmol of double stranded oligonucleotides (dsODN) were transfected. Cells were harvested after 72 hrs for genomic DNA using Agencourt DNAdvance reagent kit. 400 ng of purified gDNA which was then fragmented to an average of 500 bp and ligated with adaptors using NEBNext Ultra II FS DNA Library Prep kit following manufacturer's instructions. Two rounds of nested anchored PCR from the oligo tag to the ligated adaptor sequence were performed to amplify targeted DNA, and the amplified library was purified, size-selected, and sequenced using Illumina Miseq V2 PE300. Sequencing data was analyzed using the published iGUIDE pipeline, with the addition of a downsampling step which ensures an unbiased comparison across samples.

### Example 1

In contrast to mammals, convenient recombineering-edit tools are available for bacteria, e.g., the phage lambda Red and RecE/T. Microbial recombineering has two major steps: template DNA is chewed back by exonucleases (Exo), then the single-strand annealing protein (SSAP) supports homology directed repair by the template, optionally facilitated by nuclease inhibitor. A system for RNA-guided targeting of RecE/T recombineering activities was developed and achieved kilobase (kb) human gene-editing without DNA cutting.

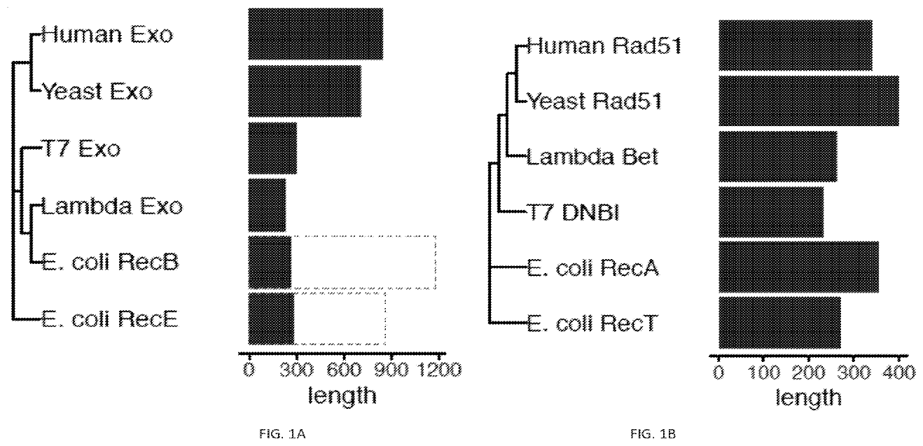

Candidate microbial systems with recombineering activities were surveyed. Two lines of reasoning guided the search: 1) Orthogonality: prioritizing proteins with minimal resemblance to mammalian repair enzymes; 2) Parsimony: focusing on systems with fewest interdependent components. Three protein families were identified: lambda Red, RecE/T, and phage T7 gp6 (Exo) and gp2.5 (SSAP) recombination machinery. Based on phylogenetic reconstruction, RecE/T proteins were determined to be the most distant from eukaryotic recombination proteins and among the most compact (FIG. 1). Thus, RecE/T systems were utilized for downstream analysis.

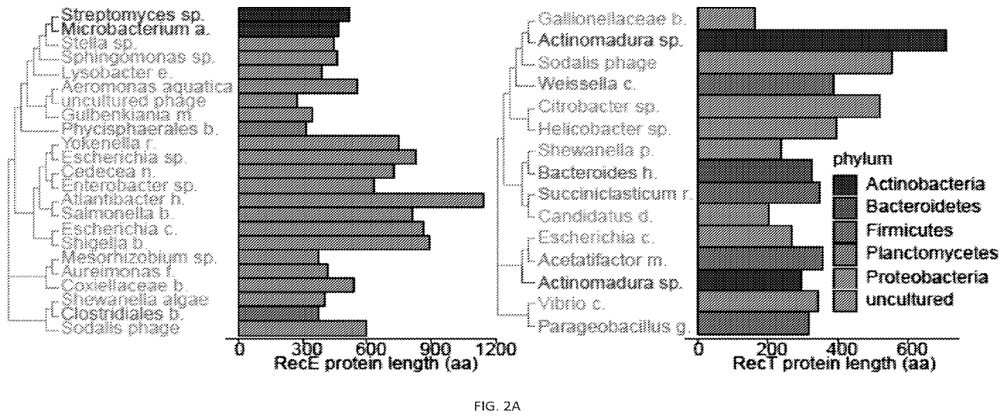

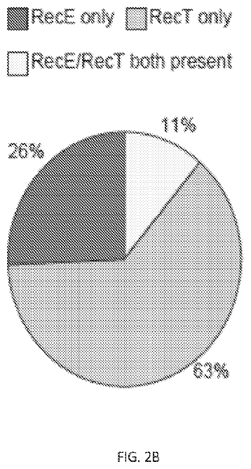

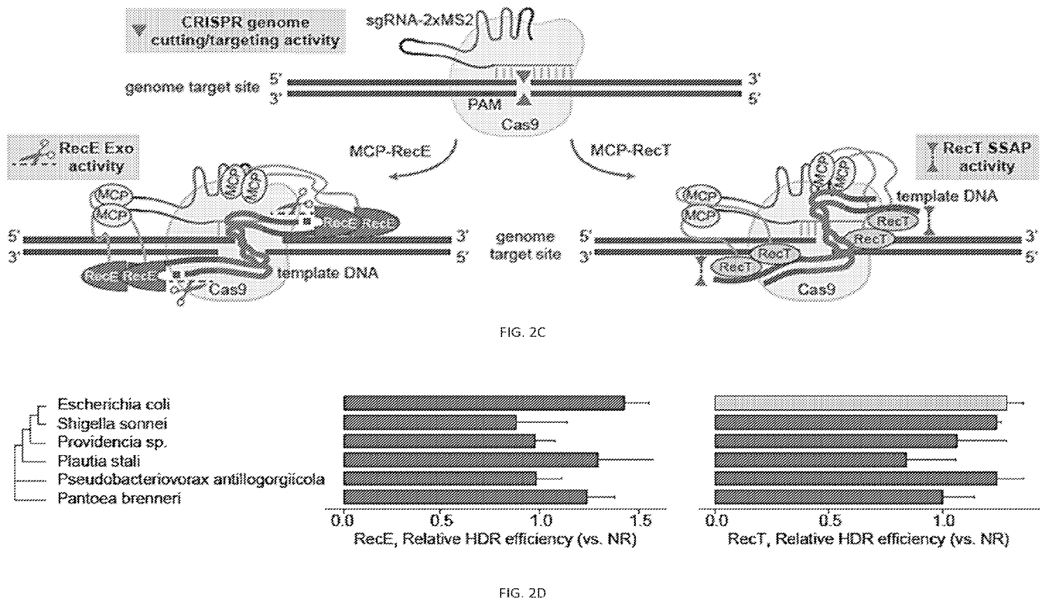

The NCBI protein database was systematically searched for RecE/T homologs. To develop a portable tool, evolutionary relationships and lengths were examined (FIG. 2A). Co-occurrence analysis revealed that most RecE/T systems have only one of the two proteins (FIG. 2B). As prophage integration could be imprecise, the 11% of species harboring both homologs were prioritized as evidence for intact functionality.

The top 12 candidates were codon-optimized and MS2 coat protein (MCP) fusions were constructed to recruit these RecE/T homologs, hereafter termed “recombinator”, to wild-type Streptococcus pyogenes Cas9 (wtCas9) via MS2 RNA aptamers. To understand their respective molecular effects as Exo and SSAP, each was tested independently (FIG. 2C). Initial results revealed Escherichia coli RecE/T proteins (simplified as RecE and RecT) as promising candidates, as determined by genome knock-in assays (FIG. 2D). While RecT is only 269 amino acid (AA) long, RecE was truncated from AA587 (RecE_587) and the carboxy terminus domain (RecE_CTD) based on functional studies (Muyrers, J. P., Genes Dev. (2000); 14, 1971-1982, incorporated herein by reference).

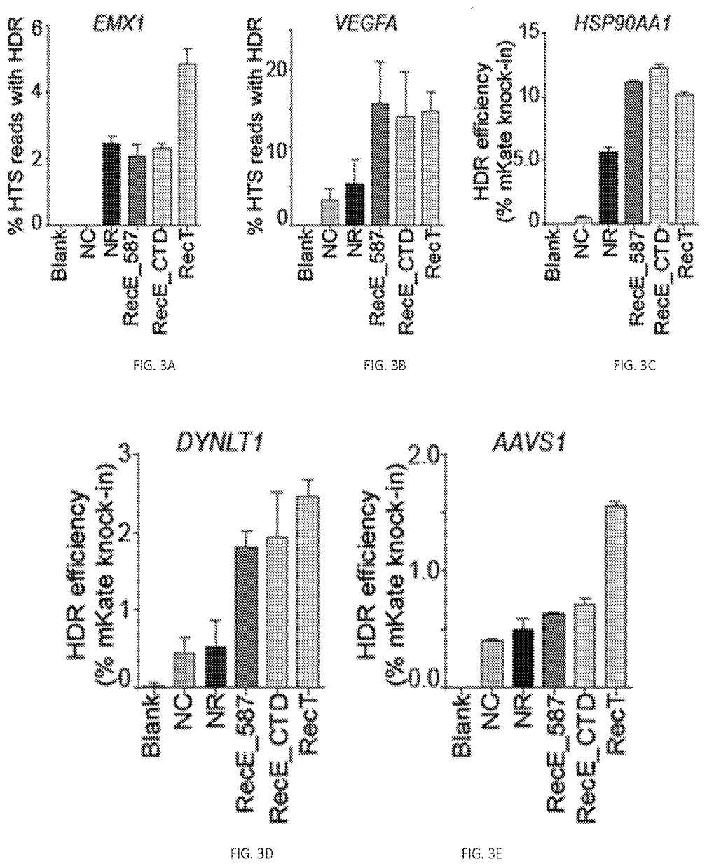

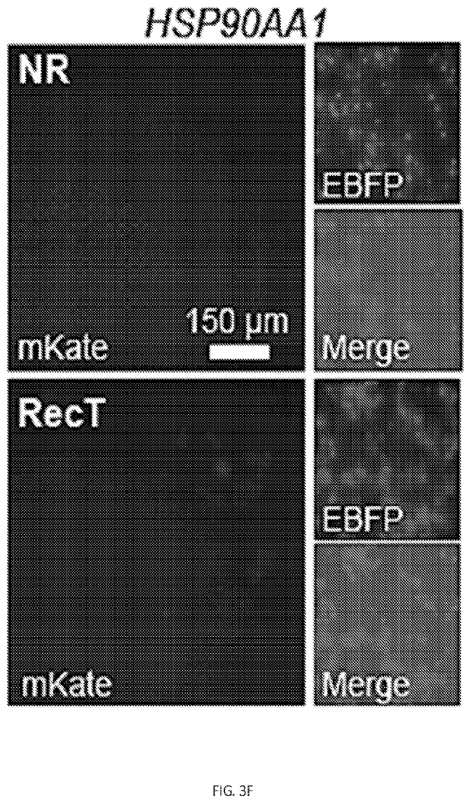

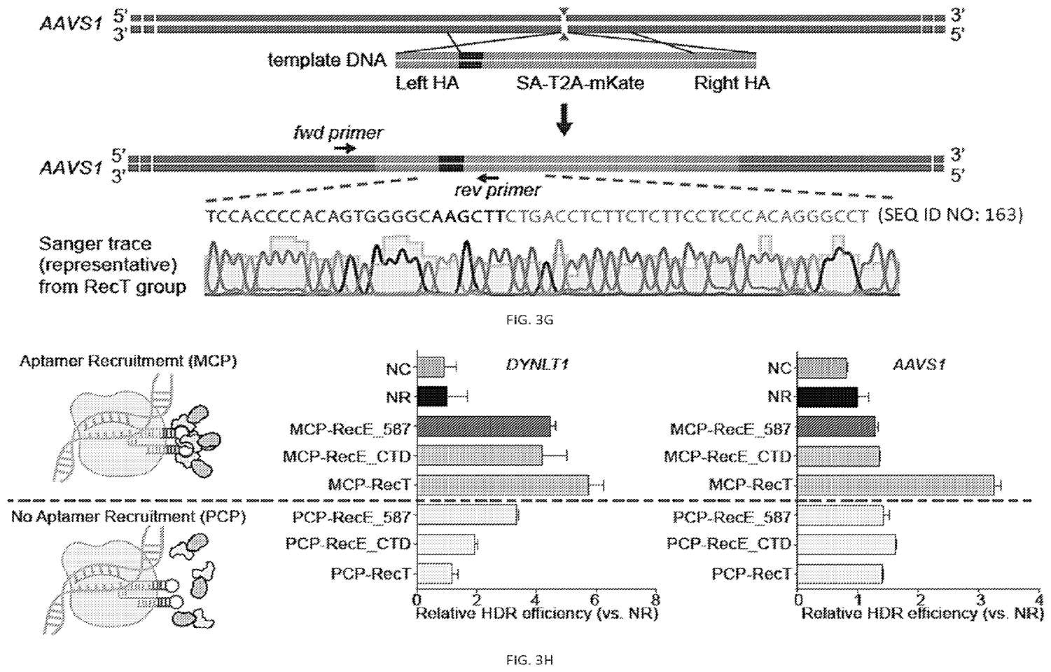

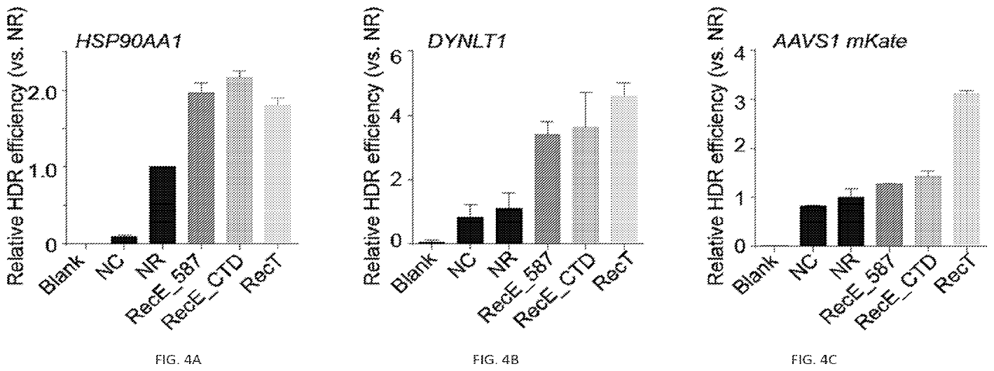

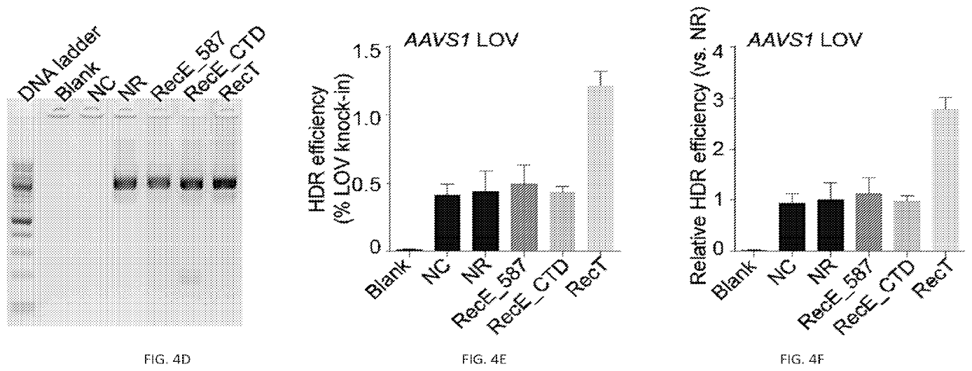

To validate RecE/T recombineering in human cells, homology directed repair (HDR) was measured at five genomic sites with two templates. While the RecE variants (RecE_587, RecE_CTD) demonstrated variable increases in knock-in efficiency, RecT significantly enhanced HDR in all cases, replacing ˜16 bp sequences at EMX1 and VEGFA, and knocking-in ˜1 kb cassette at HSP90AA1, DYNLT1, AAVS1 (FIGS. 3A-E, FIG. 4). These results were verified using imaging (FIG. 3F) and junction sites were sequenced using Sanger sequencing to confirm precise insertion (FIG. 3G). To test if these activities are truly sequence-specific, a no-recruitment control with the PP7 coat protein (PCP) that recognizes PP7 aptamers not MS2 aptamers was employed. RecE had activities without recruitment, whereas RecT showed efficiency increases in a recruitment-dependent manner (FIG. 3H). Without being bound by theory, this may be explained by RecE exonuclease activity acting promiscuously (FIG. 2C). The RecE/T recombineering-edit (REDIT) tools was termed as REDITv1, with REDITv1_RecT as the preferred variant.

### Example 2

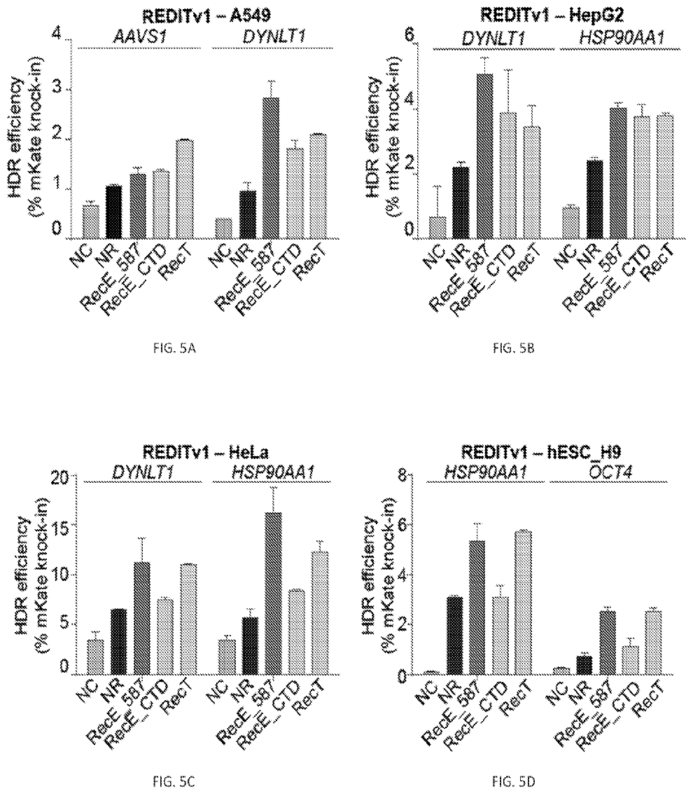

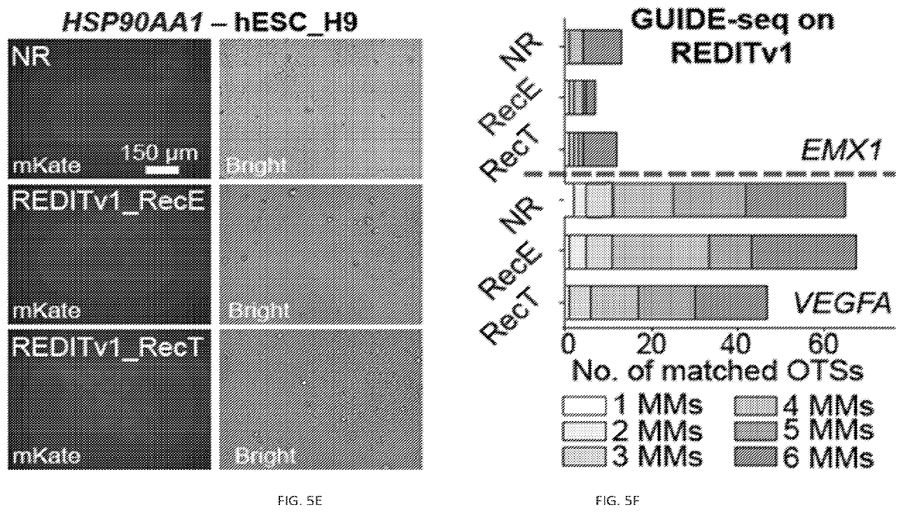

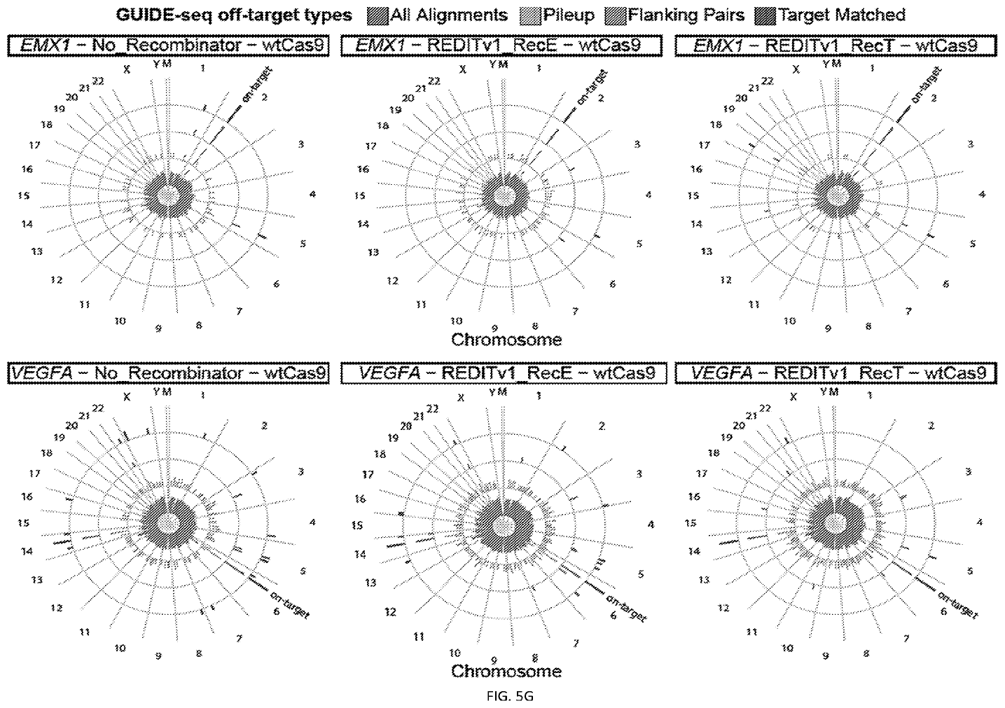

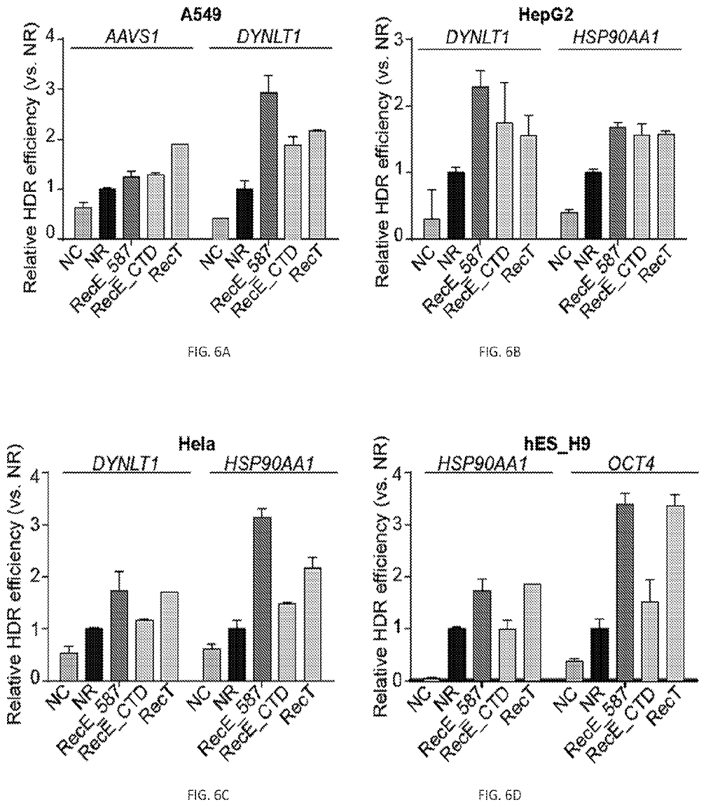

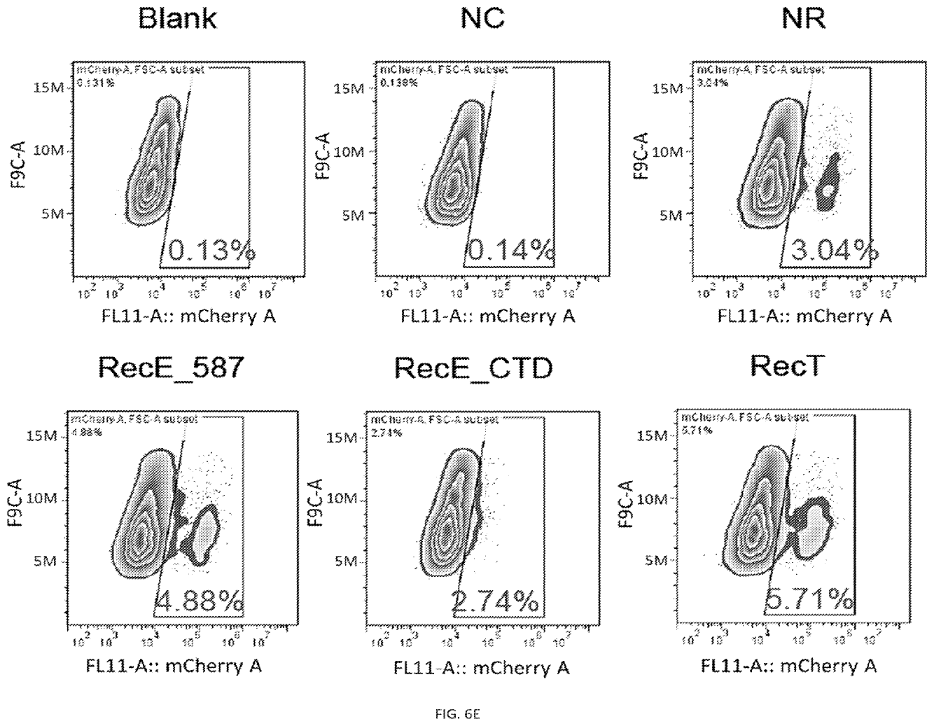

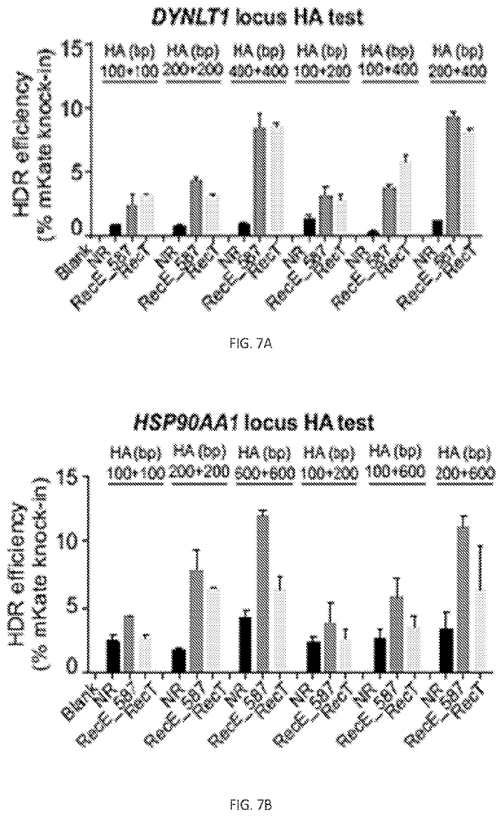

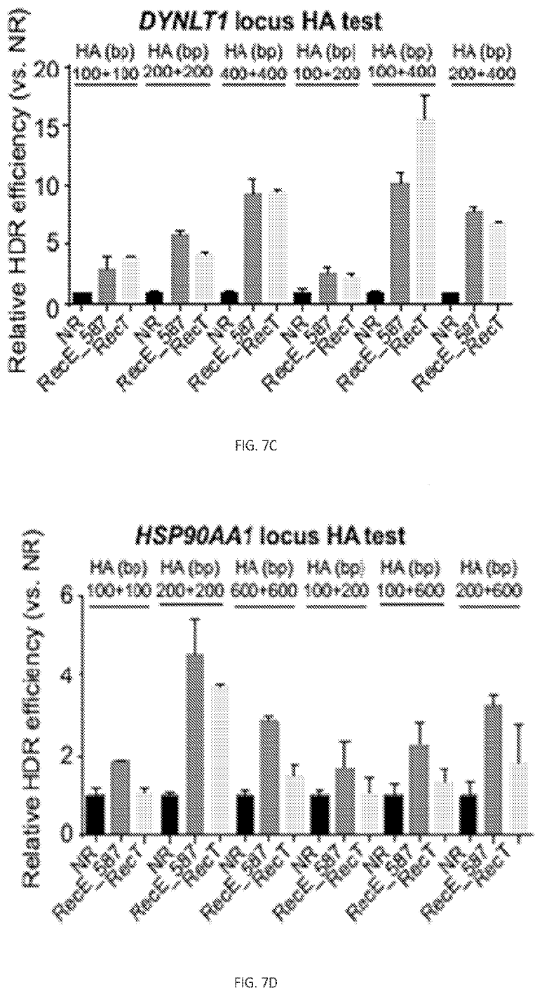

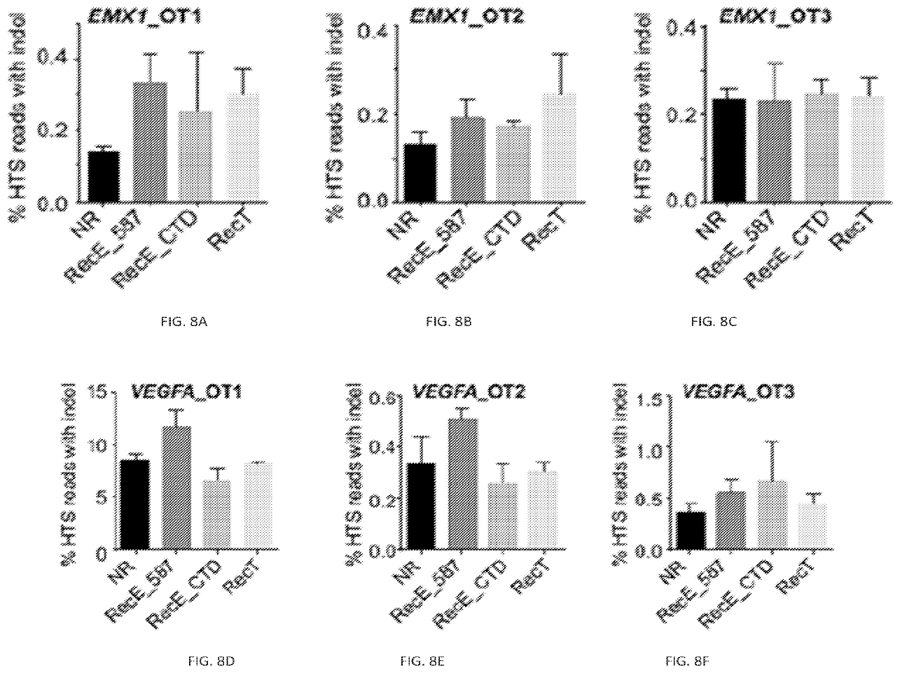

Three tests on REDITv1 were performed to explore: 1) activity across cell types, 2) optimal designs of HDR template, and 3) specificity. REDITv1 activity was robust across multiple genomic sites in HEK, A549, HepG2, and HeLa cells (FIGS. 5A-C, FIGS. 6A-C). Noticeably, in human embryonic stem cells (hESCs), REDITv1 exhibited consistent increases of kilobase knock-in efficiency at HSP90AA1 and OCT4, with up to 3.5-fold improvement relative to Cas9-HDR (FIGS. 5D-E, FIGS. 6D-E). Different template designs were also tested. REDITv1 performed efficient kilobase editing using HA length as short as 200 bp total, with longer HA supporting higher efficiency. It achieved up to 10% efficiency (without selection) for kb-scale knock-in, a 5-fold increase over Cas9-HDR and significantly higher than the 1-2% typical efficiency (FIG. 7). Lastly, the accuracy of REDITv1 accuracy was determined using deep sequencing of predicted off-target sites (OTSs) and GUIDE-seq. Although REDITv1 did not increase off-target effects, detectable OTSs remained at previously reported sites for EMX1 and VEGFA (FIGS. 5F-G, FIG. 8). In short, REDITv1 showcased kilobase-scale genome recombineering but retained the off-target issues, with REDITv1_RecT having the highest efficiency.

### Example 3

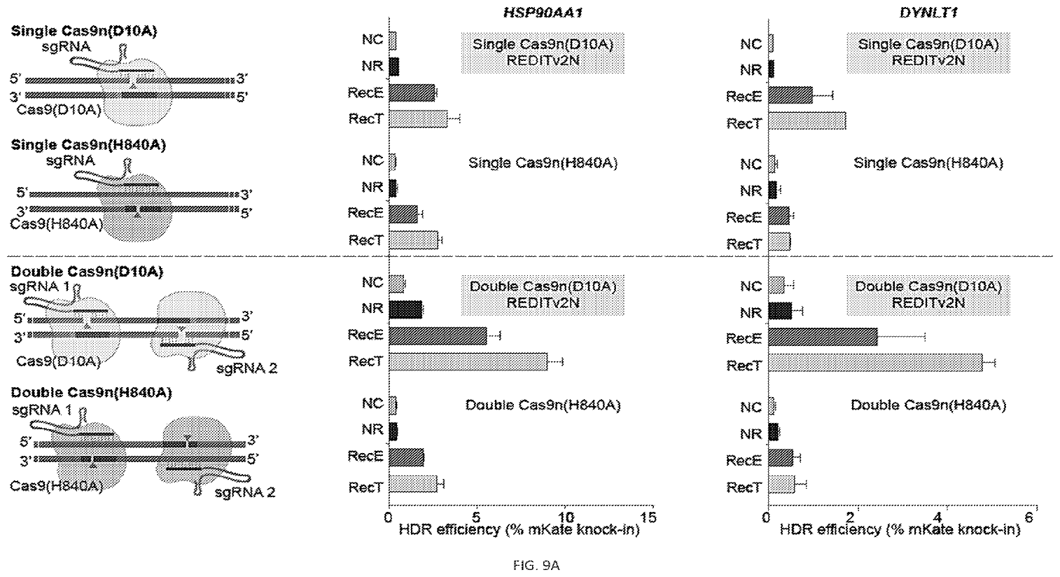

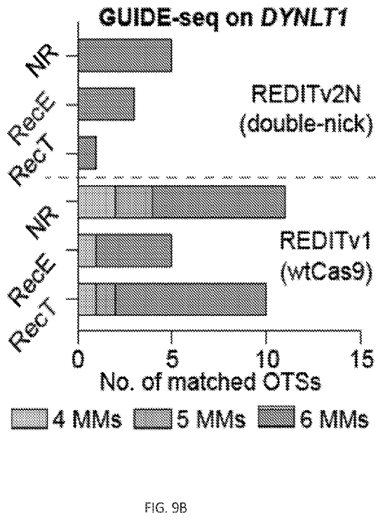

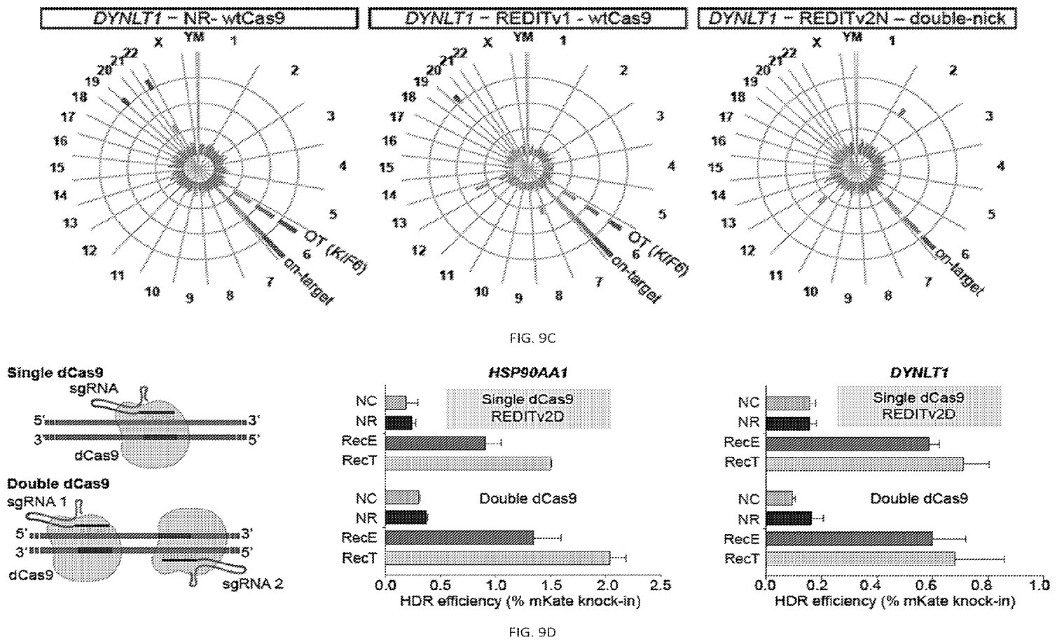

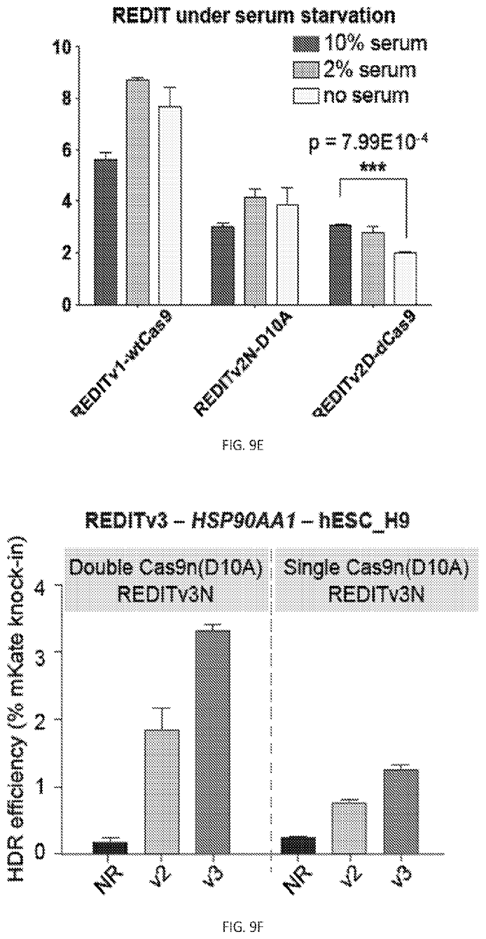

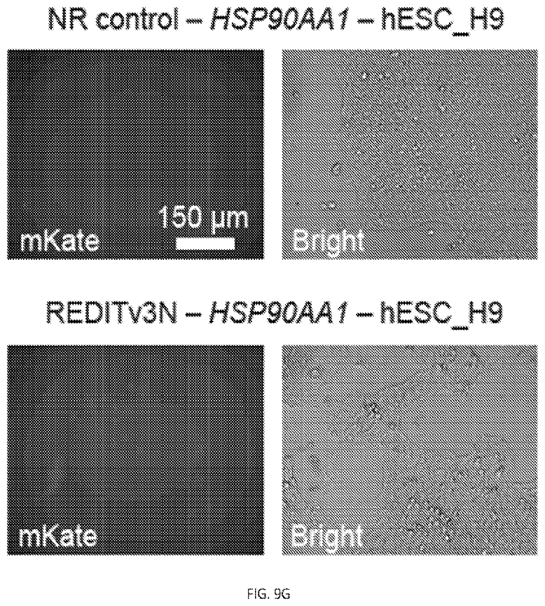

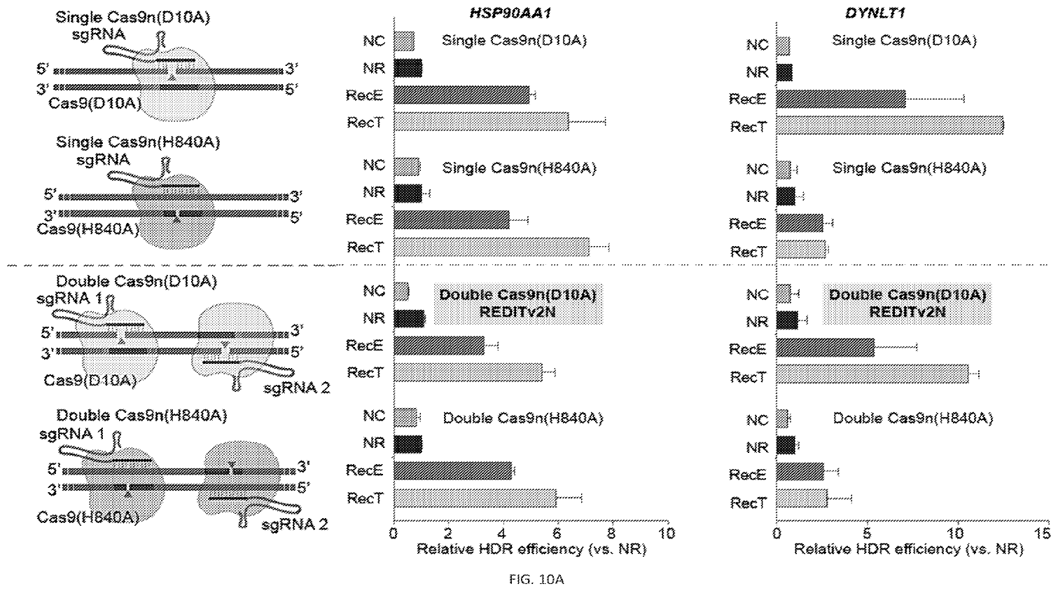

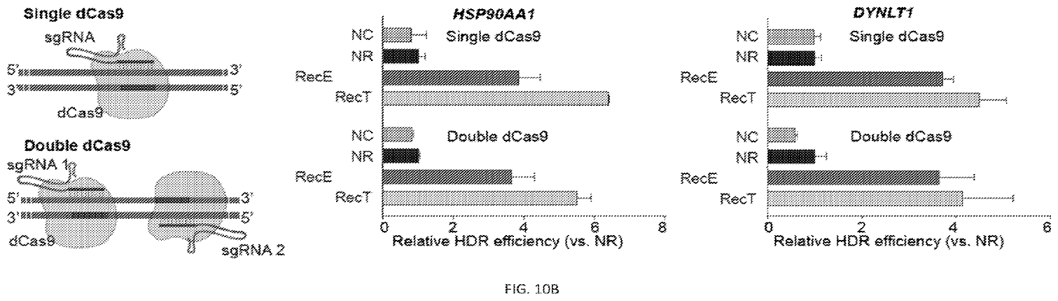

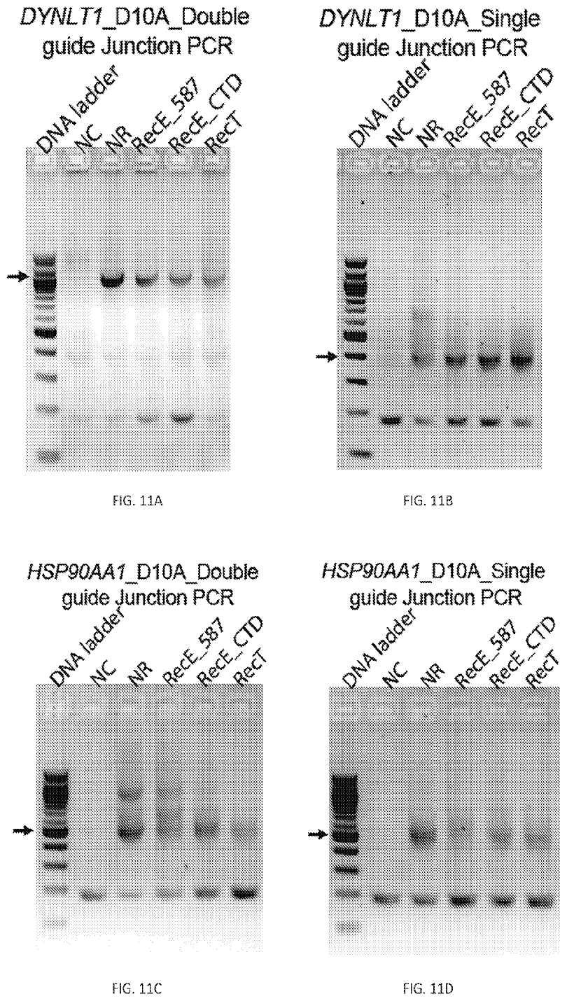

To alleviate unwanted edits, a version of REDIT with non-cutting Cas9 nickases (Cas9n) was assessed. A similar strategy was previously employed (Ran, F. A., et al., Cell (2013), 154: 1380-1389, incorporated herein by reference) to address off-target issues but had low HDR efficiency. REDIT was tested to determine if this system could overcome the limitation of endogenous repair and promote nicking-mediated recombination. Indeed, the nickase version demonstrated higher efficiencies, with the best results from Cas9n(D10A) with single- and double-nicking. This Cas9n(D10A) variant was designated REDITv2N (FIG. 9A). A 5%-10% knock-in without selection was observed using REDITv2N double-nicking, comparable to REDITv1 using wtCas9 (FIG. 9A, FIG. 10A). Junction sequencing confirmed the precision of knock-in for all targets (FIG. 11). This result represented 6- to 10-fold improvement over Cas9n-HDR. Even with single-nicking REDITv2N, a ˜2% efficiency for 1 kb knock-in was observed, a level considerably higher than the 0.46% HDR efficiency in previous report (Cong, L. et al., Science. 339, 819-823, incorporated herein by reference) using regular single-nicking Cas9n and a less-challenging 12-bp knock-in template (FIG. 9A).

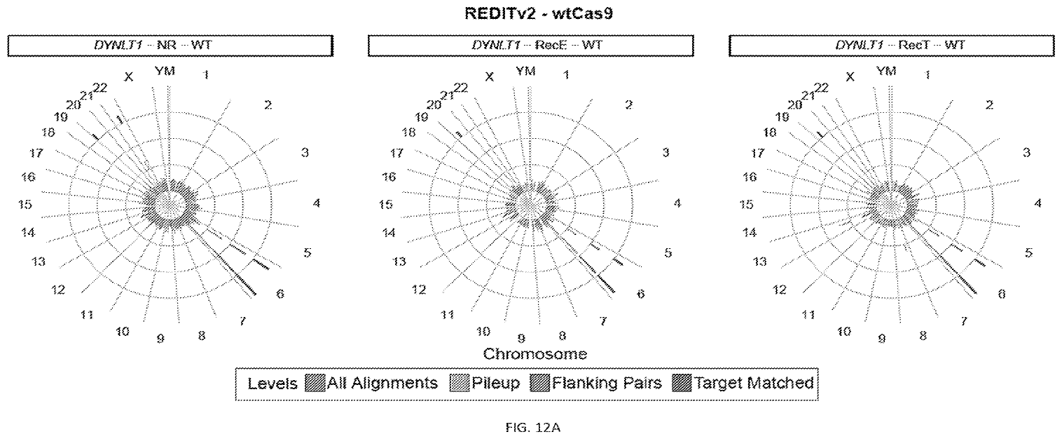

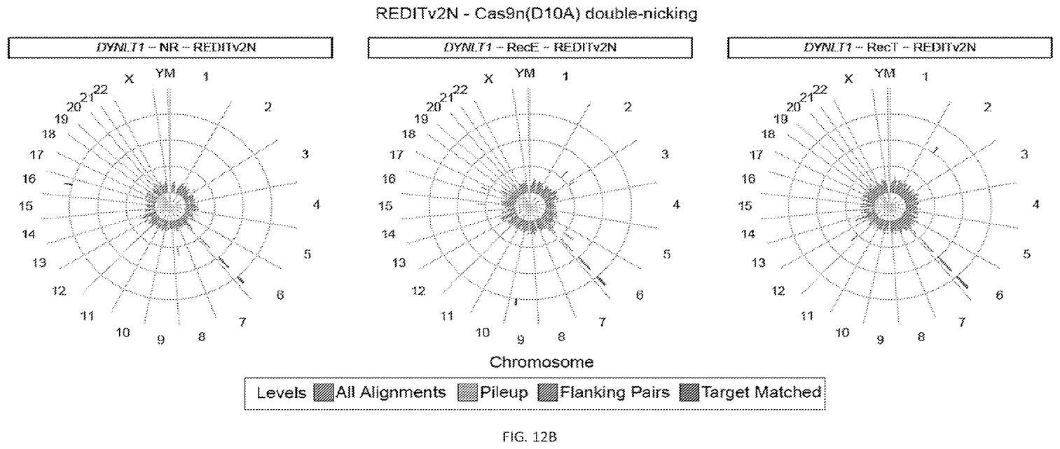

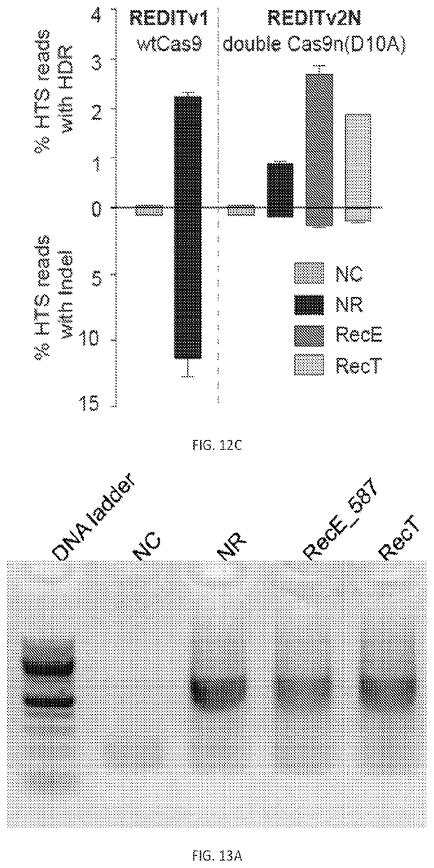

The off-target activity of REDITv2N was investigated using GUIDE-seq. Results showed minimal off-target cleavage and a reduction of OTSs by ˜90% compared to REDITv1 (FIG. 9B). Specifically, for DYNLT1-targeting guides, the most abundant KIF6 OTS was significantly enriched in REDITv1 group but disappeared when using REDITv2N (FIG. 9C). REDITv2N was highly accurate (FIGS. 9B-C, FIG. 12).

Another byproduct of HDR editing is on-target insertion-deletions (indels). They could drastically lower yields of gene-editing, especially for long sequences. Indel formation was measured in an EMX1 knock-in experiment using deep sequencing. REDITv2N increased HDR to the same efficiency as its counterpart using wtCas9 (FIG. 12C, top), with a reduction of unwanted on-target indels by 92% (FIG. 12C, bottom).

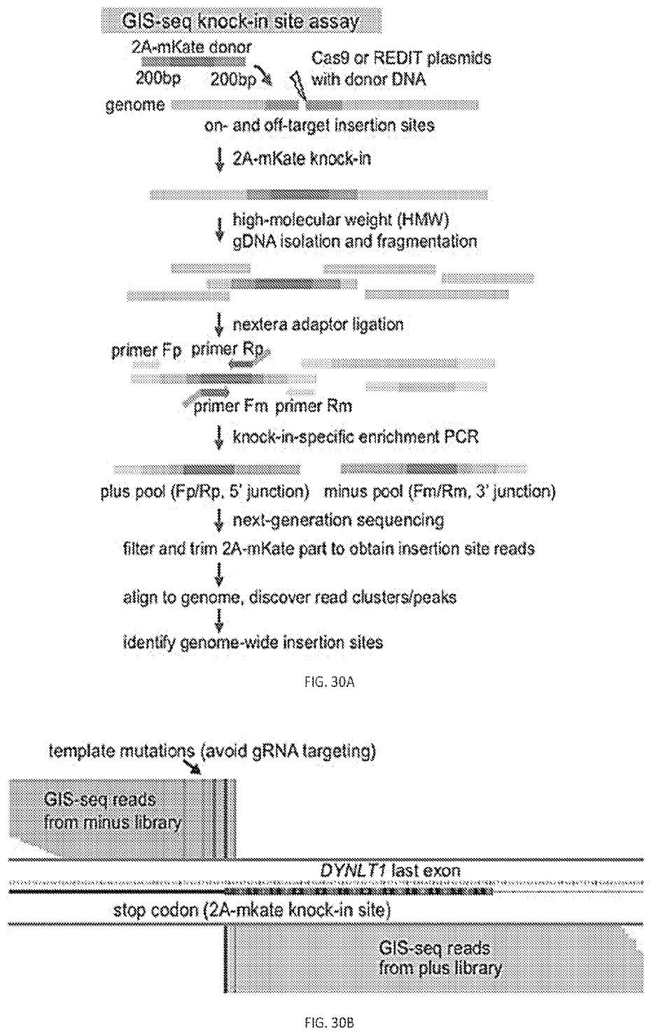

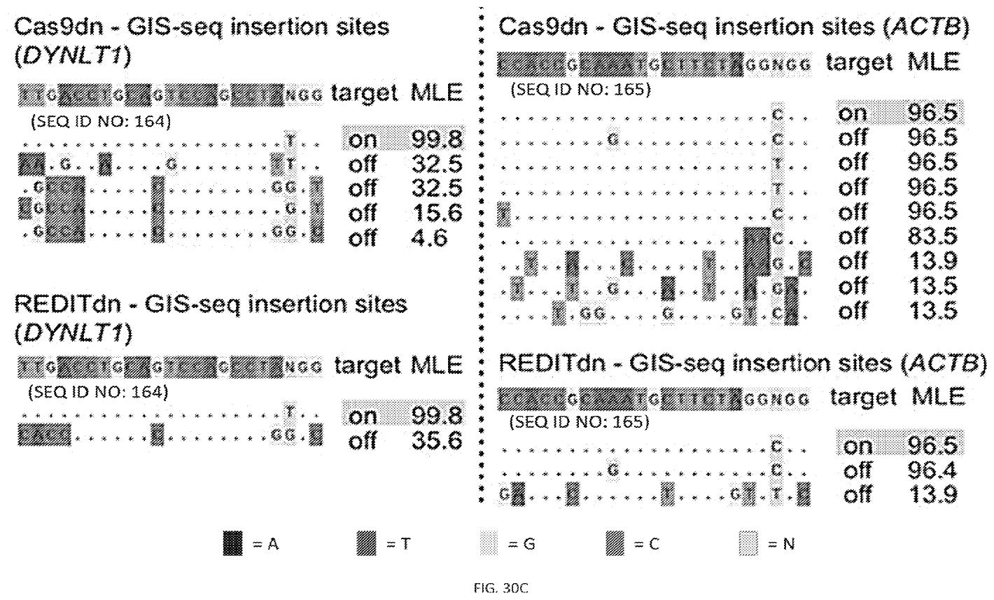

Concepts from GUIDE-seq, LAM-PCR, and TLA were used to develop an NGS-based assay to identify genome-wide insertion sites (GIS), or GIS-seq (FIG. 30A). Using GIS-seq, NGS read clusters/peaks representing knock-in insertion sites were obtained (FIG. 30B), showing representative reads from the on-target site). GIS-seq was applied to DYNLT1 and ACTB loci to measure the knock-in accuracy. Sequencing results indicated that, when considering sites with high confidence based on maximum likelihood estimation, REDIT had less off-target insertion sites identified compared with Cas9 (FIG. 30C). Together, the clonal Sanger sequencing of knock-in junctions (FIGS. 9C and 12), GUIDE-seq analysis (FIG. 9B), and GIS seq results (FIGS. 30A-30C) indicated that REDIT can be an efficient method with the ability to insert kilobase-length sequences with less unwanted editing events.

### Example 4

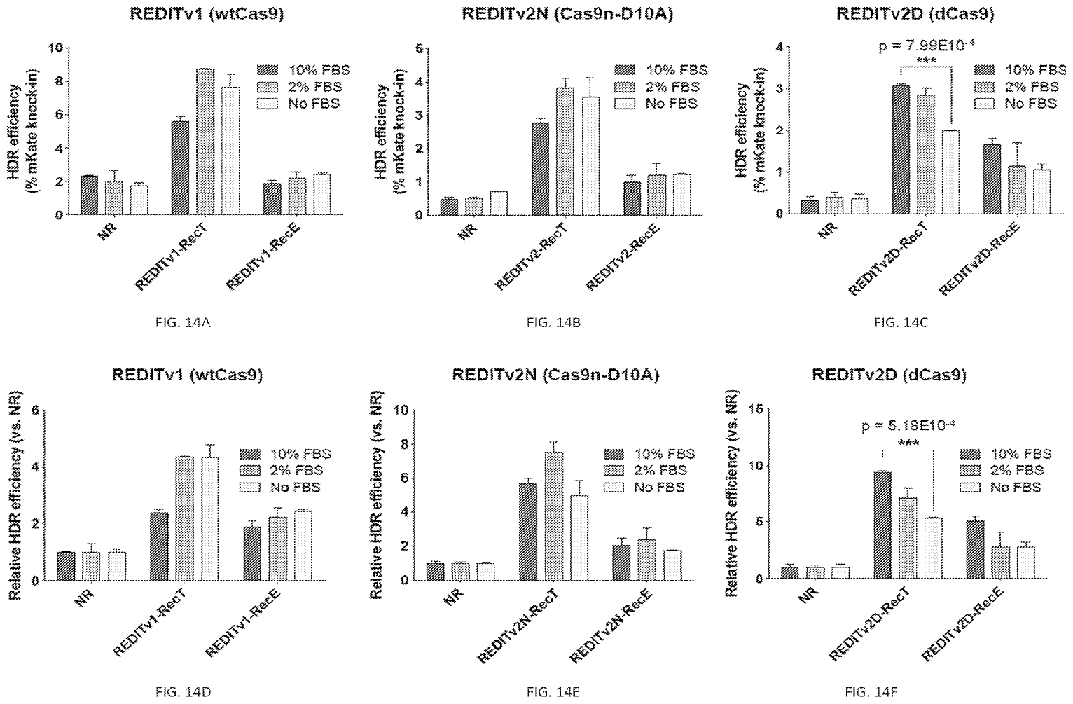

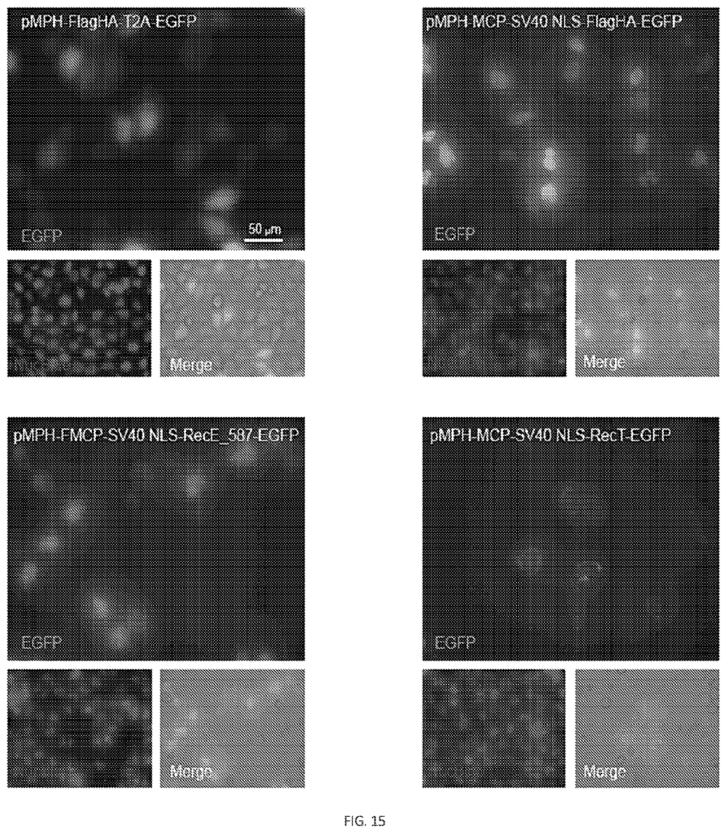

REDIT was examined for long sequence editing ability in the absence of any nicking/cutting of the target DNA. Remarkably, when using catalytically dead Cas9 (dCas9) to construct REDITv2D, an exact genomic knock-in of a kilobase cassette was observed in human cells (FIG. 9D, top, FIG. 13). While REDITv2D has lower efficiency than REDITv2N, it achieved programmable DNA-damage-free editing at kilobase-scale with 1-2% efficiency and no selection (FIG. 9D, FIG. 10B). It was hypothesized that two processes could be contributing to the REDITv2D recombineering. One possibility was via dCas9 unwinding. If dCas9 could unwind DNA as it induces sequence-specific formation of loop, a double-binding with two dCas9s would be expected to promote genome accessibility to RecE/T. However, a significant increase upon delivering two guide RNAs was not observed (FIG. 9D, bottom). Another possibility was that the unwinding of DNA during cell cycle permitted RecE/T to access the target region mediated by dCas9 binding. A 1 kb knock-in was performed with different REDIT tools at varying serum levels (10% regular, 2% reduced, and no serum). As serum starvation arrests cell proliferation, the results indicated that the cell cycle correlated positively with REDITv2D recombineering (FIG. 9E). Upon no-serum treatment, HDR efficiency only dropped in REDITv2D(dCas9) group, whereas REDITv1(wtCas9) and REDITv2N(D10A) were not affected (FIG. 9E, FIG. 14), supporting that DNA unwinding permitted RecE/T to access the target region.

### Example 5

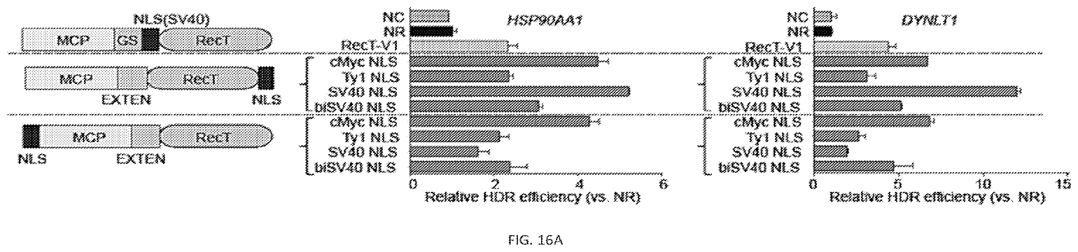

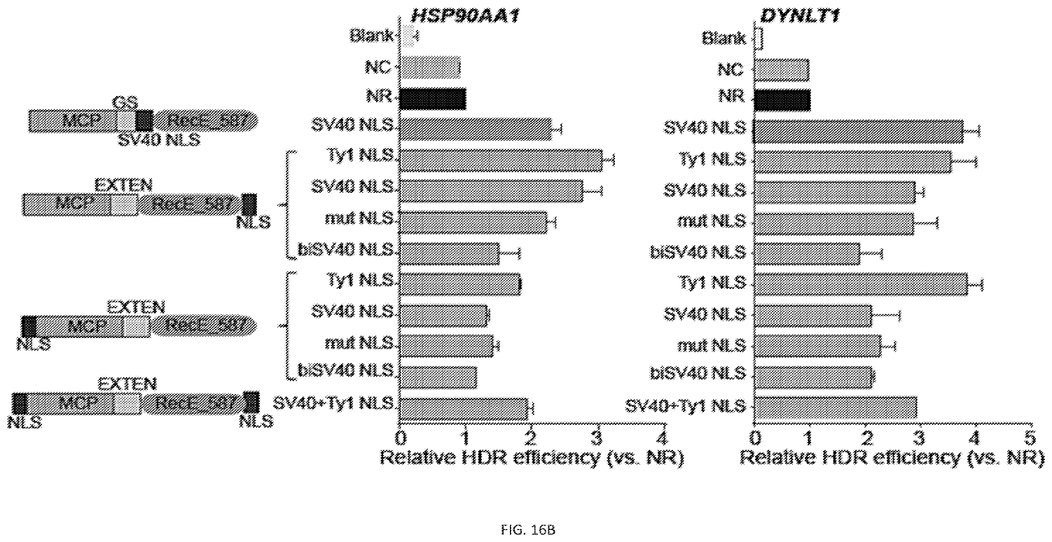

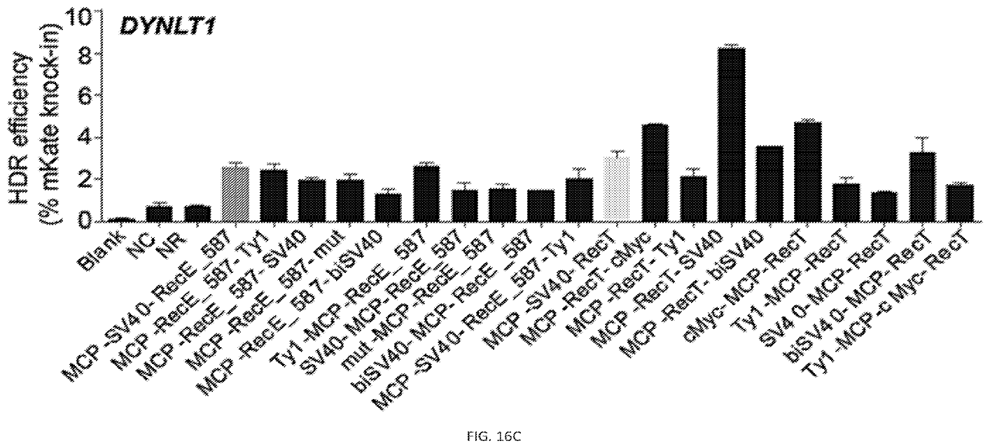

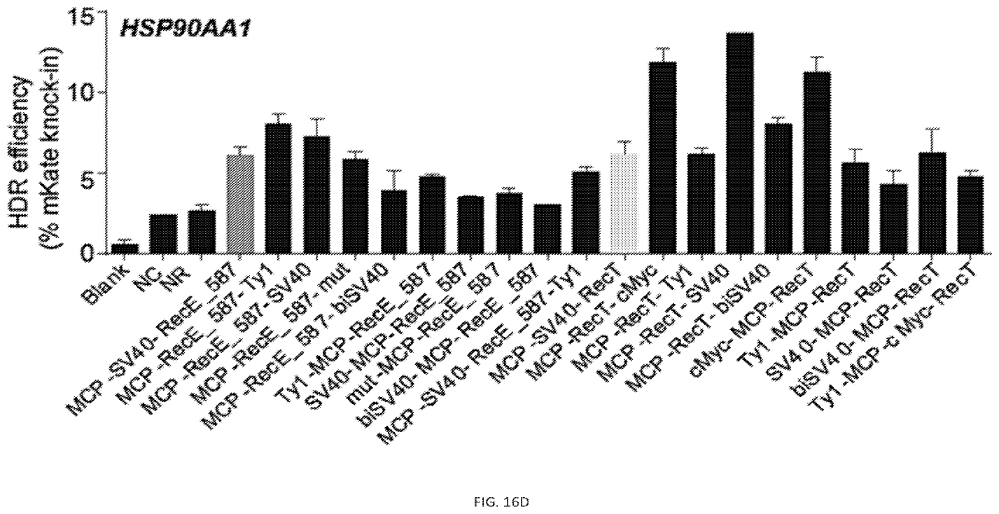

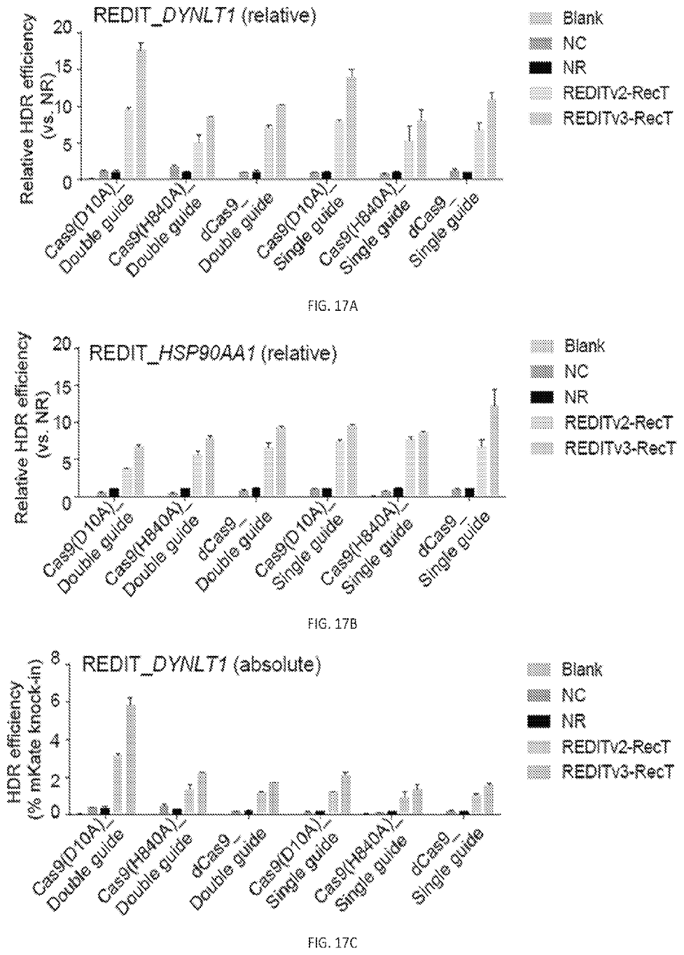

Microscopy analysis revealed incomplete nuclei-targeting of REDITv1, particularly REDITv1 RecT (FIG. 15). Hence, different designs of protein linkers and nuclear localization signals (NLSs) were tested (FIG. 15A). The extended XTEN-linker with C-terminal SV40-NLS was identified as a preferred configuration, termed REDITv3 (FIG. 16). REDITv3 further achieved a 2- to 3- fold increase of HDR efficiencies over REDITv2 across genome targets and Cas9 variants (wtCas9, Cas9n, dCas9) (FIG. 17).

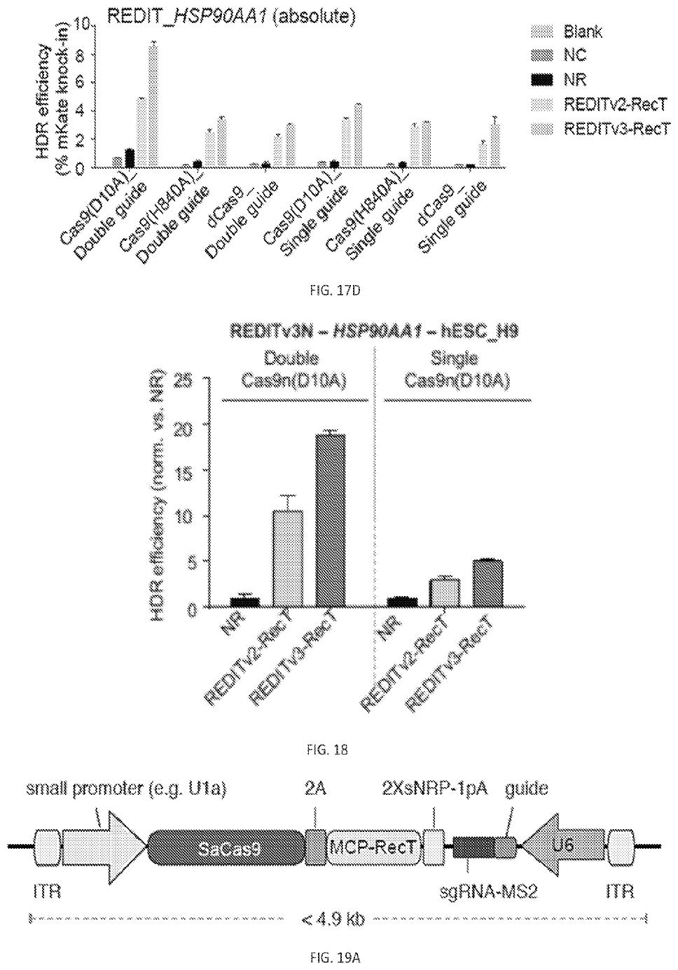

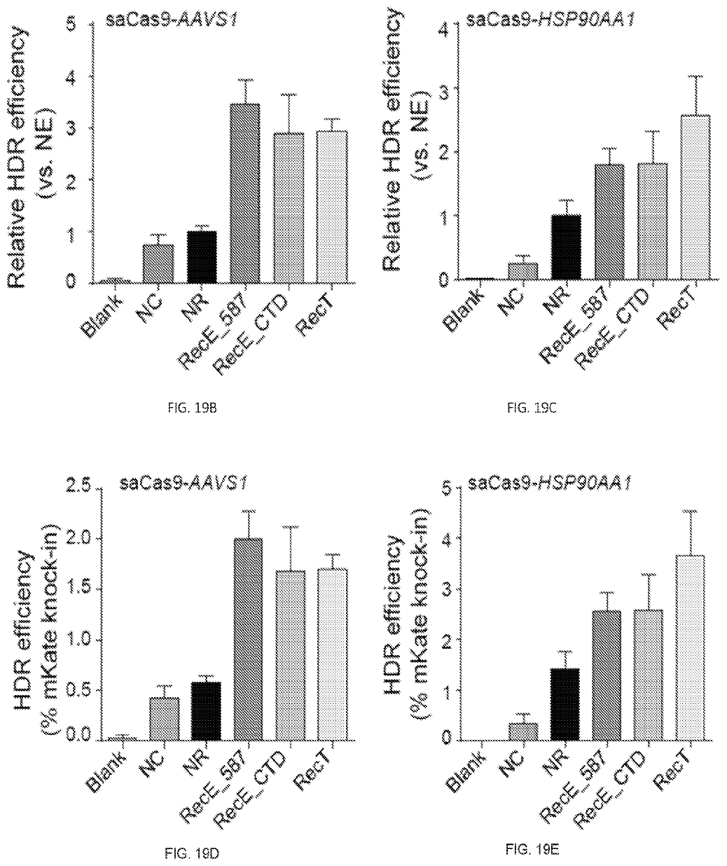

Finally, REDITv3 was utilized in hESCs to engineer kilobase knock-in alleles in human stem cells. REDITv3N single- and double-nicking designs resulted in 5-fold and 20-fold increased HDR efficiencies over no-recombinator controls, respectively (FIG. 9F). The efficacy and fidelity were confirmed via a combination of assays described for previous REDIT versions (FIGS. 9F-G, FIG. 18). Additionally, REDITv3 works effectively with Staphylococcus aureus Cas9 (SaCas9), a compact CRISPR system suitable for in vivo delivery (FIG. 19).

### Example 6

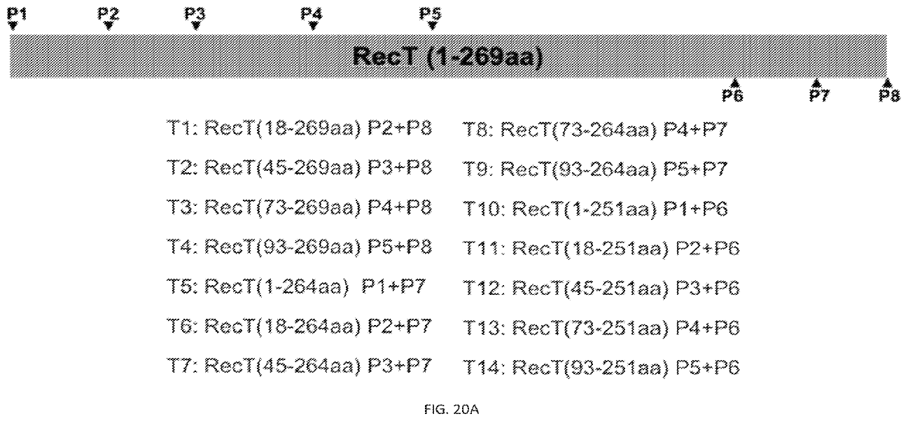

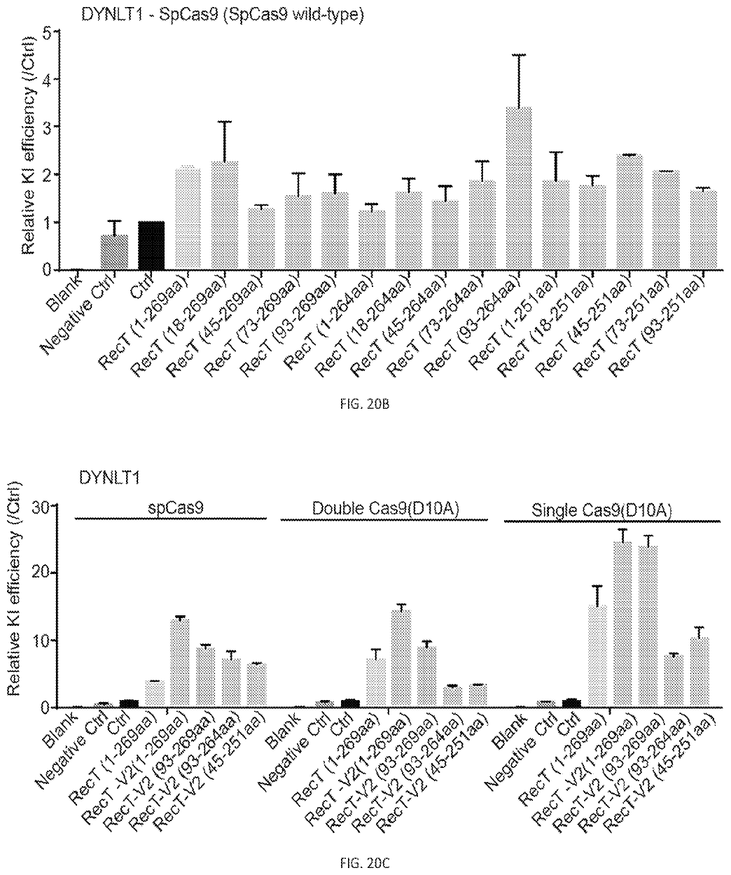

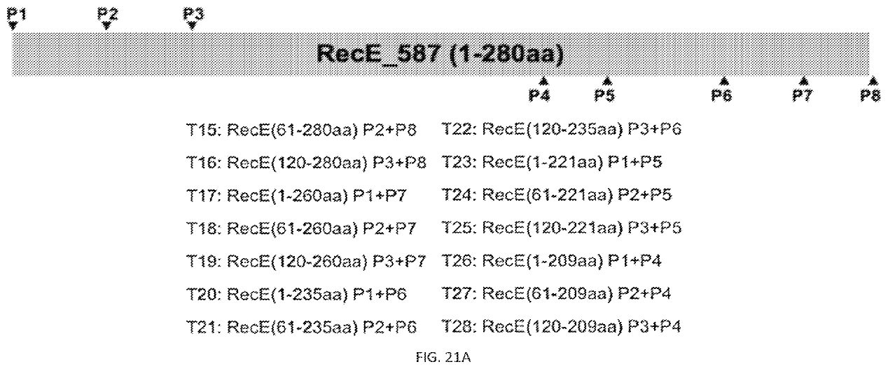

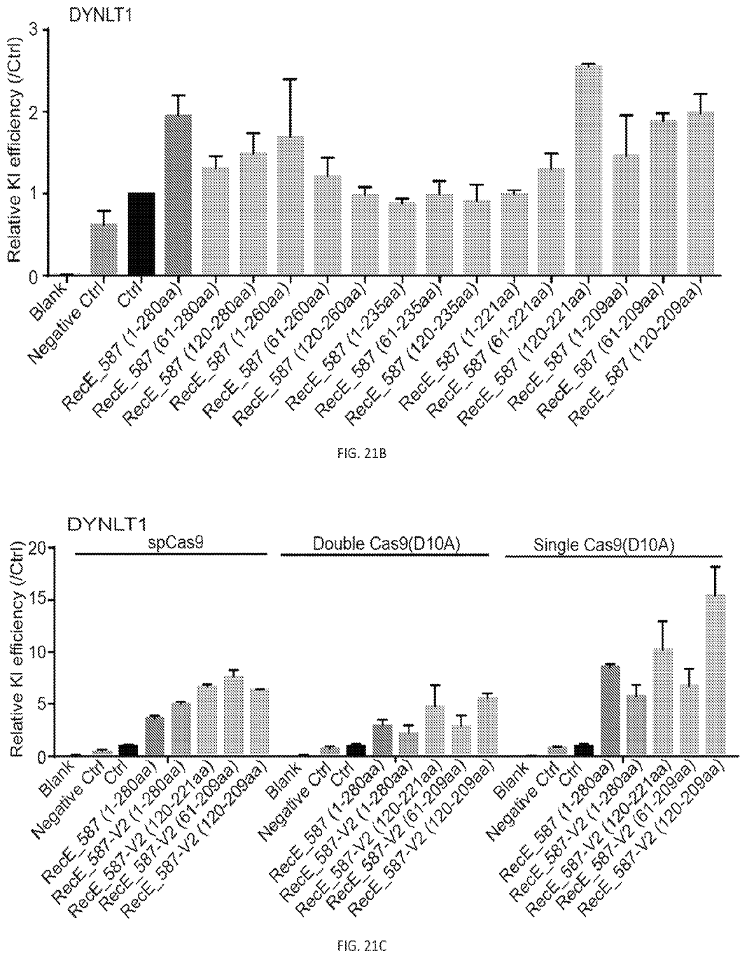

To further investigate RecT and RecE_587 variants, both RecT and RecE_587 were truncated at various lengths as shown in FIG. 20A and FIG. 21A, respectively. The resulting efficiencies were measured using an mKate knock-in assay, with both wildtype SpCas9 and Cas9n(D10A) with single- and double-nicking at the DYNLT1 locus (FIGS. 20B-C and FIGS. 21B-C, respectively). Efficiencies of the no recombination group are shown as the control.

The truncated versions of both RecT and RecE_587 retained significant recombineering activity when used with different Cas9s. In particular, compared with the full-length RecT(1-269aa), the new truncated versions such as RecT(93-264aa) are over 30% smaller yet they preserved essentially the full activities of RecT in stimulating recombination in eukaryotic cells. Similarly, compared with the full-length RecE(1-280aa), truncated versions such as RecE_587(120-221aa) and RecE_587(120-209aa) are over 60% smaller but still retained high recombination activities in human cells. These truncated versions demonstrated the potential to further engineer minimal-functional recombineering enzymes using RecE and RecT protein variants, but also provide valuable compact recombineering tools for human genome editing that is ideal for in vitro, ex vivo, and in vivo delivery given their small size.

Overall, REDIT harnessed the specificity of CRISPR genome-targeting with the efficiency of RecE/RecT recombineering. The disclosed high-efficiency, low-error system makes a powerful addition to existing CRISPR toolkits. The balanced efficiency and accuracy of REDITv3N makes it an attractive therapeutic option for knock-in of large cassette in immune and stem cells.

### Example 7

The reconstructed RecE and RecT phylogenetic trees with eukaryotic recombination enzymes from yeast and human (FIGS. 1A and 1B) show the evolutionary distance of the proteins based on sequence homology. The dotted boxes indicate the full-length E. coli RecB and E. coli RecE protein. The catalytic core domain of E. coli RecB and E. coli RecE protein (solid boxes) was used for the comparison. The gene-editing activities of these families of recombineering proteins were measured using the MS2-MCP recruitment system, where sgRNA bearing MS2 stem-loop is used with recombineering proteins fused to the MCP protein via peptide linker and with nuclear-localization signals.

Three exonuclease proteins were used: the exonuclease from phage Lambda, the RecE587 core domain of E. coli RecE protein, and the exonuclease (gene name gp6) from phage T7 (FIG. 22A). The gene-editing activity was measured using mKate knock-in assay at genomic loci (DYNLT1 and HSP90AA1).

Similar measurements were made testing the genome editing efficiencies of three single-strand DNA annealing proteins (SSAPs) from the same three species of microbes as the exonucleases, namely Bet protein from phage Lambda, RecT protein from E. coli, and SSAP (gene name gp2.5) from phage T7 (FIG. 22B).

From these results, the genome recombineering activities of all three major family of phage/microbial recombination systems was systematically measured and validated in eukaryotic cells (lambda phage exonuclease and beta proteins; E. coli prophase RecE and RecT proteins, T7 phage exonuclease gp6 and single-strand binding gp2.5 proteins). All six proteins from three systems achieved efficient gene editing to knock-in kilobase-long sequences into mammalian genome across two genomic loci. Overall, the exonucleases showed ˜3-fold higher recombination efficiency (up to 4% mKate genome knock-in) when compared with no-recombinator controls. The single-strand annealing proteins (SSAP) showed higher activities, with 4-fold to 8-fold higher gene-editing activities over the control groups. This demonstrated the general applicability and validity that microbial recombination proteins in the exonuclease and SSAP families could be engineered via the Cas9-based fusion protein system to achieve highly efficient genome recombination in mammalian cells.

### Example 8

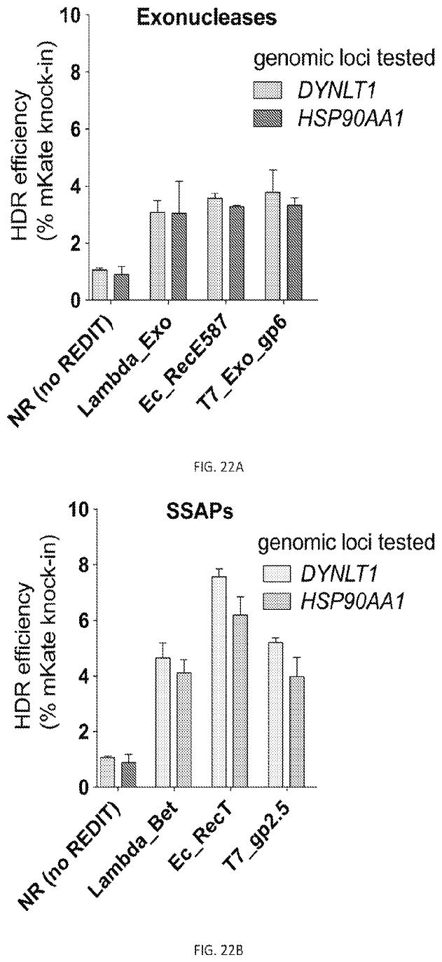

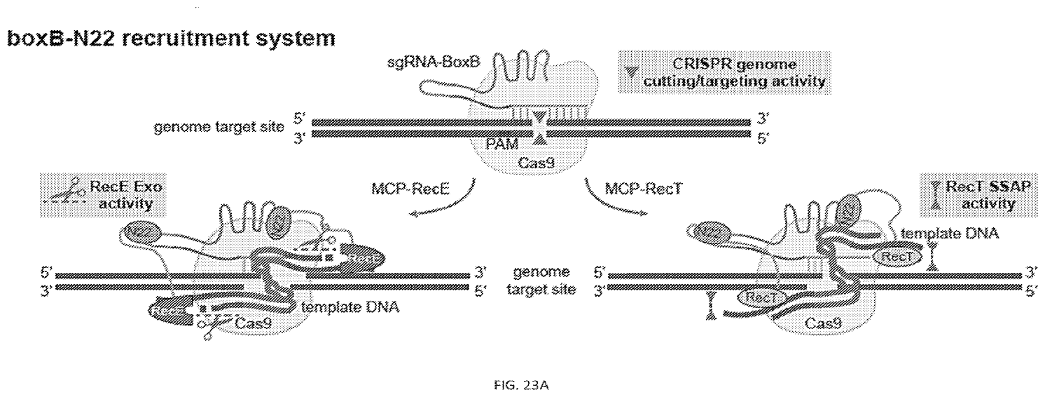

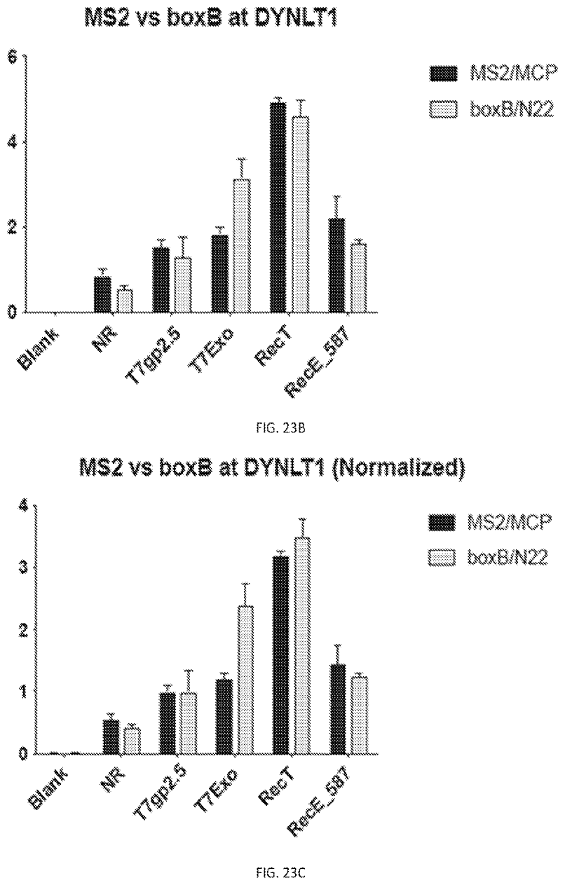

In order to demonstrate the generalizability of REDIT protein design, alternative recruitment systems were developed and tested. For a more compact REDIT system, the REDIT recombinator proteins were fused to N22 peptide and at the same time the sgRNA included boxB, the short cognizant sequence of N22 peptide, replacing MCP within the sgRNA (FIG. 23A). This boxB-N22 system demonstrated comparable editing efficiencies at the two genomic sites tested as shown in FIGS. 23B-23E with side-by-side comparisons of the MS2-MCP recruitment system.

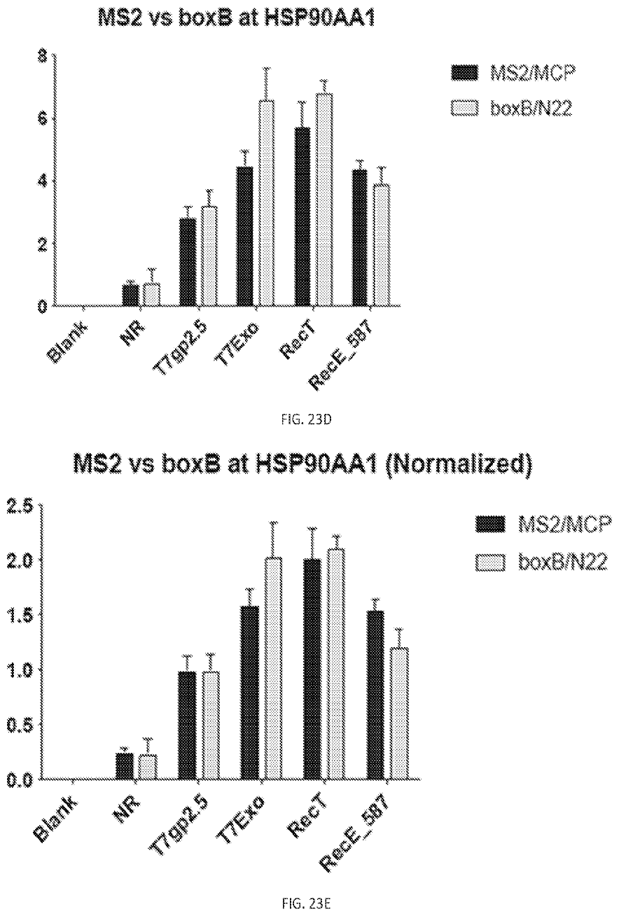

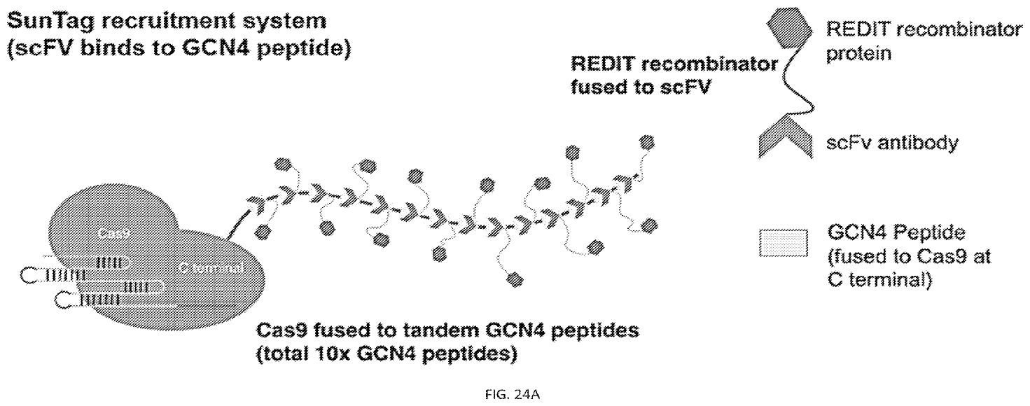

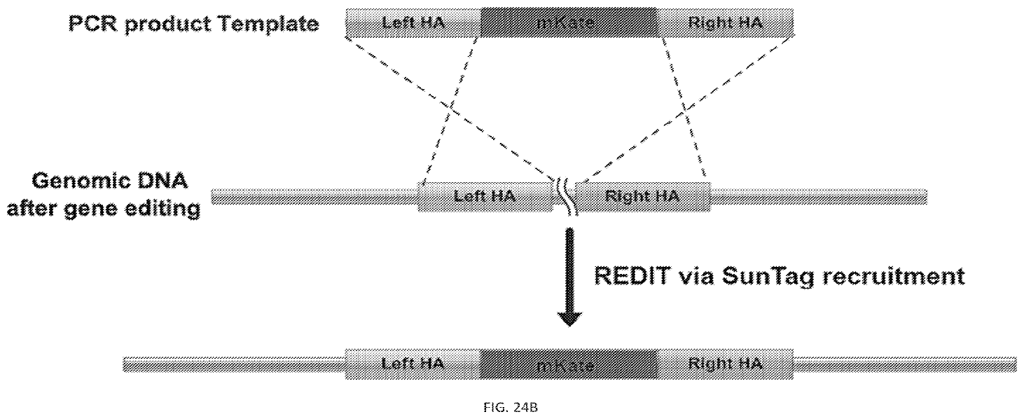

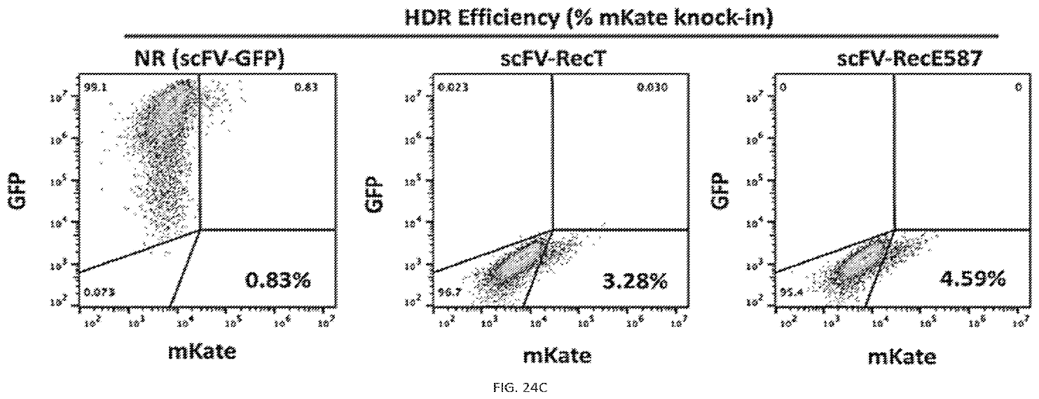

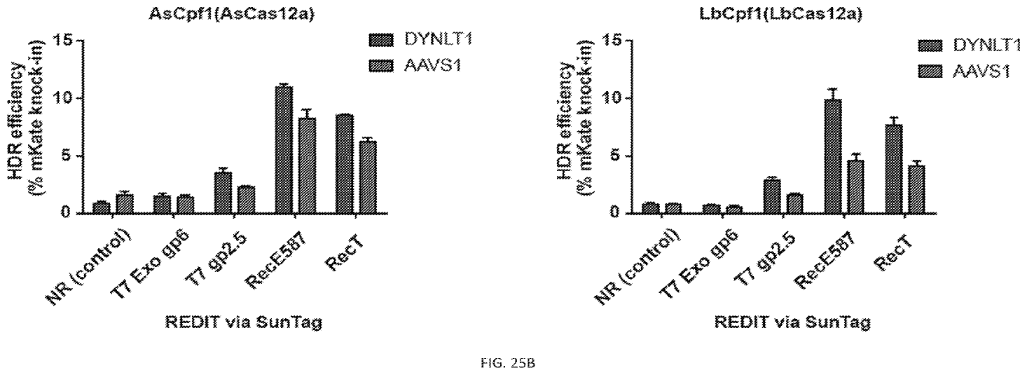

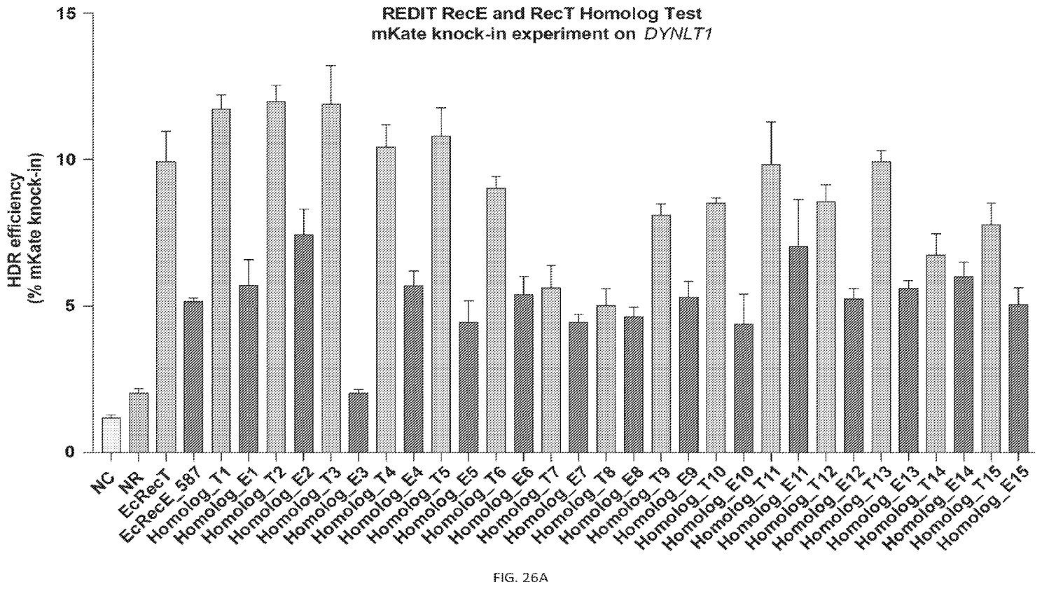

A REDIT system using SunTag recruitment, a protein-based recruitment system, was developed (FIGS. 24A and 27A). Because SunTag is based on fusion protein design, the sgRNA or guideRNAs are the same as wild-type CRISPR system. Specifically, the REDIT recombinator proteins were fused to scFV antibody peptide (replacing MCP), and the GCN4 peptide was fused in tandem fashion (10 copies of GCN4 peptide separated by linkers) to the Cas9 protein. Thus, the scFV-REDIT could be recruited to the Cas9 complex via affinity of GCN4 to scFV.

mKate knock-in experiments (FIGS. 24B and 27B) were used to measure the editing efficiencies at the DYNLT1 locus and the HSP90AA1 locus, respectively. This SunTag-based REDIT system demonstrated significant increase of gene-editing knock-in efficiency at the DYNLT1 genomic sites tested. In addition, the SunTag design significantly increased HRD efficiencies to ˜2-fold better than Cas9 but did not achieve increases as high as the MS2-aptamer.

### Example 9

In order to demonstrate the generalizability of REDIT protein design and develop versatile REDIT system applicable to a range of CRISPR enzymes, Cpf1/Cas12a based REDIT system using the SunTag recruitment design was developed (FIG. 25A). Two different Cpf1/Cas12a proteins were tested (Lachnospiraceae bacterium ND2006, LbCpf1 and Acidaminococcus sp. BV3L6) using the mKate knock-in assay as previously shown (FIG. 25B).

These results showed that the microbial recombination proteins (exonuclease and single-strand annealing proteins) could be engineered using alternative designs such as the SunTag recruitment system to perform genome editing in eukaryotic cells. These protein-based recruitment system does not require the usage of RNA aptamers or RNA-binding proteins, instead, they took advantage of fusion protein domains directly connecting to the CRISPR enzymes to recruit REDIT proteins.

In addition to the flexibility in recruitment system design, these results using Cpf1/Cas12a-type CRISPR enzymes also demonstrated the general adaptability of REDIT proteins to various CRISPR systems for genome recombination. Cpf1/Cas12a enzymes have different catalytic residues and DNA-recognition mechanisms from the Cas9 enzymes. Hence, the REDIT recombination proteins (exonucleases and single-strand annealing proteins) could function independent from the specific choices of the CRISPR enzyme components (Cas9, Cpf1/Cas12a, and others). This proved the generalizability of the REDIT system and open up possibility to use additional CRISPR enzymes (known and unknown) as components of REDIT system to achieve accurate genome editing in eukaryotic cells.

### Example 10

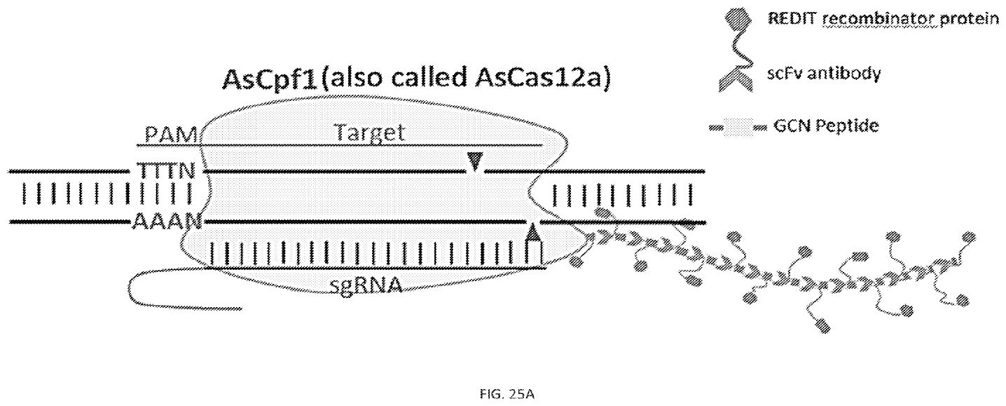

Fifteen different species of microbes having RecE/RecT proteins were selected for a screen of various RecE and RecT proteins across the microbial kingdom (Table 3). Each protein was codon-optimized and synthesized. As previously described for E. coli RecE/RecT based REDIT systems, each protein was fused via E-XTEN linker to the MCP protein with additional nuclear localization signal. mKate knock-in gene-editing assay was used to measure efficiencies at DYNLT1 locus (FIG. 26A, Table 4) and HSP90AA1 locus (FIG. 26B, Table 4). The homologs demonstrated the ability to enable and enhance precision gene-editing.

TABLE 3
RecE and RecT protein homologs

| Homolog | Source | Protein |
|---|---|---|
| T1 | Pantoea stewartii | RecT |
| E1 | Pantoea stewartii | RecE |
| T2 | Pantoea brenneri | RecT |
| E2 | Pantoea brenneri | RecE |
| T3 | Pantoea dispersa | RecT |
| E3 | Pantoea dispersa | RecE |
| T4 | Type-F symbiont of Plautia stali | RecT |
| E4 | Type-F symbiont of Plautia stali | RecE |
| T5 | Providencia stuartii | RecT |
| E5 | Providencia stuartii | RecE |
| T6 | Providencia sp. MGF014 | RecT |
| E6 | Providencia sp. MGF014 | RecE |
| T7 | Providencia alcalifaciens DSM 30120 | RecT |
| E7 | Providencia alcalifaciens DSM 30120 | RecE |
| T8 | Shewanella putrefaciens | RecT |
| E8 | Shewanella putrefaciens | RecE |
| T9 | Bacillus sp. MUM 116 | RecT |
| E9 | Bacillus sp. MUM 116 | RecE |
| T10 | Shigella sonnei | RecT |
| E10 | Shigella sonnei | RecE |
| T11 | Salmonella enterica | RecT |
| E11 | Salmonella enterica | RecE |
| T12 | Acetobacter | RecT |
| E12 | Acetobacter | RecE |
| T13 | Salmonella enterica subsp. enterica | RecT |
| | serovar Javiana str. 10721 | |
| E13 | Salmonella enterica subsp. enterica | RecE |
| | serovar Javiana str. 10721 | |
| T14 | Pseudobacteriovorax antillogorgiicola | RecT |
| E14 | Pseudobacteriovorax antillogorgiicola | RecE |
| T15 | Photobacterium sp. JCM 19050 | RecT |
| E15 | Photobacterium sp. JCM 19050 | RecE |

TABLE 4
mKate Knock-In Gene-Editing Efficiencies

| | DYNLT1 | | HSP90AA1 | |
|---|---|---|---|---|
| | Mean | | Mean | |
| | mKate+ | | mKate+ | |
| | (%) | SEM | (%) | SEM |
| NC | 1.2100 | 0.0802 | 1.7333 | 0.1245 |
| NR | 2.0500 | 0.1442 | 4.0100 | 0.2166 |
| EcRecE_587 | 5.1767 | 0.0897 | 3.7067 | 0.1784 |
| EcRecT | 9.9467 | 1.0143 | 6.5467 | 0.4646 |
| Homolog_T1 | 11.7333 | 0.4667 | 8.0733 | 0.8752 |
| Homolog_E1 | 5.7333 | 0.8503 | 7.6567 | 0.4556 |
| Homolog_T2 | 12.0000 | 0.5292 | 6.9233 | 0.4594 |
| Homolog_E2 | 7.4533 | 0.8553 | 6.4867 | 0.4359 |
| Homolog_T3 | 11.9000 | 1.3013 | 7.1200 | 0.2730 |
| Homolog_E3 | 2.0533 | 0.1020 | 6.7467 | 0.1565 |
| Homolog_T4 | 10.4433 | 0.7331 | 5.7567 | 0.8704 |
| Homolog_E4 | 5.7200 | 0.4744 | 6.2567 | 0.3339 |
| Homolog_T5 | 10.8267 | 0.9445 | 6.4300 | 0.3262 |
| Homolog_E5 | 4.4667 | 0.7116 | 6.0233 | 0.4366 |
| Homolog_T6 | 9.0533 | 0.3548 | 6.2500 | 0.4100 |
| Homolog_E6 | 5.4100 | 0.5981 | 5.9300 | 0.4708 |
| Homolog_T7 | 5.6467 | 0.7383 | 5.3700 | 0.4795 |
| Homolog_E7 | 4.4733 | 0.2444 | 5.7367 | 0.2105 |
| Homolog_T8 | 5.0400 | 0.5599 | 5.7133 | 0.4886 |
| Homolog_E8 | 4.6567 | 0.3088 | 7.0533 | 0.4388 |
| Homolog_T9 | 8.1300 | 0.3523 | 6.2000 | 0.2511 |
| Homolog_E9 | 5.3233 | 0.5233 | 5.6900 | 0.4903 |
| Homolog_T10 | 8.5333 | 0.1601 | 5.5900 | 0.2237 |
| Homolog_E10 | 4.4000 | 1.0149 | 3.5900 | 0.1442 |
| Homolog_T11 | 9.8467 | 1.4374 | 4.9233 | 0.4074 |
| Homolog_E11 | 7.0567 | 1.5872 | 3.1167 | 0.2010 |
| Homolog_T12 | 8.5900 | 0.5401 | 5.2733 | 0.2935 |
| Homolog_E12 | 5.2633 | 0.3374 | 6.0800 | 0.5164 |
| Homolog_T13 | 9.9567 | 0.3324 | 5.7200 | 0.4267 |
| Homolog_E13 | 5.6333 | 0.2360 | 5.6900 | 0.3729 |
| Homolog_T14 | 6.7700 | 0.7022 | 4.7200 | 0.3612 |
| Homolog_E14 | 6.0167 | 0.4890 | 5.7100 | 0.1793 |
| Homolog_T15 | 7.8033 | 0.7075 | 5.2333 | 0.2302 |
| Homolog_E15 | 5.0700 | 0.5543 | 6.0500 | 0.5696 |

### Example 11

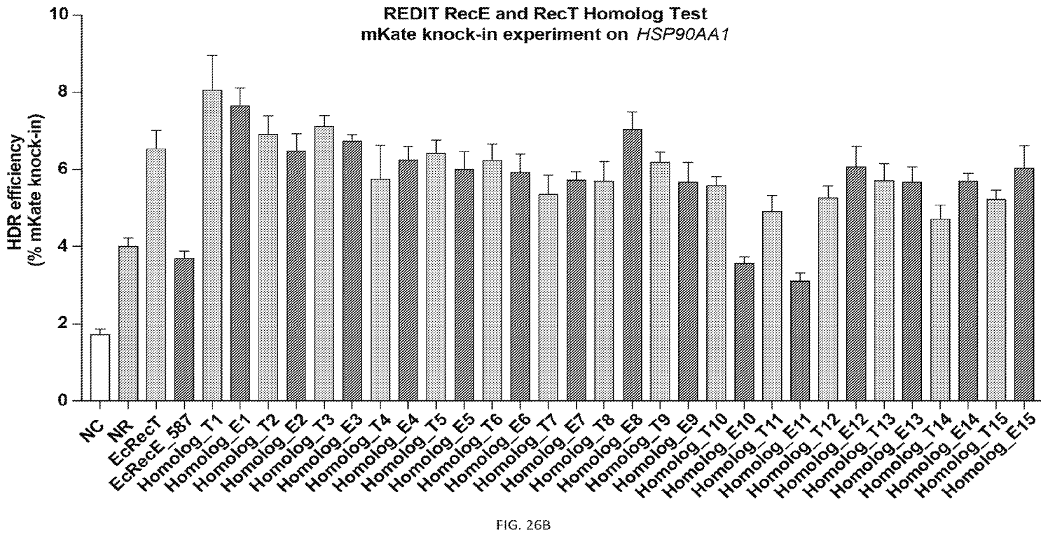

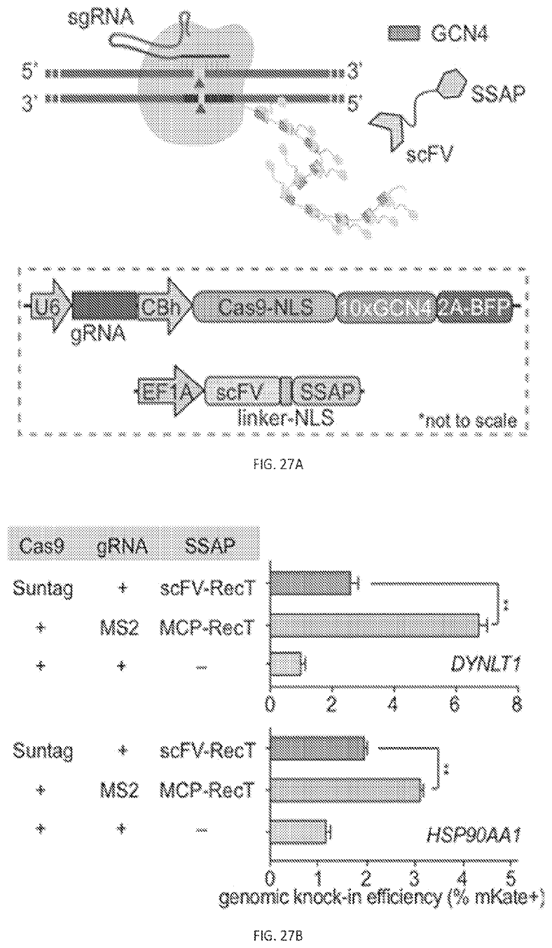

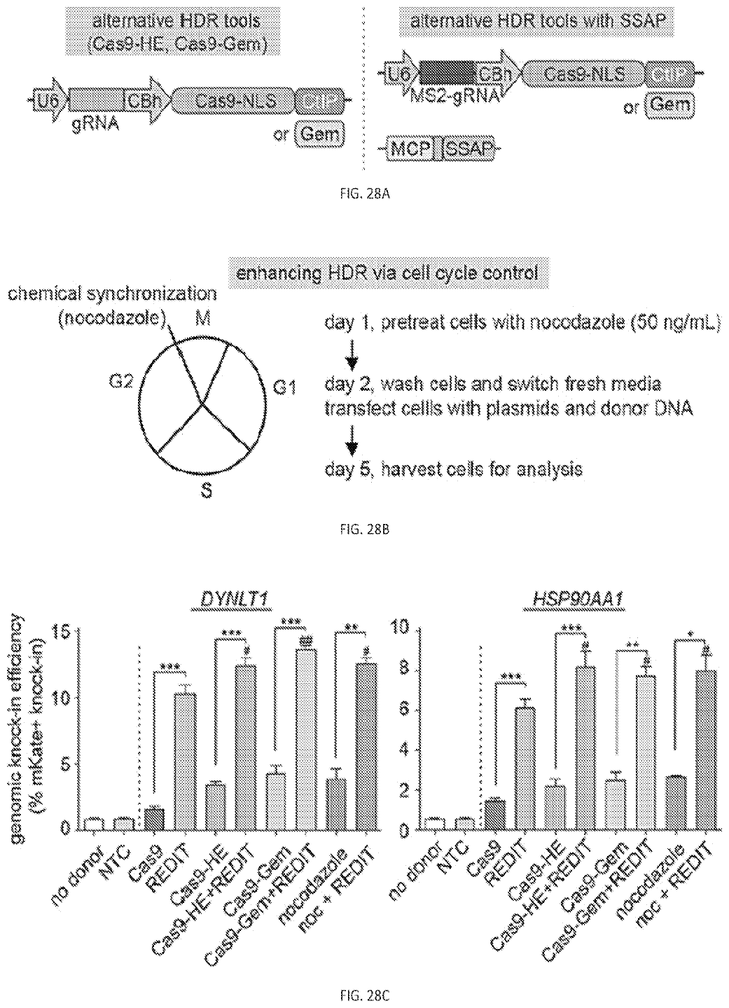

Next, to benchmark the RecT-based REDIT design, it was compared with three categories of existing HDR-enhancing tools (FIGS. 28A and 28B): DNA repair enzyme CtIP fusion with the Cas9 (Cas9-HE), a fusion of the functional domain (amino acids 1 to 110) of human Geminin protein with the Cas9 (Cas9-Gem), and a small-molecule enhancers of HDR via cell cycle control, Nocodazole. Across endogenous targets tested, the RecT-based REDIT design had favorable performance compared with three alternative strategies (FIG. 28C). Furthermore, the RecT-based REDIT design, which putatively acted through activity independently from the other approaches, may synergize with existing methods. To test this hypothesis, RecT-based REDIT design was combined with three different approaches (conveniently through the MS2-aptamer) (FIG. 28A, right). The RecT-based REDIT design could indeed further enhance the HDR-promoting activities of the tested tools (FIG. 28C).

### Example 12

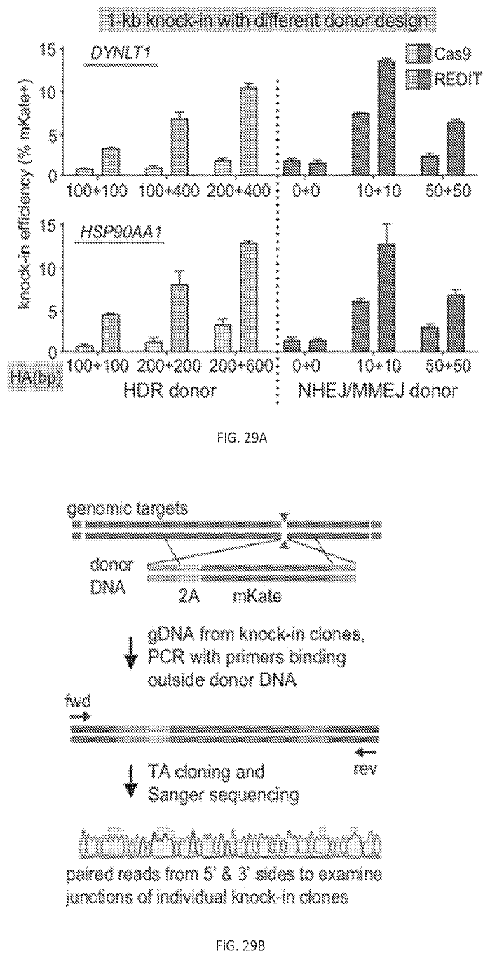

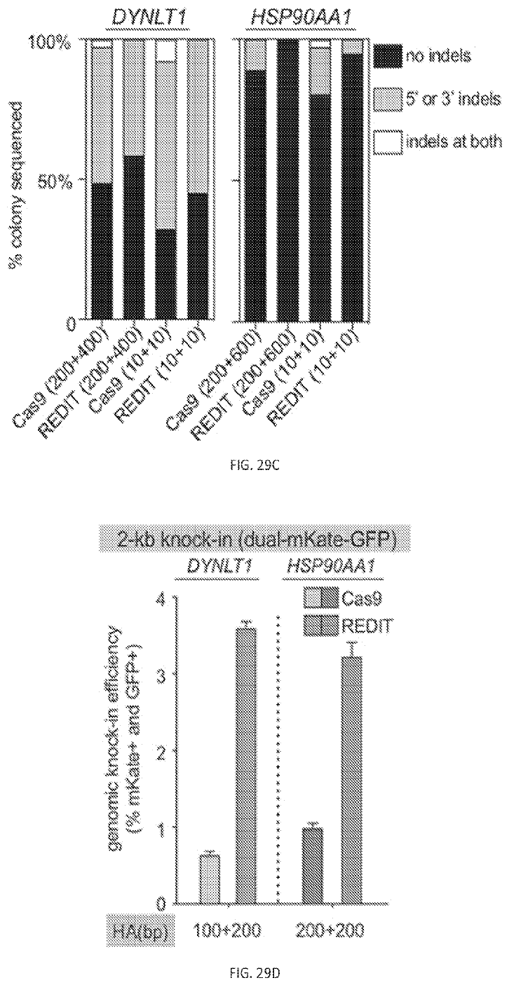

The effect of template HA lengths on the editing efficiency of REDIT was quantified when using the canonical HDR donor bearing HAs of at least 100 bp on each side (FIG. 29A, left). Higher HDR rates were observed for both Cas9 and RecT groups with increasing HA lengths, and REDIT effectively stimulated HDR over Cas9 using HA lengths as short as ˜100 bp each side. When supplied with a longer template bearing 600-800 bp total HA, RecT achieved over 10% HDR efficiencies for kb-scale knock-in without selection, significantly higher than the 2-3% efficiency when only using Cas9. Recent reports identified that using donor DNAs with shorter HAs (usually between 10 and 50 bp) could significantly stimulate knock-in efficiencies thanks to the high repair activities from the Microhomology-mediated end joining (MMEJ) pathway. Knock-in efficiencies of the REDIT-based method were compared with Cas9, using donor DNA with 0 bp (NHEJ-based), 10 bp or 50 bp (MMEJ-based) HAs. The results demonstrated that short-HA donors leveraging MMEJ mechanisms yielded higher editing efficiencies compared with HDR donors (FIG. 29A, right). At the same time, REDIT was able to enhance the knock-in efficiencies as long as there is HA present (no effect for the 0 bp NHEJ donor). This effect is particularly significant with The 10 bp donors in which there was a significant effect, were chosen for further characterization and comparison with the HDR donors.

The knock-in cells were clonally isolated and the target genomic region was amplified using primers binding completely outside of the donor DNAs for colony Sanger sequencing (FIG. 29B. Junction sequencing analysis (˜48 colonies per gene per condition) revealed varying degrees of indels at the 5′- and 3′-knock-in junctions, including at single or both junctions (FIG. 29C). Overall, HDR donors had better precision than MMEJ donors, and REDIT modestly improved the knock-in yield compared with Cas9, though junction indels were still observed.

Furthermore, the efficiencies of REDIT and Cas9 were compared when making different lengths of editing. For longer edits, 2-kb knock-in cassettes were used (FIG. 29D), and for shorter edits single-stranded oligo donors (ssODN) were used. When the knock-in sequence length was increased to ˜2-kb using a dual-mKate/GFP template, REDIT maintained its HDR-promoting activity compared with Cas9 across endogenous targets tested (FIG. 29D). For ssODN tests, at two well-established loci EMX1 and VEGFA, REDIT and Cas9 were used to introduce 12-16-bp exogenous sequences. As ssODN templates are short (<100 bp HAs on each side), next-generation sequencing (NGS) was used to quantify the editing events. Comparable levels of indels were observed between Cas9 and REDIT with improved HDR efficiencies using REDIT.

### Example 13

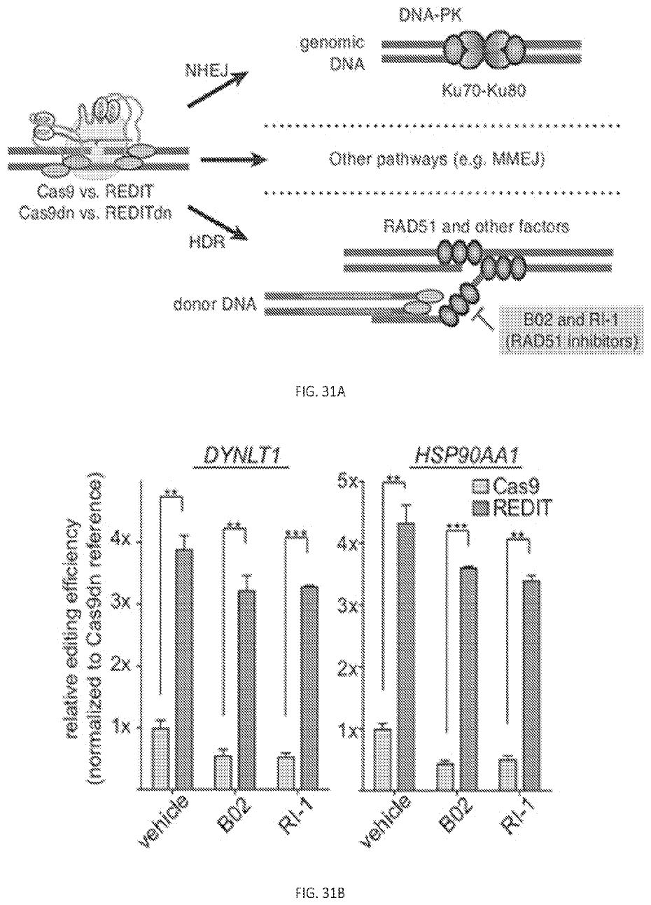

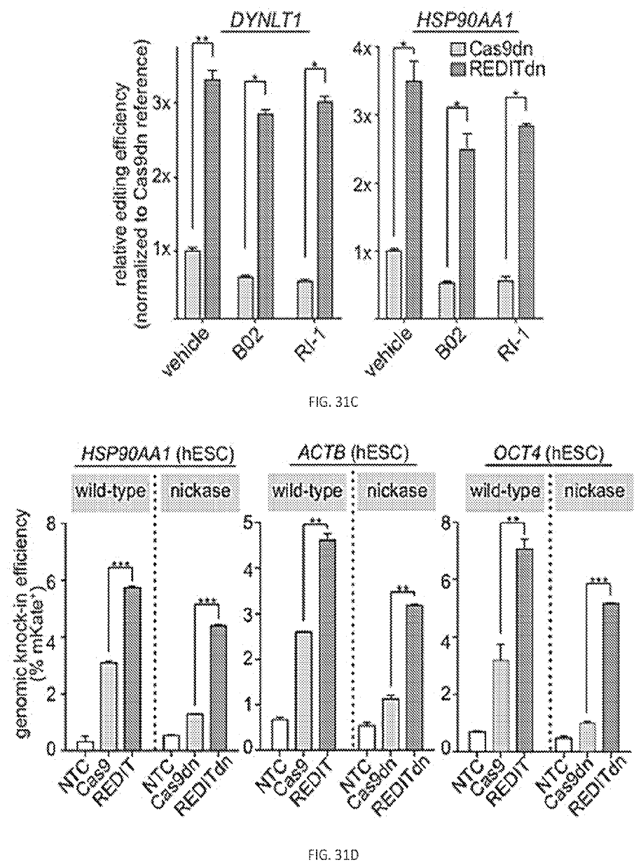

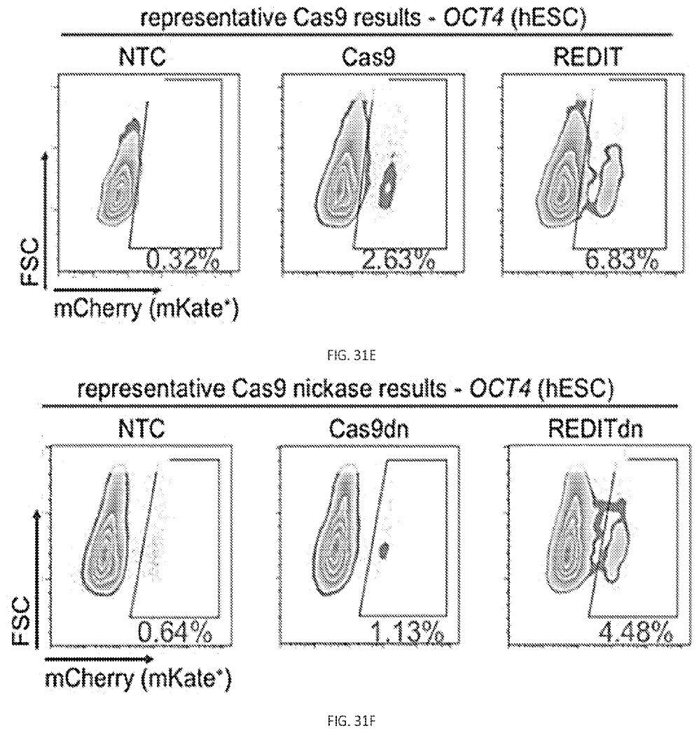

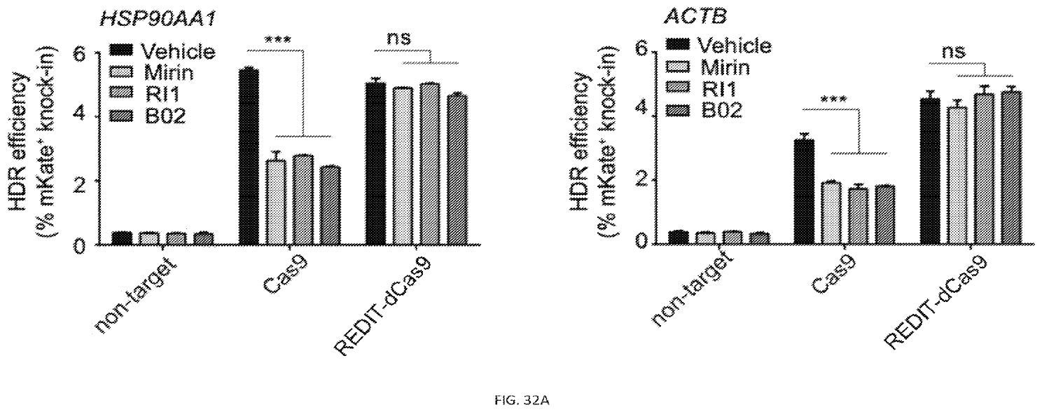

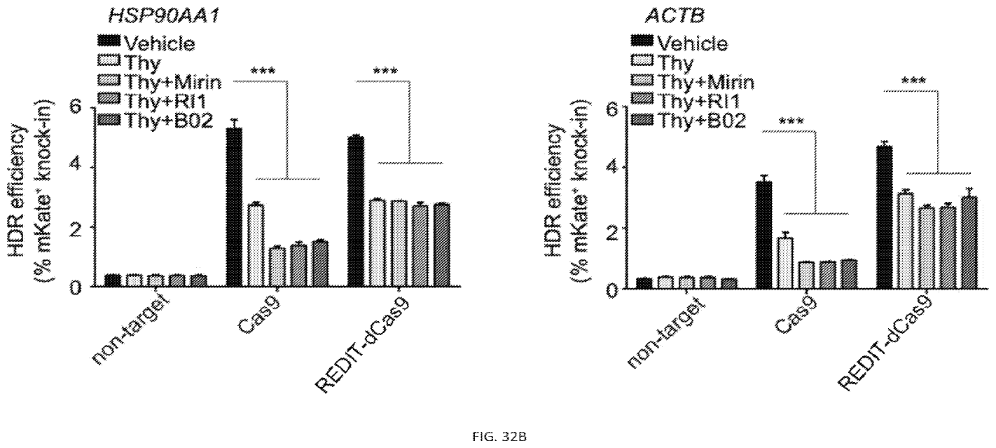

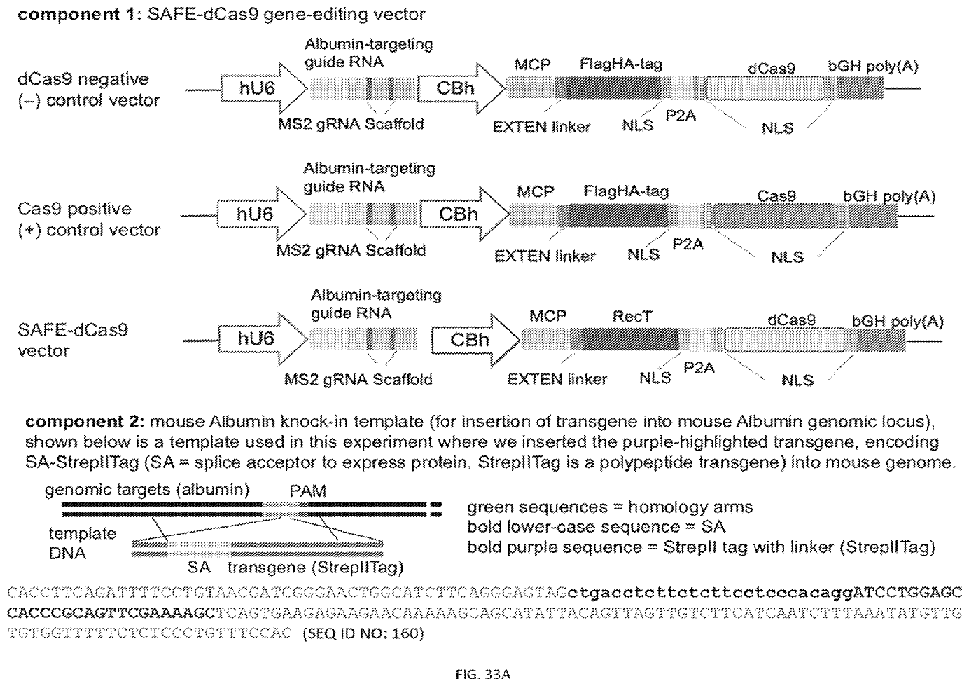

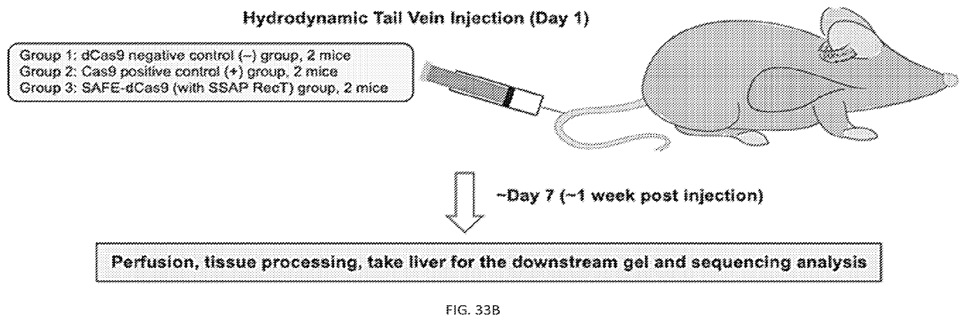

The sensitivity of REDIT's ability to promote HDR in the presence or absence of two distinctive pharmacological inhibitors of RAD51, B02 and RI-1 (FIG. 31A). As expected, for Cas9-based editing, RAD51 inhibition significantly lowered HDR efficiencies (FIGS. 31B, 31C, and 32A). Intriguingly, RAD51 inhibition decreased REDIT and REDITdn efficiencies only moderately, as both REDIT/REDITdn methods maintained significantly higher knock-in efficiencies compared with Cas9/Cas9dn under RAD51 inhibition.

Mirin, a potent chemical inhibitor of DSB repair, which has also been shown to prevent MRN complex formation, MRN-dependent ATM activation, and inhibit Mre11 exonuclease activity was also used. When treating cells with Mrining, only the editing efficiencies of Cas9 reference experiments were affected by the Miring treatment, whereas the REDIT versions were essentially the same as vehicle-treated groups across all genomic targets (FIG. 32A).

To test if cell cycle inhibition affected recombination, cells were chemically synchronized at the G1/S boundary using double Thymidine blockage (DTB). REDIT versions had reduced editing efficiencies under DTB treatment, though it maintained higher editing efficiencies under DNA repair pathway inhibition, compared with Cas9 reference experiments, when Miring RI-1, or B02 were combined with DTB treatment (FIG. 32B).

To validate REDIT in different contexts, REDIT was applied in human embryonic stem cells (hESCs) to test their ability to engineer long sequences in non-transformed human cells. Robust stimulation of HDR was observed across all three genomic sites (HSP90AA1, ACTB, OCT4/POU5F1) using REDIT and REDITdn (FIGS. 31D and 31E). Of note, REDIT and REDITdn editing used donor DNAs with 200-bp HAs on each side and achieved up to over 5% efficiency for kb-scale gene-editing without selection compared with ˜1% efficiency using non-REDIT methods. Additionally, REDIT improved knock-in efficiencies in A549 (lung-derived), HepG2 (liver-derived), and HeLa (cervix-derived) cells, demonstrating up to ˜15% kb-scale genomic knock-in without selection. This improvement was up to 4-fold higher than the Cas9 groups, supporting the potential of using REDIT methods in different cell types.

### Example 14

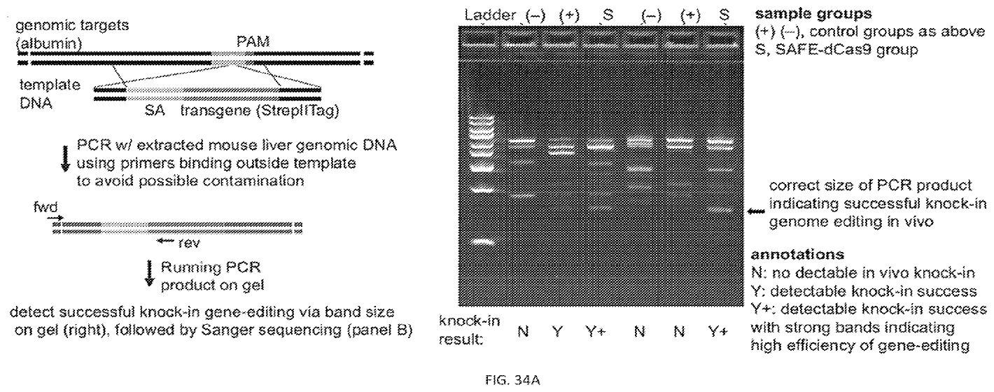

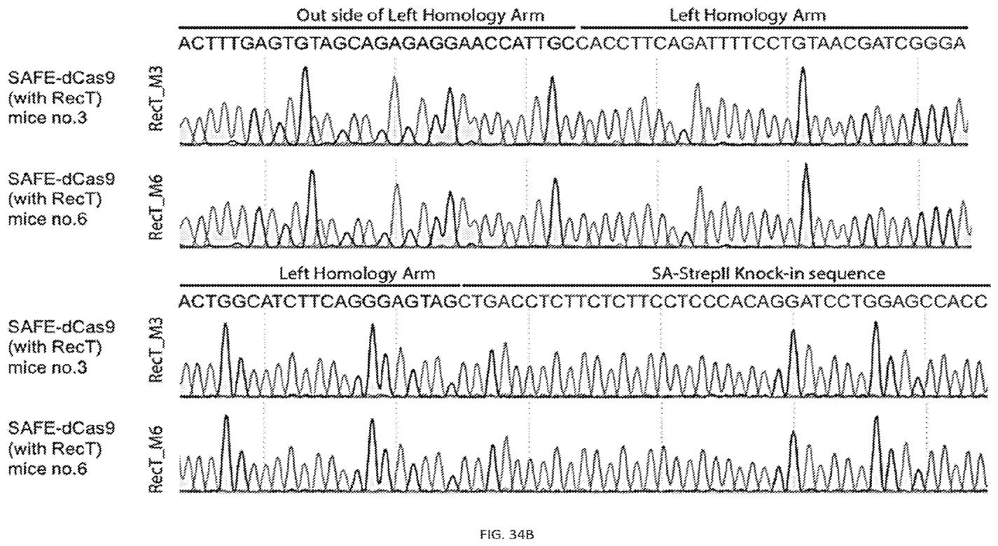

In vivo use of dCas9-EcRecT (SAFE-dCas9) was tested using cleavage free dCas9 editor via hydrodynamic tail vein injection. The gene editing vectors and template DNA used are shown in FIG. 33A. A gene editing vector (60 μg) and template DNA (60 μg) were injected via hydrodynamic tail vein injection to deliver the components to the mouse. Successful gene editing of liver hepatocytes was monitored by transgene-encoded protein expression from the albumin locus. A schematic of the experimental procedure is shown in FIG. 33B.

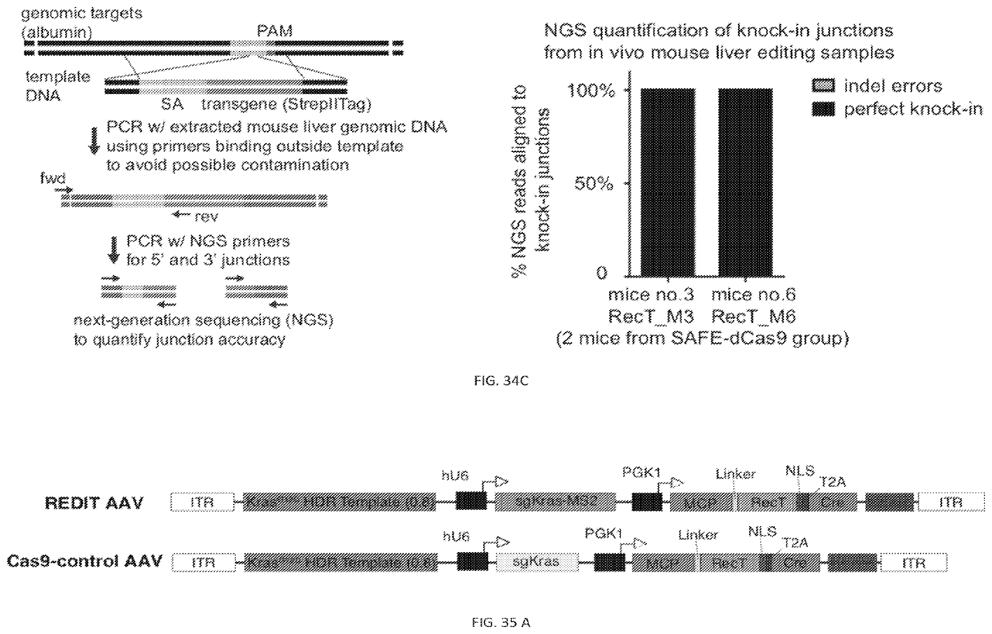

At approximately seven days after injection, the perfused mice livers were dissected. The lobes of the liver were homogenized and processed to extract liver genomic DNA from the primary hepatocytes. The extracted genomic DNA was used for three different downstream analyses: 1) PCR using knock-in-specific primers and agarose gel electrophoresis (FIG. 34A); 2) Sanger sequencing of the knock-in PCR product (FIG. 34B); 3) high-throughput deep sequencing of the knock-in junction to confirm and quantify the accuracy of gene-editing using SAFE-dCas9 in vivo (FIG. 34C). Each downstream analysis confirmed knock-in success with.

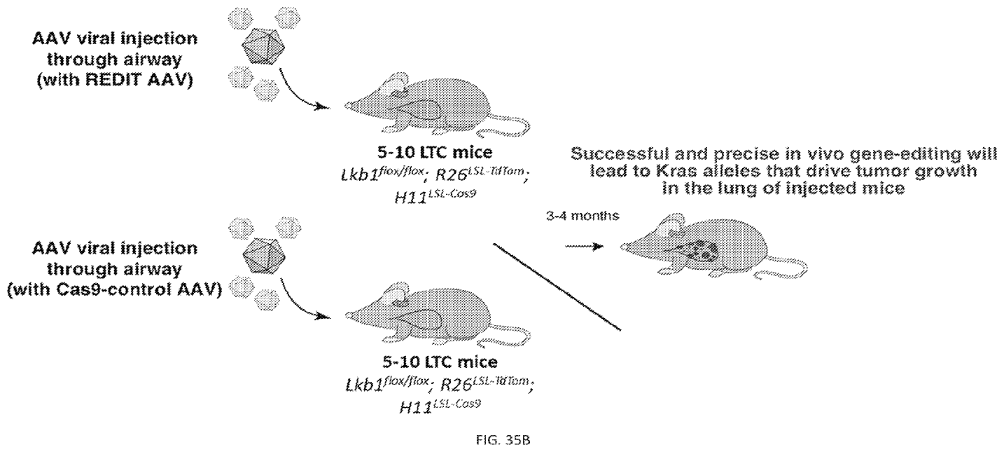

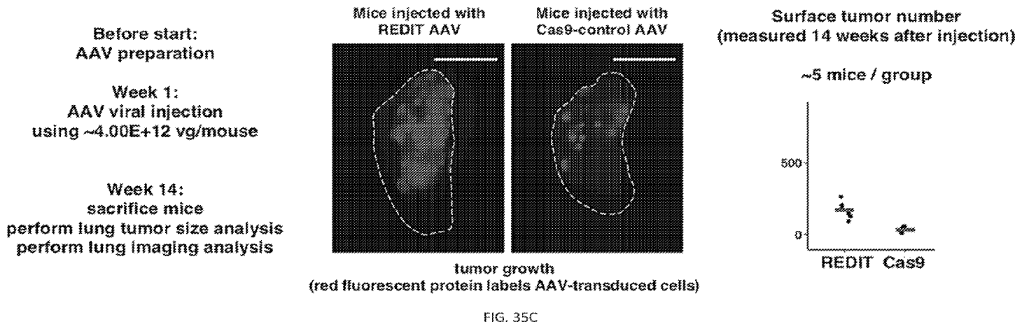

In addition, in vivo use was tested using adeno-associated virus (AAV) delivery into LTC mice lungs. LTC mice include three genome alleles: 1) Lkb1 (flox/flox) allele allows Lkb1-K0 when expressing Cre; 2) R26(LSL-TdTom) allele allows detection of AAV-transduced cells via TdTom red fluorescent protein; and 3) H11(LSL-Cas9) allele allows expression of Cas9 in AAV-transduced cells. Schematics of the REDI gene editing vector and Cas9 control vectors are shown in FIG. 35A. As shown in FIG. 35B, successful gene editing using the gene editing vector leads to Kras alleles that drive tumor growth in the lung of the treated mice.

Approximately fourteen weeks after the AAV injection, perfused mice lungs were dissected. Fixed lung tissue was used for imaging analysis to identify tumor formation from successful gene-editing (FIG. 35C). Quantification of the surface tumor number via imagining analysis showed increased gene-editing efficiencies and total number of tumors in the REDIT treated mice (FIG. 35C).

Escherichia coli RecE amino acid sequence
(SEQ ID NO: 1):
MSTKPLFLLRKAKKSSGEPDVVLWASNDFESTCATLDYLIVKSGKKLS
SYFKAVATNFPVVNDLPAEGEIDFTWSERYQLSKDSMTWELKPGAAPD
NAHYQGNTNVNGEDMTEIEENMLLPISGQELPIRWLAQHGSEKPVTHV
SRDGLQALHIARAEELPAVTALAVSHKTSLLDPLEIRELHKLVRDTDK
VFPNPGNSNLGLITAFFEAYLNADYTDRGLLTKEWMKGNRVSHITRTA
SGANAGGGNLTDRGEGFVHDLTSLARDVATGVLARSMDLDIYNLHPAH
AKRIEEIIAENKPPFSVFRDKFITMPGGLDYSRAIWASVKEAPIGIEV
IPAHVTEYLNKVLTETDHANPDPEIVDIACGRSSAPMPQRVTEEGKQD
DEEKPQPSGTTAVEQGEAETMEPDATEHHQDTQPLDAQSQVNSVDAKY
QELRAELHEARKNIPSKNPVDDDKLLAASRGEFVDGISDPNDPKWVKG
IQTRDCVYQNQPETEKTSPDMNQPEPVVQQEPEIACNACGQTGGDNCP
DCGAVMGDATYQETFDEESQVEAKENDPEEMEGAEHPHNENAGSDPHR
DCSDETGEVADPVIVEDIEPGIYYGISNENYHAGPGISKSQLDDIADT
PALYLWRKNAPVDTTKTKTLDLGTAFHCRVLEPEEFSNRFIVAPEFNR
RTNAGKEEEKAFLMECASTGKTVITAEEGRKIELMYQSVMALPLGQWL
VESAGHAESSIYWEDPETGILCRCRPDKIIPEFHWIMDVKTTADIQRF
KTAYYDYRYHVQDAFYSDGYEAQFGVQPTFVFLVASTTIECGRYPVEI
FMMGEEAKLAGQQEYHRNLRTLADCLNTDEWPAIKTLSLPRWAKEYAN
D
Escherichia coli RecE_587 amino acid sequence
(SEQ ID NO: 2):
ADPVIVEDIEPGIYYGISNENYHAGPGVSKSQLDDIADTPALYLWRKN
APVDTTKTKTLDLGTAFHCRVLEPEEFSNRFIVAPEFNRRTNSGKEEE
KAFLRECASTGKTVITAEEGRKIELMYQSVMALPLGQWLVESAGHAES
SIYWEDPETAILCRCRPDKIIPEFHWIMDVKTTADIQRFKTAYYDYRY
HVQDAFYSDGYEAQFGVQPTFVFLVASTTIECGRYPVEIFMMGEEAKL
AGQLEYHRNLRTLADCLNTDEWPAIKTLSLPRWAKEYAND*
Escherichia coli CTDRecE amino acid sequence
(SEQ ID NO: 3):
GISNENYHAGPGVSKSQLDDIADTPALYLWRKNAPVDTTKTKTLDLGT
AFHCRVLEPEEFSNRFIVAPEFNRRTNSGKEEEKAFLRECASTGKTVI
TAEEGRKIELMYQSVMALPLGQWLVESAGHAESSIYWEDPETAILCRC
RPDKIIPEFHWIMDVKTTADIQRFKTAYYDYRYHVQDAFYSDGYEAQF
GVQPTFVFLVASTTIECGRYPVEIFMMGEEAKLAGQLEYHRNLRTLAD
CLNTDEWPAIKTLSLPRWAKEYAND*
Pantoea brenneri RecE amino acid sequence
(SEQ ID NO: 4):
MQPGIYYDISNEDYHRGAGISKSQLDDIAISPAIYQWRKHAPVDEEKT
AALDLGTALHCLLLEPDEFSKRFQIGPEVNRRTTAGKEKEKEFIERCE
AEGITPITHDDNRKLKLMRDSALAHPIARWMLEAQGNAEASIYWNDRD
AGVLSRCRPDKIITEFNWCVDVKSTADIMKFQKDFYSYRYHVQDAFYS
DGYESHFHETPTFAFLAVSTSIDCGRYPVQVFIMDQQAKDAGRAEYKR
NIHTFAECLSRNEWPGIATLSLPFWAKELRNE
Type-F symbiont of Plautia stali RecE amino
acid sequence
(SEQ ID NO: 5):
MQPGIYYDISNEDYHGGPGISKSQLDDIAISPAIYQWRKHAPVDEEKT
AALDLGTALHCLLLEPDEFSKRFEIGPEVNRRTTAGKEKEKEFMERCE
AEGVTPITHDDNRKLRLMRDSAMAHPIARWMLEAQGNAEASIYWNDRD
TGVLSRCRPDKIITDFNWCVDVKSTADIIKFQKDFYSYRYHVQDAFYS
DGYESHFDETPTFAFLAVSTSIDCGRYPVQVFIMDQQAKDAGRAEYKR
NIHTFAECLSRNEWPGIATLSLPYWAKELRNE
Providencia sp. MGF014 RecE amino acid sequence
(SEQ ID NO: 6):
MKEGIYYNISNEDYHNGLGISKSQLDLINEMPAEYIWSKEAPVDEEKI
KPLEIGTALHCLLLEPDEYHKRYKIGPDVNRRTNVGKEKEKEFFDMCE
KEGITPITHDDNRKLMIMRDSALAHPIAKWCLEADGVSESSIYWTDKE
TDVLCRCRPDRIITAHNYIIDVKSSGDIEKFDYEYYNYRYHVQDAFYS
DGYKEVTGITPTFLFLVVSTKIDCGKYPVRTYVMSEEAKSAGRTAYKH
NLLTYAECLKTDEWAGIRTLSLPRWAKELRNE
Shigella sonnei RecE amino acid sequence
(SEQ ID NO: 7):
DRGLLTKEWRKGNRVSRITRTASGANAGGGNLTDRGEGFVHDLTSLAR
DIATGVLARSMDVDIYNLHPAHAKRIEEIIAENKPPFSVFRDKFITMP
GGLDYSRAIVVASVKEAPIGIEVIPAHVTAYLNKVLTETDHANPDPEI
VDIACGRSSAPMPQRVTEEGKQDDEEKLQPSGTTADEQGEAETMEPDA
TKHHQDTQPLDAQSQVNSVDAKYQELRAELHEARKNIPSKNPVDADKL
LAASRGEFVDGISDPNDPKWVKGIQTRDSVYQNQPETEKTSPDMKQPE
PVVQQEPEIAFNACGQTGGDNCPDCGAVMGDATYQETFDEENQVEAKE
NDPEEMEGAEHPHNENAGSDPHRDCSDETGEVADPVIVEDIEPGIYYG
ISNENYHAGPGVSKSQLDDIADTPALYLWRKNAPVDTTKTKTLDLGTA
FHCRVLEPEEFSNRFIVAPEFNRRTNAGKEEEKAFLMECASTGKMVIT
AEEGRKIELMYQSVMALPLGQWLVESAGHAESSIYWEDPETGILCRCR
PDKIIPEFHWIMDVKTTADIQRFKTAYYDYRYHVQDAFYSDGYEAQFG
VQPTFVFLVASTTIECGRYPVEIFMMGEEAKLAGQLEYHRNLRTLADC
LNTDEWPAIKTLSLPRWAKEYAND
Pseudobacteriovorax antillogorgiicola
RecE amino acid sequence
(SEQ ID NO: 8):
MSKLSNLKVSNSDVDTLSRIRMKEGVYRDLPIESYHQSPGYSKTSLCQ
IDKAPIYLKTKVPQKSTKSLNIGTAFHEAMEGVFKDKYVVHPDPGVNK
TTKSWKDFVKRYPKHMPLKRSEYDQVLAMYDAARSYRPFQKYHLSRGF
YESSFYWHDAVTNSLIKCRPDYITPDGMSVIDFKTTVDPSPKGFQYQA
YKYHYYVSAALTLEGIEAVTGIRPKEYLFLAVSNSAPYLTALYRASEK
EIALGDHFIRRSLLTLKTCLESGKWPGLQEEILELGLPFSGLKELREE
QEVEDEFMELVG
Escherichia coli RecT amino acid sequence
(SEQ ID NO: 9):
MTKQPPIAKADLQKTQGNRAPAAVKNSDVISFINQPSMKEQLAAALPR
HMTAERMIRIATTEIRKVPALGNCDTMSFVSAIVQCSQLGLEPGSALG
HAYLLPFGNKNEKSGKKNVQLIIGYRGMIDLARRSGQIASLSARVVRE
GDEFSFEFGLDEKLIHRPGENEDAPVTHVYAVARLKDGGTQFEVMTRK
QIELVRSLSKAGNNGPWVTHWEEMAKKTAIRRLFKYLPVSIEIQRAVS
MDEKEPLTIDPADSSVLTGEYSVIDNSEE*
Pantoea brenneri RecT amino acid sequence
(SEQ ID NO: 10):
MSNQPPIASADLQKTQQSKQVANKTPEQTLVGFMNQPAMKSQLAAALP
RHMTADRMIRIVTTEIRKTPQLAQCDQSSFIGAVVQCSQLGLEPGSAL
GHAYLLPFGNGRSKSGQSNVQLIIGYRGMIDLARRSGQIVSLSARVVR
ADDEFSFEYGLDENLVHRPGENEDAPITHVYAVARLKDGGTQFEVMTV
KQVEKVKAQSKASSNGPWVTHWEEMAKKTVIRRLFKYLPVSIEMQKAV
VLDEKAESDVDQDNASVLSAEYSVLESGDEATN
Type-F symbiont of Plautia stali RecT
amino acid sequence
(SEQ ID NO: 11):
MSNQPPIASADLQKTQQSKQVANKTPEQTLVGFMNQPAMKSQLAAALP
RHMTADRMIRIVTTEIRKTPALATCDQSSFIGAVVQCSQLGLEPGSAL
GHAYLLPFGNGRSKSGQSNVQLIIGYRGMIDLARRSGQIVSLSARVVR
ADDEFSFEYGLDENLIHRPGDNEDAPITHVYAVARLKDGGTQFEVMTA
KQVEKVKAQSKASSNGPWVTHWEEMAKKTVIRRLFKYLPVSIEMQKAV
VLDEKAESDVDQDNASVLSAEYSVLEGDGGE
Providencia sp. MGF014 RecT amino acid sequence
(SEQ ID NO: 12):
MSNPPLAQSDLQKTQGTEVKVKTKDQQLIQFINQPSMKAQLAAALPRH
MTPDRMIRIVTTEIRKTPALATCDMQSFVGAVVQCSQLGLEPGNALGH
AYLLPFGNGKAKSGQSNVQLIIGYRGMIDLARRSNQIISISARTVRQG
DNFHFEYGLNEDLTHTPSENEDSPITHVYAVARLKDGGVQFEVMTYNQ
VEKVRASSKAGQNGPWVSHWEEMAKKTVIRRLFKYLPVSIEMQKAVVL
DEKAEANVDQENATIFEGEYEEVGTDGN
Shigella sonnei RecT amino acid sequence
(SEQ ID NO: 13):
MTKQPPIAKADLQKTQENRAPAAIKNNDVISFINQPSMKEQLAAALPR
HMTAERMIRIATTEIRKVPALGNCDTMSFVSAIVQCSQLGLEPGSALG
HAYLLPFGNKNEKSGKKNVQLIIGYRGMIDLARRSGQIASLSARVVRE
GDEFNFEFGLDEKLIHRPGENEDAPVTHVYAVARLKDGGTQFEVMTRR
QIELVRSQSKAGNNGPWVTHWEEMAKKTAIRRLFKYLPVSIEIQRAVS
MDEKEPLTIDPADSSVLTGEYSVIDNSEE
Pseudobacteriovorax antillogorgiicola
RecT amino acid sequence
(SEQ ID NO: 14):
MGHLVSKTEQDYIKQHYAKGATDQEFEHFIGVCRARGLNPAANQIYFV
KYRSKDGPAKPAFILSIDSLRLIAHRTGDYAGCSEPIFTDGGKACTVT
VRRNLKSGETGNFSGMAFYDEQVQQKNGRPTSFWQSKPRTMLEKCAEA
KALRKAFPQDLGQFYIREEMPPQYDEPIQVHKPKALEEPRFSKSDLSR
RKGLNRKLSALGVDPSRFDEVATFLDGTPDRELGQKLKLWLKEAGYGV
NQ
SV40 NLS amino acid sequence
(SEQ ID NO: 16):
PKKKRKV
Ty1 NLS amino acid sequence
(SEQ ID NO: 17):
NSKKRSLEDNETEIKVSRDTWNTKNMRSLEPPRSKKRIH
c-Myc NLS amino acid sequence
(SEQ ID NO: 18):
PAAKRVKLD
biSV40 NLS amino acid sequence
(SEQ ID NO: 19):
KRTADGSEFESPKKKRKV
Mut NLS amino acid sequence
(SEQ ID NO: 20):
PEKKRRRPSGSVPVLARPSPPKAGKSSCI
Template DNA sequences (underlining marks
the replaced or inserter editing sequences)
EMX1 HDR template sequence
(SEQ ID NO: 79):
CATTCTGCCTCTCTGTATGGAAAAGAGCATGGGGCTGGCCCGTGGGGT
GGTGTCCACTTTAGGCCCTGTGGGAGATCATGGGAACCCACGCAGTGG
GTcataggctctctcatttactactcacatccactctgtgaagaagcg
attatgatctctcctctagaaaCTCGTAGAGTCCCATGTCTGCCGGCT
TCCAGAGCCTGCACTCCTCCACCTTGGCTTGGCTTTGCTGGGGCTAGA
GGAGCTAGGATGCACAGCAGCTCTGTGACCCTTTGTTTGAGAGGAACA
GGAAAACCACCCTTCTCTCTGGCCCACTGTGTCCTCTTCCTGCCCTGC
CATCCCCTTCTGTGAATGTTAGACCCATGGGAGCAGCTGGTCAGAGGG
GACCCCGGCCTGGGGCCCCTAACCCTATGTAGCCTCAGTCTTCCCATC
AGGCTCTCAGCTCAGCCTGAGTGTTGAGGCCCCAGTGGCTGCTCTGGG
GGCCTCCTGAGTTTCTCATCTGTGCCCCTCCCTCCCTGGCCCAGGTGA
AGGTGTGGTTCCAGAACCGGAGGACAAAGTACAAACGGCAGAAGCTGG
AGGAGGAAGGGCCTGAGTCCGAGCAGAAGAAGAAGGGCTCCCATCACA
TCAACCGGTGGCGCATTGCCACGAAGCAGGCCAATGGGGAGGACATCG
ATGTCACCTCCAATGACTCGGATGTACACGGTCTGCAACCACAAACCC
ACGAGGGCAGAGTGCTGCTTGCTGCTGGCCAGGCCCCTGCGTGGGCCC
AAGCTGGACTCTGGCCACTCCCTGGCCAGGCTTTGGGGAGGCCTGGAG
TCATGGCCCCACAGGGCTTGAAGCCCGGGGCCGCCATTGACAGAGGGA
CAAGCAATGGGCTGGCTGAGGCCTGGGACCACTTGGCCTTCTCCTCGG
AGAGCCTGCCTGCCTGGGCGGGCCCGCCCGCCACCGCAGCCTCCCAGC
TGCTCTCCGTGTCTCCAATCTCCCTTTTGTTTTGATGCATTTCTGTTT
TAATTTATTTTCCAGGCACCACTGTAGTTTAGTGATCCCCAGTGTCCC
CCTTCCCTATGGGAATAATAAAAGTCTCTCTCTTAATGACACGGGCAT
CCAGCTCCAGCCCCAGAGCCTGGGGTGGTAGATTCCGGCTCTGAGGGC
CAGTGGGGGCTGGTAGAGCAAACGCGTTCAGGGCCTGGGAGCCTGGGG
TGGGGTACTGGTGGAGGGGGTCAAGGGTAATTCATTAACTCCTCTCTT
TTGTTGGGGGACCCTGGTCTCTACCTCCAGCTCCACAGCAGGAGAAAC
AGGCTAGACATAGGGAAGGGCCATCCTGTATCTTGAGGGAGGACAGGC
CCAGGTCTTTCTTAACGTATTGAGAGGTGGGAATCAGGCCCAGGTAGT
TCAATGGG
VEGFA HDR template sequence
(SEQ ID NO: 80):
AGGTTTGAATCATCACGCAGGCCCTGGCCTCCACCCGCCCCCACCAGC
CCCCTGGCCTCAGTTCCCTGGCAACATCTGGGGTTGGGGGGGCAGCAG
GAACAAGGGCCTCTGTCTGCCCAGCTGCCTCCCCCTTTGGGTTTTGCC
AGACTCCACAGTGCATACGTGGGCTCCAACAGGTCCTCTTCCCTCCCA
GTCACTGACTAACCCCGGAACCACACAGCTTCCCGTTctcagctccac
aaacttggtgccaaattcttctcccctgggaagcatccctggacactt
cccaaaggaccccagtcactccagcctgttggctgccgctcactttga
tgtctgcaggccagatgagggctccagatggcacattgtcagagggac
acactgtggcccctgtgcccagccctgggctctctgtacatgaagcaa
ctccagtcccaaatatgtagctgtttgggaggtcagaaatagggggtc
caggagcaaactccccccaccccctttccaaagcccattccctcttta
gccagagccggggtgtgcagacggcagtcactagggggcgctcggcca
ccacagggaagctgggtgaatggagcgagcagcgtcttcgagagtgag
gacgtgtgtgtctgtgtgggtgagtgagtgtgCgcACTCTAGAGgtgt
CgTgttgagggcgttggagcggggagaaggccaggggtcactccagga
ttccaatagatctgtgtgtccctctccccacccgtccctgtccggctc
tccgccttcccctgcccccttcaatattcctagcaaagagggaacggc
tctcaggccctgtccgcacgtaacctcactttcctgctccctcctcgc
caatgccccgcgggcgcgtgtctctggacagagtttccgggggcggat
gggtaattttcaggctgtgaaccttggtgggggtcgagcttccccttc
attgcggcgggctGCGGGCCAGGCTTCACTGAGCGTCCGCAGAGCCCG
GGCCCGAGCCGCGTGTGGAAGGGCTGAGGCTCGCCTGTccccgccccc
cggggcgggccgggggcggggtcccggcggggcggAGCCATGCGCCCC
CCCCttttttttttAAAAGTCGGCTGGTAGCGGGGAGGATCGCGGAGG
CTTGGGGCAGCCGGGTAGCTCGGAGGTCGTGGCGCTGGGGGCTAGCAC
CAGCGCTCTGTCGGGAGGCGCAGCGGTTAGGTGGACCGGTCAGCGGAC
TCACCGGCCAGGGCGCTCGGTGCTGGAATTTGATATTCATTGATCCGG
GttttatccctcttcttttttcttaaacatttttttttAAAACTGTAT
TGTTTCTCGTTTTAATTTATTTTTGCTTGCCATTCCCCACTTGAAT
DYNLT1 HDR template sequence
(SEQ ID NO: 81):
AGTGACCTGTGTAATTATGCAGAAGAATGGAGCTGGATTACACACAGC
AAGTTCCTGCTTCTGGGACAGCTCTACTGACGGTATGATTTTCATTCA
TGTTTGTGAAGTTTTGTTGTGTGAAATATATGACTGGAAGTTTCCTAT
CTTTGAATGCAATGCATGTTTATCACCTTTTAAAACATTTAATAATAG
ACTTGCCAAGGTTCTTTGTGTAGCATAGAGATGGGTACTTGAATGTTG
GCCTTATTGTGAGTAAAACGTCGTCCCCCAGCTTTCCCTGCCGTAAAT
GCTGCTCTCTTCCCTCCCGCAGGGAGCTGCACTGTGCGATGGGAGAAT
AAGACCATGTACTGCATCGTCAGTGCCTTCGGACTGTCTATTGGAAGC
GGAGCTACTAACTTCAGCCTGCTGAAGCAGGCTGGAGACGTGGAGGAG
AACCCTGGACCTgccaccatggtgagcgagctgattaaggagaacatg
cacatgaagctgtacatggagggcaccgtgaacaaccaccacttcaag
tgcacatccgagggcgaaggcaagccctacgagggcacccagaccatg
agaatcaaggcggtcgagggcggccctctccccttcgccttcgacatc
ctggctaccagcttcatgtacggcagcaaaaccttcatcaaccacacc
cagggcatccccgacttctttaagcagtccttccccgagggcttcaca
tgggagagagtcaccacatacgaagatgggggcgtgctgaccgctacc
caggacaccagcctccaggacggctgcctcatctacaacgtcaagatc
agaggggtgaacttcccatccaacggccctgtgatgcagaagaaaaca
ctcggctgggaggcctccaccgagacactgtaccccgctgacggcggc
ctggaaggcagagccgacatggccctgaagctcgtgggcgggggccac
ctgatctgcaaccttaagaccacatacagatccaagaaacccgctaag
aacctcaagatgcccggcgtctactatgtggacaggagactggaaaga
atcaaggaggccgacaaagagacatacgtcgagcagcacgaggtggct
gtggccagatactgcgacctccctagcaaactggggcacaaacttaat
tccTAACCaGCtGTCCtGCCTATGGCCTTTCTCCTTTTGTCTCTAGTT
CATCCTCTAACCACCAGCCATGAATTCAGTGAACTCTTTTCTCATTCT
CTTTGTTTTGTGGCACTTTCACAATGTAGAGGAAAAAACCAAATGACC
GCACTGTGATGTGAATGGCACCGAAGTCAGATGAGTATCCCTGTAGGT
CACCTGCAGCCTGCGTTGCCACTTGTCTTAACTCTGAATATTTCATTT
CAAAGGTGCTAAAATCTGAAATCTGCTAGTGTGAAACTTGCTCTACTC
TCTGAAATGATTCAAATACACTAATTTTCCATACTTTATACTTTTGTT
AGAATAAATTATTCAAATCTAAAGTCTGTTGTGTTCTTCATAGTCTGC
ATAGTATCATAAACG
HSP90AA1 HDR template sequence
(SEQ ID NO: 82):
GCAGCAAAGAAACACCTGGAGATAAACCCTGACCATTCCATTATTGAG
ACCTTAAGGCAAAAGGCAGAGGCTGATAAGAACGACAAGTCTGTGAAG
GATCTGGTCATCTTGCTTTATGAAACTGCGCTCCTGTCTTCTGGCTTC
AGTCTGGAAGATCCCCAGACACATGCTAACAGGATCTACAGGATGATC
AAACTTGGTCTGGGTAAGCCTTATACTATGTAATGTTAAAAAGAAAAT
AAACACACGTGACATTGAAGAAAATGGTGAACTTTCAGTTATCCAAAC
TTGGAGCACCTTGTCCTGCTTGCTGCTTGGAGGTATTAAAGTATGttt
tttttAGGGATAAGTAAGGTCTTACAAGAGCAAAGAAATGAAATTGAG
ACTCATATGTCCTGTAATACTGTCTTGAAAGCAGATAGAAACCAAGAG
TATTACCCTAATAGCTGGCTTTAAGAAATCTTTGTAATATGAGGATTT
TATTTTGGAAACAGGTATTGATGAAGATGACCCTACTGCTGATGATAC
CAGTGCTGCTGTAACTGAAGAAATGCCACCCCTTGAAGGAGATGACGA
CACATCACGCATGGAAGAAGTAGACGGAAGCGGAGCTACTAACTTCAG
CCTGCTGAAGCAGGCTGGAGACGTGGAGGAGAACCCTGGACCTgtgag
cgagctgattaaggagaacatgcacatgaagctgtacatggagggcac
cgtgaacaaccaccacttcaagtgcacatccgagggcgaaggcaagcc
ctacgagggcacccagaccatgagaatcaaggcggtcgagggcggccc
tctccccttcgccttcgacatcctggctaccagcttcatgtacggcag
caaaaccttcatcaaccacacccagggcatccccgacttctttaagca
gtccttccccgagggcttcacatgggagagagtcaccacatacgaaga
tgggggcgtgctgaccgctacccaggacaccagcctccaggacggctg
cctcatctacaacgtcaagatcagaggggtgaacttcccatccaacgg
ccctgtgatgcagaagaaaacactcggctgggaggcctccaccgagac
actgtaccccgctgacggcggcctggaaggcagagccgacatggccct
gaagctcgtgggcgggggccacctgatctgcaaccttaagaccacata
cagatccaagaaacccgctaagaacctcaagatgcccggcgtctacta
tgtggacaggagactggaaagaatcaaggaggccgacaaagagacata
cgtcgagcagcacgaggtggctgtggccagatactgcgacctccctag
caaactggggcacaaacttaattccTAaATCTgTGGCTGAGGGATGAC
TTACCTGTTCAGTACTCTACAATTCCTCTGATAATATATTTTCAAGGA
TGTTTTTCTTTATTTTTGTTAATATTAAAAAGTCTGTATGGCATGACA
ACTACTTTAAGGGGAAGATAAGATTTCTGTCTACTAAGTGATGCTGTG
ATACCTTAGGCACTAAAGCAGAGCTAGTAATGCTTTTTGAGTTTCATG
TTGGTTTATTTTCACAGATTGGGGTAACGTGCACTGTAAGACGTATGT
AACATGATGTTAACTTTGTGGTCTAAAGTGTTTAGCTGTCAAGCCGGA
TGCCTAAGTAGACCAAATCTTGTTATTGAAGTGTTCTGAGCTGTATCT
TGATGTTTAGAAAAGTATTCGTTACATCTTGTAGGATCTACTTTTTGA
ACTTTTCATTCCCTGTAGTTGACAATTCTGCATGTACTAGTCCTCTAG
AAATAGGTTAAACTGAAGCAACTTGATGGAAGGATCTCTCCACAGGGC
TTGTTTTCCAAAGAAAAGTATTGTTTGGAGGAGCAAAGTTAAAAGCCT
ACCTAAGCATATCGTAAAGCTGTTCAAAAATAACTCAGACCCAGTCTT
GTGGA
AAVS1 HDR template sequence
(SEQ ID NO: 83):
gatgctctttccggagcacttccttctcggcgctgcaccacgtgatgt
cctctgagcggatcctccccgtgtctgggtcctctccgggcatctctc
ctccctcacccaaccccatgccgtcttcactcgctgggttcccttttc
cttctccttctggggcctgtgccatctctcgtttcttaggatggcctt
ctccgacggatgtctcccttgcgtcccgcctccccttcttgtaggcct
gcatcatcaccgtttttctggacaaccccaaagtaccccgtctccctg
gctttagccacctctccatcctcttgctttctttgcctggacaccccg
ttctcctgtggattcgggtcacctctcactcctttcatttgggcagct
cccctaccccccttacctctctagtctgtgctagctcttccagccccc
tgtcatggcatcttccaggggtccgagagctcagctagtcttcttcct
ccaacccgggcccctatgtccacttcaggacagcatgtttgctgcctc
cagggatcctgtgtccccgagctgggaccaccttatattcccagggcc
ggttaatgtggctctggttctgggtacttttatctgtcccctccaccc
cacagtggggcaagcttctgacctcttctcttcctcccacagggcctc
gagagatctggcagcggaGGAAGCGGAGCTACTAACTTCAGCCTGCTG
AAGCAGGCTGGAGACGTGGAGGAGAACCCTGGACCTgtgagcgagctg
attaaggagaacatgcacatgaagctgtacatggagggcaccgtgaac
aaccaccacttcaagtgcacatccgagggcgaaggcaagccctacgag
ggcacccagaccatgagaatcaaggcggtcgagggcggccctctcccc
ttcgccttcgacatcctggctaccagcttcatgtacggcagcaaaacc
ttcatcaaccacacccagggcatccccgacttctttaagcagtccttc
cccgagggcttcacatgggagagagtcaccacatacgaagatgggggc
gtgctgaccgctacccaggacaccagcctccaggacggctgcctcatc
tacaacgtcaagatcagaggggtgaacttcccatccaacggccctgtg
atgcagaagaaaacactcggctgggaggcctccaccgagacactgtac
cccgctgacggcggcctggaaggcagagccgacatggccctgaagctc
gtgggcgggggccacctgatctgcaaccttaagaccacatacagatcc
aagaaacccgctaagaacctcaagatgcccggcgtctactatgtggac
aggagactggaaagaatcaaggaggccgacaaagagacatacgtcgag
cagcacgaggtggctgtggccagatactgcgacctccctagcaaactg
gggcacaaacttaattccTAaactagggacaggattggtgacagaaaa
gccccatccttaggcctcctccttcctagtctcctgatattgggtcta
acccccacctcctgttaggcagattccttatctggtgacacaccccca
tttcctggagccatctctctccttgccagaacctctaaggtttgctta
cgatggagccagagaggatcctgggagggagagcttggcagggggtgg
gagggaagggggggatgcgtgacctgcccggttctcagtggccaccct
gcgctaccctctcccagaacctgagctgctctgacgcggctgtctggt
gcgtttcactgatcctggtgctgcagcttccttacacttcccaagagg
agaagcagtttggaaaaacaaaatcagaataagttggtcctgagttct
aactttggctcttcacctttctagtccccaatttatattgttcctccg
tgcgtcagttttacctgtgagataaggccagtagccagccccgtcctg
gcagggctgtggtgaggaggggggtgtccgtgtggaaaactccctttg
tgagaatggtgcgtcctaggtgttcaccaggtcgtggccgcctctact
ccctttctctttctccatccttctttccttaaagagtccccagtgcta
tctgggacatattcctccgcccagagcagggtcccgcttccctaaggc
cctgctctgggcttctgggtttgagtccttggc
OCT4 HDR template sequence
(SEQ ID NO: 84):
GCGACTATGCACAACGAGAGGATTTTGAGGCTGCTGGGTCTCCTTTCT
CAGGGGGACCAGTGTCCTTTCCTCTGGCCCCAGGGCCCCATTTTGGTA
CCCCAGGCTATGGGAGCCCTCACTTCACTGCACTGTACTCCTCGGTCC
CTTTCCCTGAGGGGGAAGCCTTTCCCCCTGTCTCCGTCACCACTCTGG
GCTCTCCCATGCATTCAAAtGGAAGCGGAGCTACTAACTTCAGCCTGC
TGAAGCAGGCTGGAGACGTGGAGGAGAACCCTGGACCTgccaccatgg
tgagcgagctgattaaggagaacatgcacatgaagctgtacatggagg
gcaccgtgaacaaccaccacttcaagtgcacatccgagggcgaaggca
agccctacgagggcacccagaccatgagaatcaaggcggtcgaagacg
gccctctccccttcaccttcgacatcctggctaccagcttcatgtacg
gcaacaaaaccttcatcaaccacacccagggcatccccgacttcttta
agcagtccttccccgagggcttcacatgggagagagtcaccacatacg
aagatgggggcgtgctgaccgctacccaggacaccagcctccaggacg
gctgcctcatctacaacgtcaagatcagaggggtgaacttcccatcca
acggccctgtgatgcagaagaaaacactcggctgggaggcctccaccg
agacactgtaccccgctgacggcggcctggaaggcagagccgacatgg
ccctgaagctcgtgggcgggggccacctgatctgcaaccttaagacca
catacagatccaagaaacccgctaagaacctcaagatgcccggcgtct
actatgtggacaggagactggaaagaatcaaggaggccgacaaagaga
catacgtcgagcagcacgaggtggctgtggccagatactgcgacctcc
ctagcaaactggggcacaaacttaattccTAaTGACTAGGAATGGGGG
ACAGGGGGAGGGGAGGAGCTAGGGAAAGAAAACCTGGAGTTTGTGCCA
GGGTTTTTGGGATTAAGTTCTTCATTCACTAAGGAAGGAATTGGGAAC
ACAAAGGGTGGGGGCAGGGGAGTTTGGGGCAACTGGTTGGAGGGAAGG
TGAAGTTCAATGATGCTCTTGATTTTAATCCCACATCATGTATCACTT
TTTTCTTAAATAAAGAAGCCTGGGACACAGTAGATAGACACACTT
Pantoea stewartii Red DNA
(SEQ ID NO: 85):
AGCAACCAGCCCCCTATCGCCTCCGCCGATCTGCAGAAGGCCAACACC
GGCAAGCAGGTGGCCAATAAGACCCCTGAGCAGACACTGGTGGGCTTC
ATGAATCAGCCAGCAATGAAGAGCCAGCTGGCCGCCGCCCTGCCAAGG
CACATGACAGCCGATCGGATGATCAGAATCGTGACCACAGAGATCCGC
AAGACCCCCGCCCTGGCCACATGCGACCAGAGCTCCTTCATCGGCGCC
GTGGTGCAGTGTTCTCAGCTGGGCCTGGAGCCTGGCAGCGCCCTGGGC
CACGCCTACCTGCTGCCATTTGGCAACGGCCGGAGCAAGTCCGGACAG
TCCAATGTGCAGCTGATCATCGGCTATAGAGGCATGATCGATCTGGCC
CGGAGATCTGGCCAGATCGTGTCTCTGAGCGCCAGGGTGGTGCGCGCA
GACGATGAGTTCTCCTTTGAGTACGGCCTGGATGAGAACCTGATCCAC
CGGCCAGGCGAGAATGAGGACGCACCCATCACCCACGTGTATGCAGTG
GCAAGACTGAAGGACGGAGGCACCCAGTTCGAAGTGATGACAGTGAAG
CAGATCGAGAAGGTGAAGGCCCAGTCCAAGGCCTCTAGCAACGGACCC
TGGGTGACCCACTGGGAGGAGATGGCCAAGAAAACCGTGATCAGGCGC
CTGTTTAAGTACCTGCCCGTGAGCATCGAGATGCAGAAGGCCGTGATC
CTGGATGAGAAGGCCGAGTCTGACGTGGATCAGGACAATGCCTCCGTG
CTGTCTGCCGAGTATAGCGTGCTGGACGGCTCCTCTGAGGAG
Pantoea stewartii RecE DNA
(SEQ ID NO: 86):
CAGCCCGGCGTGTACTATGACATCTCCAACGAGGAGTATCACGCCGGC
CCTGGCATCAGCAAGTCCCAGCTGGACGACATCGCCGTGTCCCCAGCC
ATCTTCCAGTGGAGAAAGTCTGCCCCCGTGGACGATGAGAAAACCGCC
GCCCTGGACCTGGGCACAGCCCTGCACTGCCTGCTGCTGGAGCCTGAT
GAGTTCTCCAAGAGGTTTATGATCGGCCCAGAGGTGAACCGGAGAACC
AATGCCGGCAAGCAGAAGGAGCAGGACTTCCTGGATATGTGCGAGCAG
CAGGGCATCACCCCTATCACACACGACGATAACCGGAAGCTGAGACTG
ATGAGGGACTCTGCCTTTGCCCACCCAGTGGCCAGATGGATGCTGGAG
ACAGAGGGCAAGGCCGAGGCCTCTATCTACTGGAATGACAGGGATACA
CAGATCCTGAGCAGGTGCCGCCCCGACAAGCTGATCACCGAGTTCTCT
TGGTGCGTGGACGTGAAGAGCACAGCCGACATCGGCAAGTTCCAGAAG
GACTTCTACAGCTATCGCTACCACGTGCAGGACGCCTTCTATTCCGAT
GGCTACGAGGCCCAGTTTTGCGAGGTGCCAACCTTCGCCTTTCTGGTG
GTGAGCTCCTCTATCGATTGTGGCCGGTATCCCGTGCAGGTGTTTATC
ATGGACCAGCAGGCAAAGGATGCAGGAAGGGCCGAGTATAAGCGGAAC
CTGACCACATACGCCGAGTGCCAGGCAAGGAATGAGTGGCCTGGCATC
GCCACACTGAGCCTGCCTTACTGGGCCAAGGAGATCCGGAATGTG
Pantoea brenneri RecT DNA
(SEQ ID NO: 87):
AGCAACCAGCCCCCTATCGCCTCCGCCGATCTGCAGAAAACCCAGCAG
TCCAAGCAGGTGGCCAACAAGACCCCTGAGCAGACACTGGTGGGCTTC
ATGAATCAGCCAGCAATGAAGAGCCAGCTGGCCGCCGCCCTGCCAAGG
CACATGACCGCCGATCGGATGATCAGAATCGTGACCACAGAGATCCGC
AAGACACCACAGCTGGCCCAGTGCGACCAGAGCTCCTTCATCGGCGCC
GTGGTGCAGTGTTCTCAGCTGGGCCTGGAGCCTGGCAGCGCCCTGGGC
CACGCCTACCTGCTGCCATTTGGCAACGGCCGGTCCAAGTCTGGCCAG
AGCAATGTGCAGCTGATCATCGGCTATAGAGGCATGATCGATCTGGCC
CGGAGATCCGGACAGATCGTGAGCCTGTCCGCCAGGGTGGTGCGCGCA
GACGATGAGTTCTCTTTTGAGTACGGCCTGGATGAGAACCTGGTGCAC
CGGCCAGGCGAGAATGAGGACGCACCCATCACCCACGTGTATGCAGTG
GCAAGACTGAAGGACGGAGGCACCCAGTTCGAAGTGATGACAGTGAAG
CAGGTGGAGAAGGTGAAGGCCCAGTCCAAGGCCTCTAGCAATGGCCCC
TGGGTGACCCACTGGGAGGAGATGGCCAAGAAAACCGTGATCAGGCGC
CTGTTTAAGTACCTGCCCGTGAGCATCGAGATGCAGAAGGCCGTGGTG
CTGGATGAGAAGGCCGAGTCTGACGTGGATCAGGACAACGCCTCTGTG
CTGAGCGCCGAGTATTCCGTGCTGGAGTCTGGCGACGAGGCCACAAAT
Pantoea brenneri RecE DNA
(SEQ ID NO: 88):
CAGCCTGGCATCTACTATGACATCAGCAACGAGGATTATCACAGGGGA
GCAGGCATCAGCAAGTCCCAGCTGGACGACATCGCCATCTCCCCAGCC
ATCTACCAGTGGAGAAAGCACGCCCCCGTGGACGAGGAGAAAACCGCC
GCCCTGGATCTGGGCACAGCCCTGCACTGCCTGCTGCTGGAGCCTGAC
GAGTTCTCTAAGAGGTTTCAGATCGGCCCAGAGGTGAACCGGAGAACC
ACAGCCGGCAAGGAGAAGGAGAAGGAGTTCATCGAGCGGTGCGAGGCA
GAGGGAATCACCCCAATCACACACGACGATAATAGGAAGCTGAAGCTG
ATGAGGGATTCCGCCCTGGCCCACCCAATCGCAAGGTGGATGCTGGAG
GCACAGGGAAACGCAGAGGCCTCTATCTATTGGAATGACAGAGATGCC
GGCGTGCTGAGCAGGTGCCGCCCCGACAAGATCATCACCGAGTTCAAC
TGGTGCGTGGACGTGAAGTCCACAGCCGACATCATGAAGTTCCAGAAG
GACTTCTACTCTTACAGATACCACGTGCAGGACGCCTTCTATTCCGAT
GGCTACGAGTCTCACTTTCACGAGACACCCACATTCGCCTTTCTGGCC
GTGTCTACCAGCATCGACTGCGGCAGGTATCCTGTGCAGGTGTTTATC
ATGGACCAGCAGGCAAAGGATGCAGGAAGGGCCGAGTACAAGAGAAAC
ATCCACACCTTCGCCGAGTGTCTGAGCAGGAATGAGTGGCCTGGCATC
GCCACACTGTCCCTGCCTTTTTGGGCCAAGGAGCTGCGCAATGAG
Pantoea dispersa RecT DNA
(SEQ ID NO: 89):
TCCAACCAGCCACCTCTGGCCACCGCAGATCTGCAGAAAACCCAGCAG
TCTAACCAGGTGGCCAAGACCCCTGAGCAGACACTGGTGGGCTTCATG
AATCAGCCAGCAATGAAGAGCCAGCTGGCCGCCGCCCTGCCAAGGCAC
ATGACCGCCGATCGGATGATCAGAATCGTGACCACAGAGATCCGCAAG
ACACCCGCCCTGGCCCAGTGCGACCAGAGCTCCTTCATCGGAGCAGTG
GTGCAGTGTAGCCAGCTGGGCCTGGAGCCTGGCTCCGCCCTGGGCCAC
GCCTACCTGCTGCCATTTGGCAACGGCCGGTCCAAGTCTGGCCAGAGC
AATGTGCAGCTGATCATCGGCTATAGAGGCATGATCGATCTGGCCCGG
AGATCCGGACAGATCGTGAGCCTGTCCGCCAGGGTGGTGCGCGCAGAC
GATGAGTTCTCTTTTGAGTACGGCCTGGATGAGAACCTGATCCACCGG
CCAGGCGACAATGAGTCCGCCCCCATCACCCACGTGTATGCAGTGGCA
AGACTGAAGGACGGAGGCACCCAGTTCGAAGTGATGACAGCCAAGCAG
GTGGAGAAGGTGAAGGCCCAGTCCAAGGCCTCTAGCAACGGACCCTGG
GTGACCCACTGGGAGGAGATGGCCAAGAAAACCGTGATCAGGCGCCTG
TTTAAGTACCTGCCCGTGAGCATCGAGATGCAGAAGGCCGTGGTGCTG
GACGAGAAGGCCGAGAGCGACGTGGATCAGGACAATGCCTCTGTGCTG
AGCGCCGAGTATTCCGTGCTGGAGTCTGGCACAGGCGAG
Pantoea dispersa RecE DNA
(SEQ ID NO: 90):
GAGCCAGGCATCTACTATGACATCAGCAACGAGGCCTACCACTCCGGC
CCCGGCATCAGCAAGTCCCAGCTGGACGACATCGCCAGGAGCCCTGCC
ATCTTCCAGTGGCGCAAGGACGCCCCAGTGGATACCGAGAAAACCAAG
GCCCTGGACCTGGGCACCGATTTCCACTGCGCCGTGCTGGAGCCAGAG
AGGTTTGCAGACATGTATCGCGTGGGCCCTGAAGTGAATCGGAGAACC
ACAGCCGGCAAGGCCGAGGAGAAGGAGTTCTTTGAGAAGTGTGAGAAG
GATGGAGCCGTGCCCATCACCCACGACGATGCACGGAAGGTGGAGCTG
ATGAGAGGCTCCGTGATGGCCCACCCTATCGCCAAGCAGATGATCGCA
GCACAGGGACACGCAGAGGCCTCTATCTACTGGCACGACGAGAGCACA
GGCAACCTGTGCCGGTGTAGACCCGACAAGTTTATCCCTGATTGGAAT
TGGATCGTGGACGTGAAAACCACAGCCGATATGAAGAAGTTCAGGCGC
GAGTTTTACGATCTGCGGTATCACGTGCAGGACGCCTTCTACACCGAT
GGCTATGCCGCCCAGTTTGGCGAGCGGCCTACCTTCGTGTTTGTGGTG
ACATCCACCACAATCGACTGCGGCAGATACCCCACCGAGGTGTTCTTT
CTGGATGAGGAGACAAAGGCCGCCGGCAGGTCTGAGTACCAGAGCAAC
CTGGTGACCTATTCCGAGTGTCTGTCTCGCAATGAGTGGCCAGGCATC
GCCACACTGTCTCTGCCCCACTGGGCCAAGGAGCTGAGGAACGTG
Type-F symbiont of Plautia stali RecT DNA
(SEQ ID NO: 91):
TCCAACCAGCCCCCTATCGCCTCTGCCGATCTGCAGAAAACCCAGCAG
TCTAAGCAGGTGGCCAACAAGACCCCTGAGCAGACACTGGTGGGCTTC
ATGAATCAGCCAGCAATGAAGTCCCAGCTGGCCGCCGCCCTGCCAAGG
CACATGACAGCCGATCGGATGATCAGAATCGTGACCACAGAGATCCGC
AAGACCCCCGCCCTGGCCACATGCGACCAGAGCTCCTTCATCGGAGCA
GTGGTGCAGTGTAGCCAGCTGGGCCTGGAGCCTGGCTCCGCCCTGGGC
CACGCCTACCTGCTGCCATTTGGCAACGGCCGGTCCAAGTCTGGCCAG
TCTAATGTGCAGCTGATCATCGGCTATAGAGGCATGATCGACCTGGCC
CGGAGAAGCGGACAGATCGTGAGCCTGTCCGCCAGGGTGGTGCGCGCA
GACGATGAGTTCTCCTTTGAGTACGGCCTGGATGAGAACCTGATCCAC
CGGCCAGGCGATAATGAGGACGCCCCCATCACCCACGTGTATGCAGTG
GCAAGACTGAAGGACGGAGGCACCCAGTTCGAAGTGATGACAGCCAAG
CAGGTGGAGAAGGTGAAGGCCCAGAGCAAGGCCTCTAGCAACGGACCC
TGGGTGACCCACTGGGAGGAGATGGCCAAGAAAACCGTGATCAGGCGC
CTGTTTAAGTACCTGCCCGTGAGCATCGAGATGCAGAAGGCCGTGGTG
CTGGATGAGAAGGCCGAGAGCGACGTGGATCAGGACAATGCCTCTGTG
CTGAGCGCCGAGTATTCCGTGCTGGAGGGCGACGGCGGCGAG
Type-F symbiont of Plautia stali RecE DNA
(SEQ ID NO: 92):
CAGCCTGGCATCTACTATGACATCAGCAACGAGGATTATCACGGCGGC
CCTGGCATCAGCAAGTCCCAGCTGGACGACATCGCCATCTCCCCAGCC
ATCTACCAGTGGAGGAAGCACGCCCCCGTGGACGAGGAGAAAACCGCC
GCCCTGGATCTGGGCACAGCCCTGCACTGCCTGCTGCTGGAGCCTGAC
GAGTTCTCTAAGAGATTTGAGATCGGCCCAGAGGTGAACCGGAGAACC
ACAGCCGGCAAGGAGAAGGAGAAGGAGTTCATGGAGAGGTGTGAGGCA
GAGGGAGTGACCCCTATCACACACGACGATAATCGGAAGCTGAGACTG
ATGAGGGATAGCGCAATGGCCCACCCAATCGCCAGATGGATGCTGGAG
GCACAGGGAAACGCAGAGGCCTCTATCTATTGGAATGACAGGGATACC
GGCGTGCTGAGCAGGTGCCGCCCCGACAAGATCATCACCGACTTCAAC
TGGTGCGTGGACGTGAAGTCCACAGCCGACATCATCAAGTTCCAGAAG
GACTTTTACTCTTATCGCTACCACGTGCAGGACGCCTTCTATTCCGAT
GGCTACGAGTCTCACTTTGACGAGACACCAACATTCGCCTTTCTGGCC
GTGTCTACAAGCATCGATTGCGGCCGGTATCCCGTGCAGGTGTTCATC
ATGGACCAGCAGGCAAAGGATGCAGGAAGGGCCGAGTACAAGCGGAAC
ATCCACACCTTTGCCGAGTGTCTGAGCCGCAATGAGTGGCCTGGCATC
GCCACACTGTCCCTGCCTTACTGGGCCAAGGAGCTGCGGAATGAG
Providencia stuartii RecT DNA
(SEQ ID NO: 93):
AGCAACCCACCTCTGGCCCAGGCAGACCTGCAGAAAACCCAGGGCACA
GAGGTGAAGGAGAAAACCAAGGATCAGATGCTGGTGGAGCTGATCAAT
AAGCCTTCCATGAAGGCACAGCTGGCCGCCGCCCTGCCAAGGCACATG
ACACCCGACCGGATGATCAGAATCGTGACCACAGAGATCAGAAAGACC
CCCGCCCTGGCCACATGCGATATGCAGAGCTTCGTGGGAGCAGTGGTG
CAGTGTTCCCAGCTGGGCCTGGAGCCTGGCAACGCCCTGGGACACGCC
TACCTGCTGCCTTTTGGCAACGGCAAGTCTAAGAGCGGCCAGTCTAAT
GTGCAGCTGATCATCGGCTATCGGGGCATGATCGACCTGGCCCGGAGA
AGCGGCCAGATCGTGTCCATCTCTGCCAGGACCGTGCGCCAGGGCGAT
AACTTCCACTTTGAGTACGGCCTGAACGAGAATCTGACCCACGTGCCT
GGCGAGAATGAGGACTCTCCAATCACACACGTGTACGCAGTGGCAAGG
CTGAAGGATGGAGGCGTGCAGTTCGAAGTGATGACCTATAACCAGATC
GAGAAGGTGCGCGCCAGCTCCAAGGCAGGACAGAATGGACCCTGGGTG
AGCCACTGGGAGGAGATGGCCAAGAAAACCGTGATCAGGCGCCTGTTC
AAGTACCTGCCCGTGTCTATCGAGATGCAGAAGGCCGTGATCCTGGAC
GAGAAGGCCGAGGCCAACATCGATCAGGAGAATGCCACCATCTTTGAG
GGCGAGTATGAGGAAGTGGGCACAGACGGCAAG
Providencia stuartii RecE DNA
(SEQ ID NO: 94):
GAGGGCATCTACTATAACATCAGCAATGAGGACTACCACAACGGCCTG
GGCATCTCCAAGTCTCAGCTGGATCTGATCAATGAGATGCCTGCCGAG
TATATCTGGTCCAAGGAGGCCCCCGTGGACGAGGAGAAGATCAAGCCT
CTGGAGATCGGCACCGCCCTGCACTGCCTGCTGCTGGAGCCAGACGAG
TACCACAAGAGATATAAGATCGGCCCCGATGTGAACCGGAGAACAAAT
GCCGGCAAGGAGAAGGAGAAGGAGTTCTTTGATATGTGCGAGAAGGAG
GGCATCACCCCCATCACACACGACGATAACCGGAAGCTGATGATCATG
AGAGACTCTGCCCTGGCCCACCCTATCGCCAAGTGGTGTCTGGAGGCC
GATGGCGTGAGCGAGAGCTCCATCTACTGGACCGACAAGGAGACAGAT
GTGCTGTGCAGGTGTCGCCCAGACCGCATCATCACCGCCCACAACTAC
ATCGTGGATGTGAAGTCTAGCGGCGACATCGAGAAGTTCGATTACGAG
TACTACAACTACAGATACCACGTGCAGGACGCCTTTTACTCCGATGGC
TATAAGGAGGTGACCGGCATCACCCCTACATTCCTGTTTCTGGTGGTG
TCTACCAAGATCGACTGCGGCAAGTACCCCGTGCGGACCTACGTGATG
AGCGAGGAGGCAAAGTCCGCCGGAAGGACCGCCTACAAGCACAACCTG
CTGACCTATGCCGAGTGTCTGAAAACCGATGAGTGGGCCGGCATCAGG
ACACTGTCTCTGCCCAGATGGGCAAAGGAGCTGCGGAATGAG
Providencia sp. MGF014 RecT DNA
(SEQ ID NO: 95):
TCTAACCCCCCTCTGGCCCAGAGCGACCTGCAGAAAACCCAGGGCACA
GAGGTGAAGGTGAAAACCAAGGATCAGCAGCTGATCCAGTTCATCAAT
CAGCCTTCTATGAAGGCACAGCTGGCCGCCGCCCTGCCAAGGCACATG
ACACCCGACCGGATGATCAGAATCGTGACCACAGAGATCAGAAAGACC
CCCGCCCTGGCCACATGCGATATGCAGTCCTTCGTGGGCGCCGTGGTG
CAGTGTTCTCAGCTGGGCCTGGAGCCTGGCAACGCCCTGGGACACGCC
TACCTGCTGCCTTTTGGCAACGGCAAGGCCAAGTCCGGCCAGTCTAAT
GTGCAGCTGATCATCGGCTATCGGGGCATGATCGACCTGGCCCGGAGA
TCCAACCAGATCATCTCTATCAGCGCCAGGACCGTGCGCCAGGGCGAT
AACTTCCACTTTGAGTACGGCCTGAATGAGGACCTGACCCACACACCT
AGCGAGAATGAGGATTCCCCAATCACCCACGTGTACGCAGTGGCAAGG
CTGAAGGACGGAGGCGTGCAGTTTGAAGTGATGACATATAACCAGGTG
GAGAAGGTGCGCGCCAGCTCCAAGGCAGGACAGAATGGACCCTGGGTG
AGCCACTGGGAGGAGATGGCCAAGAAAACCGTGATCAGGCGCCTGTTC
AAGTACCTGCCCGTGTCCATCGAGATGCAGAAGGCAGTGGTGCTGGAC
GAGAAGGCAGAGGCCAACGTGGATCAGGAGAATGCCACCATCTTTGAG
GGCGAGTATGAGGAAGTGGGCACAGATGGCAAT
Providencia sp. MGF014 RecE DNA
(SEQ ID NO: 96):
AAGGAGGGCATCTACTATAACATCAGCAATGAGGACTACCACAACGGC
CTGGGCATCTCCAAGTCTCAGCTGGATCTGATCAATGAGATGCCTGCC
GAGTATATCTGGTCCAAGGAGGCCCCCGTGGACGAGGAGAAGATCAAG
CCTCTGGAGATCGGCACCGCCCTGCACTGCCTGCTGCTGGAGCCAGAC
GAGTACCACAAGAGATATAAGATCGGCCCCGATGTGAACCGGAGAACA
AATGTGGGCAAGGAGAAGGAGAAGGAGTTCTTTGATATGTGCGAGAAG
GAGGGCATCACCCCCATCACACACGACGATAACCGGAAGCTGATGATC
ATGAGAGACTCTGCCCTGGCCCACCCTATCGCCAAGTGGTGTCTGGAG
GCCGATGGCGTGAGCGAGAGCTCCATCTACTGGACCGACAAGGAGACA
GATGTGCTGTGCAGGTGTCGCCCAGACCGCATCATCACCGCCCACAAC
TACATCATCGATGTGAAGTCTAGCGGCGACATCGAGAAGTTCGATTAC
GAGTACTACAACTACAGATACCACGTGCAGGACGCCTTTTACTCCGAT
GGCTATAAGGAGGTGACCGGCATCACCCCTACATTCCTGTTTCTGGTG
GTGTCTACCAAGATCGACTGCGGCAAGTACCCCGTGCGGACCTACGTG
ATGAGCGAGGAGGCAAAGTCCGCCGGAAGGACCGCCTACAAGCACAAC
CTGCTGACCTATGCCGAGTGTCTGAAAACCGATGAGTGGGCCGGCATC
AGGACACTGTCTCTGCCCAGATGGGCAAAGGAGCTGCGGAATGAG
Shewanella putrefaciens RecT DNA
(SEQ ID NO: 97):
CAGACCGCACAGGTGAAGCTGAGCGTGCCCCACCAGCAGGTGTACCAG
GACAACTTCAATTATCTGAGCTCCCAGGTGGTGGGCCACCTGGTGGAT
CTGAACGAGGAGATCGGCTACCTGAACCAGATCGTGTTTAATTCTCTG
AGCACCGCCTCTCCCCTGGACGTGGCAGCACCTTGGAGCGTGTACGGC
CTGCTGCTGAACGTGTGCCGGCTGGGCCTGTCCCTGAATCCAGAGAAG
AAGCTGGCCTATGTGATGCCCTCCTGGTCTGAGACAGGCGAGATCATC
ATGAAGCTGTACCCCGGCTATAGGGGCGAGATCGCCATCGCCTCTAAC
TTCAATGTGATCAAGAACGCCAATGCCGTGCTGGTGTATGAGAACGAT
CACTTCCGCATCCAGGCAGCAACCGGCGAGATCGAGCACTTTGTGACA
AGCCTGTCCATCGACCCTAGGGTGCGCGGAGCATGCAGCGGAGGCTAC
TGTCGGTCCGTGCTGATGGATAATACAATCCAGATCTCTTATCTGAGC
ATCGAGGAGATGAACGCCATCGCCCAGAATCAGATCGAGGCCAACATG
GGCAATACCCCTTGGAACTCCATCTGGCGGACAGAGATGAATAGAGTG
GCCCTGTACCGGAGAGCAGCAAAGGACTGGAGGCAGCTGATCAAGGCC
ACCCCAGAGATCCAGTCCGCCCTGTCTGATACAGAGTAT
Shewanella putrefaciens RecE DNA
(SEQ ID NO: 98):
GGCACCGCCCTGGCCCAGACAATCAGCCTGGACTGGCAGGATACCATCC
AGCCAGCATACACAGCCTCCGGCAAGCCTAACTTCCTGAATGCCCAGG
GCGAGATCGTGGAGGGCATCTACACCGATCTGCCTAATTCCGTGTATC
ACGCCCTGGACGCACACAGCTCCACCGGCATCAAGACATTCGCCAAGG
GCCGCCACCACTACTTTCGGCAGTATCTGTCTGACGTGTGCCGGCAGA
GAACAAAGCAGCAGGAGTACACCTTCGACGCCGGCACCTACGGCCACA
TGCTGGTGCTGGAGCCAGAGAACTTCCACGGCAACTTCATGAGGAACC
CCGTGCCTGACGATTTTCCAGACATCGAGCTGATCGAGAGCATCCCAC
AGCTGAAGGCCGCCCTGGCCAAGAGCAACCTGCCCGTGTCCGGAGCAA
AGGCCGCCCTGATCGAGAGACTGTACGCCTTCGACCCATCCCTGCCCC
TGTTTGAGAAGATGAGGGAGAAGGCCATCACCGACTATCTGGATCTGC
GCTACGCCAAGTATCTGCGGACCGACGTGGAGCTGGATGAGATGGCCA
CATTCTACGGCATCGATACCTCTCAGACACGGGAGAAGAAGATCGAGG
AGATCCTGGCCATCTCTCCTAGCCAGCCAATCTGGGAGAAGCTGATCA
GCCAGCACGTGATCGACCACATCGTGTGGGACGATGCCATGAGGGTGG
AGAGATCCACCAGGGCCCACCCTAAGGCAGACTGGCTGATCTCTGATG
GCTATGCCGAGCTGACAATCATCGCAAGGTGCCCAACCACCGGCCTGC
TGCTGAAGGTGCGGTTTGACTGGCTGAGGAATGATGCCATCGGCGTGG
ACTTCAAGACCACACTGTCTACCAACCCCACAAAGTTTGGCTACCAGA
TCAAGGACCTGCGGTATGATCTGCAGCAGGTGTTCTACTGTTATGTGG
CCAATCTGGCCGGCATCCCTGTGAAGCACTTCTGCTTTGTGGCCACCG
AGTACAAGGACGCCGATAACTGTGAGACATTTGAGCTGTCTCACAAGA
AAGTGATCGAGAGCACCGAGGAGATGTTCGACCTGCTGGATGAGTTTA
AGGAGGCCCTGACCTCCGGCAATTGGTATGGCCACGACAGGTCCCGCT
CTACATGGGTCATCGAGGTG
Bacillus sp. MUM 116 RecT DNA
(SEQ ID NO: 99):
AGCAAGCAGCTGACCACAGTGAATACCCAGGCCGTGGTGGGCACATTC
TCCCAGGCCGAGCTGGATACCCTGAAGCAGACAATCGCCAAGGGCACC
ACAAACGAGCAGTTCGCCCTGTTTGTGCAGACCTGCGCCAACTCTAGG
CTGAATCCATTTCTGAACCACATCCACTGTATCGTGTATAACGGCAAG
GAGGGCGCCACCATGAGCCTGCAGATCGCAGTGGAGGGCATCCTGTAC
CTGGCACGCAAGACAGACGGCTATAAGGGCATCGAGTGCCAGCTGATC
CACGAGAATGACGAGTTCAAGTTTGATGCCAAGTCCAAGGAGGTGGAT
CACCAGATCGGATTCCCCAGGGGCAACGTGATCGGAGGATATGCAATC
GCAAAGAGGGAGGGCTTTGACGATGTGGTGGTGCTGATGGAGTCTAAC
GAGGTGGACCACATGCTGAAGGGCCGGAATGGCCACATGTGGAGAGAC
TGGTTCAACGatatgtttaagaagcacatcatgaagcgggccgccaag
ctgcagtacggcatcgagatcgcAGAGGACGAGACAGTGAGCAGCGGA
CCTAGCGTGGATAATATCCCAGAGTATAAGCCACAGCCCCGGAAGGAC
ATCACACCCAACCAGGACGTGATCGATGCCCCCCCTCAGCAGCCTAAG
CAGGACGATGAGGCCGCCAAGCTGAAGGCCGCCAGATCTGAGGTGAGC
AAGAAGTTCAAGAAGCTGGGCATCGTGAAGGAGGATCAGACCGAGTAC
GTGGAGAAGCACGTGCCTGGCTTCAAGGGCACACTGTCCGACTTTATC
GGCCTGTCTCAGCTGCTGGATCTGAATATCGAGGCCCAGGAGGCCCAG
TCCGCCGACGGCGATCTGCTGGAC
Bacillus sp. MUM 116 RecEDNA
(SEQ ID NO: 100):
ACCTACGCCGCCGACGAGACACTGGTGCAGCTGCTGCTGTCCGTGGAT
GGCAAGCAGCTGCTGCTGGGAAGGGGCCTGAAGAAGGGCAAGGCCCAG
TACTATATCAATGAGGTGCCATCTAAGGCCAAGGAGTTCGAGGAGATC
CGGGACCAGCTGTTTGACAAGGATCTGTTCATGTCCCTGTTTAACCCC
TCTTACTTCTTTACCCTGCACTGGGAGAAGCAGAGGGCCATGATGCTG
AAGTATGTGACAGCCCCCGTGTCTAAGGAGGTGCTGAAGAATCTGCCT
GAGGCCCAGTCCGAGGTGCTGGAGAGATACCTGAAGAAGCACTCTCTG
GTGGATCTGGAGAAGATCCACAAGGACAACAAGAATAAGCAGGATAAG
GCCTATATCTCTGCCCAGAGCAGGACCAACACACTGAAGGAGCAGCTG
ATGCAGCTGACCGAGGAGAAGCTGGACATCGATTCCATCAAGGCCGAG
CTGGCCCACATCGACATGCAGGTCATCGAGCTGGAGAAGCAGATGGAT
ACAGCCTTCGAGAAGAACCAGGCCTTTAATCTGCAGGCCCAGATCAGG
AATCTGCAGGACAAGATCGAGATGAGCAAGGAGCGGTGGCCCTCCCTG
AAGAACGAAGTGATCGAGGATACCTGCCGGACATGCAAGCGGCCCCTG
GACGAGGATAGCGTGGAGGCCGTGAAGGCCGACAAGGATAATCGGATC
GCCGAGTACAAGGCCAAGCACAACTCCCTGGTGTCTCAGAGAAATGAG
CTGAAGGAGCAGCTGAACACCATCGAGTATATCGACGTGACAGAGCTG
AGAGAGCAGATCAAGGAGCTGGATGAGTCCGGACAGCCTCTGAGGGAG
CAGGTGCGCATCTACAGCCAGTATCAGAATCTGGACACCCAGGTGAAG
TCCGCCGAGGCAGACGAGAACGGCATCCTGCAGGATCTGAAGGCCTCT
ATCTTCATCCTGGATAGCATCAAGGCCTTTAGGGGCAAGGAGGCCGAG
ATGCAGGCCGAGAAGGTGCAGGCCCTGTTCACCACACTGAGCGTGCGC
CTGTTTAAGCAGAATAAGGGCGACGGCGAGATCAAGCCAGATTTCGAG
ATCGAGATGAACGACAAGCCCTATCGGACCCTGAGCCTGTCCGAGGGC
ATCCGGGCAGGCCTGGAGCTGCGGGACGTGCTGAGCCAGCAGTCCGAG
CTGGTGACCCCTACATTCGTGGATAATGCCGAGTCTATCACCAGCTTC
AAGCAGCCAAACGGCCAGCTGATCATCAGCCGGGTGGTGGCAGGACAG
GAGCTGAAGATCGAGGCCGTGAGCGAG
Shigella sonnei RecT DNA
(SEQ ID NO: 101):
ACCAAGCAGCCCCCTATCGCCAAGGCCGACCTGCAGAAAACCCAGGAG
AACAGGGCACCAGCAGCCATCAAGAACAATGATGTGATCTCCTTTATC
AATCAGCCCTCTATGAAGGAGCAGCTGGCCGCCGCCCTGCCTAGGCAC
ATGACCGCCGAGAGGATGATCCGCATCGCCACCACAGAGATCCGCAAG
GTGCCTGCCCTGGGCAACTGCGACACAATGAGCTTCGTGAGCGCCATC
GTGCAGTGTAGCCAGCTGGGCCTGGAGCCAGGCTCCGCCCTGGGCCAC
GCCTACCTGCTGCCCTTCGGCAACAAGAATGAGAAGTCCGGCAAGAAG
AATGTGCAGCTGATCATCGGCTATAGGGGCATGATCGATCTGGCCCGG
AGATCTGGCCAGATCGCCTCTCTGAGCGCCAGAGTGGTGCGGGAGGGC
GACGAGTTCAACTTTGAGTTCGGCCTGGATGAGAAGCTGATCCACCGG
CCTGGCGAGAATGAGGACGCCCCAGTGACCCACGTGTACGCAGTGGCC
AGACTGAAGGATGGCGGCACCCAGTTTGAAGTGATGACAAGGCGCCAG
ATCGAGCTGGTGAGGTCCCAGTCTAAGGCCGGCAACAATGGCCCTTGG
GTGACCCACTGGGAGGAGATGGCCAAGAAAACCGCCATCCGGAGACTG
TTCAAGTACCTGCCAGTGTCTATCGAGATCCAGCGCGCCGTGAGCATG
GACGAGAAGGAGCCACTGACCATCGACCCCGCCGATAGCTCCGTGCTG
ACAGGCGAGTATTCTGTGATCGATAACAGCGAGGAG
Shigella sonnei RecE DNA
(SEQ ID NO: 102):
GATCGCGGCCTGCTGACAAAGGAGTGGAGGAAGGGAAACCGGGTGAGC
CGGATCACCAGGACAGCCAGCGGAGCAAACGCAGGAGGAGGAAATCTG
ACCGACAGAGGCGAGGGCTTCGTGCACGATCTGACAAGCCTGGCCCGC
GACATCGCAACCGGCGTGCTGGCCCGGAGCATGGACGTGGACATCTAC
AACCTGCACCCTGCCCACGCCAAGAGGATCGAGGAGATCATCGCCGAG
AATAAGCCCCCTTTCAGCGTGTTTAGAGACAAGTTTATCACAATGCCA
GGCGGCCTGGACTACTCCAGGGCCATCGTGGTGGCCTCTGTGAAGGAG
GCCCCAATCGGCATCGAAGTGATCCCCGCCCACGTGACCGCCTATCTG
AACAAGGTGCTGACCGAGACAGACCACGCCAATCCAGATCCCGAGATC
GTGGACATCGCATGCGGCAGAAGCTCCGCCCCTATGCCACAGAGGGTG
ACCGAGGAGGGCAAGCAGGACGATGAGGAGAAGCTGCAGCCTTCTGGC
ACCACAGCAGATGAGCAGGGAGAGGCAGAGACAATGGAGCCAGACGCC
ACAAAGCACCACCAGGATACCCAGCCTCTGGACGCCCAGAGCCAGGTG
AACAGCGTGGATGCCAAGTATCAGGAGCTGAGAGCCGAGCTGCACGAG
GCCAGGAAGAACATCCCTTCCAAGAATCCAGTGGACGCAGATAAGCTG
CTGGCCGCCTCTCGCGGCGAGTTCGTGGACGGCATCAGCGACCCAAAC
GATCCCAAGTGGGTGAAGGGCATCCAGACACGGGATTCCGTGTACCAG
AATCAGCCTGAGACAGAGAAAACCAGCCCCGACATGAAGCAGCCAGAG
CCTGTGGTGCAGCAGGAGCCTGAGATCGCCTTCAACGCCTGCGGACAG
ACCGGCGGCGACAATTGCCCAGATTGTGGCGCCGTGATGGGCGATGCC
ACCTATCAGGAGACATTTGACGAGGAGAACCAGGTGGAGGCCAAGGAG
AATGATCCTGAGGAGATGGAGGGCGCCGAGCACCCACACAACGAGAAT
GCCGGCAGCGACCCCCACAGAGACTGTTCCGATGAGACAGGCGAGGTG
GCCGATCCCGTGATCGTGGAGGACATCGAGCCTGGCATCTACTATGGC
ATCAGCAACGAGAATTACCACGCAGGCCCCGGCGTGTCCAAGTCTCAG
CTGGACGACATCGCCGACACACCTGCCCTGTATCTGTGGAGGAAGAAC
GCCCCAGTGGATACCACAAAGACCAAGACACTGGACCTGGGCACCGCA
TTCCACTGCCGCGTGCTGGAGCCAGAGGAGTTCAGCAATCGGTTTATC
GTGGCCCCCGAGTTCAACCGGAGAACAAATGCCGGCAAGGAGGAGGAG
AAGGCCTTTCTGATGGAGTGTGCCTCCACAGGCAAGATGGTCATCACC
GCCGAGGAGGGCAGAAAGATCGAGCTGATGTACCAGTCTGTGATGGCA
CTGCCACTGGGACAGTGGCTGGTGGAGAGCGCCGGACACGCAGAGTCT
AGCATCTATTGGGAGGACCCCGAGACAGGCATCCTGTGCAGGTGTCGC
CCCGACAAGATCATCCCTGAGTTCCACTGGATCATGGACGTGAAAACC
ACAGCCGACATCCAGCGGTTCAAGACAGCCTACTATGATTACAGGTAT
CACGTGCAGGATGCCTTCTACTCCGACGGCTATGAGGCCCAGTTTGGC
GTGCAGCCCACCTTCGTGTTTCTGGTGGCCTCTACCACAATCGAGTGC
GGCAGATACCCCGTGGAGATCTTTATGATGGGAGAGGAGGCAAAGCTG
GCCGGACAGCTGGAGTATCACCGCAACCTGCGGACACTGGCCGATTGT
CTGAATACCGACGAGTGGCCAGCCATCAAGACCCTGTCCCTGCCCAGA
TGGGCAAAGGAGTACGCCAACGAC
Salmonella enterica RecT DNA
(SEQ ID NO: 103):
ACCAAGCAGCCCCCTATCGCCAAGGCCGACCTGCAGAAAACCCAGGGA
AACAGGGCACCTGCAGCAGTGAATGACAAGGATGTGCTGTGCGTGATC
AACAGCCCTGCCATGAAGGCACAGCTGGCCGCCGCCCTGCCAAGGCAC
ATGACCGCCGAGAGGATGATCCGCATCGCCACCACAGAGATCAGGAAG
GTGCCAGAGCTGCGCAACTGCGACAGCACCAGCTTCATCGGCGCCATC
GTGCAGTGTTCTCAGCTGGGCCTGGAGCCCGGCAGCGCCCTGGGCCAC
GCCTACCTGCTGCCTTTTGGCAATGGCAAGGCCAAGAACGGCAAGAAG
AATGTGCAGCTGATCATCGGCTATCGGGGCATGATCGATCTGGCCCGG
AGATCTGGCCAGATCATCTCCCTGAGCGCCAGAGTGGTGCGGGAGTGT
GACGAGTTCTCCTACGAGCTGGGCCTGGATGAGAAGCTGGTGCACCGG
CCAGGCGAGAACGAGGACGCACCCATCACCCACGTGTATGCCGTGGCC
AAGCTGAAGGATGGCGGCGTGCAGTTTGAAGTGATGACCAAGAAGCAG
GTGGAGAAGGTGAGAGATACACACTCCAAGGCCGCCAAGAATGCCGCC
TCTAAGGGCGCCAGCTCCATCTGGGACGAGCACTTCGAGGATATGGCC
AAGAAAACCGTGATCCGGAAGCTGTTTAAGTACCTGCCCGTGAGCATC
GAGATCCAGAGAGCCGTGAGCATGGACGGCAAGGAGGTGGAGACAATC
AACCCAGACGACATCAGCGTGATCGCCGGCGAGTATTCCGTGATCGAT
AATCCCGAGGAG
Salmonella enterica RecE DNA(SEQ ID NO: 104):
GATCGCGGCCTGCTGACAAAGGAGTGGAGGAAGGGAAACCGGGTGAGC
CGGATCACCAGGACAGCCAGCGGAGCAAACGCAGGAGGAGGAAATCTG
ACCGACAGAGGCGAGGGCTTCGTGCACGATCTGACAAGCCTGGCCCGC
GACGTGGCAACCGGCGTGCTGGCCCGGAGCATGGACGTGGACATCTAC
AACCTGCACCCTGCCCACGCCAAGAGGGTGGAGGAGATCATCGCCGAG
AATAAGCCCCCTTTCAGCGTGTTTAGAGACAAGTTTATCACAATGCCT
GGCGGCCTGGACTACTCCAGGGCCATCGTGGTGGCCTCTGTGAAGGAG
GCCCCTATCGGCATCGAAGTGATCCCAGCCCACGTGACCGAGTATCTG
AACAAGGTGCTGACCGAGACAGACCACGCCAATCCAGATCCCGAGATC
GTGGACATCGCATGCGGCAGAAGCTCCGCCCCTATGCCACAGAGGGTG
ACCGAGGAGGGCAAGCAGGACGATGAGGAGAAGCCCCAGCCTTCTGGA
GCTATGGCCGACGAGCAGGCAACCGCAGAGACAGTGGAGCCAAACGCC
ACAGAGCACCACCAGAATACCCAGCCCCTGGATGCCCAGAGCCAGGTG
AACTCCGTGGACGCCAAGTATCAGGAGCTGAGAGCCGAGCTGCAGGAG
GCCAGGAAGAACATCCCCTCCAAGAATCCTGTGGACGCAGATAAGCTG
CTGGCCGCCTCTCGCGGCGAGTTCGTGGATGGCATCAGCGACCCTAAC
GATCCAAAGTGGGTGAAGGGCATCCAGACACGGGATTCCGTGTACCAG
AATCAGCCCGAGACAGAGAAGATCTCTCCTGACGCCAAGCAGCCAGAG
CCCGTGGTGCAGCAGGAGCCCGAGACAGTGTGCAACGCCTGTGGACAG
ACCGGCGGCGACAATTGCCCTGATTGTGGCGCCGTGATGGGCGACGCC
ACATATCAGGAGACATTCGGCGAGGAGAATCAGGTGGAGGCCAAGGAG
AAGGACCCCGAGGAGATGGAGGGAGCAGAGCACCCTCACAACGAGAAT
GCCGGCAGCGACCCACACAGAGACTGTTCCGATGAGACAGGCGAGGTG
GCCGATCCAGTGATCGTGGAGGACATCGAGCCTGGCATCTACTATGGC
ATCAGCAACGAGAATTACCACGCAGGCCCCGGCGTGTCCAAGTCTCAG
CTGGACGACATCGCCGACACACCCGCCCTGTATCTGTGGAGGAAGAAC
GCCCCTGTGGATACCACAAAGACCAAGACACTGGACCTGGGCACCGCA
TTCCACTGCCGCGTGCTGGAGCCTGAGGAGTTCAGCAATCGGTTTATC
GTGGCCCCAGAGTTCAACCGGAGAACAAATGCCGGCAAGGAGGAGGAG
AAGGCCTTTCTGATGGAGTGTGCCTCCACCGGCAAGACAGTGATCACC
GCCGAGGAGGGCAGAAAGATCGAGCTGATGTACCAGTCTGTGATGGCA
CTGCCTCTGGGACAGTGGCTGGTGGAGAGCGCCGGACACGCAGAGTCT
AGCATCTATTGGGAGGACCCCGAGACAGGCATCCTGTGCAGGTGTCGC
CCAGACAAGATCATCCCCGAGTTCCACTGGATCATGGACGTGAAAACC
ACAGCCGACATCCAGCGGTTCAAGACAGCCTACTATGATTACAGGTAT
CACGTGCAGGATGCCTTCTACTCCGACGGCTATGAGGCCCAGTTTGGC
GTGCAGCCAACCTTCGTGTTTCTGGTGGCCTCTACCACAGTGGAGTGC
GGCAGATACCCCGTGGAGATCTTTATGATGGGAGAGGAGGCAAAGCTG
GCCGGACAGCAGGAGTATCACCGCAACCTGCGGACACTGGCCGATTGT
CTGAATACCGACGAGTGGCCTGCCATCAAGACCCTGTCCCTGCCACGG
TGGGCCAAGGAGTACGCCAACGAC
Acetobacter Red DNA
(SEQ ID NO: 105):
AACGCCCCCCAGAAGCAGAATACCAGAGCCGCCGTGAAGAAGATCAGC
CCTCAGGAGTTCGCCGAGCAGTTTGCCGCCATCATCCCACAGGTGAAG
TCCGTGCTGCCCGCCCACGTGACCTTCGAGAAGTTTGAGCGGGTGGTG
AGACTGGCCGTGCGGAAGAACCCTGACCTGCTGACATGCTCCCCAGCC
TCTCTGTTCATGGCATGTATCCAGGCAGCCTCCGACGGCCTGCTGCCT
GATGGAAGGGAGGGAGCAATCGTGAGCCGGTGGAGCTCCAAGAAGAGC
TGCAACGAGGCCTCCTGGATGCCAATGGTGGCCGGCCTGATGAAGCTG
GCCCGGAACAGCGGCGACATCGCCAGCATCTCTAGCCAGGTGGTGTTC
GAGGGCGAGCACTTTAGAGTGGTGCTGGGCGACGAGGAGAGGATCGAG
CACGAGCGCGATCTGGGCAAGACCGGCGGCAAGATCGTGGCAGCCTAC
GCCGTGGCAAGGCTGAAGGACGGCAGCGATCCAATCCGCGAGATCATG
TCCTGGGGCCAGATCGAGAAGATCAGAAACACAAATAAGAAGTGGGAG
TGGGGACCCTGGAAGGCCTGGGAGGACGAGATGGCCAGAAAGACCGTG
ATCCGGAGACTGGCCAAGAGACTGCCCATGTCTACAGATAAGGAGGGA
GAGAGGCTGCGCAGCGCCATCGAGAGGATCGACTCCCTGGTGGACATC
TCTGCCAACGTGGACGCACCTCAGATCGCAGCAGACGATGAGTTTGCC
GCCGCCGCCCACGGCGTGGAGCCACAGCAGATCGCAGCACCTGACCTG
ATCGGCCGCCTGGCCCAGATGCAGTCCCTGGAGCAGGTGCAGGACATC
GAGCCCCAGGTGTCTCACGCCATCCAGGAGGCCGACAAGAGGGGCGAC
AGCGATACAGCCAATGCCCTGGATGCCGCCCTGCAGAGCGCCCTGTCC
CGCACCTCTACAGCCAAGGAGGAGGTGCCTGCC
Acetobacter RecE DNA
(SEQ ID NO: 106):
GTGATCTCTAAGAGCGGCATCTACGACCTGACCAACGAGCAGTATCAC
GCCGATCCTTGCCCAGAGATGTCCCTGAGCTCCTCTGGAGCCAGGGAC
CTGCTGAGCTCCTGTCCTGCCAAGTTCATCGCCGCCAAGCAGCTGCCA
CAGCAGAATAAGAGGTGCTTTGACATCGGCTCTGCCGGACACCTGATG
GTGCTGGAGCCACACCTGTTCGACCAGAAGGTGTGCGAGATCAAGCAC
CCTGATTGGCGCACAAAGGCAGCAAAGGAGGAGCGGGACGCCGCCTAC
GCCGAGGGAAGAATCCCCCTGCTGAGCCGCGAGGTGGAGGACATCAGG
GCAATGCACTCCGTGGTGTGGAGAGATTCTCTGGGAGCCAGGGCCTTC
AGCGGAGGCAAGGCAGAGCAGTCCCTGGTGTGGCGCGACGAGGAGTTT
GGCATCTGGTGCCGGCTGCGGCCCGATTACGTGCCTAACAATGCCGTG
CGGATCTTCGACTATAAGACCGCCACAAACGGCTCCCCCGATGCCTTT
ATGAAGGAGATCTACAATCGGGGCTATCACCAGCAGGCCGCCTGGTAT
CTGGACGGATATGAGGCAGTGACCGGCCACAGGCCACGCGAGTTCTGG
TTTGTGGTGCAGGAGAAAACCGCCCCCTTCCTGCTGTCTTTCTTTCAG
ATGGATGAGATGAGCCTGGAGATCGGCCGGACCCTGAACAGACAGGCC
AAGGGCATCTTTGCCTGGTGCCTGCGCAACAATTGTTGGCCAGGCTAT
CAGCCCGAGGTGGATGGCAAGGTGAGATTCTTTACCACATCTCCCCCT
GCCTGGCTGGTGAGGGAGTACGAGTTTAAGAATGAGCACGGCGCCTAT
GAGCCACCCGAGATCAAGCGGAAGGAGGTGGCC
Salmonella enterica subsp. enterica serovar
Javiana str. 10721 RecT DNA
(SEQ ID NO: 107):
CCAAAGCAGCCCCCTATCGCCAAGGCAGACCTGCAGAAAACCCAGGGA
GCACGGACCCCAACAGCAGTGAAGAACAATAACGATGTGATCTCCTTT
ATCAATCAGCCTTCTATGAAGGAGCAGCTGGCCGCCGCCCTGCCAAGG
CACATGACCGCCGAGCGGATGATCAGAATCGCCACCACAGAGATCAGG
AAGGTGCCCGCCCTGGGCGACTGCGATACAATGTCTTTTGTGAGCGCC
ATCGTGCAGTGTAGCCAGCTGGGCCTGGAGCCTGGCGGCGCCCTGGGC
CACGCCTACCTGCTGCCTTTCGGCAATCGGAACGAGAAGTCCGGCAAG
AAGAATGTGCAGCTGATCATCGGCTATAGAGGCATGATCGACCTGGCC
CGGAGATCCGGACAGATCGCCAGCCTGTCCGCCAGGGTGGTGCGCGAG
GGCGACGATTTCTCTTTTGAGTTCGGCCTGGAGGAGAAGCTGGTGCAC
AGGCCAGGCGAGAACGAGGACGCCCCCGTGACCCACGTGTACGCAGTG
GCACGCCTGAAGGATGGAGGCACCCAGTTTGAAGTGATGACACGGAAG
CAGATCGAGCTGGTGAGAGCCCAGTCTAAGGCCGGCAATAACGGCCCT
TGGGTGACCCACTGGGAGGAGATGGCCAAGAAAACCGCCATCAGGCGC
CTGTTCAAGTACCTGCCCGTGAGCATCGAGATCCAGAGGGCCGTGAGC
ATGGATGAGAAGGAGACACTGACAATCGACCCAGCCGATGCCAGCGTG
ATCACCGGCGAGTATTCCGTGGTGGAGAATGCCGGCGTGGAGGAGAAC
GTGACAGCC
Salmonella enterica subsp. enterica serovar
Javiana str. 10721 RecE DNA
(SEQ ID NO: 108):
TACTATGACATCCCAAACGAGGCCTACCACGCAGGCCCCGGCGTGTCT
AAGAGCCAGCTGGACGACATCGCCGATACCCCCGCCATCTATCTGTGG
CGGAAGAATGCCCCTGTGGACACCGAGAAAACCAAGTCCCTGGATACC
GGCACAGCCTTCCACTGCAGGGTGCTGGAGCCAGAGGAGTTCAGCAAG
CGGTTCATCATCGCCCCCGAGTTCAACCGGAGAACCTCCGCCGGCAAG
GAGGAGGAGAAAACCTTCCTGGAGGAGTGTACCCGGACAGGCAGAACC
GTGCTGACAGCCGAGGAGGGCAGGAAGATCGAGCTGATGTACCAGTCC
GTGATGGCACTGCCACTGGGACAGTGGCTGGTGGAGTCTGCCGGCTAC
GCCGAGAGCTCCGTGTATTGGGAGGACCCTGAGACAGGCATCCTGTGC
CGGTGTAGACCCGATAAGATCATCCCTGAGTTCCACTGGATCATGGAC
GTGAAAACCACAGCCGACATCCAGAGGTTTCGCACCGCCTACTATGAC
TACAGATACCACGTGCAGGACGCCTTCTACTCTGATGGCTATAGAGCC
CAGTTTGGCGAGATCCCTACATTCGTGTTTCTGGTGGCCAGCACCACA
GCAGAGTGCGGCAGATACCCCGTGGAGATCTTTATGATGGGAGAGGAC
GCAAAGCTGGCCGGACAGCGCGAGTATAGGCGCAATCTGCAGACCCTG
GCCGAGTGTCTGAACAATGATGAGTGGCCTGCCATCAAGACACTGTCT
CTGCCACGGTGGGCCAAGGAGAACGCCAATGCC
Pseudobacteriovorax antillogorgiicola RecT DNA
(SEQ ID NO: 109):
GGCCACCTGGTGAGCAAGACCGAGCAGGATTACATCAAGCAGCACTAT
GCCAAGGGCGCCACAGACCAGGAGTTCGAGCACTTTATCGGCGTGTGC
AGGGCCAGAGGCCTGAACCCAGCCGCCAATCAGATCTACTTCGTGAAG
TATCGGTCCAAGGATGGACCAGCAAAGCCAGCCTTTATCCTGTCTATC
GACAGCCTGAGGCTGATCGCACACCGCACCGGCGATTACGCAGGATGC
TCTGAGCCCATCTTCACAGACGGCGGCAAGGCCTGTACCGTGACAGTG
CGGAGAAACCTGAAGAGCGGCGAGACAGGCAATTTCTCCGGCATGGCC
TTTTATGACGAGCAGGTGCAGCAGAAGAACGGCCGGCCTACCTCCTTT
TGGCAGTCTAAGCCAAGAACAATGCTGGAGAAGTGTGCAGAGGCAAAG
GCCCTGAGGAAGGCCTTCCCTCAGGATCTGGGCCAGTTTTACATCAGA
GAGGAGATGCCCCCTCAGTATGACGAGCCTATCCAGGTGCACAAGCCA
AAGGCCCTGGAGGAGCCCAGGTTCAGCAAGTCCGATCTGTCCAGGCGC
AAGGGCCTGAACAGGAAGCTGTCTGCCCTGGGAGTGGACCCCAGCCGC
TTCGATGAGGTGGCCACCTTTCTGGACGGCACACCTGATCGCGAGCTG
GGCCAGAAGCTGAAGCTGTGGCTGAAGGAGGCCGGCTACGGCGTGAAT
CAG
Pseudobacteriovorax antillogorgiicola RecE DNA
(SEQ ID NO: 110):
AGCAAGCTGTCCAACCTGAAGGTGTCTAATAGCGACGTGGATACACTG
AGCCGGATCAGAATGAAGGAGGGCGTGTATCGGGACCTGCCAATCGAG
AGCTACCACCAGTCCCCCGGCTATTCTAAGACCAGCCTGTGCCAGATC
GATAAGGCCCCTATCTACCTGAAAACCAAGGTGCCACAGAAGTCCACA
AAGTCTCTGAACATCGGCACCGCCTTCCACGAGGCTATGGAGGGCGTG
TTTAAGGACAAGTATGTGGTGCACCCCGATCCTGGCGTGAATAAGACC
ACAAAGTCTTGGAAGGACTTCGTGAAGAGGTATCCTAAGCACATGCCA
CTGAAGCGCAGCGAGTACGACCAGGTGCTGGCCATGTACGATGCCGCC
CGGTCTTATAGACCTTTTCAGAAGTACCACCTGAGCCGGGGCTTCTAC
GAGAGCTCCTTTTATTGGCACGATGCCGTGACAAACAGCCTGATCAAG
TGCAGACCCGACTATATCACCCCTGATGGCATGAGCGTGATCGACTTC
AAGACCACAGTGGACCCCAGCCCCAAGGGCTTTCAGTACCAGGCCTAC
AAGTATCACTACTACGTGAGCGCCGCCCTGACCCTGGAGGGAATCGAG
GCAGTGACCGGCATCAGGCCAAAGGAGTACCTGTTCCTGGCCGTGTCC
AATTCTGCCCCATACCTGACCGCCCTGTATCGCGCCTCTGAGAAGGAG
ATCGCCCTGGGCGACCACTTTATCCGGCGGAGCCTGCTGACCCTGAAA
ACCTGTCTGGAGTCTGGCAAGTGGCCCGGCCTGCAGGAGGAGATCCTG
GAGCTGGGCCTGCCTTTCTCCGGCCTGAAGGAGCTGAGAGAGGAGCAG
GAGGTGGAGGATGAGTTTATGGAGCTGGTGGGC
Photobacterium sp. JCM 19050 RecT DNA
(SEQ ID NO: 111):
AACACCGACATGATCGCCATGCCCCCTTCTCCAGCCATCAGCATGCTG
GACACAAGCAAGCTGGATGTGATGGTGCGGGCAGCAGAGCTGATGTCC
CAGGCCGTGGTCATGGTGCCCGACCACTTCAAGGGCAAGCCAGCCGAT
TGCCTGGCAGTGGTCATGCAGGCAGACCAGTGGGGCATGAACCCCTTT
ACCGTGGCCCAGAAAACCCACCTGGTGAGCGGCACCCTGGGATACGAG
TCCCAGCTGGTGAATGCCGTGATCAGCTCCTCTAAGGCCATCAAGGGC
CGGTTCCACTATGAGTGGTCTGATGGCTGGGAGAGACTGGCCGGCAAG
GTGCAGTACGTGAAGGAGTCTCGGCAGAGAAAGGGCCAGCAGGGCAGC
TATCAGGTGACCGTGGCCAAGCCAACATGGAAGCCAGAGGACGAGCAG
GGCCTGTGGGTGCGGTGTGGAGCCGTGCTGGCCGGAGAGAAGGACATC
ACATGGGGCCCTAAGCTGTACCTGGCCAGCGTGCTGGTGCGGAACAGC
GAGCTGTGGACCACAAAGCCCTACCAGCAGGCCGCCTATACCGCCCTG
AAGGATTGGTCCCGCCTGTATACACCTGCCGTGATGCAGGGCTCTATG
ACCGGCAAGAGCTGGTCCCTGACAGGCAGGCTGATCAGCCCCCGC
Photobacterium sp. JCM 19050 RecE DNA
(SEQ ID NO: 112):
GCCGAGCGGGTGAGAACCTATCAGCGGGACGCCGTGTTCGCACACGAG
CTGAAGGCCGAGTTTGATGAGGCCGTGGAGAACGGCAAGACCGGCGTG
ACACTGGAGGACCAGGCCAGGGCCAAGAGGATGGTGCACGAGGCCACC
ACAAACCCCGCCTCTCGGAATTGGTTCAGATACGACGGAGAGCTGGCC
GCATGCGAGAGGAGCTATTTTTGGCGCGATGAGGAGGCAGGCCTGGTG
CTGAAGGCCAGGCCTGACAAGGAGATCGGCAACAATCTGATCGATGTG
AAGTCCATCGAGGTGCCAACCGACGTGTGCGCCTGTGATCTGAACGCC
TATATCAATCGGCAGATCGAGAAGAGAGGCTACCACATCTCCGCCGCC
CACTATCTGTCTGGCACAGGCAAGGACCGCTTCTTTTGGATCTTCATC
AATAAGGTGAAGGGCTACGAGTGGGTGGCAATCGTGGAGGCCTCTCCC
CTGCACATCGAGCTGGGCACCTATGAGGTGCTGGAGGGCCTGCGGAGC
ATCGCCAGCTCCACAAAGGAGGCAGATTACCCAGCACCTCTGTCCCAC
CCTGTGAACGAGAGAGGCATCCCACAGCCCCTGATGTCTAATCTGAGC
ACATACGCCATGAAGAGGCTGGAGCAGTTTCGCGAGCTG
Providencia alcalifaciens DSM 30120 RecT DNA
(SEQ ID NO: 113):
AAGGCACAGCTGGCCGCCGCCCTGCCTAAGCACATCACCAGCGACCGG
ATGATCAGAATCGTGTCCACCGAGATCAGAAAGACCCCATCTCTGGCC
AACTGCGACATCCAGAGCTTCATCGGCGCCGTGGTGCAGTGTTCTCAG
CTGGGCCTGGAGCCAGGCAACGCCCTGGGACACGCCTACCTGCTGCCC
TTTGGCAATGGCAAGTCCGACAACGGCAAGTCTAATGTGCAGCTGATC
ATCGGCTATCGGGGCATGATCGATCTGGCCCGGAGAAGCGGCCAGATC
ATCTCTATCAGCGCCAGGACCGTGCGCCAGGGCGACAACTTCCACTTT
GAGTACGGCCTGAACGAGAATCTGACCCACATCCCCGAGGGCAATGAG
GACTCCCCTATCACACACGTGTACGCAGTGGCACGGCTGAAGGATGAG
GGCGTGCAGTTCGAAGTGATGACATATAACCAGATCGAGAAGGTGAGA
GATAGCTCCAAGGCCGGCAAGAATGGCCCCTGGGTGACCCACTGGGAG
GAGATGGCCAAGAAAACCGTGATCAGGCGCCTGTTTAAGTACCTGCCC
GTGAGCATCGAGATGCAGAAGGCCGTGATCCTGGACGAGAAGGCCGAG
GCCAATATCGAGCAGGATCACTCCGCCATCTTCGAGGCCGAGTTTGAG
GAGGTGGACTCTAACGGCAAT
Providencia alcalifaciens DSM 30120 RecE DNA
(SEQ ID NO: 114):
AACGAGGGCATCTACTATGACATCTCTAATGAGGACTATCACCACGGC
CTGGGCATCTCTAAGAGCCAGCTGGATCTGATCGACGAGAGCCCCGCC
GATTTCATCTGGCACCGGGATGCCCCTGTGGACAACGAGAAAACCAAG
GCCCTGGATTTTGGCACAGCCCTGCACTGCCTGCTGCTGGAGCCAGAC
GAGTTCCAGAAGAGGTTTCGCATCGCCCCCGAGGTGAACCGGAGAACA
AATGCCGGCAAGGAGCAGGAGAAGGAGTTCCTGGAGATGTGCGAGAAG
GAGAATATCACCCCCATCACAAACGAGGATAATAGGAAGCTGTCTCTG
ATGAAGGACAGCGCAATGGCCCACCCTATCGCCCGCTGGTGTCTGGAG
GCCAAGGGCATCGCCGAGAGCTCCATCTATTGGAAGGACAAGGATACA
GACATCCTGTGCCGGTGTAGACCAGACAAGCTGATCGAGGAGCACCAC
TGGCTGGTGGATGTGAAGTCCACCGCCGACATCCAGAAGTTCGAGCGG
TCTATGTACGAGTATAGATACCACGTGCAGGATTCCTTTTATTCTGAC
GGCTACAAGAGCCTGACAGGCGAGATGCCCGTGTTCGTGTTCCTGGCC
GTGTCCACCGTGATCAACTGCGGCAGATACCCCGTGCGGGTGTTCGTG
CTGGACGAGCAGGCAAAGTCCGTGGGACGGATCACCTATAAGCAGAAT
CTGTTTACATACGCCGAGTGTCTGAAAACCGACGAGTGGGCCGGCATC
AGAACCCTGAGCCTGCCCTCCTGGGCAAAGGAGCTGAAGCACGAGCAC
ACCACAGCCTCT
Pantoea stewartii Red Protein
(SEQ ID NO: 115):
MSNQPPIASADLQKANTGKQVANKTPEQTLVGFMNQPAMKSQLAAALP
RHMTADRMIRIVTTEIRKTPALATCDQSSFIGAVVQCSQLGLEPGSAL
GHAYLLPFGNGRSKSGQSNVQLIIGYRGMIDLARRSGQIVSLSARVVR
ADDEFSFEYGLDENLIHRPGENEDAPITHVYAVARLKDGGTQFEVMTV
KQIEKVKAQSKASSNGPWVTHWEEMAKKTVIRRLFKYLPVSIEMQKAV
ILDEKAESDVDQDNASVLSAEYSVLDGSSEE
Pantoea stewartii RecE Protein
(SEQ ID NO: 116):
MQPGVYYDISNEEYHAGPGISKSQLDDIAVSPAIFQWRKSAPVDDEKT
AALDLGTALHCLLLEPDEFSKRFMIGPEVNRRTNAGKQKEQDFLDMCE
QQGITPITHDDNRKLRLMRDSAFAHPVARWMLETEGKAEASIYWNDRD
TQILSRCRPDKLITEFSWCVDVKSTADIGKFQKDFYSYRYHVQDAFYS
DGYEAQFCEVPTFAFLVVSSSIDCGRYPVQVFIMDQQAKDAGRAEYKR
NLTTYAECQARNEWPGIATLSLPYWAKEIRNV
Pantoea brenneri Red Protein
(SEQ ID NO: 117):
MSNQPPIASADLQKTQQSKQVANKTPEQTLVGFMNQPAMKSQLAAALP
RHMTADRMIRIVTTEIRKTPQLAQCDQSSFIGAVVQCSQLGLEPGSAL
GHAYLLPFGNGRSKSGQSNVQLIIGYRGMIDLARRSGQIVSLSARVVR
ADDEFSFEYGLDENLVHRPGENEDAPITHVYAVARLKDGGTQFEVMTV
KQVEKVKAQSKASSNGPWVTHWEEMAKKTVIRRLFKYLPVSIEMQKAV
VLDEKAESDVDQDNASVLSAEYSVLESGDEATN
Pantoea brenneri RecE Protein
(SEQ ID NO: 118):
MQPGIYYDISNEDYHRGAGISKSQLDDIAISPAIYQWRKHAPVDEEKT
AALDLGTALHCLLLEPDEFSKRFQIGPEVNRRTTAGKEKEKEFIERCE
AEGITPITHDDNRKLKLMRDSALAHPIARWMLEAQGNAEASIYWNDRD
AGVLSRCRPDKIITEFNWCVDVKSTADIMKFQKDFYSYRYHVQDAFYS
DGYESHFHETPTFAFLAVSTSIDCGRYPVQVFIMDQQAKDAGRAEYKR
NIHTFAECLSRNEWPGIATLSLPFWAKELRNE
Pantoea dispersa Red Protein
(SEQ ID NO: 119):
MSNQPPLATADLQKTQQSNQVAKTPEQTLVGFMNQPAMKSQLAAALPR
HMTADRMIRIVTTEIRKTPALAQCDQSSFIGAVVQCSQLGLEPGSALG
HAYLLPFGNGRSKSGQSNVQLIIGYRGMIDLARRSGQIVSLSARVVRA
DDEFSFEYGLDENLIHRPGDNESAPITHVYAVARLKDGGTQFEVMTAK
QVEKVKAQSKASSNGPWVTHWEEMAKKTVIRRLFKYLPVSIEMQKAVV
LDEKAESDVDQDNASVLSAEYSVLESGTGE
Pantoea dispersa RecE Protein
(SEQ ID NO: 120):
MEPGIYYDISNEAYHSGPGISKSQLDDIARSPAIFQWRKDAPVDTEKT
KALDLGTDFHCAVLEPERFADMYRVGPEVNRRTTAGKAEEKEFFEKCE
KDGAVPITHDDARKVELMRGSVMAHPIAKQMIAAQGHAEASIYWHDES
TGNLCRCRPDKFIPDWNWIVDVKTTADMKKFRREFYDLRYHVQDAFYT
DGYAAQFGERPTFVFVVTSTTIDCGRYPTEVFFLDEETKAAGRSEYQS
NLVTYSECLSRNEWPGIATLSLPHWAKELRNV
Type-F symbiont of Plautia stali Red Protein
(SEQ ID NO: 121):
MSNQPPIASADLQKTQQSKQVANKTPEQTLVGFMNQPAMKSQLAAALP
RHMTADRMIRIVTTEIRKTPALATCDQSSFIGAVVQCSQLGLEPGSAL
GHAYLLPFGNGRSKSGQSNVQLIIGYRGMIDLARRSGQIVSLSARVVR
ADDEFSFEYGLDENLIHRPGDNEDAPITHVYAVARLKDGGTQFEVMTA
KQVEKVKAQSKASSNGPWVTHWEEMAKKTVIRRLFKYLPVSIEMQKAV
VLDEKAESDVDQDNASVLSAEYSVLEGDGGE
Type-F symbiont of Plautia stali RecE Protein
(SEQ ID NO: 122):
MQPGIYYDISNEDYHGGPGISKSQLDDIAISPAIYQWRKHAPVDEEKT
AALDLGTALHCLLLEPDEFSKRFEIGPEVNRRTTAGKEKEKEFMERCE
AEGVTPITHDDNRKLRLMRDSAMAHPIARWMLEAQGNAEASIYWNDRD
TGVLSRCRPDKIITDFNWCVDVKSTADIIKFQKDFYSYRYHVQDAFYS
DGYESHFDETPTFAFLAVSTSIDCGRYPVQVFIMDQQAKDAGRAEYKR
NIHTFAECLSRNEWPGIATLSLPYWAKELRNE
Providencia stuartii Red Protein
(SEQ ID NO: 123):
MSNPPLAQADLQKTQGTEVKEKTKDQMLVELINKPSMKAQLAAALPRH
MTPDRMIRIVTTEIRKTPALATCDMQSFVGAVVQCSQLGLEPGNALGH
AYLLPFGNGKSKSGQSNVQLIIGYRGMIDLARRSGQIVSISARTVRQG
DNFHFEYGLNENLTHVPGENEDSPITHVYAVARLKDGGVQFEVMTYNQ
IEKVRASSKAGQNGPWVSHWEEMAKKTVIRRLFKYLPVSIEMQKAVIL
DEKAEANIDQENATIFEGEYEEVGTDGK
Providencia stuartii RecE Protein
(SEQ ID NO: 124):
EGIYYNISNEDYHNGLGISKSQLDLINEMPAEYIWSKEAPVDEEKIKP
LEIGTALHCLLLEPDEYHKRYKIGPDVNRRTNAGKEKEKEFFDMCEKE
GITPITHDDNRKLMIMRDSALAHPIAKWCLEADGVSESSIYWTDKETD
VLCRCRPDRIITAHNYIVDVKSSGDIEKFDYEYYNYRYHVQDAFYSDG
YKEVTGITPTFLFLVVSTKIDCGKYPVRTYVMSEEAKSAGRTAYKHNL
LTYAECLKTDEWAGIRTLSLPRWAKELRNE
Providencia sp. MGF014 Red Protein
(SEQ ID NO: 125):
MSNPPLAQSDLQKTQGTEVKVKTKDQQLIQFINQPSMKAQLAAALPRH
MTPDRMIRIVTTEIRKTPALATCDMQSFVGAVVQCSQLGLEPGNALGH
AYLLPFGNGKAKSGQSNVQLIIGYRGMIDLARRSNQIISISARTVRQG
DNFHFEYGLNEDLTHTPSENEDSPITHVYAVARLKDGGVQFEVMTYNQ
VEKVRASSKAGQNGPWVSHWEEMAKKTVIRRLFKYLPVSIEMQKAVVL
DEKAEANVDQENATIFEGEYEEVGTDGN
Providencia sp. MGF014 RecE Protein
(SEQ ID NO: 126):
MKEGIYYNISNEDYHNGLGISKSQLDLINEMPAEYIWSKEAPVDEEKI
KPLEIGTALHCLLLEPDEYHKRYKIGPDVNRRTNVGKEKEKEFFDMCE
KEGITPITHDDNRKLMIMRDSALAHPIAKWCLEADGVSESSIYWTDKE
TDVLCRCRPDRIITAHNYIIDVKSSGDIEKFDYEYYNYRYHVQDAFYS
DGYKEVTGITPTFLFLVVSTKIDCGKYPVRTYVMSEEAKSAGRTAYKH
NLLTYAECLKTDEWAGIRTLSLPRWAKELRNE
Shewanella putrefaciens RecT Protein
(SEQ ID NO: 127):
MQTAQVKLSVPHQQVYQDNFNYLSSQVVGHLVDLNEEIGYLNQIVFNS
LSTASPLDVAAPWSVYGLLLNVCRLGLSLNPEKKLAYVMPSWSETGEI
IMKLYPGYRGEIAIASNFNVIKNANAVLVYENDHFRIQAATGEIEHFV
TSLSIDPRVRGACSGGYCRSVLMDNTIQISYLSIEEMNAIAQNQIEAN
MGNTPWNSIWRTEMNRVALYRRAAKDWRQLIKATPEIQSALSDTEY
Shewanella putrefaciens RecE Protein
(SEQ ID NO: 128):
MGTALAQTISLDWQDTIQPAYTASGKPNFLNAQGEIVEGIYTDLPNSV
YHALDAHSSTGIKTFAKGRHHYFRQYLSDVCRQRTKQQEYTFDAGTYG
HMLVLEPENFHGNFMRNPVPDDFPDIELIESIPQLKAALAKSNLPVSG
AKAALIERLYAFDPSLPLFEKMREKAITDYLDLRYAKYLRTDVELDEM
ATFYGIDTSQTREKKIEEILAISPSQPIWEKLISQHVIDHIVWDDAMR
VERSTRAHPKADWLISDGYAELTIIARCPTTGLLLKVRFDWLRNDAIG
VDFKTTLSTNPTKFGYQIKDLRYDLQQVFYCYVANLAGIPVKHFCFVA
TEYKDADNCETFELSHKKVIESTEEMFDLLDEFKEALTSGNWYGHDRS
RSTWVIEV
Bacillus sp. MUM 116 Red Protein
(SEQ ID NO: 129):
MSKQLTTVNTQAVVGTFSQAELDTLKQTIAKGTTNEQFALFVQTCANS
RLNPFLNHIHCIVYNGKEGATMSLQIAVEGILYLARKTDGYKGIECQL
IHENDEFKFDAKSKEVDHQIGFPRGNVIGGYAIAKREGFDDVVVLMES
NEVDHMLKGRNGHMWRDWFNDMFKKHIMKRAAKLQYGIEIAEDETVSS
GPSVDNIPEYKPQPRKDITPNQDVIDAPPQQPKQDDEAAKLKAARSEV
SKKFKKLGIVKEDQTEYVEKHVPGFKGTLSDFIGLSQLLDLNIEAQEA
QSADGDLLD
Bacillus sp. MUM 116 RecEProtein
(SEQ ID NO: 130):
MTYAADETLVQLLLSVDGKQLLLGRGLKKGKAQYYINEVPSKAKEFEE
IRDQLFDKDLFMSLFNPSYFFTLHWEKQRAMMLKYVTAPVSKEVLKNL
PEAQSEVLERYLKKHSLVDLEKIHKDNKNKQDKAYISAQSRTNTLKEQ
LMQLTEEKLDIDSIKAELAHIDMQVIELEKQMDTAFEKNQAFNLQAQI
RNLQDKIEMSKERWPSLKNEVIEDTCRTCKRPLDEDSVEAVKADKDNR
IAEYKAKHNSLVSQRNELKEQLNTIEYIDVTELREQIKELDESGQPLR
EQVRIYSQYQNLDTQVKSAEADENGILQDLKASIFILDSIKAFRGKEA
EMQAEKVQALFTTLSVRLFKQNKGDGEIKPDFEIEMNDKPYRTLSLSE
GIRAGLELRDVLSQQSELVTPTFVDNAESITSFKQPNGQLIISRVVAG
QELKIEAVSE
Shigella sonnei Red Protein
(SEQ ID NO: 131):
MTKQPPIAKADLQKTQENRAPAAIKNNDVISFINQPSMKEQLAAALPR
HMTAERMIRIATTEIRKVPALGNCDTMSFVSAIVQCSQLGLEPGSALG
HAYLLPFGNKNEKSGKKNVQLIIGYRGMIDLARRSGQIASLSARVVRE
GDEFNFEFGLDEKLIHRPGENEDAPVTHVYAVARLKDGGTQFEVMTRR
QIELVRSQSKAGNNGPWVTHWEEMAKKTAIRRLFKYLPVSIEIQRAVS
MDEKEPLTIDPADSSVLTGEYSVIDNSEE
Shigella sonnei RecE Protein
(SEQ ID NO: 132):
DRGLLTKEWRKGNRVSRITRTASGANAGGGNLTDRGEGFVHDLTSLAR
DIATGVLARSMDVDIYNLHPAHAKRIEEIIAENKPPFSVFRDKFITMP
GGLDYSRAIVVASVKEAPIGIEVIPAHVTAYLNKVLTETDHANPDPEI
VDIACGRSSAPMPQRVTEEGKQDDEEKLQPSGTTADEQGEAETMEPDA
TKHHQDTQPLDAQSQVNSVDAKYQELRAELHEARKNIPSKNPVDADKL
LAASRGEFVDGISDPNDPKWVKGIQTRDSVYQNQPETEKTSPDMKQPE
PVVQQEPEIAFNACGQTGGDNCPDCGAVMGDATYQETFDEENQVEAKE
NDPEEMEGAEHPHNENAGSDPHRDCSDETGEVADPVIVEDIEPGIYYG
ISNENYHAGPGVSKSQLDDIADTPALYLWRKNAPVDTTKTKTLDLGTA
FHCRVLEPEEFSNRFIVAPEFNRRTNAGKEEEKAFLMECASTGKMVIT
AEEGRKIELMYQSVMALPLGQWLVESAGHAESSIYWEDPETGILCRCR
PDKIIPEFHWIMDVKTTADIQRFKTAYYDYRYHVQDAFYSDGYEAQFG
VQPTFVFLVASTTIECGRYPVEIFMMGEEAKLAGQLEYHRNLRTLADC
LNTDEWPAIKTLSLPRWAKEYAND
Salmonella enterica RecT Protein
(SEQ ID NO: 133):
MTKQPPIAKADLQKTQGNRAPAAVNDKDVLCVINSPAMKAQLAAALPR
HMTAERMIRIATTEIRKVPELRNCDSTSFIGAIVQCSQLGLEPGSALG
HAYLLPFGNGKAKNGKKNVQLIIGYRGMIDLARRSGQIISLSARVVRE
CDEFSYELGLDEKLVHRPGENEDAPITHVYAVAKLKDGGVQFEVMTKK
QVEKVRDTHSKAAKNAASKGASSIWDEHFEDMAKKTVIRKLFKYLPVS
IEIQRAVSMDGKEVETINPDDISVIAGEYSVIDNPEE
Salmonella enterica RecE Protein
(SEQ ID NO: 134):
DRGLLTKEWRKGNRVSRITRTASGANAGGGNLTDRGEGFVHDLTSLAR
DVATGVLARSMDVDIYNLHPAHAKRVEEIIAENKPPFSVFRDKFITMP
GGLDYSRAIVVASVKEAPIGIEVIPAHVTEYLNKVLTETDHANPDPEI
VDIACGRSSAPMPQRVTEEGKQDDEEKPQPSGAMADEQATAETVEPNA
TEHHQNTQPLDAQSQVNSVDAKYQELRAELQEARKNIPSKNPVDADKL
LAASRGEFVDGISDPNDPKWVKGIQTRDSVYQNQPETEKISPDAKQPE
PVVQQEPETVCNACGQTGGDNCPDCGAVMGDATYQETFGEENQVEAKE
KDPEEMEGAEHPHNENAGSDPHRDCSDETGEVADPVIVEDIEPGIYYG
ISNENYHAGPGVSKSQLDDIADTPALYLWRKNAPVDTTKTKTLDLGTA
FHCRVLEPEEFSNRFIVAPEFNRRTNAGKEEEKAFLMECASTGKTVIT
AEEGRKIELMYQSVMALPLGQWLVESAGHAESSIYWEDPETGILCRCR
PDKIIPEFHWIMDVKTTADIQRFKTAYYDYRYHVQDAFYSDGYEAQFG
VQPTFVFLVASTTVECGRYPVEIFMMGEEAKLAGQQEYHRNLRTLADC
LNTDEWPAIKTLSLPRWAKEYAND
Acetobacter RecT Protein
(SEQ ID NO: 135):
MNAPQKQNTRAAVKKISPQEFAEQFAAIIPQVKSVLPAHVTFEKFERV
VRLAVRKNPDLLTCSPASLFMACIQAASDGLLPDGREGAIVSRWSSKK
SCNEASWMPMVAGLMKLARNSGDIASISSQVVFEGEHFRVVLGDEERI
EHERDLGKTGGKIVAAYAVARLKDGSDPIREIMSWGQIEKIRNTNKKW
EWGPWKAWEDEMARKTVIRRLAKRLPMSTDKEGERLRSAIERIDSLVD
ISANVDAPQIAADDEFAAAAHGVEPQQIAAPDLIGRLAQMQSLEQVQD
IEPQVSHAIQEADKRGDSDTANALDAALQSALSRTSTAKEEVPA
Acetobacter RecE Protein
(SEQ ID NO: 136):
MVISKSGIYDLTNEQYHADPCPEMSLSSSGARDLLSSCPAKFIAAKQL
PQQNKRCFDIGSAGHLMVLEPHLFDQKVCEIKHPDWRTKAAKEERDAA
YAEGRIPLLSREVEDIRAMHSVVWRDSLGARAFSGGKAEQSLVWRDEE
FGIWCRLRPDYVPNNAVRIFDYKTATNGSPDAFMKEIYNRGYHQQAAW
YLDGYEAVTGHRPREFWFVVQEKTAPFLLSFFQMDEMSLEIGRTLNRQ
AKGIFAWCLRNNCWPGYQPEVDGKVRFFTTSPPAWLVREYEFKNEHGA
YEPPEIKRKEVA
Salmonella enterica subsp. enterica serovar
Javiana str. 10721 RecT Protein
(SEQ ID NO: 137):
MPKQPPIAKADLQKTQGARTPTAVKNNNDVISFINQPSMKEQLAAALP
RHMTAERMIRIATTEIRKVPALGDCDTMSFVSAIVQCSQLGLEPGGAL
GHAYLLPFGNRNEKSGKKNVQLIIGYRGMIDLARRSGQIASLSARVVR
EGDDFSFEFGLEEKLVHRPGENEDAPVTHVYAVARLKDGGTQFEVMTR
KQIELVRAQSKAGNNGPWVTHWEEMAKKTAIRRLFKYLPVSIEIQRAV
SMDEKETLTIDPADASVITGEYSVVENAGVEENVTA
Salmonella enterica subsp. enterica serovar
Javiana str. 10721 RecE Protein
(SEQ ID NO: 138):
MYYDIPNEAYHAGPGVSKSQLDDIADTPAIYLWRKNAPVDTEKTKSLD
TGTAFHCRVLEPEEFSKRFIIAPEFNRRTSAGKEEEKTFLEECTRTGR
TVLTAEEGRKIELMYQSVMALPLGQWLVESAGYAESSVYWEDPETGIL
CRCRPDKIIPEFHWIMDVKTTADIQRFRTAYYDYRYHVQDAFYSDGYR
AQFGEIPTFVFLVASTTAECGRYPVEIFMMGEDAKLAGQREYRRNLQT
LAECLNNDEWPAIKTLSLPRWAKENANA
Pseudobacteriovorax antillogorgiicola RecT Protein
(SEQ ID NO: 139):
MGHLVSKTEQDYIKQHYAKGATDQEFEHFIGVCRARGLNPAANQIYFV
KYRSKDGPAKPAFILSIDSLRLIAHRTGDYAGCSEPIFTDGGKACTVT
VRRNLKSGETGNFSGMAFYDEQVQQKNGRPTSFWQSKPRTMLEKCAEA
KALRKAFPQDLGQFYIREEMPPQYDEPIQVHKPKALEEPRFSKSDLSR
RKGLNRKLSALGVDPSRFDEVATFLDGTPDRELGQKLKLWLKEAGYGV
NQ
Pseudobacteriovorax antillogorgiicola RecE Protein
(SEQ ID NO: 140):
MSKLSNLKVSNSDVDTLSRIRMKEGVYRDLPIESYHQSPGYSKTSLCQ
IDKAPIYLKTKVPQKSTKSLNIGTAFHEAMEGVFKDKYVVHPDPGVNK
TTKSWKDFVKRYPKHMPLKRSEYDQVLAMYDAARSYRPFQKYHLSRGF
YESSFYWHDAVTNSLIKCRPDYITPDGMSVIDFKTTVDPSPKGFQYQA
YKYHYYVSAALTLEGIEAVTGIRPKEYLFLAVSNSAPYLTALYRASEK
EIALGDHFIRRSLLTLKTCLESGKWPGLQEEILELGLPFSGLKELREE
QEVEDEFMELVG
Photobacterium sp. JCM 19050 Red Protein
(SEQ ID NO: 141):
MNTDMIAMPPSPAISMLDTSKLDVMVRAAELMSQAVVMVPDHFKGKPA
DCLAVVMQADQWGMNPFTVAQKTHLVSGTLGYESQLVNAVISSSKAIK
GRFHYEWSDGWERLAGKVQYVKESRQRKGQQGSYQVTVAKPTWKPEDE
QGLWVRCGAVLAGEKDITWGPKLYLASVLVRNSELWTTKPYQQAAYTA
LKDWSRLYTPAVMQGSMTGKSWSLTGRLISPR
Photobacterium sp. JCM 19050 RecE Protein
(SEQ ID NO: 142):
MAERVRTYQRDAVFAHELKAEFDEAVENGKTGVTLEDQARAKRMVHEA
TTNPASRNWFRYDGELAACERSYFWRDEEAGLVLKARPDKEIGNNLID
VKSIEVPTDVCACDLNAYINRQIEKRGYHISAAHYLSGTGKDRFFWIF
INKVKGYEWVAIVEASPLHIELGTYEVLEGLRSIASSTKEADYPAPLS
HPVNERGIPQPLMSNLSTYAMKRLEQFREL
Providencia alcalifaciens DSM 30120 Red Protein
(SEQ ID NO: 143):
MKAQLAAALPKHITSDRMIRIVSTEIRKTPSLANCDIQSFIGAVVQCS
QLGLEPGNALGHAYLLPFGNGKSDNGKSNVQLIIGYRGMIDLARRSGQ
IISISARTVRQGDNFHFEYGLNENLTHIPEGNEDSPITHVYAVARLKD
EGVQFEVMTYNQIEKVRDSSKAGKNGPWVTHWEEMAKKTVIRRLFKYL
PVSIEMQKAVILDEKAEANIEQDHSAIFEAEFEEVDSNGN
Providencia alcalifaciens DSM 30120 RecE Protein
(SEQ ID NO: 144):
MNEGIYYDISNEDYHHGLGISKSQLDLIDESPADFIWHRDAPVDNEKT
KALDFGTALHCLLLEPDEFQKRFRIAPEVNRRTNAGKEQEKEFLEMCE
KENITPITNEDNRKLSLMKDSAMAHPIARWCLEAKGIAESSIYWKDKD
TDILCRCRPDKLIEEHHWLVDVKSTADIQKFERSMYEYRYHVQDSFYS
DGYKSLTGEMPVFVFLAVSTVINCGRYPVRVFVLDEQAKSVGRITYKQ
NLFTYAECLKTDEWAGIRTLSLPSWAKELKHEHTTAS
Mouse Albumin knock-in sense template
(SEQ ID NO: 160)
CACCTTCAGATTTTCCTGTAACGATCGGGAACTGGCATCTTCAGGGAG
TAGctgacctcttctcttcctcccacaggATCCTGGAGCCACCCGCAG
TTCGAAAAGCTCAGTGAAGAGAAGAACAAAAAGCAGCATATTACAGTT
AGTTGTCTTCATCAATCTTTAAATATGTTGTGTGGTTTTTCTCTCCCT
GTTTCCAC
Mouse Albumin knock-in anti-sense template
(SEQ ID NO: 161)
GTGGAAACAGGGAGAGAAAAACCACACAACATATTTAAAGATTGATGA
AGACAACTAACTGTAATATGCTGCTTTTTGTTCTTCTCTTCACTGAGC
TTTTCGAACTGCGGGTGGCTCCAGGATcctgtgggaggaagagaagag
gtcagCTACTCCCTGAAGATGCCAGTTCCCGATCGTTACAGGAAAATC
TGAAGGTG
(SEQ ID NO: 162)
ACTTTGAGTGTAGCAGAGAGGAACCATTGCCACCTTCAGATTTTCCTG
TAACGATCGGGAACTGGCATCTTCAGGGAGTAGCTGACCTCTTCTCTT
CCTCCCACAGGATCCTGGAGCCACC

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A system comprising:
a Cas protein;
a nucleic acid molecule comprising a guide RNA sequence that is complementary to a target DNA sequence; and
a microbial recombination protein,
wherein the microbial recombination protein is selected from the group consisting of RecE, RecT, lambda exonuclease, Bet protein, exonuclease gp6, single-stranded DNA-binding protein gp2.5, or a derivative or variant thereof.

2. The system of claim 1, further comprising a recruitment system comprising.
at least one aptamer sequence; and
an aptamer binding protein functionally linked to the microbial recombination protein as part of a fusion protein.

3. The system of claim 2, wherein the at least one aptamer sequence is an RNA aptamer sequence or a peptide aptamer sequence.

4. The system of claim 3, wherein the nucleic acid molecule comprises the at least one RNA aptamer sequence.

5. The system of claim 4, wherein the nucleic acid molecule comprises two RNA aptamer sequences.

6. The system of claim 5, wherein the two RNA aptamer sequences comprise the same sequence.

7. The system of any of claims 2-6, wherein the aptamer binding protein comprises a MS2 coat protein, or a functional derivative or variant thereof.

8. The system of any of claims 2-6, wherein the aptamer binding protein comprises phage N peptide, or a functional derivative or variant thereof.

9. The system of claim 3, wherein the at least one peptide aptamer sequence is conjugated to the Cas protein.

10. The system of claim 9, wherein the at least one peptide aptamer sequence comprises between 1 and 24 peptide aptamer sequences.

11. The system of claim 9 or 10, wherein the aptamer sequences comprise the same sequence.

12. The system of any of claim 2-3 or 9-11, wherein the aptamer sequence comprises a GCN4 peptide sequence.

13. The system of any of claims 2-12, wherein the microbial recombination protein N-terminus is linked to the aptamer binding protein C-terminus.

14. The system of any of claims 2-13, wherein the fusion protein further comprises a linker between the microbial recombination protein and the aptamer binding protein.

15. The system of claim 14, wherein the linker comprises the amino acid sequence of SEQ ID NO: 15.

16. The system of any of claims 2-15, wherein the fusion protein further comprises a nuclear localization sequence.

17. The system of claim 16, wherein the nuclear localization sequence comprises the amino acid sequence of SEQ ID NO: 16.

18. The system of claim 16 or claim 17, wherein the nuclear localization sequence is on the microbial recombination protein C-terminus.

19. The system of any of claims 1-18, wherein the RecE or RecT recombination protein is derived from E. coli.

20. The system of any of claims 1-19, wherein the microbial recombination protein comprises RecE, or derivative or variant thereof.

21. The system of any of claims 1-20, wherein the RecE, or derivative or variant thereof, comprises an amino acid sequence with at least 70% similarity to amino acid sequences selected from the group consisting of SEQ ID NOs: 1-8.

22. The system of any of claims 1-21, wherein the RecE, or derivative or variant thereof, comprises an amino acid sequence with at least 70% similarity to amino acid sequences selected from the group consisting of SEQ ID NOs: 1-3.

23. The system of any of claims 1-19, wherein the fusion protein comprises RecT, or derivative or variant thereof.

24. The system of any of claim 1-19 or 23, wherein the RecT, or derivative or variant thereof, comprises an amino acid sequence with at least 70% similarity to amino acid sequences selected from the group consisting of SEQ ID NOs: 9-14.

25. The system of any of claim 1-19 or 23-24, wherein the RecT, or derivative or variant thereof, comprises an amino acid sequence with at least 70% similarity to amino acid sequences selected from the group consisting of SEQ ID NO: 9.

26. The system of any of claims 1-25, wherein the Cas protein is catalytically dead.

27. The system of any of claims 1-26, wherein the Cas protein is Cas9 or Cas12a.

28. The system of any of claim 27, wherein the Cas9 protein is wild-type Streptococcus pyogenes Cas9 or a wild-type Staphylococcus aureus Cas9.

29. The system of any of claims 27-28, wherein the Cas9 protein is a Cas9 nickase.

30. The system of claim 29, wherein the Cas9 nickase is wild-type Streptococcus pyogenes Cas9 with an amino acid substation at position 10 of D10A.

31. The system of any of claims 1-30, further comprising donor nucleic acid.

32. The system of any of claims 1-31, wherein the target DNA sequence is a genomic DNA sequence in a host cell.

33. A composition comprising:
a polynucleotide comprising a nucleic acid sequence encoding a fusion protein comprising a microbial recombination protein functionally linked to an aptamer binding protein,
wherein the microbial recombination protein is RecE, RecT, lambda exonuclease, Bet protein, exonuclease gp6, single-stranded DNA-binding protein gp2.5, or a derivative or variant thereof.

34. The composition of claim 33, further comprising at least one of:
a polynucleotide comprising a nucleic acid sequence encoding a Cas protein; and
a nucleic acid molecule comprising a guide RNA sequence that is complementary to a target DNA sequence.

35. The composition of claim 34, wherein the nucleic acid molecule further comprises at least one RNA aptamer sequence.

36. The composition of claim 34, wherein the polynucleotide comprising a nucleic acid sequence encoding a Cas protein further comprises a sequence encoding at least one peptide aptamer sequence.

37. A vector comprising a polynucleotide comprising a nucleic acid sequence encoding a fusion protein comprising a microbial recombination protein functionally linked to an aptamer binding protein,
wherein the microbial recombination protein is RecE, RecT, lambda exonuclease, Bet protein, exonuclease gp6, single-stranded DNA-binding protein gp2.5, or a derivative or variant thereof.

38. The vector of claim 37, further comprising at least one of:
a polynucleotide comprising a nucleic acid sequence encoding a Cas protein; and
a nucleic acid molecule comprising a guide RNA sequence that is complementary to a target DNA sequence.

39. The vector of claim 38, wherein the nucleic acid molecule further comprises at least one RNA aptamer sequence.

40. The vector of claim 38, wherein the polynucleotide comprising a nucleic acid sequence encoding a Cas protein further comprises a sequence encoding at least one peptide aptamer sequence.

41. A eukaryotic cell comprising the system of any one of claims 1-32, the composition of any one of claims 33-36, or the vector of any of claims 37-40.

42. A method of altering a target genomic DNA sequence in a cell, comprising introducing the system of any one of claims 1-32, the composition of any one of claims 33-36, or the vector of any one of claims 37-40 into a cell comprising a target genomic DNA sequence.

43. The method of claim 42, wherein the cell is a mammalian cell.

44. The method of claim 42 or claim 43, wherein the cell is a human cell.

45. The method of any one of claims 42-44, wherein the cell is a stem cell.

46. The method of any one of claims 42-45, wherein the target genomic DNA sequence encodes a gene product.

47. The method of any one of claims 42-46, wherein the introducing into a cell comprises administering to a subject.

48. The method of claim 47, wherein the subject is a human.

49. The method of claim 47 or 48, wherein the administering comprises in vivo administration.

50. The method of claim 47 or 48, wherein the administering comprises transplantation of ex vivo treated cells comprising the system, composition, or vector.

51. Use of the system of any one of claims 1-32, the composition of any one of claims 33-36, or the vector of any one of claims 37-40 for the alteration of a target DNA sequence in a cell.
